(19) United States
(12) Patent Application Publication
(10) Pub. No.: US 2025/0170259 A1
(43) Pub. Date: May 29, 2025

(54) COMPOSITIONS AND METHODS FOR DELIVERING THERAPEUTIC POLYNUCLEOTIDES FOR EXON SKIPPING

(71) Applicants: Yale University, New Haven, CT (US); Gennao Bio, Inc., Pennington, NJ (US)

(72) Inventors: Elias Quijano, Durham, CT (US); Peter Glazer, Guilford, CT (US); Stephen Squinto, New York, NY (US); Dale Ludwig, Rockaway, NJ (US); Bruce Turner, Bryn Mawr, PA (US); Josage Dinithi Perera, New Haven, CT (US)

(21) Appl. No.: 18/841,356

(22) PCT Filed: Mar. 3, 2023
(86) PCT No.: PCT/US2023/063708

§ 371 Date: Aug. 23, 2024

Related U.S. Application Data
(60) Provisional application No. 63/316,339, filed on Mar. 3, 2022.

(51) Int. Cl. A61K 47/68; A61P 21/00; C07K 16/44; C12N 15/113

(52) U.S. Cl. CPC A61K 47/6807; A61K 47/6843; A61P 21/00; C07K 16/44; C12N 15/113

(57) ABSTRACT

Compositions and methods are provided for delivering an antisense oligonucleotide amendable to exon skipping by administering a complex formed between an antisense oligonucleotide and a 3E10 antibody or antigen-binding fragment thereof. In some instances, the complexes are stabilized and demonstrate cellular localization through a molar ratio of 3E10 antibody or antigen-binding fragment thereof to antisense oligonucleotide of at least about 2:1.

### CROSS-REFERENCE TO RELATED APPLICATION

This claims priority under 35 U.S.C. § 119 (e) to U.S. Provisional Application No. 63/316,339 filed Mar. 3, 2022, the disclosure of which is herein incorporated by reference in its entirety.

### TECHNICAL FIELD

The present disclosure relates generally to compositions and methods for delivering therapeutic polynucleotides, e.g., antisense oligonucleotides, particularly amendable for exon skipping in the human dystrophin gene, or other disease-causing genes, and pharmaceutical compositions thereof.

### BACKGROUND

Several diseases result from genetic mutations that disrupt the reading frame of an encoded polypeptide (e.g., nonsense mutations) resulting in expression of truncated polypeptides or, in some cases, no polypeptide at all. Other diseases result from genetic mutations that introduce new splice sites into a pre-mRNA or activate cryptic splice sites present in the gene, resulting in aberrant splicing and translation of dysfunctional polypeptides. Exon skipping therapies can be used to treat such diseases. Exon skipping methodologies generally use antisense oligonucleotides (ASO) that bind splice sites in pre-mRNA for exon containing a deleterious mutation or bind directly to cryptic splice sites, inducing the splicing machinery to skip over the effective exon or cryptic splice site and generate a mature mRNA that lacks the affected exon or to ignore the cryptic splice site and generate a full-length mature mRNA. Several antisense oligonucleotides currently undergoing clinical trials for conditions such as spinal muscular atrophy (SMA) and Duchenne muscular dystrophy (DMD), where antisense-mediated exon skipping can restore the open reading frame and allow the synthesis of partly or wholly functional proteins instead of non-functional ones.

Duchenne muscular dystrophy, for example, is caused by the absence of dystrophin protein due to mutations in the dystrophin (DMD) gene. The gene encoding the protein contains 79 exons spread out over more than 2 million nucleotides of DNA. Mutations disrupting the reading frame of the protein cause truncation of the translated dystrophin polypeptide, resulting in Duchenne muscular dystrophy. In September 2016, the US Food and Drug Administration (FDA) conditionally approved the first DMD antisense drug, eteplirsen (Exondys 51, SEQ ID NO: 208), which was developed to exclude exon 51 in mature DMD mRNA in patients with deleterious mutations in exon 51. Eteplirsen is an antisense oligonucleotide modified with a phosphorodiamidate morpholino oligomer (morpholino or PMO), an antisense chemistry that has been well-established in terms of its safety and effectiveness.

However, the effectiveness of polynucleotide-based therapies remains questionable. One possible explanation for the poor efficacy of many nucleic acid-based therapies, including exon-skipping therapies, is poor delivery of the therapeutic nucleic acid into the affected tissues. For instance, naked polynucleotides are readily degraded by a host of extracellular nucleases present in human tissues. Further, naked polynucleotides do not readily cross the cell membrane. Conventional approaches to overcoming these obstacles include packaging therapeutic polynucleotides into liposomal-based delivery vehicles or recombinant viral particles. However, these approaches present immunological challenges, because the viral capsids and liposomal vehicles are recognized by the host's immune system. These approaches may have other limitations, as well.

### SUMMARY

Given the background above, improved methods are needed for delivering therapeutic antisense oligonucleotides in vivo which are amendable to exon skipping. Advantageously, the present disclosure provides compositions and methods for delivering antisense oligonucleotides in vivo that are not reliant upon liposomal or viral vector based nucleic acid delivery.

In some embodiments, the advantageous properties of the compositions and methods described herein are based, at least in part, on the discovery that 3E10 antibodies or variants thereof, or antigen-binding fragments thereof, as described below, localize to several tissues, including skeletal, cardiac, and smooth muscle tissue in vivo following systemic or intramuscular administration. For instance, as described in Example 3 and illustrated in FIG. 10, following intravenous administration, both parental 3E10 antibody and 3E10 (D31N) variant antibody accumulated in skeletal muscle. Advantageously, in some embodiments, this tropism for muscle tissues is exploited in the compositions and methods described herein to deliver antisense oligonucleotides to skeletal muscle tissue for treatment of various skeletal muscle disorders.

In some embodiments, the advantageous properties of the compositions and methods described herein are based, at least in part, on the discovery that 3E10 antibodies and variants thereof, and antigen-binding fragments thereof, protect nucleic acids from degradation. For instance, as described in Example 6 and illustrated in FIGS. 13A and 13B, parental 3E10 and 3E10 (D31N) variant antibodies protected mRNA from RNAse A-mediated RNA degradation at molar ratios of 2:1 and 20:1. Advantageously, the polynucleotide protection is exploited in the compositions and methods described herein to improve the pharmacokinetic properties of therapeutic compositions delivering therapeutic polynucleotides in vivo.

In some embodiments, the advantageous properties of the compositions and methods described herein are based, at least in part, on the discovery of cellular uptake of a 3E10-Peptide Nucleic Acid (PNA) complex. For instance, as describe in Example 7 and illustrated in FIG. 14, complexing 3E10 with PNA significantly enhanced PNA delivery.

In some embodiments, the advantageous properties of the compositions and methods described herein are based, at least in part, on the discovery of exon 23 skipping in the DMD gene in myoblasts after administration of 3E10 (D31N): PNA complexes. For instance, as described in Example 9 and illustrated in FIG. 17A-17B, exon 23 skipping was observed for all 3E10 (D31N): PNA ratios of 1:1, 2:1, 3:1, 4:1, 5:1, and 10:1, respectively. Moreover, as described in Example 10 and illustrated by FIG. 18A-18B, exon skipping was observed for locked nucleic acid (LNA)-modified, 2′-O-methylated (2′OMe)-modified, as well as 2′-fluoro (2′F)-modified oligonucleotides: D31N complexes at ratios ranging from 1:1 to 5:1.

Accordingly, one aspect of the present disclosure provides a pharmaceutical composition including a therapeutically effective amount of a complex formed between an antisense oligonucleotide, and a 3E10 antibody or antigen-binding fragment thereof.

In another aspect, the disclosure provides a method for delivering an antisense oligonucleotide to a tissue of a subject in vivo, the method including parenterally administering a pharmaceutical composition, as described herein, to the subject. In some embodiments, the antisense oligonucleotide is for treating a disease or disorder including, but not limited to a skeletal muscle disorder, a neurogenetic disease, a cardiovascular disease, a metabolic disease, or a lung disorder for which a known disease-causing mutation.

In some embodiments of the methods and compositions described herein, the 3E10 antibody or antigen-binding fragment thereof includes (a) a light chain variable region (VL) complementarity determining region (CDR) 1 comprising the amino acid sequence of 3E10-VL-CDR1 (SEQ ID NO: 9), (b) a VL CDR2 comprising the amino acid sequence of 3E10-VL-CDR2 (SEQ ID NO: 10), (c) a VL CDR3 comprising the amino acid sequence of 3E10-VL-CDR3 (SEQ ID NO: 11), (d) a heavy chain variable region (VH) CDR1 comprising the amino acid sequence of 3E10-VH-CDR1a (SEQ ID NO: 16), (e) a VH CDR2 comprising the amino acid sequence of 3E10-VH-CDR2 (SEQ ID NO: 4), and (f) a VH CDR3 comprising the amino acid sequence of 3E10-VH-CDR3 (SEQ ID NO: 5).

In some embodiments of the methods and compositions described herein, the 3E10 antibody or antigen-binding fragment thereof includes (a) a light chain variable region (VL) complementarity determining region (CDR) 1 comprising an amino acid sequence having no more than two amino acid substitutions relative to 3E10-VL-CDR1 (SEQ ID NO: 9), (b) a VL CDR2 comprising an amino acid sequence having no more than two amino acid substitutions relative to 3E10-VL-CDR2 (SEQ ID NO: 10), (c) a VL CDR3 comprising an amino acid sequence having no more than two amino acid substitutions relative to 3E10-VL-CDR3 (SEQ ID NO: 11), (d) a heavy chain variable region (VH) CDR1 comprising an amino acid sequence having no more than two amino acid substitutions relative to 3E10-VH-CDR1a (SEQ ID NO: 3), (e) a VH CDR2 comprising an amino acid sequence having no more than two amino acid substitutions relative to 3E10-VH-CDR2 (SEQ ID NO: 4), and (f) a VH CDR3 comprising an amino acid sequence having no more than two amino acid substitutions relative to 3E10-VH-CDR3 (SEQ ID NO: 5).

In some embodiments of the methods and compositions described herein, the 3E10 antibody or antigen-binding fragment thereof includes (a) a light chain variable region (VL) complementarity determining region (CDR) 1 comprising the amino acid sequence of 3E10-VL-CDR1m (SEQ ID NO: 61), (b) a VL CDR2 comprising the amino acid sequence of 3E10-VL-CDR2m (SEQ ID NO: 62), (c) a VL CDR3 comprising the amino acid sequence of 3E10-VL-CDR3m (SEQ ID NO: 63), (d) a heavy chain variable region (VH) CDR1 comprising the amino acid sequence of 3E10-VH-CDR1m (SEQ ID NO: 58), (e) a VH CDR2 comprising the amino acid sequence of 3E10-VH-CDR2m (SEQ ID NO: 59), and (f) a VH CDR3 comprising the amino acid sequence of 3E10-VH-CDR3m (SEQ ID NO: 60).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates amino acid sequences for the parent 3E10 monoclonal antibody.

FIGS. 2A, 2B, and 2C illustrate amino acid sequences for the D31N variant (FIG. 2A), other CDR variants (FIG. 2B), and additionally contemplated CDR variants (FIG. 2C) of the 3E10 monoclonal antibody, in accordance with some embodiments of the present disclosure.

FIG. 3 illustrates example charge-conserved CDR variants of the 3E10 monoclonal antibody, in accordance with various embodiments of the present disclosure.

FIG. 4 illustrates example CDR variants containing a combination of amino acid substitutions, charged-conserved amino acid substitutions, and rationally-designed amino acid substitutions of the 3E10 monoclonal antibody, in accordance with various embodiments of the present disclosure.

FIG. 5 illustrates a sequence alignment of examples of humanized 3E10 heavy chain variable regions, with CDRs underlined as indicated.

FIG. 6 illustrates a sequence alignment of examples of humanized 3E10 light chain variable regions, with CDRs and putative nuclear localization signals (NLS) underlined as indicated.

FIGS. 7A, 7B, 7C, 7D, and 7E collectively illustrate a sequence alignment of example of humanized di-scFv constructs of the 3E10 monoclonal antibody.

FIG. 8 illustrates a line graph showing 3E10-mediated delivery of mRNA (bioluminescene (Photons/second)) to mouse muscles (IM) over time (days post-IM injection), in accordance with some embodiments of the present disclosure.

FIGS. 9A, 9B, and 9C collectively show fluorescently-labeled 3E10 (D31N) antibody localization in mouse skeletal muscle following intravenous administration. FIGS. 9A and 9B are images of fluorescence in mouse skeletal muscle following intravenous injections of a control composition (FIG. 9A) or fluorescently-labeled 3E10 (D31N) antibody (FIG. 9B), acquired by IVIS (Perkin Elmer) 24 hours after administration. FIG. 9C is a bar graph quantifying the fluorescence in the IVIS images.

FIG. 10 is a bar graph quantifying the fluorescence in IVIS images of dose-dependent biodistribution of 3E10-D31N to tissues 24 hours following 100 μg or 200 μg intravenous injection of 3E10-D31N labeled with VivoTag680 into mice (Perkin Elmer).

FIGS. 11A and 11B illustrate electrostatic surface potential renderings of a molecular model of a 3E10-scFv construct, revealing a putative Nucleic Acid Binding pocket (NAB1). FIG. 11A additionally shows predicted structural and electrostatic potential changes induced by amino acid substitutions at residue HC CDR1 residue 31. FIG. 11B is an illustration of molecular modeling of 3E10-scFv (Pymol) with NAB1 amino acid residues highlighted by punctate dots.

FIG. 11C illustrates mapping of the putative nucleic acid binding pocket, as identified by the molecular modeling shown in FIGS. 11A and 11B, onto the amino acid sequence of the 3E10-scFv construct.

FIGS. 12A and 12B show expression of mRNA in skeletal muscle following intramuscular administration of a 3E10 (D31N)-mRNA construct. FIG. 12A show fluorescent images of a mouse over a five-day time course following intramuscular administration of mRNA encoding a luciferase complexed with 3E10 (D31N). FIG. 12B illustrates a bar graph quantifying average radiance over all pixels, showing fluorescence in single mice in images of control mice (untreated) and mice administered the 3E10 (D31N)-mRNA construct intramuscularly.

FIGS. 13A and 13B show gel electrophoresis analysis of mRNA protection assays performed with 3E10 (D31N)-mRNA constructs prepared at 20:1 (FIG. 13A) and 2:1 (FIG. 13B) molar ratios.

FIG. 14 illustrate results of cellular uptake of a 5-TAMRA-labeled Peptide Nucleic WT-3E10: PNA complex.

FIG. 15 illustrates nucleotide sequences for the oligomers designed to cause skipping of exon 23 in dystrophin (DMD) RNA. K denotes lysine residues on PNA; superscript O denotes 2′OMe modifications; superscript F denotes 2′fluoro modifications; superscript L denotes locked nucleic acid (LNA) modifications.

FIGS. 16A and 16B illustrate RT-PCR results of exon 23 skipping after incubation (48 and 72 hrs) of a WT-3E10: PNA complex with murine myoblast cells.

FIGS. 17A and 17B illustrate RT-PCR results of exon 23 skipping after incubation (48 and 72 hrs) of a 3E10 (D31N): PNA complex with murine myoblast cells.

FIGS. 18A and 18B illustrate RT-PCR results of exon 23 skipping after 48 h incubation of A) negatively charged LNA/2′OMe-PS or LNA/2′F-PS oligomers with D31N-3E10; and B) negatively charged LNA-PS or 2′OMe-PS oligomers with D31N-3E10.

FIG. 19 illustrates amino acid sequences of humanized 3E10 variable heavy (3E10-VH) domains, in accordance with various embodiments of the present disclosure.

FIG. 20 illustrates amino acid sequences of mature humanized 3E10 heavy chains (3E10-HC), lacking a signal peptide, in accordance with various embodiments of the present disclosure.

FIG. 21 illustrates amino acid sequences of humanized 3E10 heavy chains (3E10-HC), in accordance with various embodiments of the present disclosure.

FIG. 22 illustrates amino acid sequences of humanized 3E10 variable light (3E10-VL) domains, in accordance with various embodiments of the present disclosure.

FIG. 23 illustrates amino acid sequences of mature humanized 3E10 light chains (3E10-LC), lacking a signal peptide, in accordance with various embodiments of the present disclosure.

FIG. 24 illustrates amino acid sequences of humanized 3E10 light chains (3E10-LC), in accordance with various embodiments of the present disclosure.

### DETAILED DESCRIPTION

The present disclosure provides compositions and methods for delivering therapeutic polynucleotides, e.g., antisense oligonucleotides, that are amendable to exon skipping in vivo that are not reliant upon the conventional viral-based or liposomal-based delivery methodologies associated with difficult and costly production, limited packaging capacity, and adverse immunological events. In some aspects, described in greater detail below, these compositions and methods are based on the binding between antisense oligonucleotides and an 3E10 antibody or antigen-binding fragment thereof, thus increasing the in vivo effectiveness of these complexes. In some aspects, the binding between antisense oligonucleotides and an 3E10 antibody or antigen-binding fragment thereof form complexes which can be non-covalently or covalently associated with one another.

### Definitions

The terminology used in the present disclosure is for the purpose of describing particular embodiments only and is not intended to be limiting of the invention. As used in the description of the invention and the attached claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. Unless the context requires otherwise, it will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%.

By “antigen binding domain” or “ABD” herein is meant a set of six Complementary Determining Regions (CDRs) that, when present as part of a polypeptide sequence or sequences, specifically binds a target antigen as discussed herein. Thus, a “nucleic acid binding domain” binds a nucleic acid antigen as outlined herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. Thus, in some cases, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of the hinge in the second monomer.

For all positions discussed in the present disclosure that relate to antibodies, unless otherwise noted, amino acid position numbering is according to the EU index. The EU index or EU index as in Kabat or EU numbering scheme refers to the numbering of the EU antibody. Kabat et al. collected numerous primary sequences of the variable regions of heavy chains and light chains. Based on the degree of conservation of the sequences, they classified individual primary sequences into the CDR and the framework and made a list thereof. See, SEQUENCES OF IMMUNOLOGICAL INTEREST, 5th edition, NIH publication, No. 91-3242, E. A. Kabat et al.; Edelman et al., 1969, Proc Natl Acad Sci USA 63:78-85, the contents of which are incorporated herein by reference. The modification can be an addition, deletion, or substitution.

By “target antigen” as used herein is meant the molecule that is bound specifically by the antigen binding domain comprising the variable regions of a given antibody. As discussed below, in the present case the target antigens are nucleic acids.

As described below, in some embodiments a parent polypeptide, for example an Fc parent polypeptide, is a human wild type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 75% identity with a parent protein sequence, or at least about 80% identity with a parent protein sequence, and most preferably at least about 90% identity, more preferably at least about 95%, or at least about 98%, or at least about 99% sequence identity. In some embodiments, the protein variant sequence herein has at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity with a parent protein sequence. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2, IgG3, or IgG4.

By “isotype” as used herein is meant any of the subclasses of immunoglobulins defined by the chemical and antigenic characteristics of their constant regions. It should be understood that therapeutic antibodies can also comprise hybrids of isotypes and/or subclasses.

By “Fab” or “Fab region” as used herein is meant a polypeptide that comprises the VH, CH1, VL, and CL immunoglobulin domains, generally on two different polypeptide chains (e.g. VH-CH1 on one chain and VL-CL on the other). Fab may refer to this region in isolation, or this region in the context of an antibody of the disclosure. In the context of a Fab, the Fab comprises an Fv region in addition to the CH1 and CL domains.

By “Fv” or “Fv fragment” or “Fv region” as used herein is meant a polypeptide that comprises the VL and VH domains of an ABD. Fv regions can be formatted as both Fabs (as discussed above, generally two different polypeptides that also include the constant regions as outlined above) and scFvs, where the vl and vh domains are combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g. H.X_L. Y means N- to C-terminal is vh-linker-vl, and L. Y_H.X is vl-linker-vh.

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus, the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.) Human IgG Fc domains are of particular use in the present disclosure, and can be the Fc domain from human IgG1, IgG2 or IgG4.

A “variant Fc domain” contains amino acid modifications as compared to a parental Fc domain. Thus, a “variant human IgG1 Fc domain” is one that contains amino acid modifications (generally amino acid substitutions, although in the case of ablation variants, amino acid deletions are included) as compared to the human IgG1 Fc domain. In general, variant Fc domains have at least about 80, about 85, about 90, about 95, about 97, about 98 or about 99 percent identity to the corresponding parental human IgG Fc domain (using the identity algorithms discussed below, with one embodiment utilizing the BLAST algorithm as is known in the art, using default parameters). Alternatively, the variant Fc domains can have from 1 to about 20 (e.g., 1, 2, 3, 4, 5, 6, 7, 8, 9, 10, 11, 12, 13, 14, 15, 16, 17, 18, 19 or 20) amino acid modifications as compared to the parental Fc domain. Additionally, as discussed herein, the variant Fc domains herein still retain the ability to form a dimer with another Fc domain as measured using known techniques as described herein, such as non-denaturing gel electrophoresis.

By “heavy chain constant region” herein is meant the CH1-hinge-CH2-CH3 portion of an antibody (or fragments thereof), excluding the variable heavy domain; in EU numbering of human IgG1 this is amino acids 118-447 By “heavy chain constant region fragment” herein is meant a heavy chain constant region that contains fewer amino acids from either or both of the N- and C-termini but still retains the ability to form a dimer with another heavy chain constant region.

By “variable region” or “variable domain” as used herein is meant the region of an immunoglobulin that comprises one or more Ig domains substantially encoded by any of the VK, V2, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

The antibodies and antigen-binding fragments thereof of the disclosure are recombinant antibodies that have been engineered to have the various properties described herein and are generally isolated prior to use. As used herein, the term “isolated”, when used to describe the various polypeptides described herein, refers to a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogenous host cells, and they can be isolated as well.

As used herein, a “3E10 antibody” refers to an antibody with a set of heavy chain CDRs (VH CDR1, VH CDR2, and VH CDR3), identified according to the Kabat system, comprising amino acid sequences that vary from SEQ ID NOS: 58, 59, and 60 by no more than two amino acids each, respectively, a set of light chain CDRs (VL CDR1, VL CDR2, and VL CRD3) comprising amino acid sequences that vary from SEQ ID NOS: 61, 62, and 63 by no more than two amino acids each, respectively, and that binds nucleic acids. As described herein, the 3E10 antigen is a polynucleotide.

As used herein, the term “cell-penetrating” refers to an antibody or antigen binding fragment thereof that can penetrate a cell, e.g., a mammalian cell, without the aid of an exogeneous transport vehicle, such as a liposome, or a conjugated cell-penetrating peptide. With respect to 3E10 antibodies and antigen binding fragments thereof, the cell-penetrating antibody or antigen binding fragment thereof can penetrate a cell expressing an ENT2 receptor on its cell surface in the presence of nucleic acids, e.g., non-covalently bound and/or conjugated to the 3E10 antibody or antigen binding fragment thereof, resulting in internalization of the 3E10 antibodies and antigen binding fragments thereof. In some embodiments, the cell-penetrating 3E10 antibody or antigen binding fragment thereof is conjugated to a functional molecule, e.g., a chemical agent, polynucleotide, or polypeptide.

By “modification” herein is meant an amino acid substitution, insertion, and/or deletion in a polypeptide sequence.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the parent protein, using the alignment programs described below, such as BLAST. Although amino acid sequence modifications to a 3E10 antibody or antigen binding fragment thereof as described herein may produce a protein and/or polypeptide that is referred to as a variant 3E10 antibody or antigen binding fragment thereof, such variants still fall within the classification of a 3E10 antibody or antigen binding fragment thereof as long as they maintain the CDR sequence and cell penetrating activity requirements of a 3E10 antibody or antigen binding fragment thereof.

Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. U.S.A. 85:2444 [search for similarity method]; or Altschul, S. F. et al, (1990) “Basic Local Alignment Search Tool,” J. Mol. Biol. 215:403-10, the “BLAST” algorithm, see the webpage located at URL blast.ncbi.nlm.nih.gov/Blast.cgi. When using any of the aforementioned algorithms, the default parameters (for Window length, gap penalty, etc.) are used. Unless specifically stated otherwise, sequence identity is determined using the BLAST algorithm, using default parameters.

By “enhance” or “enhancing,” or “increase” or “increasing,” or “stimulate” or “stimulating,” refers generally to the ability of one or more antisense oligomer conjugates or pharmaceutical compositions to produce or cause a greater physiological response (i.e., downstream effects) in a cell or a subject, as compared to the response caused by either no antisense oligomer conjugate or a control compound. A greater physiological response may include increased expression of a functional form of a dystrophin protein, or increased dystrophin-related biological activity in muscle tissue, among other responses apparent from the understanding in the art and the description herein. Increased muscle function can also be measured, including increases or improvements in muscle function by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. The percentage of muscle fibers that express a functional dystrophin can also be measured, including increased dystrophin expression in about 1%, 2%, 5%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of muscle fibers. For instance, it has been shown that around 40% of muscle function improvement can occur if 25-30% of fibers express dystrophin (see, e.g., DelloRusso et al, Proc Natl Acad Sci USA 99:12979-12984, 2002). An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times, including all integers and decimal points in between and above 1, e.g., 1.5, 1.6, 1.7, 1.8, etc.) the amount produced by no antisense oligomer conjugate (the absence of an agent) or a control compound.

As used herein, the terms “function” and “functional” and the like refer to a biological, enzymatic, or therapeutic function.

A “functional” dystrophin protein refers generally to a dystrophin protein having sufficient biological activity to reduce the progressive degradation of muscle tissue that is otherwise characteristic of muscular dystrophy, typically as compared to the altered or “defective” form of dystrophin protein that is present in certain subjects with Duchenne muscular dystrophy (DMD) or Becker muscular dystrophy (BMD). In certain embodiments, a functional dystrophin protein may have about 10%, 20%, 30%, 40%, 50%, 60%, 70%, 80%, 90%, or 100% (including all integers in between) of the in vitro or in vivo biological activity of wild-type dystrophin, as measured according to routine techniques in the art. As one example, dystrophin-related activity in muscle cultures in vitro can be measured according to myotube size, myofibril organization (or disorganization), contractile activity, and spontaneous clustering of acetylcholine receptors (see, e.g., Brown et al., Journal of Cell Science. 112:209-216, 1999). Animal models are also valuable resources for studying the pathogenesis of disease, and provide a means to test dystrophin-related activity. Two of the most widely used animal models for DMD research are the mdx mouse and the golden retriever muscular dystrophy (GRMD) dog, both of which are dystrophin negative (see, e.g., Collins & Morgan, Int J Exp Pathol 84:165-172, 2003). These and other animal models can be used to measure the functional activity of various dystrophin proteins. Included are truncated forms of dystrophin, such as those forms that are produced following the administration of certain of the exon-skipping antisense oligonucleotides of the present disclosure.

The term “oligonucleotide” as used herein refers to a sequence of subunits connected by intersubunit linkages. In certain instances, the term “oligonucleotide” is used in reference to an “antisense oligonucleotide.” For “antisense oligonucleotides,” each subunit consists of: (i) a ribose sugar or a derivative thereof; and (ii) a nucleobase bound thereto, such that the order of the base-pairing moieties forms a base sequence that is complementary to a target sequence in a nucleic acid (typically an RNA) by Watson-Crick base pairing, to form a nucleic acid: oligomer heteroduplex within the target sequence with the proviso that either the subunit, the intersubunit linkage, or both are not naturally occurring. In certain embodiments, the antisense oligonucleotide is a phosphorodiamidate morpholino oligomer (PMO). In other embodiments, the antisense oligonucleotide is a 2′-O-methyl phosphorothioate (2′OMe-PS). In other embodiments, the antisense oligonucleotide is a 2′-fluoro phosphorothioate (2′F-PS). In other embodiments, the antisense oligomer of the disclosure is a peptide nucleic acid (PNA), a locked nucleic acid (LNA), or a bridged nucleic acid (BNA) such as 2′-O,4′-C-ethylene-bridged nucleic acid (ENA).

Morpholinos as described herein include all stereoisomers and tautomers of the foregoing general structure. The synthesis, structures, and binding characteristics of morpholino oligomers are detailed in U.S. Pat. Nos. 5,698,685; 5,217,866; 5,142,047; 5,034,506; 5,166,315; 5,521,063; 5,506,337; 8,076,476; and 8,299,206; all of which are incorporated herein by reference.

The terms “complementary” and “complementarity” refer to two or more oligomers (i.e., each comprising a nucleobase sequence) that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given nucleobase sequence are matched to the other nucleobase sequence according to base pairing rules. For example, in some embodiments, complementarity between a given nucleobase sequence and the other nucleobase sequence may be about 70%, about 75%, about 80%, about 85%, about 90% or about 95%. Or, there may be “complete” or “perfect” (100%) complementarity between a given nucleobase sequence and the other nucleobase sequence to continue the example. The degree of complementarity between nucleobase sequences has significant effects on the efficiency and strength of hybridization between the sequences.

The terms “nucleobase” (Nu), “base pairing moiety” or “base” are used interchangeably to refer to a purine or pyrimidine base found in naturally occurring, or “native” DNA or RNA (e.g., uracil, thymine, adenine, cytosine, and guanine), as well as analogs of these naturally occurring purines and pyrimidines. These analogs may confer improved properties, such as binding affinity, to the oligomer. Exemplary analogs include hypoxanthine (the base component of inosine); 2,6-diaminopurine; 5-methyl cytosine; C5-propynyl-modified pyrimidines; 10-(9-(aminoethoxy) phenoxazinyl) (G-clamp) and the like.

The terms “mismatch” or “mismatches” refer to one or more nucleobases (whether contiguous or separate) in an oligomer nucleobase sequence that are not matched to a target pre-mRNA according to base pairing rules. While perfect complementarity is often desired, some embodiments can include one or more but preferably 6, 5, 4, 3, 2, or 1 mismatches with respect to the target pre-mRNA. Variations at any location within the oligomer are included. In certain embodiments, antisense oligomer conjugates of the disclosure include variations in nucleobase sequence near the term variations in the interior, and if present are typically within about 6, 5, 4, 3, 2, or 1 subunits of the 5′ and/or 3′ terminus.

The terms “effective amount” and “therapeutically effective amount” are used interchangeably herein and refer to an amount of therapeutic compound, such as an antisense oligomer, administered to a mammalian subject, either as a single dose or as part of a series of doses, which is effective to produce a desired therapeutic effect. For an antisense oligomer, this effect is typically brought about by inhibiting translation or natural splice-processing of a selected target sequence, or producing a clinically meaningful amount of dystrophin (statistical significance).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The terms “subject” and “patient” as used herein include any animal that exhibits a symptom, or is at risk for exhibiting a symptom, which can be treated with an antisense oligomer conjugate of the disclosure, such as a subject (or patient) that has or is at risk for having DMD or BMD, or any of the symptoms associated with these conditions (e.g., muscle fiber loss). Also included are methods of producing dystrophin in a subject (or patient) having a mutation of the dystrophin gene that is amenable to exon 23 skipping.

The phrase “targeting sequence” refers to a sequence of nucleobases of an oligomer that is complementary to a sequence of nucleotides in a target pre-mRNA. For example, in some embodiments of the disclosure, the sequence of nucleotides in the target pre-mRNA is an exon 23 annealing site in the dystrophin pre-mRNA.

As used herein, the term “treatment” of a subject (e.g., a mammal, such as a human) or a cell is any type of intervention used in an attempt to alter the natural course of the subject or cell. Treatment includes, but is not limited to, administration of an oligomer or a pharmaceutical composition thereof, and may be performed either prophylactically or subsequent to the initiation of a pathologic event or contact with an etiologic agent. Treatment includes any desirable effect on the symptoms or pathology of a disease or condition associated with the dystrophin protein, as in certain forms of muscular dystrophy, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

### 3E10 Antibodies, Variants, and Fragments Thereof

In some aspects, the present disclosure relates to the use of 3E10 antibodies, and derivatives thereof, for delivering antisense oligonucleotides amendable for exon skipping in tissues of a subject, including but not limited to skeletal muscle tissues for treatment of genetic skeletal muscle disorders. As is discussed below, the term antibody is used generally. Antibodies that find use in the present disclosure take on a number of formats as described herein, including traditional antibodies as well as antibody derivatives, fragments, and mimetics, described herein in various embodiments.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present disclosure is directed to antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M).

The light chain generally comprises two domains, the variable light domain (containing the light chain CDRs and together with the variable heavy domains forming the Fv region), and a constant light chain region (often referred to as CL or Cκ). The heavy chain comprises a variable heavy domain and a constant domain, which includes a CH1-optional hinge-Fc domain comprising a CH2-CH3.

The hypervariable region of an antibody generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs useful for the compositions and methods described herein are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3). A useful comparison of CDR numbering is described in Lafranc et al., Dev. Comp. Immunol. 27 (1): 55-77 (2003).

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present disclosure provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as nucleic acids, amino acids, or sugar side chains and usually have specific structural characteristics, as well as specific charge characteristics. A single antigen may have more than one epitope. The antibodies described herein bind to nucleic acid epitopes in a partially sequence-independent manner. That is, while the antibodies described herein bind to some polynucleotide structures and sequences with greater affinity than other nucleic acid structures and sequences, they have some general affinity for polynucleotides.

The “Fc domain” of the heavy chain includes the -CH2-CH3 domain, and optionally a hinge domain (-H-CH2-CH3). For IgG, the Fc domain comprises immunoglobulin domains CH2 and CH3 (Cy2 and Cy3) and the lower hinge region between CH1 (Cy1) and CH2 (Cy2). Although the boundaries of the Fc region may vary, the human IgG heavy chain Fc region is usually defined to include residues C226 or P230 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Thus, the “Fc domain” includes the -CH2-CH3 domain, and optionally a hinge domain (hinge-CH2-CH3). In the embodiments herein, when a scFv is attached to an Fc domain, it is generally the C-terminus of the scFv construct that is attached to all or part of the hinge of the Fc domain; for example, it is generally attached to the sequence EPKS which is the beginning of the hinge. In some embodiments, as is more fully described below, amino acid modifications are made to the Fc region, for example to alter binding to one or more FcγR receptors or to the FcRn receptor, and to enable heterodimer formation and purification, as outlined herein.

Another part of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain.

A scFv comprises a variable heavy chain, an scFv linker, and a variable light domain. In most of the constructs and sequences outlined herein, the C-terminus of the variable heavy chain is attached to the N-terminus of the scFv linker, the C-terminus of which is attached to the N-terminus of a variable light chain (N-vh-linker-vl-C) although that can be switched (N-vl-linker-vh-C).

Thus, the present disclosure relates to different antibody domains. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

In certain embodiments, the antibodies of the disclosure comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence, e.g., that of the 3E10 antibody. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene.

In one embodiment, the parent antibody has been affinity matured, as is known in the art. Structure-based methods may be employed for humanization and affinity maturation, for example as described in U.S. patent Ser. No. 11/004,590, which is incorporated herein by reference. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272 (16): 10678-10684; Rosok et al., 1996, J. Biol. Chem. 271 (37): 22611-22618; Rader et al., 1998, Proc. Natl. Acad. Sci. USA 95:8910-8915; Krauss et al., 2003, Protein Engineering 16 (10): 753-759, all of which are incorporated herein by reference. Other humanization methods may involve the grafting of only parts of the CDRs, including but not limited to methods described in U.S. patent Ser. No. 09/810,510; Tan et al., 2002, J. Immunol. 169:1119-1125; De Pascalis et al., 2002, J. Immunol. 169:3076-3084, all of which are incorporated herein by reference.

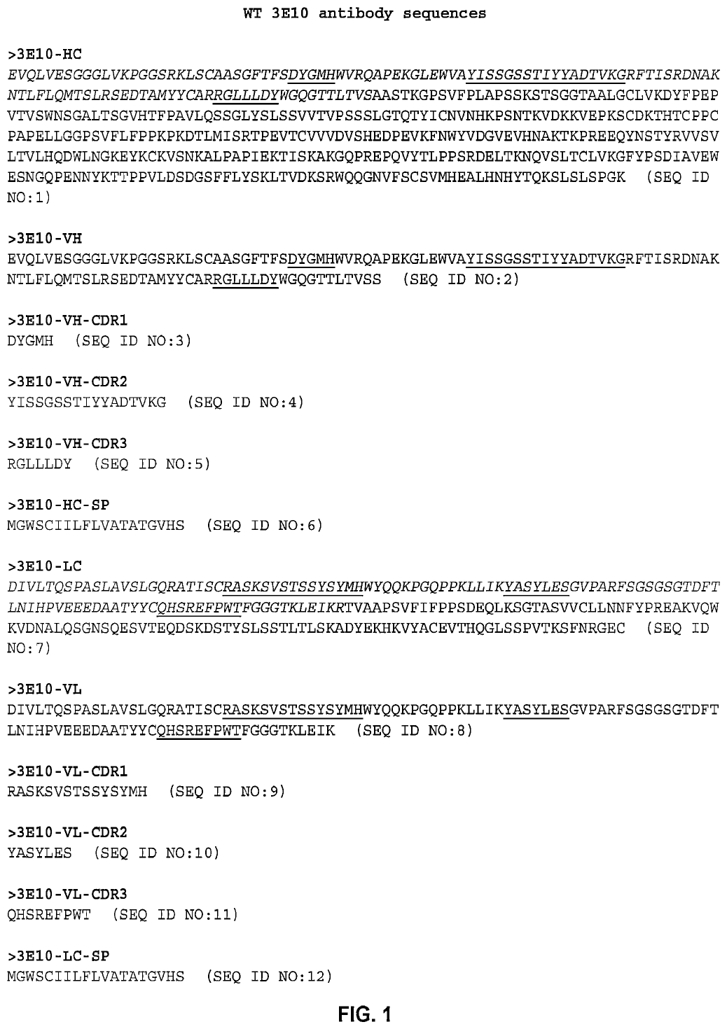

In some aspects, the disclosure relates to the use of antigen binding domains (ABDs) that bind to nucleic acids, and specifically that bind to therapeutic polynucleotides, derived from the 3E10 antibody. The amino acid sequence of the heavy and light chains of the parent 3E10 antibody are shown in FIG. 1. Accordingly, in some embodiments, the compositions described herein include a 3E10 antibody or antigen-binding fragment thereof.

In some embodiments, a 3E10 antibody or antigen-binding fragment thereof described herein includes CDR sequences corresponding to the parent 3E10 antibody, shown in FIG. 1. Accordingly, in some embodiments, the a 3E10 antibody or antigen-binding fragment thereof includes a light chain variable region (VL) complementarity determining region (CDR) 1 comprising the amino acid sequence of 3E10-VL-CDR1 (SEQ ID NO: 9), a VL CDR2 comprising the amino acid sequence of 3E10-VL-CDR2 (SEQ ID NO: 10), a VL CDR3 comprising the amino acid sequence of 3E10-VL-CDR3 (SEQ ID NO: 11), a heavy chain variable region (VH) CDR1 comprising the amino acid sequence of 3E10-VH-CDR1 (SEQ ID NO: 3), a VH CDR2 comprising the amino acid sequence of 3E10-VH-CDR2 (SEQ ID NO: 4), and a VH CDR3 comprising the amino acid sequence of 3E10-VH-CDR3 (SEQ ID NO: 5).

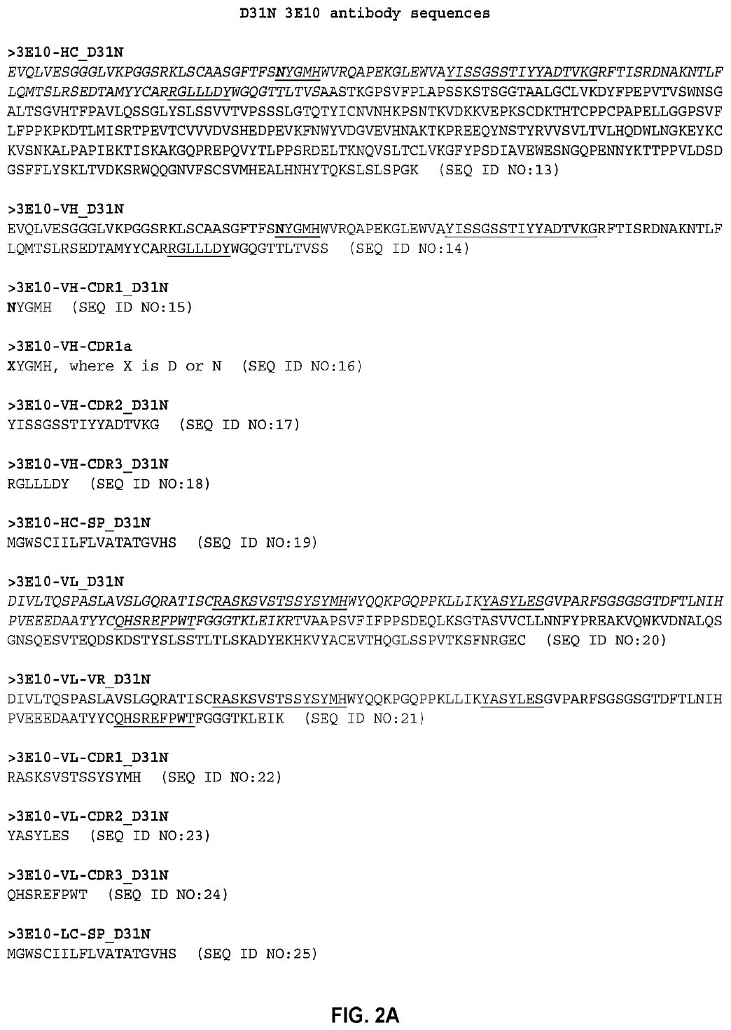

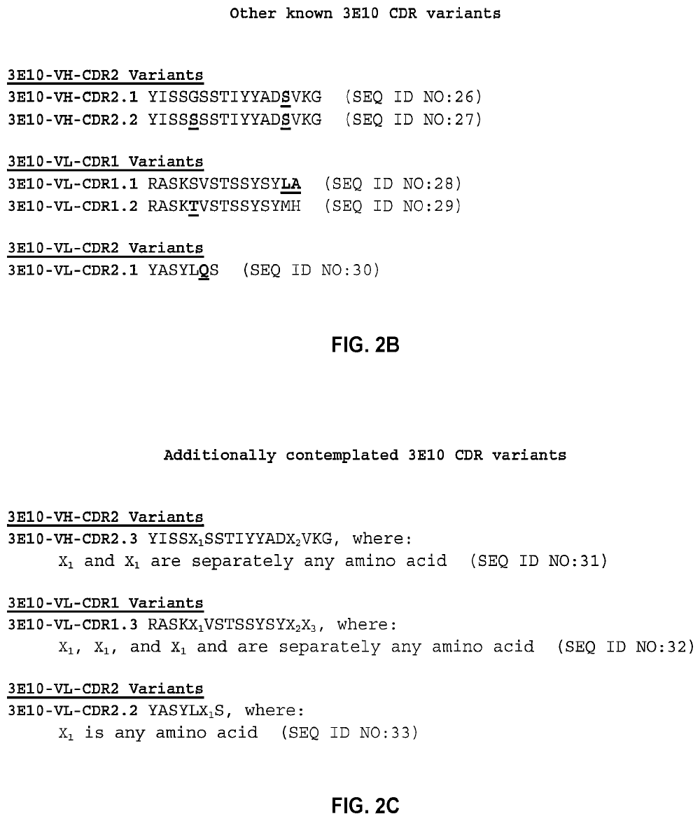

In some embodiments, a 3E10 antibody or antigen-binding fragment thereof described herein includes CDR sequences from a variant 3E10 antibody that includes a D31N amino acid substitution in the VH CDR1, as shown in FIG. 2. Accordingly, in some embodiments, the a 3E10 antibody or antigen-binding fragment thereof includes a light chain variable region (VL) complementarity determining region (CDR) 1 comprising the amino acid sequence of 3E10-VL-CDR1_D31N (SEQ ID NO: 22), a VL CDR2 comprising the amino acid sequence of 3E10-VL-CDR2_D31N (SEQ ID NO: 23), a VL CDR3 comprising the amino acid sequence of 3E10-VL-CDR3_D31N (SEQ ID NO: 24), a heavy chain variable region (VH) CDR1 comprising the amino acid sequence of 3E10-VH-CDR1_D31N (SEQ ID NO: 15), a VH CDR2 comprising the amino acid sequence of 3E10-VH-CDR2_D31N (SEQ ID NO: 17), and a VH CDR3 comprising the amino acid sequence of 3E10-VH-CDR3_D31N (SEQ ID NO: 18).

In some embodiments, a 3E10 antibody or antigen-binding fragment thereof described herein refers to CDR sequences corresponding to the parent 3E10 antibody, shown in FIG. 1, optionally including a D31N amino acid substitution in the VH CDR1. Accordingly, in some embodiments, a 3E10 antibody or antigen-binding fragment thereof includes a light chain variable region (VL) complementarity determining region (CDR) 1 comprising the amino acid sequence of 3E10-VL-CDR1 (SEQ ID NO: 9), a VL CDR2 comprising the amino acid sequence of 3E10-VL-CDR2 (SEQ ID NO: 10), a VL CDR3 comprising the amino acid sequence of 3E10-VL-CDR3 (SEQ ID NO: 11), a heavy chain variable region (VH) CDR1 comprising the amino acid sequence of 3E10-VH-CDR1a (SEQ ID NO: 16), a VH CDR2 comprising the amino acid sequence of 3E10-VH-CDR2 (SEQ ID NO: 4), and a VH CDR3 comprising the amino acid sequence of 3E10-VH-CDR3 (SEQ ID NO: 5).

In some embodiments, a 3E10 antibody or antigen-binding fragment thereof described herein includes CDR sequences corresponding to the parent 3E10 antibody, shown in FIG. 1, with a known amino acid substitution in one or more CDR. For example, FIG. 2B shows the amino acid sequence of several known VH CDR2, VL CDR1, and VL CDR2 amino acid sequences. Accordingly, in some embodiments, a 3E10 antibody or antigen-binding fragment thereof described herein includes one or more amino acid substitution, relative to the CDR sequences of the parent 3E10 (shown in FIG. 1) or 3E10-D31N variant (shown in FIG. 2), selected from a G to S substitution at position 5 of VH CDR2, a T to S substitution at position 14 of VH CDR2, an S to T substitution at position 5 of VL CDR1, an M to L substitution at position 14 of VL CDR1, an H to A substitution at position 15 of VL CDR1, and an E to Q substitution at position 6 of VL CDR2.

Accordingly, in some embodiments, a 3E10 antibody or antigen-binding fragment thereof includes VH CDR2 comprising the amino acid sequence of 3E10-VH-CDR2.1 (SEQ ID NO: 26) or 3E10-VH-CDR2.2 (SEQ ID NO: 27). In some embodiments, the 3E10 antibody or antigen-binding fragment thereof further includes VL CDRs 1-3, and VH CDRs 1 and 3 according to the parent 3E10 antibody (as shown in FIG. 1). In some embodiments, the 3E10 antibody or antigen-binding fragment thereof further includes VL CDRs 1-3, and VH CDRs 1 and 3 according to the 3E10-D31N variant (as shown in FIG. 2A). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1) or relative to the 3E10-D31N variant (as shown in FIG. 2A).

Similarly, in some embodiments, a 3E10 antibody or antigen-binding fragment thereof includes VL CDR1 comprising the amino acid sequence of 3E10-VL-CDR1.1 (SEQ ID NO: 28) or 3E10-VL-CDR1.2 (SEQ ID NO: 29). In some embodiments, the 3E10 antibody or antigen-binding fragment thereof further includes VL CDRs 2 and 3, and VH CDRs 1-3 according to the parent 3E10 antibody (as shown in FIG. 1). In some embodiments, the 3E10 antibody or antigen-binding fragment thereof further includes VL CDRs 2 and 3, and VH CDRs 1-3 according to the 3E10-D31N variant (as shown in FIG. 2A). In some embodiments, the 3E10 antibody or antigen-binding fragment thereof further includes VL CDRs 2 and 3, and VH CDRs 1-3 having one or more amino acid substitutions relative to the CDRs of the parent 3E10 antibody (as shown in FIG. 1) or relative to the 3E10-D31N variant (as shown in FIG. 2A).

Similarly, in some embodiments, a 3E10 antibody or antigen-binding fragment thereof includes VL CDR2 comprising the amino acid sequence of 3E10-VL-CDR2.1 (SEQ ID NO: 30). In some embodiments, the 3E10 antibody or antigen-binding fragment thereof further includes VL CDRs 1 and 3, and VH CDRs 1-3 according to the parent 3E10 antibody (as shown in FIG. 1). In some embodiments, the 3E10 antibody or antigen-binding fragment thereof further includes VL CDRs 1 and 3, and VH CDRs 1-3 according to the 3E10-D31N variant (as shown in FIG. 2A). In some embodiments, the 3E10 antibody or antigen-binding fragment thereof further includes VL CDRs 1 and 3, and VH CDRs 1-3 having one or more amino acid substitutions relative to the CDRs of the parent 3E10 antibody (as shown in FIG. 1) or relative to the 3E10-D31N variant (as shown in FIG. 2A).

While some of the amino acid substitutions described above are fairly conservative substitutions—e.g., an S to T substitution at position 5 of VL CDR1—other substitutions are to amino acids that have vastly different properties—e.g., an M to L substitution at position 14 of VL CDR1, an H to A substitution at position 15 of VL CDR1, and an E to Q substitution at position 6 of VL CDR2. This suggests, without being bound by theory, that at least these positions within the 3E10 CDR framework are tolerant to other amino acid substitutions.

Accordingly, in some embodiments, a 3E10 antibody or antigen-binding fragment thereof includes VH CDR2 comprising the amino acid sequence of 3E10-VH-CDR2.3 (SEQ ID NO: 31). In some embodiments, the 3E10 antibody or antigen-binding fragment thereof further includes VL CDRs 1-3, and VH CDRs 1 and 3 according to the parent 3E10 antibody (as shown in FIG. 1). In some embodiments, the 3E10 antibody or antigen-binding fragment thereof further includes VL CDRs 1-3, and VH CDRs 1 and 3 according to the 3E10-D31N variant (as shown in FIG. 2A). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1) or relative to the 3E10-D31N variant (as shown in FIG. 2A), e.g., as described herein.

Similarly, in some embodiments, a 3E10 antibody or antigen-binding fragment thereof includes VL CDR1 comprising the amino acid sequence of 3E10-VL-CDR1.3 (SEQ ID NO: 32). In some embodiments, the 3E10 antibody or antigen-binding fragment thereof further includes VL CDRs 2 and 3, and VH CDRs 1-3 according to the parent 3E10 antibody (as shown in FIG. 1). In some embodiments, the 3E10 antibody or antigen-binding fragment thereof further includes VL CDRs 2 and 3, and VH CDRs 1-3 according to the 3E10-D31N variant (as shown in FIG. 2A). In some embodiments, the 3E10 antibody or antigen-binding fragment thereof further includes VL CDRs 2 and 3, and VH CDRs 1-3 having one or more amino acid substitutions relative to the CDRs of the parent 3E10 antibody (as shown in FIG. 1) or relative to the 3E10-D31N variant (as shown in FIG. 2A), e.g., as described herein.

Similarly, in some embodiments, a 3E10 antibody or antigen-binding fragment thereof, includes VL CDR2 comprising the amino acid sequence of 3E10-VL-CDR2.2 (SEQ ID NO: 33).

In some embodiments, the 3E10 antibody or antigen-binding fragment thereof further includes VL CDRs 1 and 3, and VH CDRs 1-3 according to the parent 3E10 antibody (as shown in FIG. 1). In some embodiments, the 3E10 antibody or antigen-binding fragment thereof further includes VL CDRs 1 and 3, and VH CDRs 1-3 according to the 3E10-D31N variant (as shown in FIG. 2A). In some embodiments, the 3E10 antibody or antigen-binding fragment thereof further includes VL CDRs 1 and 3, and VH CDRs 1-3 having one or more amino acid substitutions relative to the CDRs of the parent 3E10 antibody (as shown in FIG. 1) or relative to the 3E10-D31N variant (as shown in FIG. 2A), e.g., as described herein.

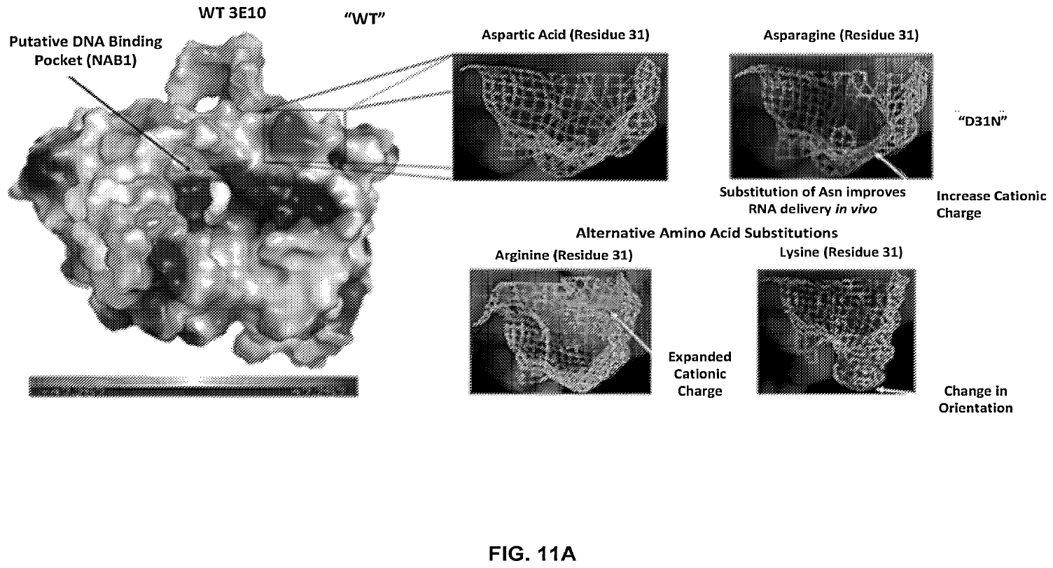

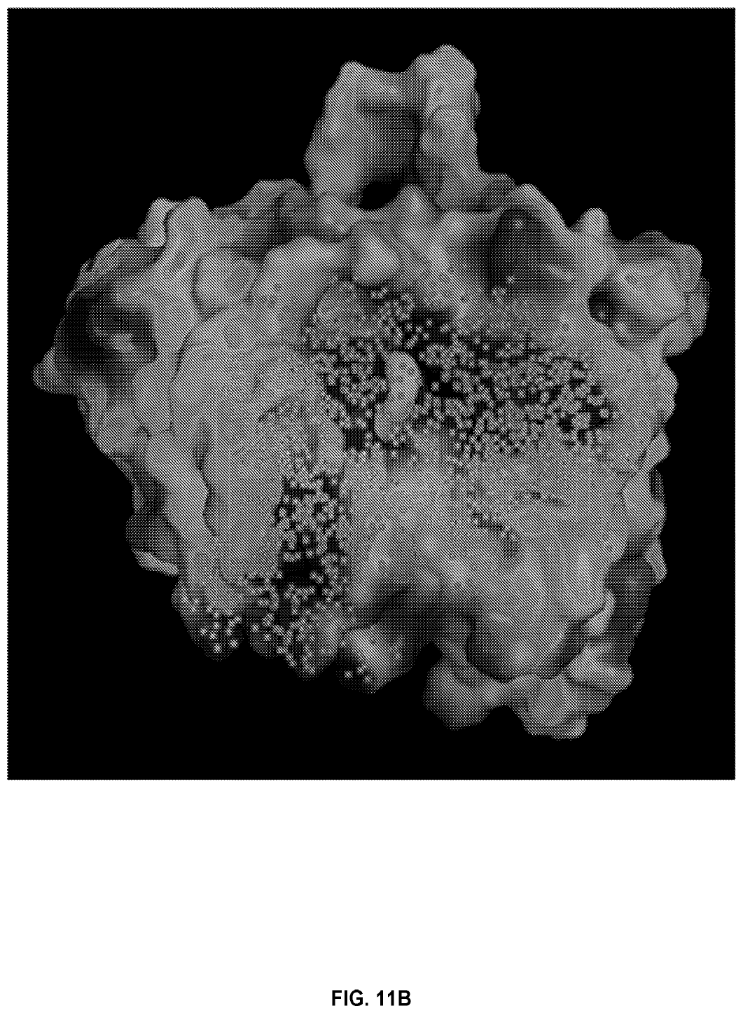

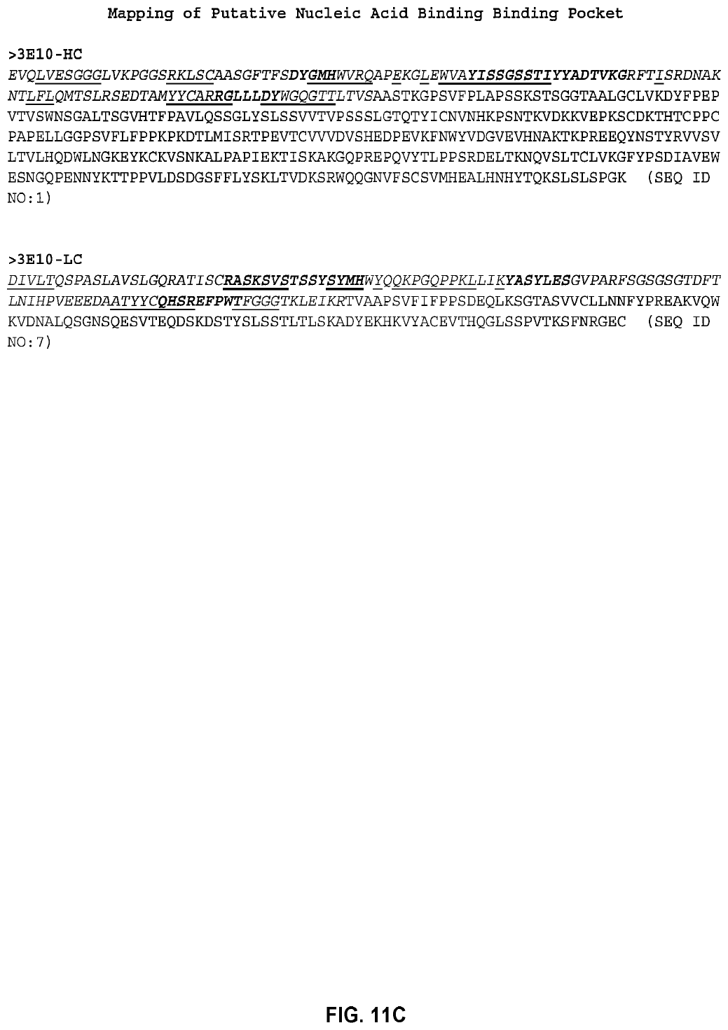

Further, because 3E10 antibodies or variants thereof, or antigen-binding fragments thereof, bind to nucleic acid in a partially sequence-independent manner, and without being bound by theory, it was contemplated that the interaction may be mediated by electrostatic interactions with the nucleotide backbone. To investigate this theory, electrostatic surface potential renderings of a molecular model of a 3E10-scFv construct—the amino acid sequence of which is illustrated in FIG. 11C—were generated, as shown in FIGS. 11A and 11B. These models revealed a putative Nucleic Acid Binding pocket (NAB1) corresponding to a large basic region on the surface of the molecule, as illustrated in FIG. 11A. The position of the non-hydrogen atoms of the amino acids contributing to the putative Nucleic Acid Binding pocket in the model are superposed in FIG. 11B, and the amino acid residues are mapped onto the sequence of the construct in FIG. 11C.

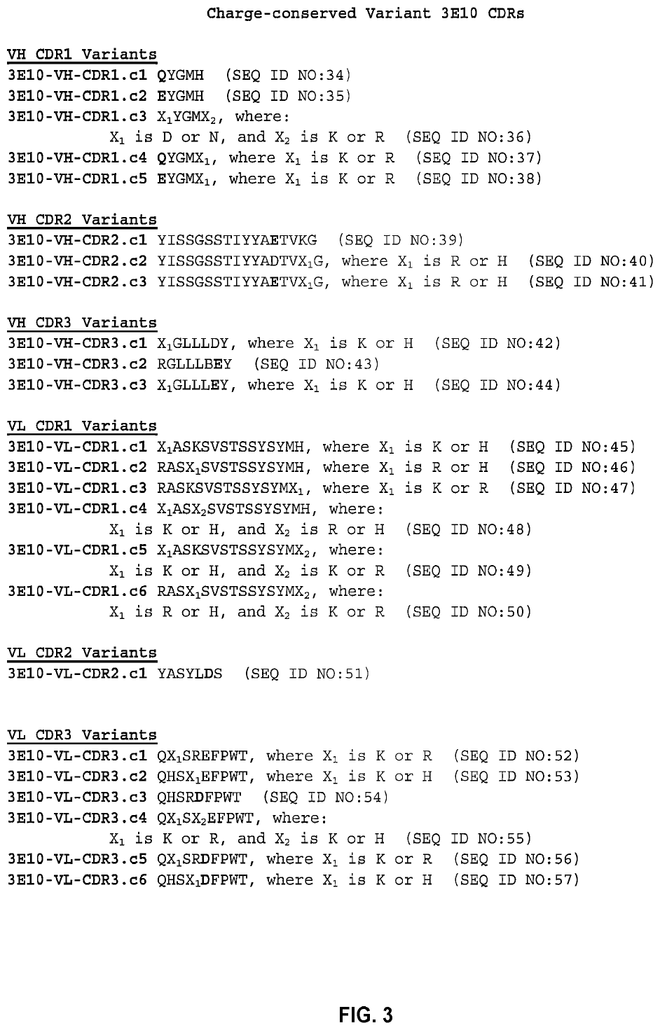

Thus, it is contemplated that amino acid substitutions within the CDRs of a 3E10 antibody or antigen-binding fragment thereof, as described herein, that maintain the electrostatic character of this putative Nucleic Acid Binding pocket will also retain the nucleic acid binding properties of the construct. Accordingly, in some embodiments, a 3E10 antibody or antigen-binding fragment thereof, includes one or more amino acid substitution of a first basic amino acid to a second basic amino acid (e.g., K, R, or H). Similarly, in some embodiments, a 3E10 antibody or antigen-binding fragment thereof, includes one or more amino acid substitution of a first acidic amino acid to a second acidic amino acid (e.g., D or E). Examples of such charge-conserved variant 3E10 CDRs are shown in FIG. 3.

Accordingly, in some embodiments, a 3E10 antibody or antigen-binding fragment thereof includes VH CDR1 comprising the amino acid sequence of 3E10-VH-CDR1.c1 (SEQ ID NO: 34), 3E10-VH-CDR1.c2 (SEQ ID NO: 35), 3E10-VH-CDR1.c3 (SEQ ID NO: 36), 3E10-VH-CDR1.c4 (SEQ ID NO: 37), or 3E10-VH-CDR1.c5 (SEQ ID NO: 38). In some embodiments, the 3E10 antibody or antigen-binding fragment thereof further includes VL CDRs 1-3, and VH CDRs 2 and 3 according to the parent 3E10 antibody (as shown in FIG. 1). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VH CDR2 comprising the amino acid sequence of 3E10-VH-CDR2.c1 (SEQ ID NO: 39), 3E10-VH-CDR2.c2 (SEQ ID NO: 40), or 3E10-VH-CDR2.c3 (SEQ ID NO: 41). In some embodiments, the 3E10 antibody or antigen-binding fragment thereof further includes VL CDRs 1-3, and VH CDRs 1 and 3 according to the parent 3E10 antibody (as shown in FIG. 1). In some embodiments, the 3E10 antibody or antigen-binding fragment thereof further includes VL CDRs 1-3, and VH CDRs 1 and 3 according to the 3E10-D31N variant (as shown in FIG. 2A). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VH CDR3 comprising the amino acid sequence of 3E10-VH-CDR3.c1 (SEQ ID NO: 42), 3E10-VH-CDR3.c2 (SEQ ID NO: 43), or 3E10-VH-CDR3.c3 (SEQ ID NO: 44). In some embodiments, the 3E10 antibody or antigen-binding fragment thereof further includes VL CDRs 1-3, and VH CDRs 1 and 2 according to the parent 3E10 antibody (as shown in FIG. 1). In some embodiments, the 3E10 antibody or antigen-binding fragment thereof further includes VL CDRs 1-3, and VH CDRs 1 and 2 according to the 3E10-D31N variant (as shown in FIG. 2A). In some embodiments, the 3E10 antibody or antigen-binding fragment thereof further includes VL CDRs 1-3, and VH CDRs 1 and 2 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR1 comprising the amino acid sequence of 3E10-VL-CDR1.c1 (SEQ ID NO: 45), 3E10-VL-CDR1.c2 (SEQ ID NO: 46), 3E10-VL-CDR1.c3 (SEQ ID NO: 47), 3E10-VL-CDR1.c4 (SEQ ID NO: 48), 3E10-VL-CDR1.c5 (SEQ ID NO: 49), or 3E10-VL-CDR1.c6 (SEQ ID NO: 50). In some embodiments, the 3E10 antibody or antigen-binding fragment thereof further includes VL CDRs 2 and 3, and VH CDRs 1-3 according to the parent 3E10 antibody (as shown in FIG. 1). In some embodiments, the 3E10 antibody or antigen-binding fragment thereof further includes VL CDRs 2 and 3, and VH CDRs 1-3 according to the 3E10-D31N variant (as shown in FIG. 2A). In some embodiments, the 3E10 antibody or antigen-binding fragment thereof further includes VL CDRs 2 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR2 comprising the amino acid sequence of 3E10-VL-CDR2.c1 (SEQ ID NO: 51). In some embodiments, the 3E10 antibody or antigen-binding fragment thereof further includes VL CDRs 1 and 3, and VH CDRs 1-3 according to the parent 3E10 antibody (as shown in FIG. 1). In some embodiments, the 3E10 antibody or antigen-binding fragment thereof further includes VL CDRs 1 and 3, and VH CDRs 1-3 according to the 3E10-D31N variant (as shown in FIG. 2A). In some embodiments, the 3E10 antibody or antigen-binding fragment thereof further includes VL CDRs 1 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR3 comprising the amino acid sequence of 3E10-VL-CDR3.c1 (SEQ ID NO: 52), 3E10-VL-CDR3.c2 (SEQ ID NO: 53), 3E10-VL-CDR3.c3 (SEQ ID NO: 54), 3E10-VL-CDR3.c4 (SEQ ID NO: 55), 3E10-VL-CDR3.c5 (SEQ ID NO: 56), or 3E10-VL-CDR3.c6 (SEQ ID NO: 57). In some embodiments, the 3E10 antibody or antigen-binding fragment thereof further includes VL CDRs 1 and 2, and VH CDRs 1-3 according to the parent 3E10 antibody (as shown in FIG. 1). In some embodiments, the 3E10 antibody or antigen-binding fragment thereof further includes VL CDRs 1 and 2, and VH CDRs 1-3 according to the 3E10-D31N variant (as shown in FIG. 2A). In some embodiments, the 3E10 antibody or antigen-binding fragment thereof further includes VL CDRs 1 and 2, and VH CDRs 1-3 having one or more amino acid substitutions relative to the CDRs of the parent 3E10 antibody (as shown in FIG. 1), e.g., as described herein.

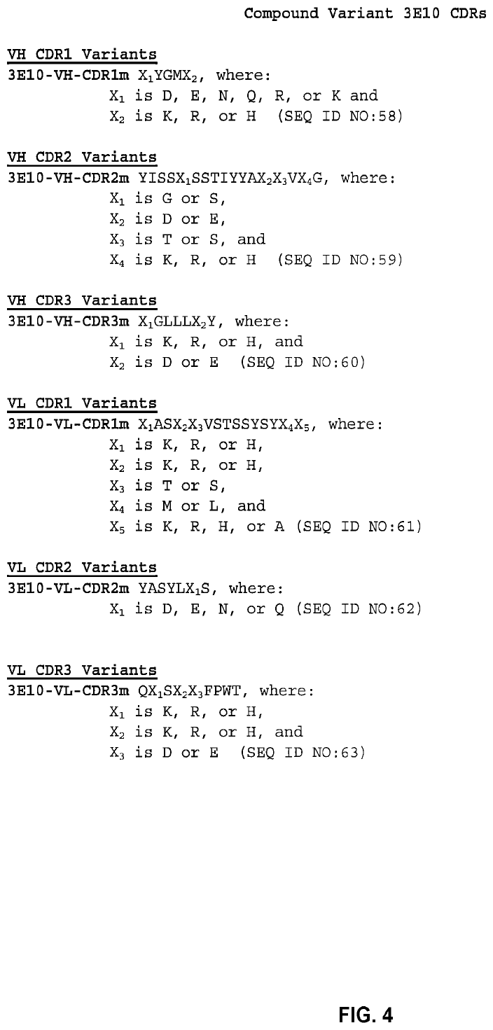

It is also contemplated that a 3E10 antibody or antigen-binding fragment thereof, as described herein, includes any combination of the 3E10 CDR amino acid substitutions described above. Examples of 3E10 variant CDR sequences that incorporate one or more of the amino acid substitutions described herein are shown in FIG. 4.

Accordingly, in some embodiments, a 3E10 antibody or antigen-binding fragment thereof includes VH CDR1 comprising the amino acid sequence of 3E10-VH-CDR1m (SEQ ID NO: 58). In some embodiments, the 3E10 antibody or antigen-binding fragment thereof further includes VL CDRs 1-3, and VH CDRs 2 and 3 according to the parent 3E10 antibody (as shown in FIG. 1). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VH CDR2 comprising the amino acid sequence of 3E10-VH-CDR2m (SEQ ID NO: 59). In some embodiments, the 3E10 antibody or antigen-binding fragment thereof further includes VL CDRs 1-3, and VH CDRs 1 and 3 according to the parent 3E10 antibody (as shown in FIG. 1). In some embodiments, the 3E10 antibody or antigen-binding fragment thereof further includes VL CDRs 1-3, and VH CDRs 1 and 3 according to the 3E10-D31N variant (as shown in FIG. 2A). In some embodiments, the 3E10 antibody or antigen-binding fragment thereof further includes VL CDRs 1-3, and VH CDRs 1 and 3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VH CDR3 comprising the amino acid sequence of 3E10-VH-CDR3m (SEQ ID NO: 60). In some embodiments, the 3E10 antibody or antigen-binding fragment thereof further includes VL CDRs 1-3, and VH CDRs 1 and 2 according to the parent 3E10 antibody (as shown in FIG. 1). In some embodiments, the 3E10 antibody or antigen-binding fragment thereof further includes VL CDRs 1-3, and VH CDRs 1 and 2 according to the 3E10-D31N variant (as shown in FIG. 2A). In some embodiments, the 3E10 antibody or antigen-binding fragment thereof further includes VL CDRs 1-3, and VH CDRs 1 and 2 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR1 comprising the amino acid sequence of 3E10-VL-CDR1m (SEQ ID NO: 61). In some embodiments, the 3E10 antibody or antigen-binding fragment thereof further includes VL CDRs 2 and 3, and VH CDRs 1-3 according to the parent 3E10 antibody (as shown in FIG. 1). In some embodiments, the 3E10 antibody or antigen-binding fragment thereof further includes VL CDRs 2 and 3, and VH CDRs 1-3 according to the 3E10-D31N variant (as shown in FIG. 2A). In some embodiments, the 3E10 antibody or antigen-binding fragment thereof further includes VL CDRs 2 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR2 comprising the amino acid sequence of 3E10-VL-CDR2m (SEQ ID NO: 62). In some embodiments, the 3E10 antibody or antigen-binding fragment thereof further includes VL CDRs 1 and 3, and VH CDRs 1-3 according to the parent 3E10 antibody (as shown in FIG. 1). In some embodiments, the 3E10 antibody or antigen-binding fragment thereof further includes VL CDRs 1 and 3, and VH CDRs 1-3 according to the 3E10-D31N variant (as shown in FIG. 2A). In some embodiments, the 3E10 antibody or antigen-binding fragment thereof further includes VL CDRs 1 and 3, and VH CDRs 1-3 having one or more amino acid substitutions relative to the CDRs of the parent 3E10 antibody (as shown in FIG. 1), e.g., as described herein.

Similarly, in some embodiments, a 3E10 antibody or antigen-binding fragment thereof includes VL CDR3 comprising the amino acid sequence of 3E10-VL-CDR3m (SEQ ID NO: 63). In some embodiments, the 3E10 antibody or antigen-binding fragment thereof further includes VL CDRs 1 and 2, and VH CDRs 1-3 according to the parent 3E10 antibody (as shown in FIG. 1). In some embodiments, the 3E10 antibody or antigen-binding fragment thereof further includes VL CDRs 1 and 2, and VH CDRs 1-3 according to the 3E10-D31N variant (as shown in FIG. 2A). In some embodiments, the 3E10 antibody or antigen-binding fragment thereof further includes VL CDRs 1 and 2, and VH CDRs 1-3 having one or more amino acid substitutions relative to the CDRs of the parent 3E10 antibody (as shown in FIG. 1), e.g., as described herein.

In some embodiments, a 3E10 antibody or antigen-binding fragment thereof described herein includes a light chain variable region (VL) complementarity determining region (CDR) 1 comprising the amino acid sequence of 3E10-VL-CDR1m (SEQ ID NO: 61), a VL CDR2 comprising the amino acid sequence of 3E10-VL-CDR2m (SEQ ID NO: 62), a VL CDR3 comprising the amino acid sequence of 3E10-VL-CDR3m (SEQ ID NO: 63), a heavy chain variable region (VH) CDR1 comprising the amino acid sequence of 3E10-VH-CDR1m (SEQ ID NO: 58), a VH CDR2 comprising the amino acid sequence of 3E10-VH-CDR2m (SEQ ID NO: 59), and a VH CDR3 comprising the amino acid sequence of 3E10-VH-CDR3m (SEQ ID NO: 60).

In some embodiments, a 3E10 antibody or antigen-binding fragment thereof described herein refers to CDR sequences having no more than one amino acid substitution relative to the parent 3E10 antibody, shown in FIG. 1, optionally including a D31N amino acid substitution in the VH CDR1. Accordingly, in some embodiments, a 3E10 antibody or antigen-binding fragment thereof includes a light chain variable region (VL) complementarity determining region (CDR) 1 comprising an amino acid sequence having no more than one amino acid substitution relative to 3E10-VL-CDR1 (SEQ ID NO: 9), a VL CDR2 comprising an amino acid sequence having no more than one amino acid substitution relative to 3E10-VL-CDR2 (SEQ ID NO: 10), a VL CDR3 comprising an amino acid sequence having no more than one amino acid substitution relative to 3E10-VL-CDR3 (SEQ ID NO: 11), a heavy chain variable region (VH) CDR1 comprising an amino acid sequence having no more than one amino acid substitution relative to 3E10-VH-CDR1a (SEQ ID NO: 16), a VH CDR2 comprising an amino acid sequence having no more than one amino acid substitution relative to 3E10-VH-CDR2 (SEQ ID NO: 5), and a VH CDR3 comprising an amino acid sequence having no more than one amino acid substitution relative to 3E10-VH-CDR3 (SEQ ID NO: 6).

In some embodiments, a 3E10 antibody or antigen-binding fragment thereof described herein refers to CDR sequences having no more than two amino acid substitution relative to the parent 3E10 antibody, shown in FIG. 1, optionally including a D31N amino acid substitution in the VH CDR1. Accordingly, in some embodiments, a 3E10 antibody or antigen-binding fragment thereof includes a light chain variable region (VL) complementarity determining region (CDR) 1 comprising an amino acid sequence having no more than two amino acid substitutions relative to 3E10-VL-CDR1 (SEQ ID NO: 9), a VL CDR2 comprising an amino acid sequence having no more than two amino acid substitutions relative to 3E10-VL-CDR2 (SEQ ID NO: 10), a VL CDR3 comprising an amino acid sequence having no more than two amino acid substitutions relative to 3E10-VL-CDR3 (SEQ ID NO: 11), a heavy chain variable region (VH) CDR1 comprising an amino acid sequence having no more than two amino acid substitutions relative to 3E10-VH-CDR1a (SEQ ID NO: 16), a VH CDR2 comprising an amino acid sequence having no more than two amino acid substitutions relative to 3E10-VH-CDR2 (SEQ ID NO: 4), and a VH CDR3 comprising an amino acid sequence having no more than two amino acid substitutions relative to 3E10-VH-CDR3 (SEQ ID NO: 5).

Other variants of a 3E10 antibody or antigen-binding fragment thereof are also known in the art, as disclosed for example, in Zack, et al., J. Immunol., 157 (5): 2082-8 (1996). For example, amino acid position 31 of the heavy chain variable region of 3E10 has been determined to be influential in the ability of the antibody and fragments thereof to penetrate nuclei and bind to DNA (bolded in SEQ ID NOs: 1, 2, and 13). A D31N mutation (bolded in SEQ ID NOs: 2 and 13) in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)). In some embodiments, the antibody has the D31N substitution.

Although generally referred to herein as “antigen binding fragments” of a 3E10 antibody,” it will be appreciated that fragments and binding proteins, including antigen-binding fragments, variants, and fusion proteins such as scFv, di-scFv, tr-scFv, and other single chain variable fragments, and other cell-penetrating, nucleic acid transporting molecules disclosed herein are encompassed by the phrase are also expressly provided for use in compositions and methods disclosed herein. Thus, the antibodies and other binding proteins are also referred to herein as cell-penetrating.

A humanized 3E10 antibody or antigen binding fragment thereof is capable of being transported into the cytoplasm and/or nucleus of the cells without the aid of a carrier or conjugate. For example, the monoclonal antibody 3E10 and active fragments thereof that are transported in vivo to the nucleus of mammalian cells without cytotoxic effect are disclosed in U.S. Pat. Nos. 4,812,397 and 7,189,396 to Richard Weisbart.

In some embodiments, a humanized 3E10 antibody or antigen binding fragment thereof binds and/or inhibits Rad51. See, e.g., Turchick, et al., Nucleic Acids Res., 45 (20): 11782-11799 (2017), US 2021/0340280, and US 2021/033881, the contents of which are incorporated by reference herein, in its entirety

Humanized 3E10 antibodies and ENT2 binding fragments thereof that can be used in the in the compositions and methods described herein include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the humanized 3E10 antibodies and ENT2 binding fragments thereof typically contain at least the CDRs necessary to maintain DNA binding and/or interfere with DNA repair.

The 3E10 antibody is typically a monoclonal 3E10, or a variant, derivative, fragment, fusion, or humanized form thereof that binds the same or different epitope(s) as 3E10.

A deposit according to the terms of the Budapest Treaty of a hybridoma cell line producing monoclonal antibody 3E10 was received on Sep. 6, 2000, and accepted by, American Type Culture Collection (ATCC), 10801 University Blvd., Manassas, VA 20110-2209, USA, and given Patent Deposit Number PTA-2439.

Thus, the antibody may have the same or different epitope specificity as monoclonal antibody 3E10 produced by ATCC No. PTA 2439 hybridoma. The antibody can have the paratope of monoclonal antibody 3E10. The antibody can be a single chain variable fragment of 3E10, or a variant, e.g., a conservative variant thereof. For example, the antibody can be a single chain variable fragment of 3E10 (3E10 Fv), or a variant thereof.

Generally, a humanized antibody is the result of a process in which the sequence of a parental antibody from a non-human species is modified to increase the overall similarity of the parental antibody to human antibodies, while retaining antigen binding activity of the parental antibody. Generally, the process involves identifying a human antibody, sometimes referred to as a scaffold antibody, and then either (i) replacing amino acids in the parent (non-human) antibody with equivalent amino acids from the scaffold (human) antibody, e.g., framework amino acids having little to no effect on antigen binding or (ii) replacing amino acids in the scaffold (human) antibody with equivalent amino acids from the parent (non-human) antibody, e.g., CDRs and other amino acids with significant effects on antigen binding. Various methods for humanization are known in the art, including framework-homology-based humanization, germline humanization, complementary determining regions (CDR)-homology-based humanization, and specificity determining residues (SDR) grafting. For a review of these methods see, for example, Safdari Y. et al., Biotechnology and Genetic Engineering Reviews, 29:2, 175-86 (2013).

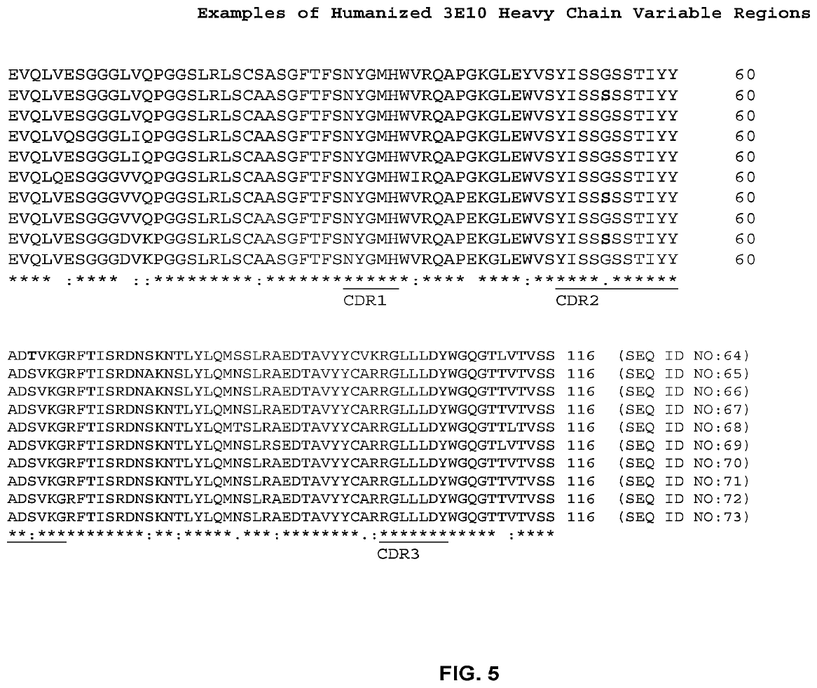

Exemplary 3E10 humanized sequences are discussed in WO 2015/106290, WO 2016/033324, WO 2019/018426, and WO/2019/018428, and provided in FIGS. 5 (Humanized 3E10 Heavy Chain Variable Regions) and 6 (Humanized 3E10 Light Chain Variable Regions).

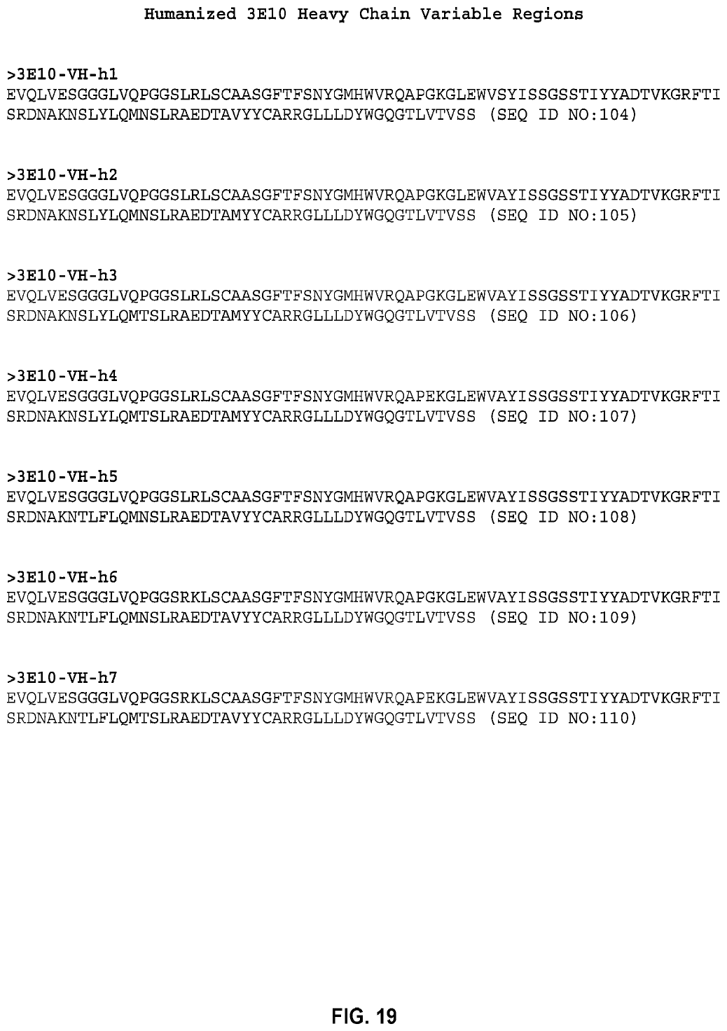

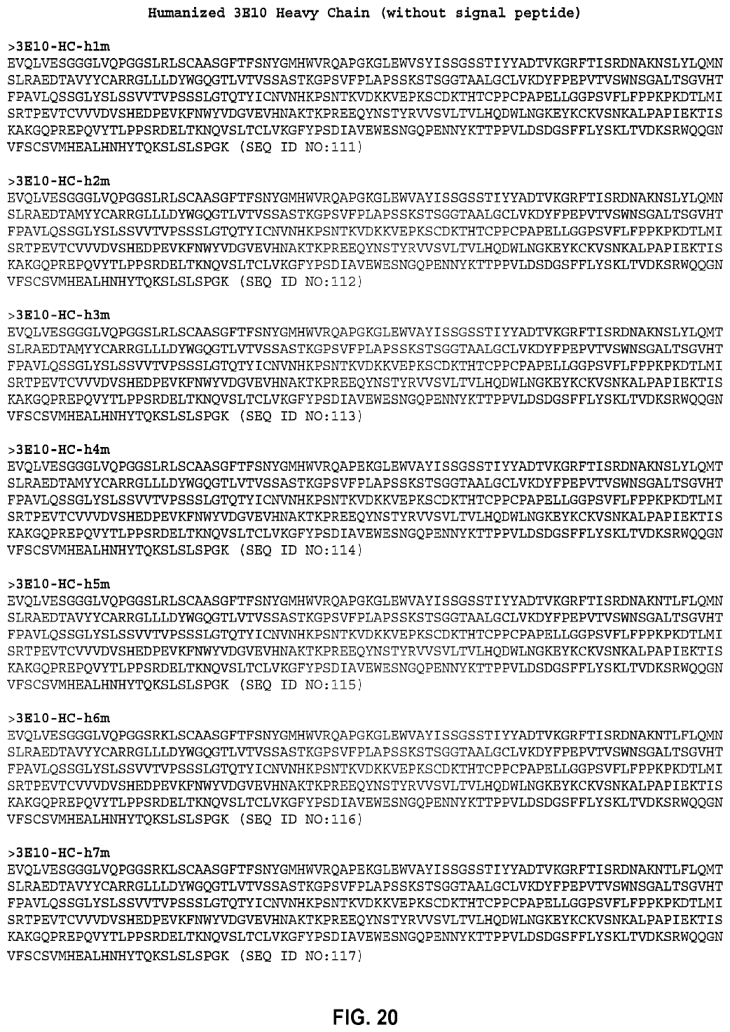

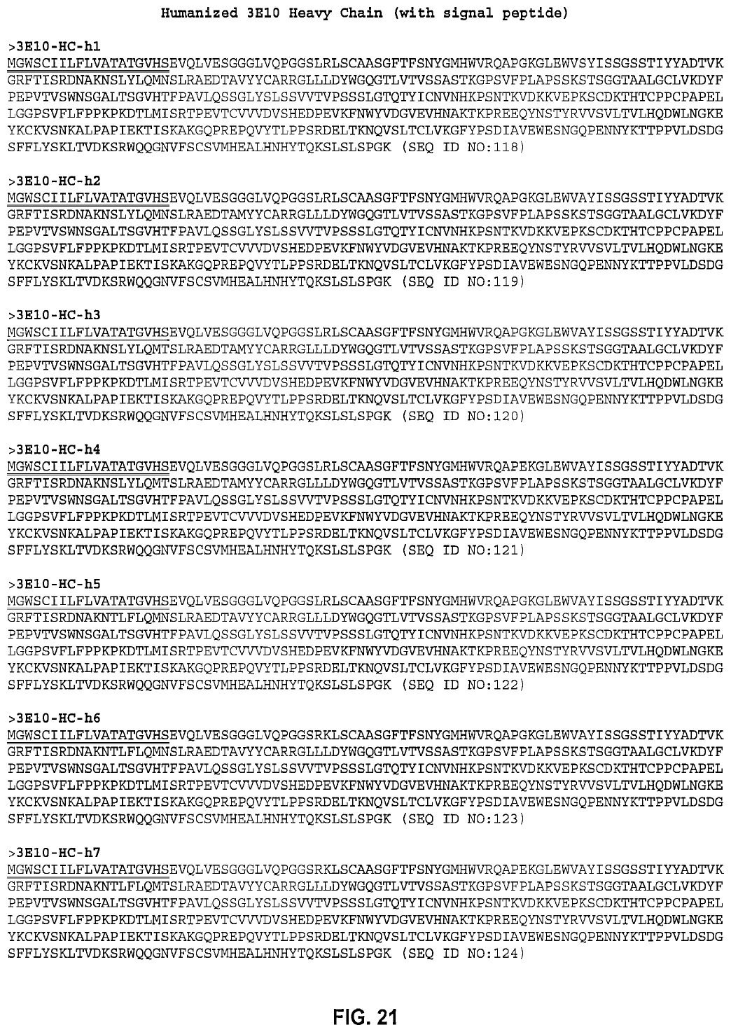

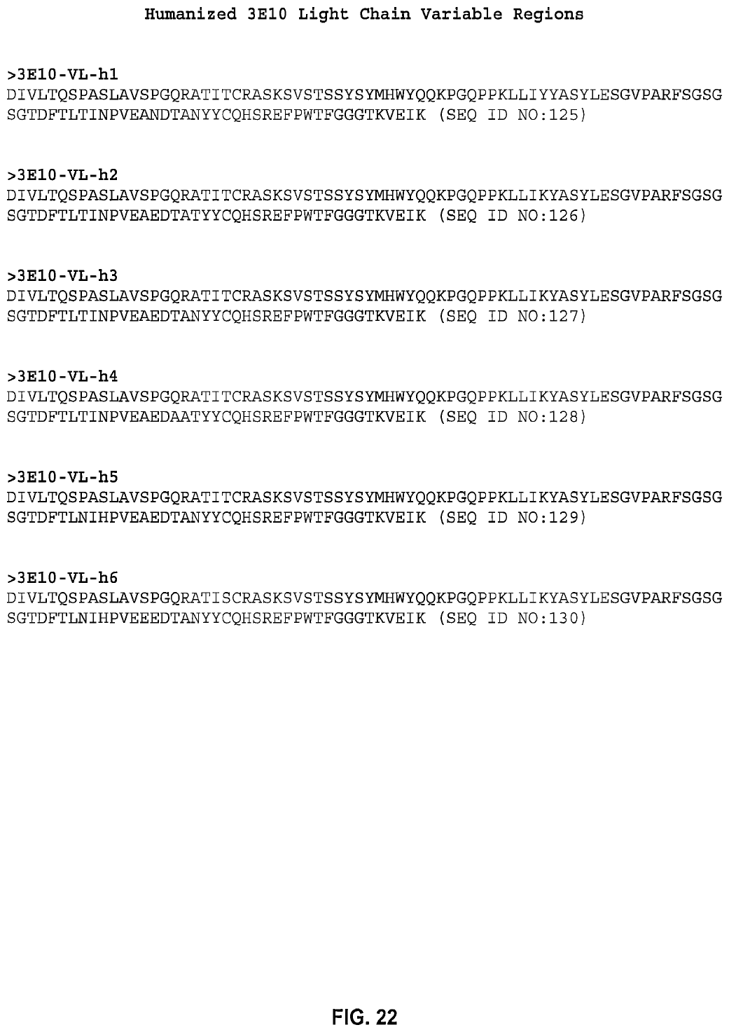

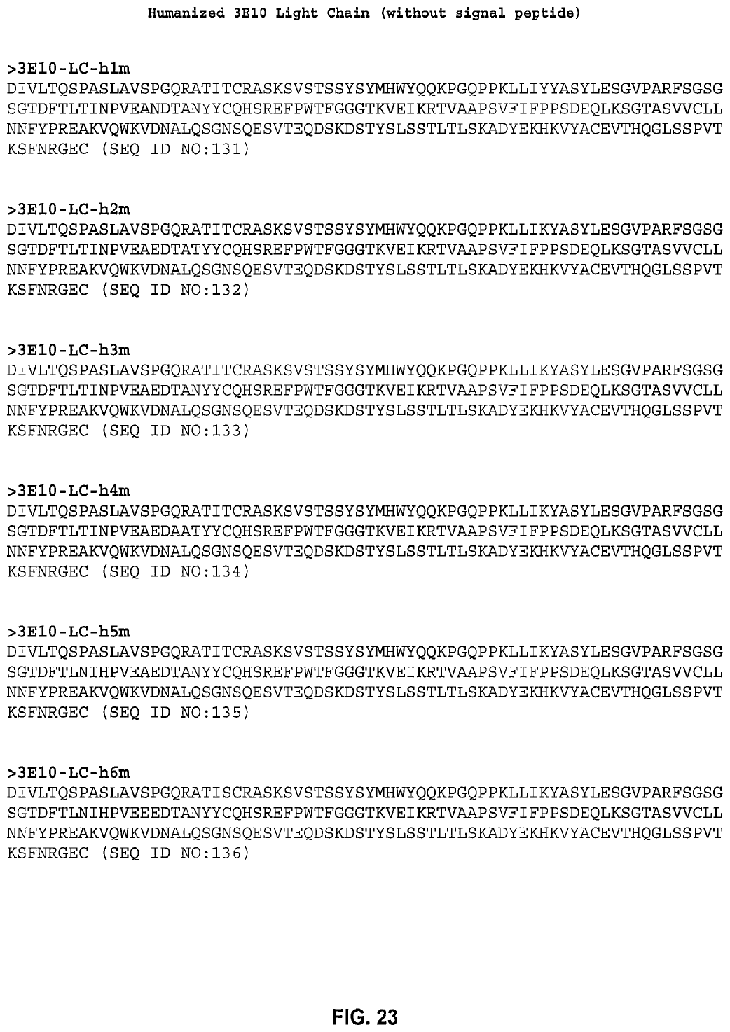

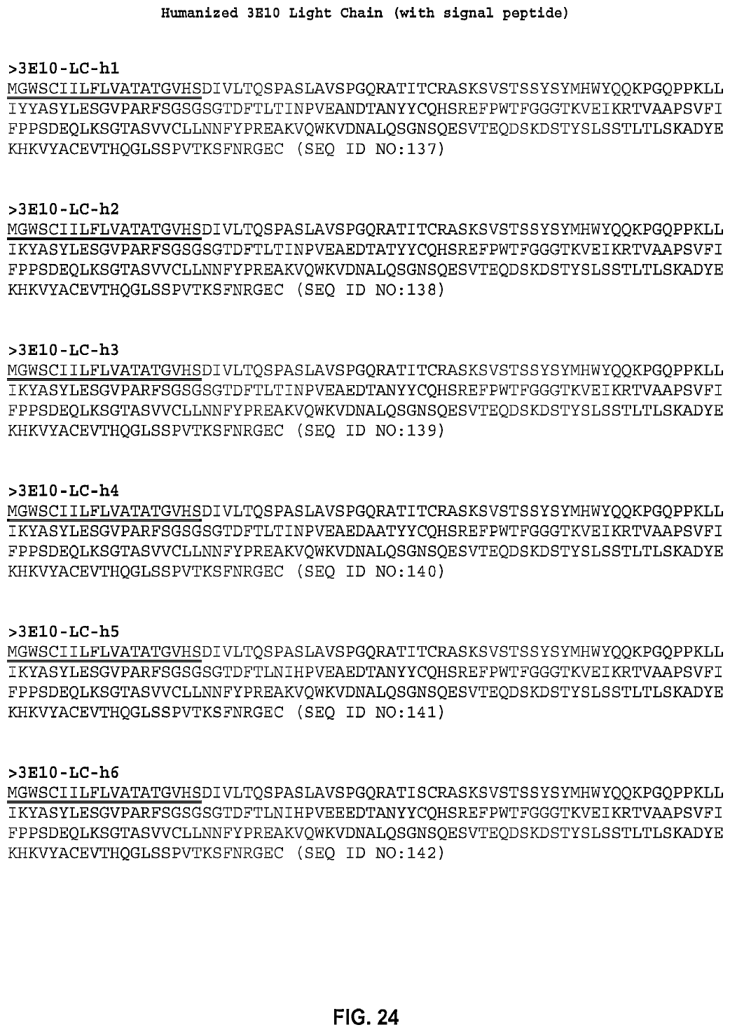

In some embodiments, a humanized 3E10 antibody or antigen-binding fragment thereof has a sequence with high sequence identity, e.g., at least 95% identity, at least 96% identity, at least 97% identity, at least 99% identity, at least 99.5% identity, or 100% identity with a humanized 3E10 variable light domains and/or humanized 3E10 variable heavy domains shown in FIG. 19 (heavy chain variable regions), FIG. 20 (heavy chain without signal sequence), FIG. 21 (heavy chain with signal peptide), FIG. 22 (light chain variable regions), FIG. 23 (light chain without signal sequence), and/or FIG. 24 (light chain with signal peptide). These variable light and variable heavy domains can be combined in any of the possible 42 combinations (each of the seven variable light domains with each of the variable heavy domains) to form humanized 3E10 antibodies and nucleic acid binding fragments (e.g., scFvs) thereof. Twenty-two (22) antibodies incorporating different combinations of these humanized VL and VH sequences were made, all of which bound nucleic acids. Further, when human leukemia cells were exposed to complexes formed between these antibodies and a RIG-I agonist polynucleotide, all of the complexes were able to generate a Type I IFN response, suggesting that all of the tested antibodies were able to deliver functional polynucleotides into the cells and affect a RIG-I mediated response (data not shown).

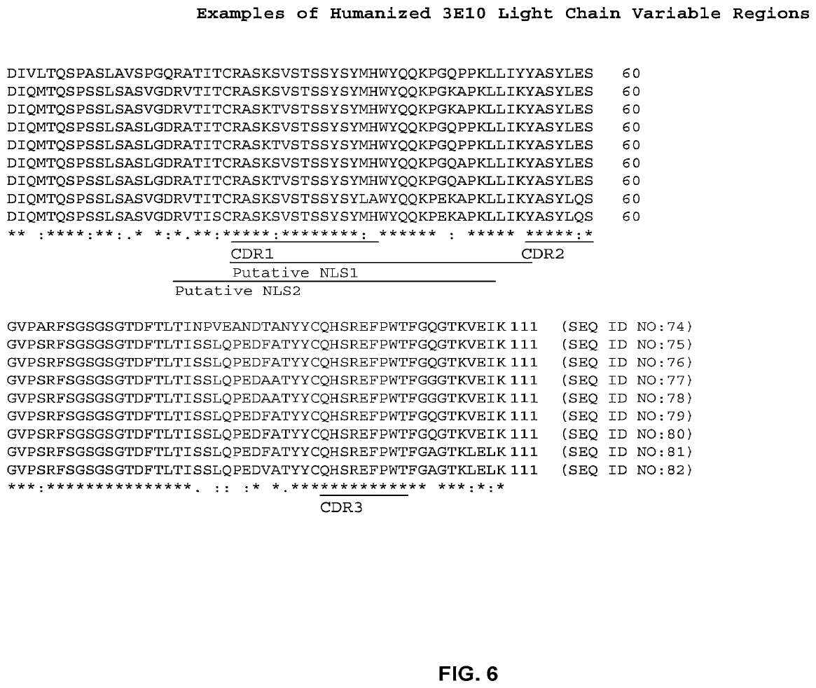

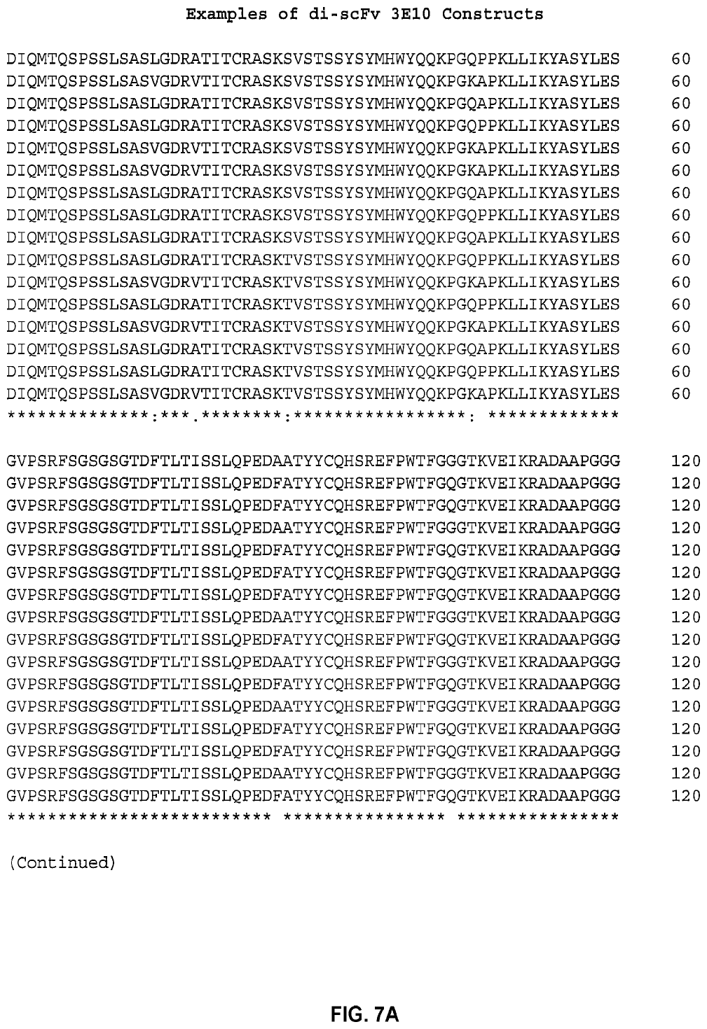

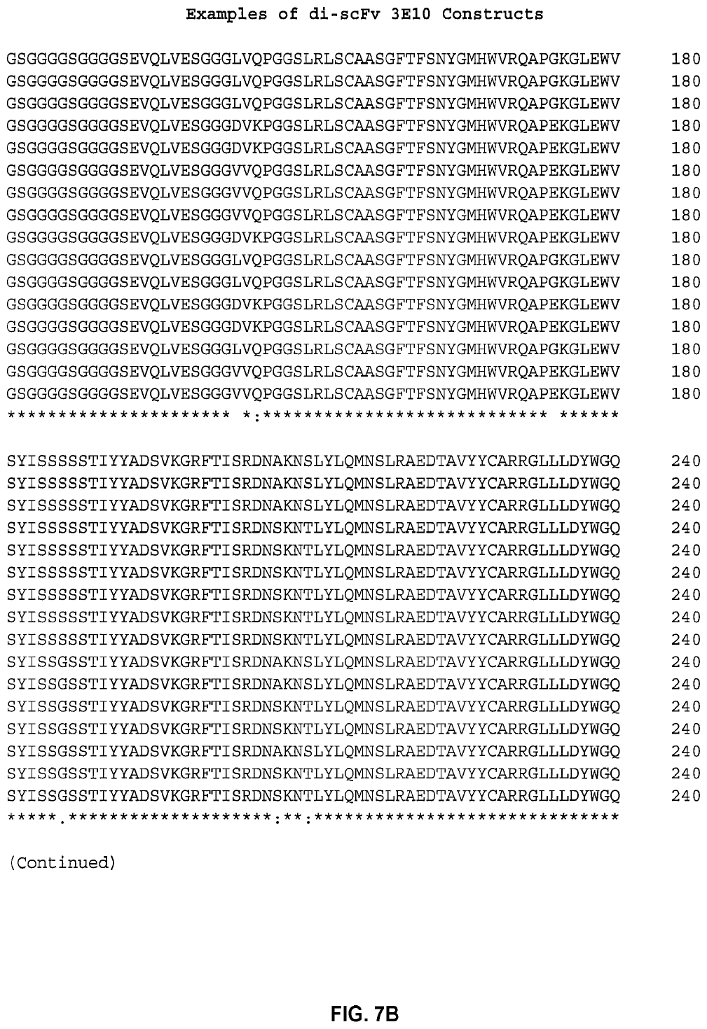

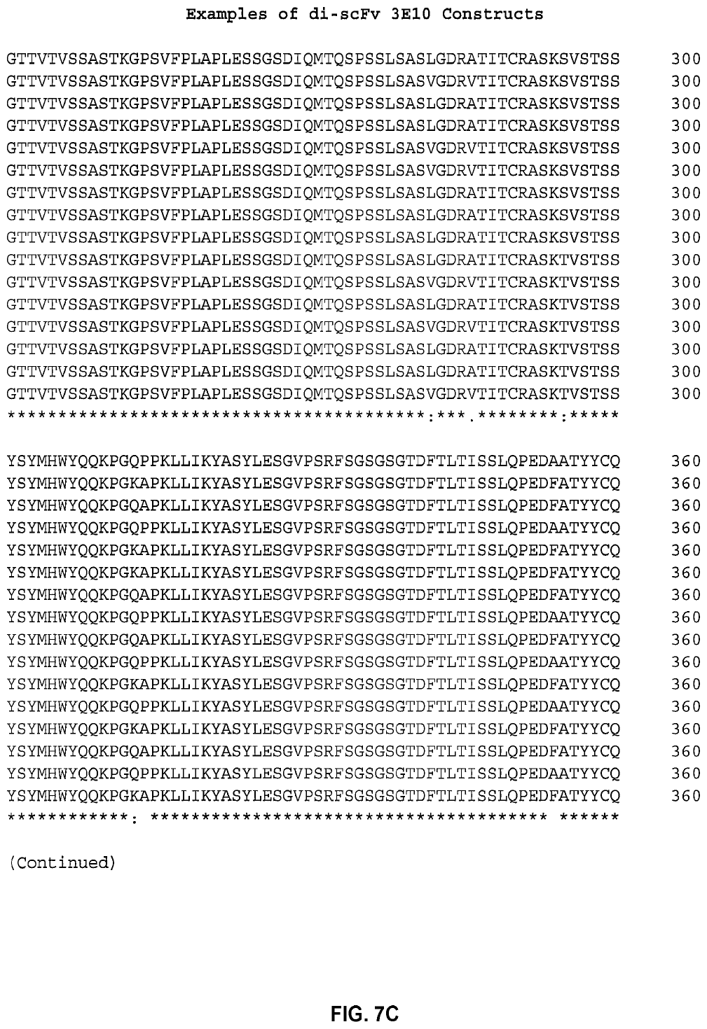

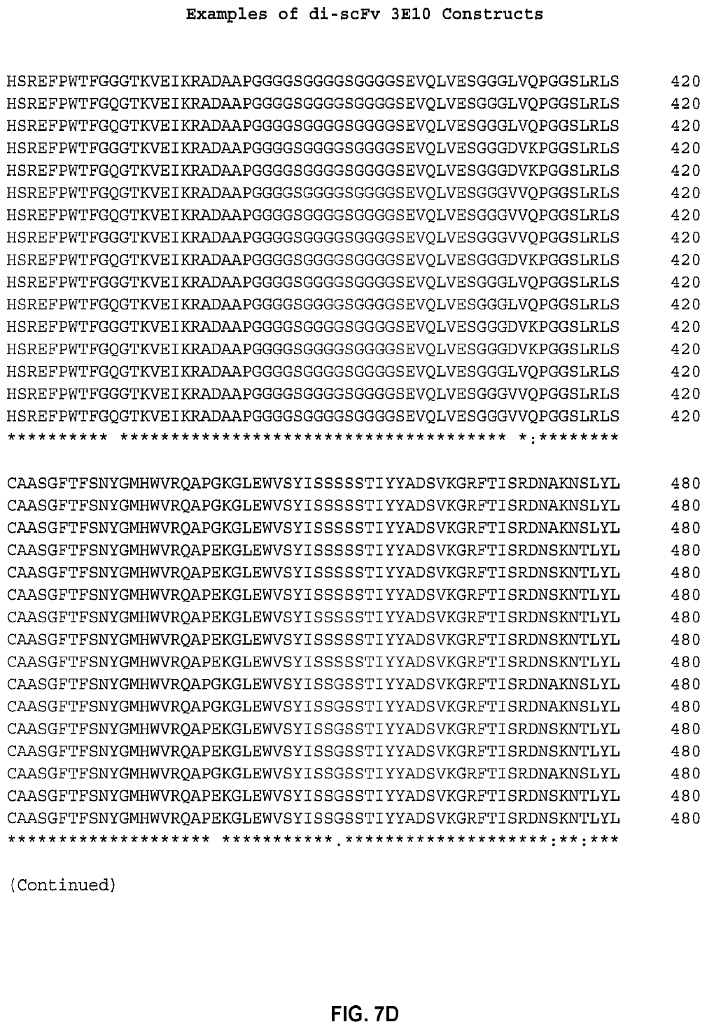

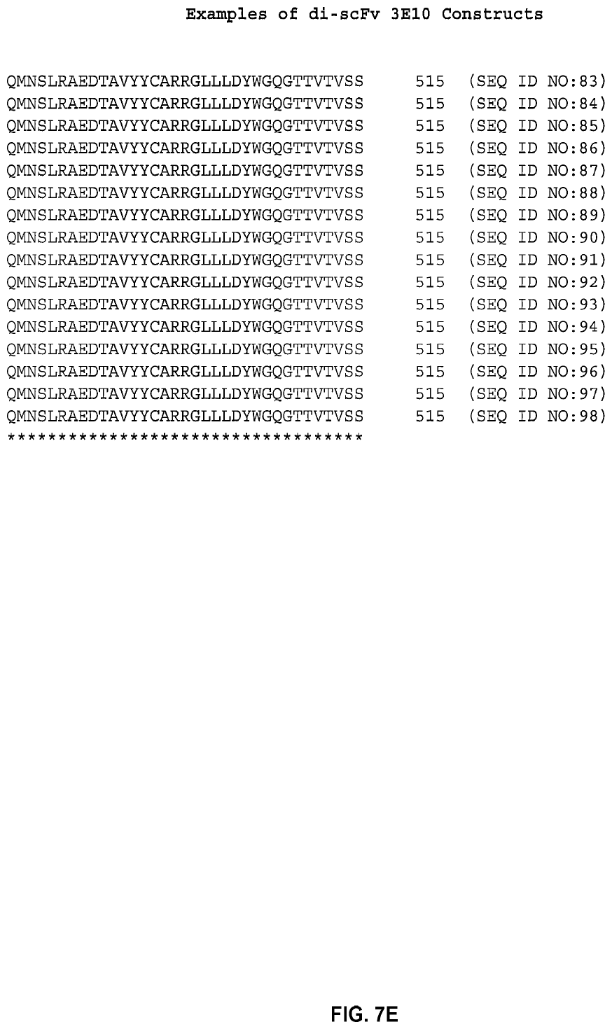

Accordingly, in some embodiments the disclosure provides humanized 3E10 antibodies and antigen-binding fragments thereof that incorporate any combination of the humanized VL and VH sequences shown in FIGS. 5-10, as well as VL and VH sequences having sequence identity thereto, e.g., having at least 90%, 91%, 92%, 93%, 94%, 95%, 96%, 97%, 98%, or 99% identity to a VH or VL sequence shown in FIGS. 19-24.

In some embodiments, a humanized 3E10 antibody or antigen binding fragment thereof, described herein includes a light chain variable domain (3E10-VL) comprising an amino acid sequence that is at least 95%, 96%, 97%, 98%, 99%, 99.5%, or 100% identical to an amino acid sequence selected from the group consisting of 3E10-VL-h1 (SEQ ID NO:125), 3E10-VL-h2 (SEQ ID NO:126), 3E10-VL-h3 (SEQ ID NO:127), 3E10-VL-h4 (SEQ ID NO:128), 3E10-VL-h5 (SEQ ID NO:129), and 3E10-VL-h6 (SEQ ID NO:130) and a heavy chain variable domain (3E10-VH) comprising an amino acid sequence that is at least 95%, 96%, 97%, 98%, 99%, 99.5%, or 100% identical to an amino acid sequence selected from the group consisting of 3E10-VH-h1 (SEQ ID NO:104), 3E10-VH-h2 (SEQ ID NO:105), 3E10-VH-h3 (SEQ ID NO:106), 3E10-VH-h4 (SEQ ID NO:107), 3E10-VH-h5 (SEQ ID NO:108), 3E10-VH-h6 (SEQ ID NO: 109), and 3E10-VH-h7 (SEQ ID NO:110).

In some embodiments, the sequence of the 3E10-VL is at least 95% identical to 3E10-VL-h6 (SEQ ID NO:130). In some embodiments, the sequence of the 3E10-VL is at least 96% identical to 3E10-VL-h6 (SEQ ID NO:130). In some embodiments, the sequence of the 3E10-VL is at least 97% identical to 3E10-VL-h6 (SEQ ID NO:130). In some embodiments, the sequence of the 3E10-VL is at least 98% identical to 3E10-VL-h6 (SEQ ID NO:130). In some embodiments, the sequence of the 3E10-VL is at least 99% identical to 3E10-VL-h6 (SEQ ID NO: 130). In some embodiments, the sequence of the 3E10-VL is 3E10-VL-h6 (SEQ ID NO: 130).

In some embodiments, the sequence of the 3E10-VH is at least 95% identical to 3E10-VH-h6 (SEQ ID NO:109). In some embodiments, the sequence of the 3E10-VH is at least 96% identical to 3E10-VH-h6 (SEQ ID NO:109). In some embodiments, the sequence of the 3E10-VH is at least 97% identical to 3E10-VH-h6 (SEQ ID NO:109). In some embodiments, the sequence of the 3E10-VH is at least 98% identical to 3E10-VH-h6 (SEQ ID NO:109). In some embodiments, the sequence of the 3E10-VH is at least 99% identical to 3E10-VH-h6 (SEQ ID NO: 109). In some embodiments, the sequence of the 3E10-VH is 3E10-VH-h6 (SEQ ID NO: 109).

In some embodiments, a humanized 3E10 antibody or antigen binding fragment thereof, described herein includes a light chain (3E10-LC) comprising an amino acid sequence that is at least 95%, 96%, 97%, 98%, 99%, 99.5%, or 100% identical to an amino acid sequence selected from the group consisting of 3E10-LC-h1m (SEQ ID NO:131), 3E10-LC-h2m (SEQ ID NO: 132), 3E10-LC-h3m (SEQ ID NO:133), 3E10-LC-h4m (SEQ ID NO: 134), 3E10-LC-h5m (SEQ ID NO:135), and 3E10-LC-h6m (SEQ ID NO:136) and a heavy chain (3E10-HC) comprising an amino acid sequence that is at least 95%, 96%, 97%, 98%, 99%, 99.5%, or 100% identical to an amino acid sequence selected from the group consisting of 3E10-HC-h1m (SEQ ID NO: 111), 3E10-HC-h2m (SEQ ID NO:112), 3E10-HC-h3m (SEQ ID NO:113), 3E10-HC-h4m (SEQ ID NO:114), 3E10-HC-h5m (SEQ ID NO:115), 3E10-HC-h6m (SEQ ID NO:116), and 3E10-HC-h7m (SEQ ID NO:117).

In some embodiments, the sequence of the 3E10-LC is at least 95% identical to 3E10-LC-h6m (SEQ ID NO:136). In some embodiments, the sequence of the 3E10-LC is at least 96% identical to 3E10-LC-h6m (SEQ ID NO:136). In some embodiments, the sequence of the 3E10-LC is at least 97% identical to 3E10-LC-h6m (SEQ ID NO:136). In some embodiments, the sequence of the 3E10-LC is at least 98% identical to 3E10-LC-h6m (SEQ ID NO:136). In some embodiments, the sequence of the 3E10-LC is at least 99% identical to 3E10-LC-h6m (SEQ ID NO: 136). In some embodiments, the sequence of the 3E10-LC is 3E10-LC-h6m (SEQ ID NO: 136).

In some embodiments, the sequence of the 3E10-HC is at least 95% identical to 3E10-HC-h6m (SEQ ID NO:116). In some embodiments, the sequence of the 3E10-HC is at least 96% identical to 3E10-HC-h6m (SEQ ID NO:116). In some embodiments, the sequence of the 3E10-HC is at least 97% identical to 3E10-HC-h6m (SEQ ID NO:116). In some embodiments, the sequence of the 3E10-HC is at least 98% identical to 3E10-HC-h6m (SEQ ID NO:116). In some embodiments, the sequence of the 3E10-HC is at least 99% identical to 3E10-HC-h6m (SEQ ID NO: 116). In some embodiments, the sequence of the 3E10-HC is 3E10-HC-h6m (SEQ ID NO: 116).

In some embodiments, a humanized 3E10 antibody or antigen binding fragment thereof described herein, includes a light chain (3E10-LC) comprising an amino acid sequence that is at least 95% identical to an amino acid sequence selected from the group consisting of 3E10-LC-h1 (SEQ ID NO:137), 3E10-LC-h2 (SEQ ID NO:138), 3E10-LC-h3 (SEQ ID NO:139), 3E10-LC-h4 (SEQ ID NO:140), 3E10-LC-h5 (SEQ ID NO:141), and 3E10-LC-h6 (SEQ ID NO:142) and a heavy chain (3E10-HC) comprising an amino acid sequence that is at least 95% identical to an amino acid sequence selected from the group consisting of 3E10-HC-h1 (SEQ ID NO:118), 3E10-HC-h2 (SEQ ID NO:119), 3E10-HC-h3 (SEQ ID NO:120), 3E10-HC-h4 (SEQ ID NO: 121), 3E10-HC-h5 (SEQ ID NO:122), 3E10-HC-h6 (SEQ ID NO:123), and 3E10-HC-h7 (SEQ ID NO:124).

In some embodiments, the sequence of the 3E10-LC is at least 95% identical to 3E10-LC-h6 (SEQ ID NO:142). In some embodiments, the sequence of the 3E10-LC is at least 96% identical to 3E10-LC-h6 (SEQ ID NO:142). In some embodiments, the sequence of the 3E10-LC is at least 97% identical to 3E10-LC-h6 (SEQ ID NO: 142). In some embodiments, the sequence of the 3E10-LC is at least 98% identical to 3E10-LC-h6 (SEQ ID NO:142). In some embodiments, the sequence of the 3E10-LC is at least 99% identical to 3E10-LC-h6 (SEQ ID NO: 142). In some embodiments, the sequence of the 3E10-LC is 3E10-LC-h6 (SEQ ID NO: 142).

In some embodiments, the sequence of the 3E10-HC is at least 95% identical to 3E10-HC-h6 (SEQ ID NO:123). In some embodiments, the sequence of the 3E10-HC is at least 96% identical to 3E10-HC-h6 (SEQ ID NO:123). In some embodiments, the sequence of the 3E10-HC is at least 97% identical to 3E10-HC-h6 (SEQ ID NO:123). In some embodiments, the sequence of the 3E10-HC is at least 98% identical to 3E10-HC-h6 (SEQ ID NO:123). In some embodiments, the sequence of the 3E10-HC is at least 99% identical to 3E10-HC-h6 (SEQ ID NO: 123). In some embodiments, the sequence of the 3E10-HC is 3E10-HC-h6 (SEQ ID NO: 123).

In some embodiments, a humanized 3E10 antibody or antigen binding fragment thereof described herein comprises a combination of a heavy chain variable domain (VH) and a light chain variable domain (VL) comprising amino acid sequences having at least 95%, 96%, 97%, 98%, 99%, 99.5%, or 100% sequence identity to a pair of VL and VH selected from 3E10-VH-h1 and 3E10-VL-h1, 3E10-VH-h1 and 3E10-VL-h2, 3E10-VH-h1 and 3E10-VL-h3, 3E10-VH-h1 and 3E10-VL-h4, 3E10-VH-h2 and 3E10-VL-h1, 3E10-VH-h2 and 3E10-VL-h2, 3E10-VH-h2 and 3E10-VL-h3, 3E10-VH-h2 and 3E10-VL-h4, 3E10-VH-h3 and 3E10-VL-h1, 3E10-VH-h3 and 3E10-VL-h2, 3E10-VH-h3 and 3E10-VL-h3, 3E10-VH-h3 and 3E10-VL-h4, 3E10-VH-h4 and 3E10-VL-h1, 3E10-VH-h4 and 3E10-VL-h2, 3E10-VH-h4 and 3E10-VL-h3, 3E10-VH-h4 and 3E10-VL-h4, 3E10-VH-h5 and 3E10-VL-h5, 3E10-VH-h5 and 3E10-VL-h6, 3E10-VH-h6 and 3E10-VL-h5, 3E10-VH-h6 and 3E10-VL-h6, 3E10-VH-h7 and 3E10-VL-h5, and 3E10-VH-h7 and 3E10-VL-h6.

In some embodiments, a humanized 3E10 antibody or antigen binding fragment thereof described herein comprises a heavy chain variable domain (VH) comprising an amino acid sequence having at least 95%, 96%, 97%, 98%, 99%, 99.5%, or 100% sequence identity to 3E10-VH-h6 (SEQ ID NO:109) and a light chain variable domain (VL) comprising an amino acid sequence having at least 95%, 96%, 97%, 98%, 99%, 99.5%, or 100% sequence identity to 3E10-VL-h6 (SEQ ID NO:130).

In some embodiments, a humanized 3E10 antibody or antigen binding fragment thereof described herein includes a light chain variable domain (3E10-VL) comprising an amino acid sequence that is at least 90%, 91%, 92%, 93%, 94%, 95%, 96%, 97%, 98%, 99%, 99.5%, or 100% identical to an amino acid sequence selected from the group consisting of 3E10-VL-h1 (SEQ ID NO:125), 3E10-VL-h2 (SEQ ID NO:126), 3E10-VL-h3 (SEQ ID NO:127), 3E10-VL-h4 (SEQ ID NO:128), 3E10-VL-h5 (SEQ ID NO:129), and 3E10-VL-h6 (SEQ ID NO:130), where the light chain variable domain (3E10-VL) comprises one or more amino acid residues selected from proline (Pro) at position 15, threonine (Thr) at position 22, tyrosine (Tyr) at position 49, Thr at position 74, asparagine (Asn) at position 76, alanine (Ala) at position 80, Asn at position 81, Thr at position 83, Asn at position 85, and valine (Val) at position 104, of the 3E10-VL according to Kabat numbering, and a set of 3E10-VL CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), and where the antibody includes a set of 3E10-VL CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In some embodiments, the humanized 3E10 antibody or antigen binding fragment thereof includes a set of 3E10-VL CDRs comprising no more than 5 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs comprising no more than 4 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs comprising no more than 3 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO: 11). In some embodiments, the humanized 3E10 antibody or antigen binding fragment thereof includes a set of 3E10-VL CDRs comprising no more than 2 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs comprising no more than 1 amino acid substitution relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In some embodiments, the humanized 3E10 antibody or antigen binding fragment thereof has a lysine (Lys) residue at position 49 of the 3E10-VL according to Kabat numbering. In some embodiments, the humanized 3E10 antibody or antigen binding fragment thereof has a glutamic acid (Glu) residue at position 81 of the 3E10-VL according to Kabat numbering. In some embodiments, the humanized 3E10 antibody or antigen binding fragment thereof has a proline (Pro) residue at position 15 of the 3E10-VL according to Kabat numbering. In some embodiments, the humanized 3E10 antibody or antigen binding fragment thereof has a valine (Val) residue at position 104 of the 3E10-VL according to Kabat numbering.

In some embodiments, a humanized 3E10 antibody or antigen binding fragment thereof described herein includes a heavy chain variable domain (3E10-VH) comprising an amino acid sequence that is at least 90%, 91%, 92%, 93%, 94%, 95%, 96%, 97%, 98%, 99%, 99.5%, or 100% identical to an amino acid sequence selected from the group consisting of 3E10-VH-h1 (SEQ ID NO:104), 3E10-VH-h2 (SEQ ID NO:105), 3E10-VH-h3 (SEQ ID NO:106), 3E10-VH-h4 (SEQ ID NO:107), 3E10-VH-h5 (SEQ ID NO:108), 3E10-VH-h6 (SEQ ID NO:109), and 3E10-VH-h7 (SEQ ID NO:110), where the heavy chain variable domain (3E10-VH) comprises one or more amino acid residues selected from glutamine (Gln) at position 13, leucine (Leu) at position 18, arginine (Arg) at position 19, glycine (Gly) at position 42, serine (Ser) at position 49, Ser at position 77, tyrosine (Tyr) at position 79, Asn at position 82, Ala at position 84, Val at position 89, leucine (Leu) at position 108, Val at position 109, and Ser at position 113, of the 3E10-VH according to Kabat numbering, and where the antibody includes a set of 3E10-VH CDRs collectively having no more than 6 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5).

In some embodiments, the humanized 3E10 antibody or antigen binding fragment thereof includes a set of 3E10-VH CDRs comprising no more than 5 amino acid substitutions relative to the set of CDRs having the amino acid sequences of 3E10-VH-CDR1_D31N (SEQ ID NO: 15), 3E10-VH-CDR2 (SEQ ID NO:4), and 3E10-VH-CDR3 (SEQ ID NO:5). In some embodiments, the humanized 3E10 antibody or antigen binding fragment thereof includes a set of 3E10-VH CDRs comprising no more than 4 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes a set of 3E10-VH CDRs comprising no more than 3 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes set of 3E10-VH CDRs comprising no more than 2 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes a set of 3E10-VH CDRs comprising no more than 1 amino acid substitution relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO: 5). In some embodiments, the humanized 3E10 antibody or antigen binding fragment thereof includes a set of 3E10-VH CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO: 5).

In some embodiments, the humanized 3E10 antibody or antigen binding fragment thereof has an arginine (Arg) residue at position 18 of the 3E10-VH according to Kabat numbering. In some embodiments, the humanized 3E10 antibody or antigen binding fragment thereof has a (Lys) residue at position 19 of the 3E10-VH according to Kabat numbering. In some embodiments, the humanized 3E10 antibody or antigen binding fragment thereof has an alanine (Ala) residue at position 49 of the 3E10-VH according to Kabat numbering. In some embodiments, the humanized 3E10 antibody or antigen binding fragment thereof with a glutamine (Gln) residue at position 13 of the 3E10-VH according to Kabat numbering. In some embodiments, the humanized 3E10 antibody or antigen binding fragment thereof has a leucine (Leu) residue at position 108 of the 3E10-VH according to the Kabat numbering. In some embodiments, the humanized 3E10 antibody or antigen binding fragment thereof with a Val residue at position 109 of the 3E10-VH according to Kabat numbering. In some embodiments, the humanized 3E10 antibody or antigen binding fragment thereof has a serine (Ser) residue at position 113 of the 3E10-VH according to Kabat numbering.

### Cell Penetration and Nuclear Localization

The disclosed compositions and methods typically utilize antibodies that maintain the ability to penetrate cells, and optionally nuclei.

The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur. J. Immunol. 38, 3178-3190 (2008), Yanase et al., J. Clin. Invest. 100, 25-31 (1997)). 3E10 penetrates cells in an Fc-independent mechanism (as evidenced by the ability of 3E10 fragments lacking an Fc to penetrate cells) but involves presence of the nucleoside transporter ENT2 (Weisbart et al., Scientific Reports volume 5, Article number: 12022 (2015), Zack et al., J. Immunol. 157, 2082-2088 (1996), Hansen et al., J. Biol. Chem. 282, 20790-20793 (2007)). Thus, in some embodiments, the antibodies utilized in the disclosed compositions and methods are ones that penetrates cells in an Fc-independent mechanism but involves presence of the nucleoside transporter ENT2.

Mutations in 3E10 that interfere with its ability to bind DNA may render the antibody incapable of nuclear penetration. Thus, typically the disclosed variants and humanized forms of the antibody maintain the ability to bind nucleic acids, particularly DNA. In addition, 3E10 scFv has previously been shown capable of penetrating into living cells and nucleic in an ENT2-dependent manner, with efficiency of uptake impaired in ENT2-deficient cells (Hansen, et al., J. Biol. Chem. 282, 20790-20793 (2007)). Thus, in some embodiments, the disclosed variants and humanized forms of the antibody maintain the ability to penetrate into cell nuclei in an ENT-dependent, preferably ENT2-dependent manner.

As discussed in US 2021/0054102 and US 2021/0137960, some humanized 3E10 variant were found to penetrate cell nuclei more efficiently than the original murine 3E10 (D31N) di-scFv, while others were found to have lost the ability to penetrate nuclei. In particular, variants 10 and 13 penetrated nuclei very well compared to the murine antibody.

Potential bipartite nuclear localization signals (NLS) in humanized 3E10 VL have been identified and may include part or all of the following sequences:

(SEQ ID NO: 143)
RASKSVSTSSYSYMHWYQQKPGQPPKLLIKY;
(SEQ ID NO: 144)
RASKTVSTSSYSYMHWYQQKPGQPPKLLIKY;
or
(SEQ ID NO: 145)
RVTITCRASKSVSTSSYSYMHWYQQKPGKAPKL.

An example consensus NLS can be, or include, (X)RASKTVSTSSYSYMHWYQQKPGQPPKLL(X)KY (where (X)=any residue, but preferentially is a basic residue (R or K) (SEQ ID NO: 146) or a variant thereof with at least 60, 65, 70, 75, 80, 85, 90, 95, 96, 97, 98, 99 percent sequence identity to SEQ ID NO:147.

Thus, in some embodiments, particularly where nuclear importation is important, the disclosed antibodies may include the sequence of any one of SEQ ID NOs: 143-147, or fragments and variants thereof (e.g., at least 70, 75, 80, 85, 90, 95, 96, 97, 98, 99, or 100% amino acid sequence identity with any one of SEQ ID NOs: 143-147) that can translocate into the nucleus of a cell.

Presence of an NLS indicates that a humanized 3E10 antibody or antigen binding fragment thereof may cross the nuclear envelope via the nuclear import pathway. In some embodiments, the NLS improves importation by interacting with one or more members of the import pathway. Thus, in some embodiments, the NLS can bind to importin-β, an importin-β/importin-α heterodimer, or a combination thereof.

### Nucleic Acid Binding

In some embodiments, the disclosed compositions and methods utilize humanized 3E10 antibodies and ENT2-binding fragments thereof that maintain the ability to bind nucleic acids such as DNA, RNA.

The Examples below illustrate molecular modeling of wild type 3E10 sequences and additional 3E10 variants. Molecular modeling of 3E10 (Pymol) revealed a putative Nucleic Acid Binding pocket (NAB1) (See, e.g., FIGS. 11A and 11B, and illustrated with underlining the sequences below).

WT HEAVY CHAIN scFv SEQUENCE
(SEQ ID NO: 148)
E VQLVESGGGL VKPGGSRKLS CAASGFTESD YGMHWVRQAP
EKGLEWVAYI SSGSSTIYYA DTVKGRFTIS RDNAKNTLFL
QMTSLRSEDT AMYYCARRGL LLDYWGQGTT LTVS
LIGHT CHAIN scFv SEQUENCE
(SEQ ID NO:149)
D IVLTQSPASL AVSLGQRATI SCRASKSVST SSYSYMHWYQ
QKPGQPPKLL IKYASYLESG VPARFSGSGS GTDFTLNIHP
VEEEDAATYY CQHSREFPWT FGGGTKLEIK RADAAPGGGG
SGGGGSGGGGS

In some embodiments, the disclosed humanized 3E10 antibodies include some or all of the underlined NAB1 sequences. In some embodiments, the humanized 3E10 antibodies include a variant sequence that has an altered ability of bind nucleic acids. In some embodiments, the mutations (e.g., substitutions, insertions, and/or deletions) in the NAB1 improve binding of the antibody to nucleic acids such as DNA, RNA, or a combination thereof. In some embodiments, the mutations are conservative substitutions. In some embodiments, the mutations increase the cationic charge of the NAB1 pocket.

As discussed and exemplified herein, mutation of aspartic acid at residue 31 of CDR1 to asparagine increased the cationic charge of this residue and enhanced nucleic acid binding and delivery in vivo (3E10-D31N).

Additional example variants include mutation of aspartic acid at residue 31 of CDR1 to arginine (3E10-D31R), which modeling indicates expands cationic charge, or lysine (3E10-D31K) which modeling indicates changes charge orientation. Thus, in some embodiments, the 3E10 binding protein includes a D31R or D31K substitution.

Additional example variants include mutation of arginine (R) 96 to asparagine (N), and/or serine(S) 30 to aspartic acid (D) alone or in combination with D31N, D31R, or D31K.

All of the sequences disclosed herein having the residue corresponding to 3E10 D31 or N31, are expressly disclosed with a D31R or D31K or N31R or N31K substitution.

Molecular modeling of 3E10 (Pymol) revealed a putative Nucleic Acid Binding pocket (NAB1) (FIGS. 11A-11B). Mutation of aspartic acid at residue 31 of CDR1 to asparagine increased the cationic charge of this residue and enhanced nucleic acid binding and delivery in vivo (3E10-D31N).

Mutation of aspartic acid at residue 31 of CDR1 to arginine (3E10-D31R), further expanded the cationic charge while mutation to lysine (3E10-D31K) changed charge orientation (FIG. 11A).

NAB1 amino acids predicted from molecular modeling have been underlined in the heavy and light chain sequences above. FIG. 11B is an illustration showing molecular modeling of 3E10-scFv (Pymol) with NAB1 amino acid residues illustrated with punctate dots.

All of the sequences disclosed herein having the residue corresponding with R96 are expressly disclosed with R96N substitution.

All of the sequence disclosed herein having the residue corresponding to S30 are expressly disclosed with S30D.

Any of the substitutions can be included in any combination. The sequence having two or three substitutions at any combination of residues 31, 30, and 96 are expressly provided.

In particular embodiments, the sequence has 31N, 31K, or 31R alone or in combination with 30D, and without the R96N substitution. Thus, in some embodiments, the residue corresponding to 96 is not N, and in more specific embodiments remains R.

### Antisense Oligonucleotides

To identify and select antisense oligonucleotides suitable for use in the modulation of exon skipping, a nucleic acid sequence whose function is to be modulated must first be identified. This may be, for example, a gene (or mRNA transcribed form the gene) whose expression is associated with a particular disorder or disease state, e.g., Duchenne muscular dystrophy. Within the context of the present disclosure, preferred target site(s) are those involved in mRNA splicing (i.e., splice donor sites, splice acceptor sites, or exonic splicing enhancer elements). Splicing branch points and exon recognition sequences or splice enhancers are also potential target sites for modulation of mRNA splicing. The disclosure provides antisense oligonucleotides capable of binding to a selected target in the pre-mRNA to induce efficient and consistent exon skipping.

The antisense oligonucleotides and pre-mRNA are complementary to each other when a sufficient number of corresponding positions in each molecule are occupied by nucleotides which can hydrogen bond with each other. Thus, the term “complementary” is used to indicate a sufficient degree of complementarity or precise pairing such that stable and specific binding occurs between the oligonucleotide and the DNA or RNA target. It is understood in the art that the sequence of an antisense molecule need not be 100% complementary to that of its target sequence to interfere with the normal function of the target DNA or RNA as well as to avoid non-specific binding of the antisense oligonucleotide to non-target sequences under conditions in which specific binding is desired, i.e., under physiological conditions in the case of in vivo assays or therapeutic treatment.

The length of an antisense oligonucleotide may vary so long as it is capable of binding selectively to the intended location within the pre-mRNA molecule. The length of such sequences can be determined in accordance with selection procedures described herein. Generally, the antisense oligomer will be from about 10 nucleotides in length up to about 50 nucleotides in length. It will be appreciated however that any length of nucleotides within this range may be used in the method. Preferably, the length of the antisense molecule is between 17 to 30 nucleotides in length.

To avoid degradation of pre-mRNA during duplex formation with the antisense oligomers, the antisense oligomers used in the method may be adapted to minimize or prevent cleavage by endogenous RNase H. This property is highly preferred as the treatment of the RNA with the unmethylated oligonucleotides either intracellularly or in crude extracts that contain RNase H leads to degradation of the pre-mRNA: antisense oligomer duplexes. Any form of modified antisense molecules that is capable of bypassing or not inducing such degradation may be used in the present method. An example of antisense oligomer which when duplexed with RNA are not cleaved by cellular RNase His 2′-O-methyl derivatives. 2′-O-methyl-oligoribonucleotides are very stable in a cellular environment and in animal tissues, and their duplexes with RNA have higher Tm values than their ribo- or deoxyribo-counterparts.

Antisense oligonucleotides that do not activate RNase H can be made in accordance with known techniques, see, e.g., U.S. Pat. No. 5,149,797. Such antisense oligonucleotides, which may be deoxyribonucleotide or ribonucleotide sequences, simply contain any structural modification which sterically hinders or prevents binding of RNase H to a duplex molecule containing the oligonucleotide as one member thereof, which structural modification does not substantially hinder or disrupt duplex formation. Because the portions of the oligonucleotide involved in duplex formation are substantially different from those portions involved in RNase H binding thereto, numerous antisense molecules that do not activate RNase H are available. For example, such antisense oligonucleotides wherein at least one, or all, of the internucleotide bridging phosphate residues are modified phosphates, such as methyl phosphonates, methyl phosphorothioates, phosphoromorpholidates, phosphoropiperazidates and phosphoramidates. For example, every other one of the internucleotide bridging phosphate residues may be modified as described. In another non-limiting example, such antisense oligonucleotides are oligonucleotides wherein at least one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

The present disclosure appreciates other antisense oligonucleotides including, but not limited to, oligonucleotide mimetics.

Specific examples of preferred antisense compounds useful in this disclosure include oligonucleotides containing modified backbones or non-natural inter-nucleoside linkages. Oligonucleotides having modified backbones include those that retain a phosphorus atom in the backbone and those that do not have a phosphorus atom in the backbone. For the purposes of this disclosure, and referenced in the art, modified oligonucleotides that do not have a phosphorus atom in their inter-nucleoside backbone can also be considered to be oligonucleosides.

In some embodiments, the oligonucleotide mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA).

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligomers obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications. The backbone is uncharged, resulting in PNA/DNA or PNA/RNA duplexes that exhibit greater than normal thermal stability. PNAs are not recognized by nucleases or proteases.

Modified oligonucleotides may also contain one or more substituted sugar moieties. Oligonucleotides may also include nucleobase (often referred to in the art simply as “base”) modifications or substitutions. Certain nucleo-bases are particularly useful for increasing the binding affinity of the oligomeric compounds of the disclosure. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the disclosure involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

It is not necessary for all positions in a given compound to be uniformly modified, and in fact more than one of the aforementioned modifications may be incorporated in a single compound or even at a single nucleoside within an oligonucleotide. The present disclosure also includes antisense that are chimeric compounds.

“Chimeric” antisense compounds or “chimeras,” in the context of this invention, are antisense molecules, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the increased resistance to nuclease degradation, increased cellular uptake, and an additional region for increased binding affinity for the target nucleic acid.

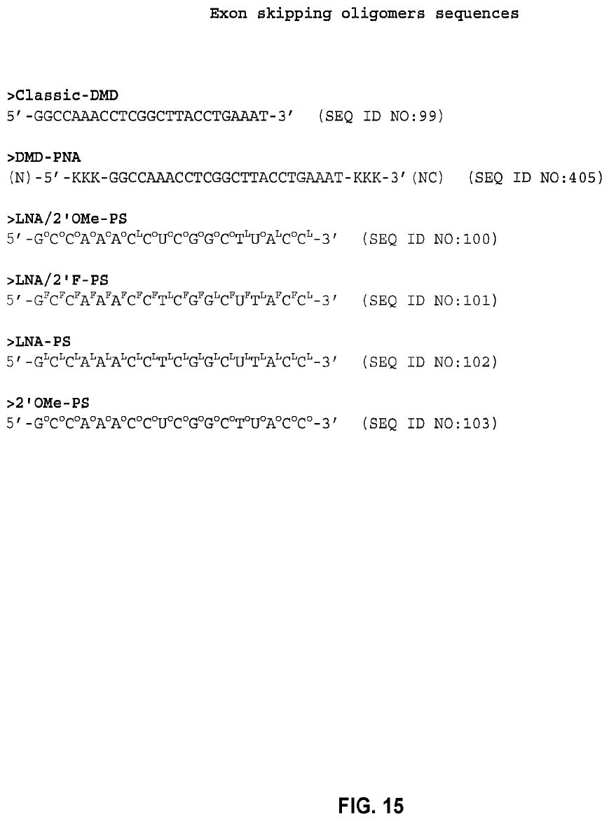

In some embodiments, the exon-skipping inducing oligonucleotides have a sequence selected from SEQ ID NO:99-103, as detailed in FIG. 15, or any variation thereof disclosed herein. In other embodiments, the exon-skipping inducing oligonucleotides can be selected from the group consisting of SEQ ID NO:150-398, as detailed in Table 2. It should be understood that the oligonucleotides disclosed in Table 2 are exemplary in nature, and in no way limiting the present invention.

### 3E10/3E10 Variant-Therapeutic Antisense Oligonucleotide Compositions

In one aspect, the present disclosure provides pharmaceutical compositions including a complex formed between a therapeutic oligonucleotide, e.g., as described above, and a 3E10 antibody or antigen-binding fragment thereof, as described herein.

In some embodiments, a pharmaceutical composition described herein has a molar ratio of 3E10 antibody or antigen-binding fragment thereof to therapeutic oligonucleotide of at least 2:1. As reported in Example 6, the use of molar ratios of 3E10 antibody or antigen-binding fragment thereof to therapeutic oligonucleotides in the compositions described herein protects the therapeutic oligonucleotide from degradation.

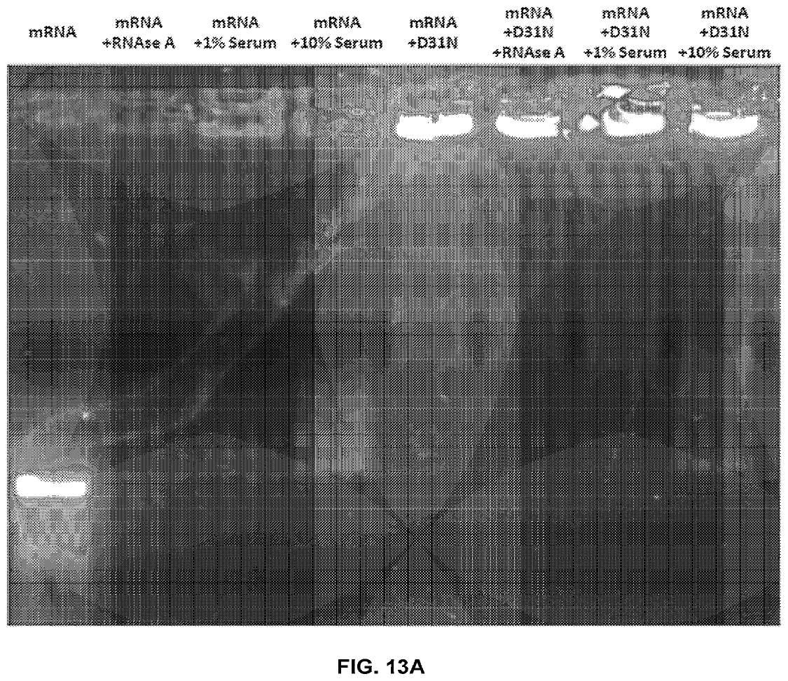

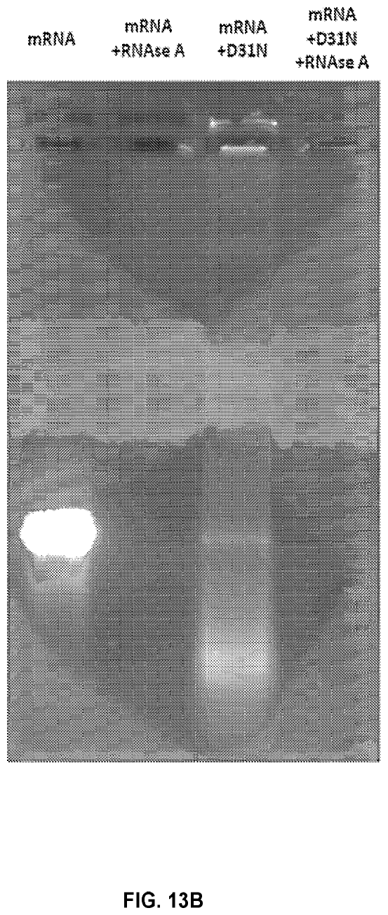

Further, as illustrated in FIGS. 13A and 13B, while parental 3E10 and 3E10 (D31N) variant antibodies protected mRNA from RNAse A-mediated RNA degradation at molar ratios of 2:1 and 20:1, the protection afforded by the 20:1 molar ratio exceeded the protection afforded at 2:1. Accordingly, in some embodiments, a pharmaceutical composition described herein has a molar ratio of 3E10 antibody or antigen-binding fragment thereof to therapeutic oligonucleotide that is at least 2:1. In some embodiments, a pharmaceutical composition described herein has a molar ratio of 3E10 antibody or antigen-binding fragment thereof to therapeutic oligonucleotide that is least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, at least 20:1, at least 21:1, at least 22:1, at least 23:1, at least 24:1, at least 25:1, at least 26:1, at least 27:1, at least 28:1, at least 29:1, at least 30:1, at least 31:1, at least 32:1, at least 33:1, at least 34:1, at least 35:1, at least 36:1, at least 37:1, at least 38:1, at least 39:1, at least 40:1, at least 41:1, at least 42:1, at least 43:1, at least 44:1, at least 45:1, or greater.

In some embodiments, a pharmaceutical composition described herein has a molar ratio of 3E10 antibody or antigen-binding fragment thereof to therapeutic oligonucleotide that is no more than 50:1. In some embodiments, a pharmaceutical composition described herein has a molar ratio of 3E10 antibody or antigen-binding fragment thereof to therapeutic oligonucleotide that is no more than 50:1, no more than 49:1, no more than 48:1, no more than 47:1, no more than 46:1, no more than 45:1, no more than 44:1, no more than 43:1, no more than 42:1, no more than 41:1, no more than 40:1, no more than 39:1, no more than 38:1, no more than 37:1, no more than 36:1, no more than 35:1, no more than 34:1, no more than 33:1, no more than 32:1, no more than 31:1, no more than 30:1, no more than 29:1, no more than 28:1, no more than 27:1, no more than 26:1, no more than 25:1, no more than 24:1, no more than 23:1, no more than 22:1, no more than 21:1, no more than 20:1, no more than 19:1, no more than 18:1, no more than 17:1, no more than 16:1, no more than 15:1, no more than 14:1, no more than 13:1, no more than 12:1, no more than 11:1, no more than 10:1, no more than 9:1, no more than 8:1, no more than 7:1, no more than 6:1, no more than 5:1, or less.

In some embodiments, a pharmaceutical composition described herein has a molar ratio of 3E10 antibody or antigen-binding fragment thereof to therapeutic oligonucleotide that is of from about 2:1 to about 50:1. In some embodiments, a pharmaceutical composition described herein has a molar ratio of 3E10 antibody or antigen-binding fragment thereof to therapeutic oligonucleotide that is of from about 2:1 to about 40:1. In some embodiments, a pharmaceutical composition described herein has a molar ratio of 3E10 antibody or antigen-binding fragment thereof to therapeutic oligonucleotide that is of from about 2:1 to about 30:1. In some embodiments, a pharmaceutical composition described herein has a molar ratio of 3E10 antibody or antigen-binding fragment thereof to therapeutic oligonucleotide that is of from about 2:1 to about 25:1. In some embodiments, a pharmaceutical composition described herein has a molar ratio of 3E10 antibody or antigen-binding fragment thereof to therapeutic oligonucleotide that is of from about 2:1 to about 20:1. In some embodiments, a pharmaceutical composition described herein has a molar ratio of 3E10 antibody or antigen-binding fragment thereof to therapeutic oligonucleotide that is of from about 2:1 to about 15:1. In some embodiments, a pharmaceutical composition described herein has a molar ratio of 3E10 antibody or antigen-binding fragment thereof to therapeutic oligonucleotide that is of from about 2:1 to about 10:1. In some embodiments, a pharmaceutical composition described herein has a molar ratio of 3E10 antibody or antigen-binding fragment thereof to therapeutic oligonucleotide that is of from about 2:1 to about 7.5:1. In some embodiments, a pharmaceutical composition described herein has a molar ratio of 3E10 antibody or antigen-binding fragment thereof to therapeutic oligonucleotide that is of from about 2:1 to about 5:1. In some embodiments, a pharmaceutical composition described herein has a molar ratio of 3E10 antibody or antigen-binding fragment thereof to therapeutic oligonucleotide that is of from about 2:1 to about 5:1. In some embodiments, a pharmaceutical composition described herein has a molar ratio of 3E10 antibody or antigen-binding fragment thereof to therapeutic oligonucleotide that is of from about 2:1 to about 3:1.

In some embodiments, a pharmaceutical composition described herein has a molar ratio of 3E10 antibody or antigen-binding fragment thereof to therapeutic oligonucleotide that is of from about 5:1 to about 50:1. In some embodiments, a pharmaceutical composition described herein has a molar ratio of 3E10 antibody or antigen-binding fragment thereof to therapeutic oligonucleotide that is of from about 5:1 to about 40:1. In some embodiments, a pharmaceutical composition described herein has a molar ratio of 3E10 antibody or antigen-binding fragment thereof to therapeutic oligonucleotide that is of from about 5:1 to about 30:1. In some embodiments, a pharmaceutical composition described herein has a molar ratio of 3E10 antibody or antigen-binding fragment thereof to therapeutic oligonucleotide that is of from about 5:1 to about 25:1. In some embodiments, a pharmaceutical composition described herein has a molar ratio of 3E10 antibody or antigen-binding fragment thereof to therapeutic oligonucleotide that is of from about 5:1 to about 20:1. In some embodiments, a pharmaceutical composition described herein has a molar ratio of 3E10 antibody or antigen-binding fragment thereof to therapeutic oligonucleotide that is of from about 5:1 to about 15:1. In some embodiments, a pharmaceutical composition described herein has a molar ratio of 3E10 antibody or antigen-binding fragment thereof to therapeutic oligonucleotide that is of from about 5:1 to about 10:1. In some embodiments, a pharmaceutical composition described herein has a molar ratio of 3E10 antibody or antigen-binding fragment thereof to therapeutic oligonucleotide that is of from about 5:1 to about 7.5:1.

In some embodiments, a pharmaceutical composition described herein has a molar ratio of 3E10 antibody or antigen-binding fragment thereof to therapeutic oligonucleotide that is of from about 10:1 to about 50:1. In some embodiments, a pharmaceutical composition described herein has a molar ratio of 3E10 antibody or antigen-binding fragment thereof to therapeutic oligonucleotide that is of from about 10:1 to about 40:1. In some embodiments, a pharmaceutical composition described herein has a molar ratio of 3E10 antibody or antigen-binding fragment thereof to therapeutic oligonucleotide that is of from about 10:1 to about 30:1. In some embodiments, a pharmaceutical composition described herein has a molar ratio of 3E10 antibody or antigen-binding fragment thereof to therapeutic oligonucleotide that is of from about 10:1 to about 25:1. In some embodiments, a pharmaceutical composition described herein has a molar ratio of 3E10 antibody or antigen-binding fragment thereof to therapeutic oligonucleotide that is of from about 10:1 to about 20:1. In some embodiments, a pharmaceutical composition described herein has a molar ratio of 3E10 antibody or antigen-binding fragment thereof to therapeutic oligonucleotide that is of from about 10:1 to about 15:1.

In some embodiments, a pharmaceutical composition described herein has a molar ratio of 3E10 antibody or antigen-binding fragment thereof to therapeutic oligonucleotide that is of from about 15:1 to about 50:1. In some embodiments, a pharmaceutical composition described herein has a molar ratio of 3E10 antibody or antigen-binding fragment thereof to therapeutic oligonucleotide that is of from about 15:1 to about 40:1. In some embodiments, a pharmaceutical composition described herein has a molar ratio of 3E10 antibody or antigen-binding fragment thereof to therapeutic oligonucleotide that is of from about 15:1 to about 30:1. In some embodiments, a pharmaceutical composition described herein has a molar ratio of 3E10 antibody or antigen-binding fragment thereof to therapeutic oligonucleotide that is of from about 15:1 to about 25:1. In some embodiments, a pharmaceutical composition described herein has a molar ratio of 3E10 antibody or antigen-binding fragment thereof to therapeutic oligonucleotide that is of from about 15:1 to about 20:1.

In some embodiments, a pharmaceutical composition described herein has a molar ratio of 3E10 antibody or antigen-binding fragment thereof to therapeutic oligonucleotide that is of from about 20:1 to about 50:1. In some embodiments, a pharmaceutical composition described herein has a molar ratio of 3E10 antibody or antigen-binding fragment thereof to therapeutic oligonucleotide that is of from about 20:1 to about 40:1. In some embodiments, a pharmaceutical composition described herein has a molar ratio of 3E10 antibody or antigen-binding fragment thereof to therapeutic oligonucleotide that is of from about 20:1 to about 30:1. In some embodiments, a pharmaceutical composition described herein has a molar ratio of 3E10 antibody or antigen-binding fragment thereof to therapeutic oligonucleotide that is of from about 20:1 to about 25:1.

In some embodiments, a pharmaceutical composition described herein has a molar ratio of 3E10 antibody or antigen-binding fragment thereof to therapeutic oligonucleotide that is of from about 25:1 to about 50:1. In some embodiments, a pharmaceutical composition described herein has a molar ratio of 3E10 antibody or antigen-binding fragment thereof to therapeutic oligonucleotide that is of from about 25:1 to about 40:1. In some embodiments, a pharmaceutical composition described herein has a molar ratio of 3E10 antibody or antigen-binding fragment thereof to therapeutic oligonucleotide that is of from about 25:1 to about 30:1. In some embodiments, a pharmaceutical composition described herein has a molar ratio of 3E10 antibody or antigen-binding fragment thereof to therapeutic oligonucleotide that is of from about 30:1 to about 50:1. In some embodiments, a pharmaceutical composition described herein has a molar ratio of 3E10 antibody or antigen-binding fragment thereof to therapeutic oligonucleotide that is of from about 30:1 to about 40:1. In yet other embodiments, other ranges falling with the range of about 2:1 to about 50:1 are contemplated.

In some embodiments, a pharmaceutical composition described herein has a molar ratio of 3E10 antibody or antigen-binding fragment thereof to therapeutic oligonucleotide that is of from 2:1 to 50:1, from 2:1 to 40:1, from 2:1 to 30:1, from 2:1 to 25:1, from 2:1 to 20:1, from 2:1 to 15:1, from 2:1 to 10:1, from 2:1 to 7.5:1, from 2:1 to 5:1, from 5:1 to 50:1, from 5:1 to 40:1, from 5:1 to 30:1, from 5:1 to 25:1, from 5:1 to 20:1, from 5:1 to 15:1, from 5:1 to 10:1, from 5:1 to 7.5:1, from 10:1 to 50:1, from 10:1 to 40:1, from 10:1 to 30:1, from 10:1 to 25:1, from 10:1 to 20:1, from 10:1 to 15:1, from 15:1 to 50:1, from 15:1 to 40:1, from 15:1 to 30:1, from 15:1 to 25:1, from 15:1 to 20:1, from 20:1 to 50:1, from 20:1 to 40:1, from 20:1 to 30:1, from 20:1 to 25:1, from 25:1 to 50:1, from 25:1 to 40:1, from 25:1 to 30:1, from 30: to 50:1, from 30:1 to 40:1, or from 40:1 to 50:1. In yet other embodiments, other ranges falling with the range of from 2:1 to 50:1 are contemplated.

In some embodiments, a pharmaceutical composition described herein has a molar ratio of 3E10 antibody or antigen-binding fragment thereof to therapeutic oligonucleotide that is of from about 1:1 to about 50:1. In some embodiments, a pharmaceutical composition described herein has a molar ratio of 3E10 antibody or antigen-binding fragment thereof to therapeutic oligonucleotide that is of from about 1:1 to about 30:1. In some embodiments, a pharmaceutical composition described herein has a molar ratio of 3E10 antibody or antigen-binding fragment thereof to therapeutic oligonucleotide that is of from about 1:1 to about 20:1. In some embodiments, a pharmaceutical composition described herein has a molar ratio of 3E10 antibody or antigen-binding fragment thereof to therapeutic oligonucleotide that is of from about 1:1 to about 10:1. In some embodiments, a pharmaceutical composition described herein has a molar ratio of 3E10 antibody or antigen-binding fragment thereof to therapeutic oligonucleotide that is of from about 1:1 to about 5:1.

In some embodiments, a pharmaceutical composition described herein has a molar ratio of 3E10 antibody or antigen-binding fragment thereof to therapeutic oligonucleotide that is of from 1:50 to 10:1. Particularly, in some embodiments where the therapeutic polynucleotide is smaller, e.g., less than 1000 nucleotides, less than 500 nucleotides, less than 250 nucleotides, less than 100 nucleotides, or less than 50 polynucleotides, the ratio of antibody to polynucleotide closer to 1:1, or the polynucleotide is in molar excess to the antibody. For example, in some embodiments, the composition comprises a molar ratio of (i) 3E10 antibody or antigen-binding fragment thereof to (ii) therapeutic polynucleotide of no more than 10:1. In some embodiments, the composition comprises a molar ratio of (i) 3E10 antibody or antigen-binding fragment thereof to (ii) therapeutic polynucleotide of no more than 1:1. In some embodiments, the composition comprises a molar ratio of (i) 3E10 antibody or antigen-binding fragment thereof to (ii) therapeutic polynucleotide of no more than 1:3. In some embodiments, the composition comprises a molar ratio of (i) 3E10 antibody or antigen-binding fragment thereof to (ii) therapeutic polynucleotide of no more than 1:5. In some embodiments, the composition comprises a molar ratio of (i) 3E10 antibody or antigen-binding fragment thereof to (ii) therapeutic polynucleotide of no more than 1:10. In some embodiments, the composition comprises a molar ratio of (i) 3E10 antibody or antigen-binding fragment thereof to (ii) therapeutic polynucleotide of at least 1:50. In some embodiments, the composition comprises a molar ratio of (i) 3E10 antibody or antigen-binding fragment thereof to (ii) therapeutic polynucleotide of at least 1:25.

Because 3E10 antibodies or variants thereof, or antigen-binding fragments thereof localize to tissues in vivo following systemic administration, the compositions of the present disclosure can be formulated for, and subsequently administered by, one of many common administrative routes. In some embodiments, the pharmaceutical composition is formulated for parenteral administration. In some embodiments, the parenteral administration is intramuscular administration, intravenous administration, or subcutaneous administration.

### Diseases

The compositions described herein are well suited for the delivery of antisense oligonucleotides useful for treating various genetic diseases. Examples of proteins, and their associated genes, that are mutated in various neurogenetic, cardiovascular, metabolic, cancer, musculoskeletal, and lung diseases are presented in Table 1. Generally, sequences encoding, or complementary to sequences encoding, any one of these proteins, and variants thereof retaining a function of the full-length protein, can be included in the therapeutic polynucleotides disclosed herein. Accordingly, in some embodiments, the polypeptide is selected from the group consisting of ataxia telangiectasia mutated (ATM), phosphomannomutase 2 (PMM2), microtubule-associated protein tau (MAPT), Niemann-Pick C1 (NPC1), Neurofibromin (NF1), Merlin (NF2), Oligomeric plasma membrane (MLC1), Proteolipid protein (PLP1), Inhibitor kinase complex-associated protein (IKBKAP aka ELP1), Survival of motor neuron (SMN2), Dystrophia myotonica protein kinase (DMPK), Cellular nucleic acid-binding protein (CNBP/ZNF9), Jagged canonical Notch ligand 1 (JAG1), TSC complex subunit 2 (TSC2), usherin (USH2A), adenosine deaminase RNA specific (ADAR), ATPase Na+/K+ transporting subunit alpha 2 (ATP1A2), prickle planar cell polarity protein 2 (PRICKLE2), SET domain containing 5 (SETD5), eukaryotic translation initiation factor 2B subunit epsilon (EIF2B5), eukaryotic translation initiation factor 2B alpha (EIF2B1), eukaryotic translation initiation factor 2B subunit beta (EIF2B2), peroxisomal bio-genesis factor 1 (PEX1), syntaxin binding protein 1 (STXBP1), proline rich transmembrane protein 2 (PRRT2), syntaxin 1B (STX1B), retinoic acid induced 1 (RAI1), transcription factor 4 (TCF4), calcium voltage-gated channel subunit alpha 1 A (CACNA1A), DNA methyltransferase 1 (DNMT1), SH3 and multiple ankyrin repeat domain 3 (SHANK3), arylsulfatase A (ARSA), Prelamin A (LMNA), dystrophin (DMD), myostatin (MSTN), phenylalanine hydroxylase (PAH), and Apolipoprotein B (APOB).

### Neurogenetic Diseases

Neurogenetic diseases are typically characterized by mutations that affect the splicing process The brain expresses a relatively higher number of alternatively spliced genes, some of which were found to be linked to several neurological, neuromuscular, and neurodegenerative diseases. Because the etiology of many different forms of neurogenetic diseases have been well characterized, gene therapies offer an attractive option for treating these diseases. In fact, there are approved therapies and ongoing clinical trials for such gene therapies for several neurogenetic diseases (Siva K, Covello G, Denti M A. Exon-skipping antisense oligonucleotides to correct missplicing in neurogenetic diseases. Nucleic Acid Ther. 2014 February; 24 (1): 69-86).

### CDG

One such disorder for which a gene therapy is being developed is congenital disorder of glycosylation (CDG). CDG is an autosomal recessive disorder that affects glycan synthesis. The most prevalent form of CDG, type 1a (OMIM 212065), has an incidence of 1 in 50,000 to 1 in 100,000 individuals, and is caused by mutations in PMM2 gene, which is located on chromosome 16p13 and the gene encodes phosphomannomutase 2 protein (PMM2), a key enzyme that controls the synthesis of GDP-mannose which is essential for the generation of N-glycans. Mutations in the PMM2 gene lead to the hypoglycosylation of different proteins in different tissues (Dupré et al. 2000, Glycobiology 10, 1277-1128). Since the total lack of the PMM2 gene product is lethal, no patient with two copies of any inactivating mutation has ever been recorded. In one study, two different 25-nt-long phosphorodiamidate morpholino oligomers (PMOs) were designed, complementary to the 5′ or 3′ cryptic splice sites of the pseudoexon in intron 7 and both these PMOs were transfected at the same time into patient's fibroblasts carrying the c.640-15479C>T mutation in heterozygosity with missense mutationc.691G>A. The results showed that there was a 100% restoration of the correctly spliced mRNA. The levels of PMM2 protein after transfection increased from 9% to 23% of the quantity detected for the control cell line. PMM2 enzymatic activity was rescued almost to 50% that of the control fibroblasts (Vega et al., 2009, Hum. Mutat. 30 (5); 795-803).

In some embodiments, the methods and compositions described herein are useful for treating CDG, by delivering an antisense oligonucleotide capable of inducing skipping of an exon in PMM2 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, or 8 target regions of the PMM2 gene and induce exon skipping in at least one of exon 1, 2, 3, 4, 5, 6, 7, or 8. In some embodiments, the disclosure relates to antisense oligonucleotides complementary to an exon 2 target region of the PMM2 pre-mRNA designated as an annealing site. In some embodiments, the disclosure relates to antisense oligonucleotides complementary to an exon 3 target region of the PMM2 pre-mRNA designated as an annealing site. In some embodiments, the disclosure relates to antisense oligonucleotides complementary to an exon 4 target region of the PMM2 pre-mRNA designated as an annealing site. In some embodiments, the disclosure relates to antisense oligonucleotides complementary to an exon 5 target region of the PMM2 pre-mRNA designated as an annealing site. In some embodiments, the disclosure relates to antisense oligonucleotides complementary to an exon 6 target region of the PMM2 pre-mRNA designated as an annealing site. In some embodiments, the disclosure relates to antisense oligonucleotides complementary to an exon 7 target region of the PMM2 pre-mRNA designated as an annealing site.

In some embodiments, antisense oligonucleotides of the disclosure target PMM2 pre-mRNA and induces skipping of exon 1, 2, 3, 4, 5, 6, 7, and/or 8, so it is excluded or skipped from the mature, spliced mRNA transcript. By skipping exon 1, 2, 3, 4, 5, 6, 7, and/or 8, the disrupted reading frame is restored to an in-frame mutation. While CDG is comprised of various genetic subtypes, antisense oligonucleotides of the disclosure were specifically designed to skip exon 1, 2, 3, 4, 5, 6, 7, and/or 8, of PMM2 pre-mRNA (Vuillaumier-Barrot et al. Hum Mutat. 1999; 14 (6); 543-544; Gonazlez-Dominguez et al., Mol Genet and Metab Rep 2021, 28; 100781).

In some embodiments, the nucleobase sequence of an antisense oligonucleotides that induces exon 1, 2, 3, 4, 5, 6, 7, and/or 8, skipping is designed to be complementary to a specific target sequence within exon 1, 2, 3, 4, 5, 6, 7, and/or 8, of PMM2 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-TAGCTGCAAAGCAAGTGAAGCGGAC-3′ (SEQ ID NO: 150) or 5′-ATCACAAACACAACCTACCTCAGGC-3′ (SEQ ID NO:151) to target the PMM2 gene (Table 2).

### FD

A congenital neurodevelopmental disease is familial dysautonomia (OMIM 223900) which is characterized by unusually low numbers of neurons in the sensory and autonomic nervous systems. The resulting symptoms of patients include gastrointestinal dysfunction, scoliosis, and pain insensitivity. This disease is especially prevalent in the Ashkenazi Jewish population, where 1/3600 live births present familial dysautonomia. The genetic cause of familial dysautonomia was localized to a dysfunctional region spanning 177 kb on chromosome 9q31. The IKBKAP gene, one of the five genes identified in that region, was found to have a single-base mutation in over 99.5% of cases of observed familial dysautonomia (Slaugenhaupt S A et al. American Journal of Human Genetics. 68 (3): 598-605; U.S. Pat. No. 10,344,282, herein incorporated by reference in its entirety). The single-base mutation within the IKBKAP gene, is a transition from cytosine to thymine, and is present in the 5′ splice donor site of intron 20 in the IKBKAP pre-mRNA. This prevents recruitment of splicing machinery, and thus exon 19 is spliced directly to exon 21 in the final mRNA product-exon 20 is removed from the pre-mRNA with the introns.

In some embodiments, the methods and compositions described herein are useful for treating familial dysautonomia, by delivering an antisense oligonucleotide capable of inducing skipping of an exon in IKBKAP pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 target regions of the IKBKAP gene and induce exon skipping in at least one of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37. In some embodiments, the disclosure relates to antisense oligonucleotides complementary to an exon 20 target region of the IKBKAP pre-mRNA designated as an annealing site.

In some embodiments, antisense oligonucleotides of the disclosure target IKBKAP pre-mRNA and induces skipping of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and/or 37, so it is excluded or skipped from the mature, spliced mRNA transcript. By skipping exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and/or 37, the disrupted reading frame is restored to an in-frame mutation. While familial dysautonomia is comprised of several genetic subtypes, antisense oligonucleotides of the disclosure were specifically designed to skip exon 19, 20, or 26 of IKBKAP pre-mRNA (Axelrod and Gold-von Simson, Orph J of Rare Dis, 2007, 2:39; Dietrich and Dragatsis, Genet Mol Biol 2016, 39 (4): 497-514).

In some embodiments, the nucleobase sequence of an antisense oligonucleotides that induces exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and/or 37, skipping is designed to be complementary to a specific target sequence within exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and/or 37, of IKBKAP pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence selected from SEQ ID NOs: 152-156 to target the IKBKAP gene (Table 2).

### PBD1A

Zellweger syndrome (PBD1A; OMIM 214100) is caused by homozygous or compound heterozygous mutation in the PEX1 gene on chromosome 7q21-q22. Zellweger syndrome is an autosomal recessive systemic disorder characterized clinically by severe neurologic dysfunction, craniofacial abnormalities, and liver dysfunction, and biochemically by the absence of peroxisomes. There are two common PEX1 gene mutations found in people with Zellweger spectrum disorder. One mutation replaces the amino acid glycine with the amino acid aspartic acid at position 843 in Pex1p (written as Gly843 Asp or G843D). This mutation leads to reduced levels of the protein. Individuals who have the G843D mutation tend to have signs and symptoms that are at the less-severe end of the condition spectrum. The other common mutation, which is known as the 1700fs mutation, leads to the production of an abnormally short, nonfunctional Pex1p. Individuals who have the 1700fs mutation often have signs and symptoms that are at the severe end of the condition spectrum. Mutations in the PEX1 gene that cause Zellweger spectrum disorder reduce or eliminate the activity of the Pex1p protein. Without enough functional Pex1p, enzymes are not properly imported into peroxisomes. As a result, cells contain empty peroxisomes that cannot carry out their usual functions. The severe end of the condition spectrum is caused by the absence of functional peroxisomes within cells. The less severe end of the condition spectrum results from mutations that allow some peroxisomes to form (European Pat. Appl. No. EP4104867 A2, incorporated herein by reference in its entirety).

In some embodiments, the methods and compositions described herein are useful for treating Zellweger syndrome, by delivering an antisense oligonucleotide capable of inducing skipping of an exon in PEX1 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, 19, 20, 21, 22, or 23, target regions of the PEX1 gene and induce exon skipping in at least one of exon 1, 2, 3, 4, 5, 6, 7, 8, 9, 10, 11, 12, 13, 14, 15, 16, 17, 18, 19, 20, 21, 22, or 23. In some embodiments, the disclosure relates to antisense oligonucleotides complementary to an exon 10 target region of the PEX1 pre-mRNA designated as an annealing site. In some embodiments, the disclosure relates to antisense oligonucleotides complementary to an exon 13 target region of the PEX1 pre-mRNA designated as an annealing site. In some embodiments, the disclosure relates to antisense oligonucleotides complementary to an exon 14 target region of the PEX1 pre-mRNA designated as an annealing site. In some embodiments, the disclosure relates to antisense oligonucleotides complementary to an exon 18 target region of the PEX1 pre-mRNA designated as an annealing site. In some embodiments, the disclosure relates to antisense oligonucleotides complementary to an exon 19 target region of the PEX1 pre-mRNA designated as an annealing site. In some embodiments, the disclosure relates to antisense oligonucleotides complementary to an exon 20 target region of the PEX1 pre-mRNA designated as an annealing site. In some embodiments, the disclosure relates to antisense oligonucleotides complementary to an exon 21 target region of the PEX1 pre-mRNA designated as an annealing site (European Pat. Appl. No. EP4104867 A2).

In some embodiments, antisense oligonucleotides of the disclosure target PEX1 pre-mRNA and induces skipping of exon 1, 2, 3, 4, 5, 6, 7, 8, 9, 10, 11, 12, 13, 14, 15, 16, 17, 18, 19, 20, 21, 22, and/or 23, so it is excluded or skipped from the mature, spliced mRNA transcript. By skipping exon 1, 2, 3, 4, 5, 6, 7, 8, 9, 10, 11, 12, 13, 14, 15, 16, 17, 18, 19, 20, 21, 22, and/or 23, the disrupted reading frame is restored to an in-frame mutation. While Zellweger syndrome is comprised of several genetic subtypes, antisense oligonucleotides of the disclosure were specifically designed to skip exon 10, 13, 14, 18, 19, 20, or 21 of PEX1 pre-mRNA (Crane et al., 2005, Hum Mutat 26 (3): 167-175).

In some embodiments, the nucleobase sequence of an antisense oligonucleotides that induces exon 1, 2, 3, 4, 5, 6, 7, 8, 9, 10, 11, 12, 13, 14, 15, 16, 17, 18, 19, 20, 21, 22, and/or 23, skipping is designed to be complementary to a specific target sequence within exon 1, 2, 3, 4, 5, 6, 7, 8, 9, 10, 11, 12, 13, 14, 15, 16, 17, 18, 19, 20, 21, 22, and/or 23, of PEX1 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence selected from SEQ ID NOs: 328-330 to target the PEX1gene (Table 2).

### MRD23

Autosomal dominant mental retardation-23 (MRD23; 615761) is caused by heterozygous mutation in the SETD5 gene on chromosome 3p25. Mental retardation, autosomal dominant 23 (MRD23) is a disorder characterized by significantly below average general intellectual functioning associated with impairments in adaptive behavior and manifested during the developmental period. MRD23 patients manifest moderate to severe intellectual disability with additional variable features of brachycephaly, a low hairline, depressed nasal bridge, prominent high nasal root, tubular nose, upslanting palpebral fissures, long and smooth philtrum, micrognathia, thin upper lip, and crowded teeth. Behavioral problems, including obsessive-compulsive disorder, hand flapping with ritualized behavior, and autism, are prominent features. The disease is caused by mutations affecting the gene represented in this entry (European Pat. Appl. No. EP4104867 A2).

In some embodiments, the methods and compositions described herein are useful for treating MRD23, by delivering an antisense oligonucleotide capable of inducing skipping of an exon in SETD5 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, 19, 20, 21, 22, or 23, target regions of the SETD5 gene and induce exon skipping in at least one of exon 1, 2, 3, 4, 5, 6, 7, 8, 9, 10, 11, 12, 13, 14, 15, 16, 17, 18, 19, 20, 21, 22, or 23. In some embodiments, the disclosure relates to antisense oligonucleotides complementary to an exon 4 target region of the SETD5 pre-mRNA designated as an annealing site. In some embodiments, the disclosure relates to antisense oligonucleotides complementary to an exon 4 target region of the SETD5 pre-mRNA designated as an annealing site. In some embodiments, the disclosure relates to antisense oligonucleotides complementary to an exon 5 target region of the SETD5 pre-mRNA designated as an annealing site (European Pat. Appl. No. EP4104867 A2).

In some embodiments, antisense oligonucleotides of the disclosure target SETD5 pre-mRNA and induces skipping of exon 1, 2, 3, 4, 5, 6, 7, 8, 9, 10, 11, 12, 13, 14, 15, 16, 17, 18, 19, 20, 21, 22, and/or 23, so it is excluded or skipped from the mature, spliced mRNA transcript. By skipping exon 1, 2, 3, 4, 5, 6, 7, 8, 9, 10, 11, 12, 13, 14, 15, 16, 17, 18, 19, 20, 21, 22, and/or 23, the disrupted reading frame is restored to an in-frame mutation. While MRD23 is comprised of several genetic subtypes, antisense oligonucleotides of the disclosure were specifically designed to skip exon 4, 5, 7, 9, or 14 of SETD5 pre-mRNA (European Pat. Appl. No. EP4104867 A2; Crippa et al., 2020, Front Neurol 11:631; Kuechler et al., 2014, Eur J of Genet 23:753-760).

In some embodiments, the nucleobase sequence of an antisense oligonucleotides that induces exon 1, 2, 3, 4, 5, 6, 7, 8, 9, 10, 11, 12, 13, 14, 15, 16, 17, 18, 19, 20, 21, 22, and/or 23, skipping is designed to be complementary to a specific target sequence within exon 1, 2, 3, 4, 5, 6, 7, 8, 9, 10, 11, 12, 13, 14, 15, 16, 17, 18, 19, 20, 21, 22, and/or 23, of SETD5 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-gccuccacag auucaggg-3′ (SEQ ID NOs: 324) to target the SETD5 gene (Table 2).

### EPM5

Epilepsy, progressive myoclonic 5 (EPM5; 607459) is a neurodegenerative disorder characterized by myoclonic seizures and variable neurologic symptoms including cognitive decline and persistent movement abnormalities. In some cases, a heterozygosity for a complex mutation in the PRICKLE2 gene, a 443G-A transition, resulting in an Arg148-to-His (R148H) substitution, and a 457G-A transition, resulting in a Val153-to-Ile (V1531) substitution can be identified in a progressive myoclonic epilepsy patient. In some cases, a heterozygous 1813G-T transversion in the PRICKLE2 gene, resulting in a Val605-to-Phe (V605F) substitution can be identified in a progressive myoclonic epilepsy patient (European Pat. Appl. No. EP4104867 A2).

In some embodiments, the methods and compositions described herein are useful for treating EPM5, by delivering an antisense oligonucleotide capable of inducing skipping of an exon in PRICKLE2 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, or 8 target regions of the PRICKLE2 gene and induce exon skipping in at least one of exon 1, 2, 3, 4, 5, 6, 7, or 8. In some embodiments, the disclosure relates to antisense oligonucleotides complementary to an exon 4 target region of the PRICKLE2 pre-mRNA designated as an annealing site. In some embodiments, the disclosure relates to antisense oligonucleotides complementary to an exon 5 target region of the PRICKLE2 pre-mRNA designated as an annealing site (European Pat. Appl. No. EP4104867 A2).

In some embodiments, antisense oligonucleotides of the disclosure target PRICKLE2 pre-mRNA and induces skipping of exon 1, 2, 3, 4, 5, 6, 7, and/or 8, so it is excluded or skipped from the mature, spliced mRNA transcript. By skipping exon 1, 2, 3, 4, 5, 6, 7, and/or 8, the disrupted reading frame is restored to an in-frame mutation. While EPM5 is comprised of several genetic subtypes, antisense oligonucleotides of the disclosure were specifically designed to skip exon 4, or 5 pre-mRNA (European Pat. Appl. No. EP4104867 A2).

In some embodiments, the nucleobase sequence of an antisense oligonucleotides that induces exon 1, 2, 3, 4, 5, 6, 7, and/or 8 skipping is designed to be complementary to a specific target sequence within exon 1, 2, 3, 4, 5, 6, 7, and/or 8 of PRICKLE2 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-agggaguugc aauaucga-3′ (SEQ ID NOs: 323) to target the PRICKLE2 gene (Table 2).

### FHM1

At least 20 mutations in the CACNA1A gene have been identified in people with familial hemiplegic migraine type 1 (FHM1; OMIM 141500). FHM1 is characterized by an aura of hemiplegia that is always associated with at least one other aura symptom (e.g., hemianopsia, hemisensory deficit, aphasia). Most of the mutations that cause FHM1 change single amino acids in the CaV2.1 channel. The most common mutation, which has been found in more than a dozen affected families, replaces the amino acid threonine with the amino acid methionine at protein position 666 (European Pat. Appl. No. EP4104867 A2).

In some embodiments, the methods and compositions described herein are useful for treating FHM1, by delivering an antisense oligonucleotide capable of inducing skipping of an exon in CACNA1A pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47 target regions of the CACNA1A gene and induce exon skipping in at least one of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47. In some embodiments, the disclosure relates to antisense oligonucleotides complementary to an exon 14 target region of the CACNA1A pre-mRNA designated as an annealing site. In some embodiments, the disclosure relates to antisense oligonucleotides complementary to an exon 15 target region of the CACNA1A pre-mRNA designated as an annealing site. In some embodiments, the disclosure relates to antisense oligonucleotides complementary to an exon 29 target region of the CACNA1A pre-mRNA designated as an annealing site. In some embodiments, the disclosure relates to antisense oligonucleotides complementary to an exon 30 target region of the CACNA1A pre-mRNA designated as an annealing site. In some embodiments, the disclosure relates to antisense oligonucleotides complementary to an exon 36 target region of the CACNA1A pre-mRNA designated as an annealing site. In some embodiments, the disclosure relates to antisense oligonucleotides complementary to an exon 37 target region of the CACNA1A pre-mRNA designated as an annealing site (European Pat. Appl. No. EP4104867 A2).

In some embodiments, antisense oligonucleotides of the disclosure target CACNA1A pre-mRNA and induces skipping of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, and/or 47 so it is excluded or skipped from the mature, spliced mRNA transcript. By skipping exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, and/or 47, the disrupted reading frame is restored to an in-frame mutation. While FHM1 is comprised of several genetic subtypes, antisense oligonucleotides of the disclosure were specifically designed to skip exon 14, 15, 29, 30, 36, or 37 pre-mRNA (European Pat. Appl. No. EP4104867 A2).

In some embodiments, the nucleobase sequence of an antisense oligonucleotides that induces exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, and/or 47 skipping is designed to be complementary to a specific target sequence within exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, and/or 47 of CACNA1A pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence selected from SEQ ID NO:s: 341-346 to target the CACNA1A gene (Table 2).

### AHC

Alternating hemiplegia of childhood (OMIM 104290) is an autosomal dominant condition. Alternating hemiplegia of childhood can result from new mutations in the gene and occur in people with no history of the disorder in their family. The primary feature of this condition is recurrent episodes of temporary paralysis, often affecting one side of the body (hemiplegia). The known ATP1A2 gene mutation associated with this condition replaces a single amino acid in Na+/K+ ATPase: the amino acid threonine is replaced with the amino acid asparagine at protein position 378. This genetic change can impair the protein's ability to transport ions (European Pat. Appl. No. EP4104867 A2).

In some embodiments, the methods and compositions described herein are useful for treating alternating hemiplegia of childhood, by delivering an antisense oligonucleotide capable of inducing skipping of an exon in ATP1A2 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, 19, 20, 21, 22, or 23 target regions of the ATP1A2 gene and induce exon skipping in at least one of exon 1, 2, 3, 4, 5, 6, 7, 8, 9, 10, 11, 12, 13, 14, 15, 16, 17, 18, 19, 20, 21, 22, or 23. In some embodiments, the disclosure relates to antisense oligonucleotides complementary to an exon 22 target region of the ATP1A2 pre-mRNA designated as an annealing site. In some embodiments, the disclosure relates to antisense oligonucleotides complementary to an exon 23 target region of the ATP1A2 pre-mRNA designated as an annealing site (European Pat. Appl. No. EP4104867 A2).

In some embodiments, antisense oligonucleotides of the disclosure target ATP1A2 pre-mRNA and induces skipping of exon 1, 2, 3, 4, 5, 6, 7, 8, 9, 10, 11, 12, 13, 14, 15, 16, 17, 18, 19, 20, 21, 22, and/or 23 so it is excluded or skipped from the mature, spliced mRNA transcript. By skipping exon 1, 2, 3, 4, 5, 6, 7, 8, 9, 10, 11, 12, 13, 14, 15, 16, 17, 18, 19, 20, 21, 22, and/or 23, the disrupted reading frame is restored to an in-frame mutation. While alternating hemiplegia of childhood is comprised of several genetic subtypes, antisense oligonucleotides of the disclosure were specifically designed to skip exon 22, or 23 pre-mRNA (European Pat. Appl. No. EP4104867 A2).

In some embodiments, the nucleobase sequence of an antisense oligonucleotides that induces exon 1, 2, 3, 4, 5, 6, 7, 8, 9, 10, 11, 12, 13, 14, 15, 16, 17, 18, 19, 20, 21, 22, and/or 23 skipping is designed to be complementary to a specific target sequence within exon 1, 2, 3, 4, 5, 6, 7, 8, 9, 10, 11, 12, 13, 14, 15, 16, 17, 18, 19, 20, 21, 22, and/or 23 of ATP1A2 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-ggcgcagaac caccaggu-3′ (SEQ ID NOs: 322) to target the ATP1A2 gene (Table 2).

### AGS6

Aicardi-Goutieres syndrome-6 (AGS6; OMIM 615010) can be caused by homozygous, compound heterozygous, or heterozygous mutation in the ADAR gene on chromosome 1q21.3. Aicardi-Goutières syndrome (AGS) manifests as an early-onset encephalopathy that usually, but not always, results in severe intellectual and physical handicap. A subgroup of infants with AGS present at birth with abnormal neurologic findings, hepatosplenomegaly, elevated liver enzymes, and thrombocytopenia, a picture highly suggestive of congenital infection. Otherwise, most affected infants present at variable times after the first few weeks of life, frequently after a period of apparently normal development. Typically, they demonstrate the subacute onset of a severe encephalopathy characterized by extreme irritability, intermittent sterile pyrexias, loss of skills, and slowing of head growth. Over time, as many as 40% develop chilblain skin lesions on the fingers, toes, and ears. It is becoming apparent that atypical, sometimes milder, cases of AGS exist, and thus the true extent of the phenotype associated with mutation of the AGS-related genes is not yet known. For example, mutation of ADAR has recently been associated with a clinical presentation of acute bilateral striatal necrosis (European Pat. Appl. No. EP4104867 A2).

In some embodiments, the methods and compositions described herein are useful for treating AGS6, by delivering an antisense oligonucleotide capable of inducing skipping of an exon in ADAR pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, or 15 target regions of the ADAR gene and induce exon skipping in at least one of exon 1, 2, 3, 4, 5, 6, 7, 8, 9, 10, 11, 12, 13, 14, or 15. In some embodiments, the disclosure relates to antisense oligonucleotides complementary to an exon 2 target region of the ADAR pre-mRNA designated as an annealing site. In some embodiments, the disclosure relates to antisense oligonucleotides complementary to an exon 3 target region of the ADAR pre-mRNA designated as an annealing site (European Pat. Appl. No. EP4104867 A2).

In some embodiments, antisense oligonucleotides of the disclosure target ADAR pre-mRNA and induces skipping of exon 1, 2, 3, 4, 5, 6, 7, 8, 9, 10, 11, 12, 13, 14, and/or 15 so it is excluded or skipped from the mature, spliced mRNA transcript. By skipping exon 1, 2, 3, 4, 5, 6, 7, 8, 9, 10, 11, 12, 13, 14, and/or 15, the disrupted reading frame is restored to an in-frame mutation. While AGS6 is comprised of several genetic subtypes, antisense oligonucleotides of the disclosure were specifically designed to skip exon 2, or 3 pre-mRNA (European Pat. Appl. No. EP4104867 A2).

In some embodiments, the nucleobase sequence of an antisense oligonucleotides that induces exon 1, 2, 3, 4, 5, 6, 7, 8, 9, 10, 11, 12, 13, 14, and/or 15 skipping is designed to be complementary to a specific target sequence within exon 1, 2, 3, 4, 5, 6, 7, 8, 9, 10, 11, 12, 13, 14, and/or 15 of ADAR pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence selected from SEQ ID NOs: 317-321 to target the ADAR gene (Table 2).

### EIEEF4

Epileptic encephalopathy, early infantile, 4 (EIEE4; OMIM 612164) is a severe form of epilepsy characterized by frequent tonic seizures or spasms beginning in infancy with a specific EEG finding of suppression-burst patterns, characterized by high-voltage bursts alternating with almost flat suppression phases. Affected individuals can have neonatal or infantile onset of seizures, profound mental retardation, and MRI evidence of brain hypomyelination. In some cases, in a patient with early infantile epileptic encephalopathy-4, a heterozygous 1631G-A transition in the STXBP1 gene, resulting in a gly544-to-Asp (G544D) substitution can be identified. (European Pat. Appl. No. EP4104867 A2)

In some embodiments, the methods and compositions described herein are useful for treating EIEEF4, by delivering an antisense oligonucleotide capable of inducing skipping of an exon in STXBP1 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, or 19 target regions of the STXBP1 gene and induce exon skipping in at least one of exon 1, 2, 3, 4, 5, 6, 7, 8, 9, 10, 11, 12, 13, 14, 15, 16, 17, 18, or 19. In some embodiments, the disclosure relates to antisense oligonucleotides complementary to an exon 6 target region of the STXBP1 pre-mRNA designated as an annealing site. In some embodiments, the disclosure relates to antisense oligonucleotides complementary to an exon 7 target region of the STXBP1 pre-mRNA designated as an annealing site (European Pat. Appl. No. EP4104867 A2).

In some embodiments, antisense oligonucleotides of the disclosure target STXBP1 pre-mRNA and induces skipping of exon 1, 2, 3, 4, 5, 6, 7, 8, 9, 10, 11, 12, 13, 14, 15, 16, 17, 18, and/or 19 so it is excluded or skipped from the mature, spliced mRNA transcript. By skipping exon 1, 2, 3, 4, 5, 6, 7, 8, 9, 10, 11, 12, 13, 14, 15, 16, 17, 18, and/or 19, the disrupted reading frame is restored to an in-frame mutation. While EIEEF4 is comprised of several genetic subtypes, antisense oligonucleotides of the disclosure were specifically designed to skip exon 6, or 7 pre-mRNA (European Pat. Appl. No. EP4104867 A2).

In some embodiments, the nucleobase sequence of an antisense oligonucleotides that induces exon 1, 2, 3, 4, 5, 6, 7, 8, 9, 10, 11, 12, 13, 14, 15, 16, 17, 18, and/or 19 skipping is designed to be complementary to a specific target sequence within exon 1, 2, 3, 4, 5, 6, 7, 8, 9, 10, 11, 12, 13, 14, 15, 16, 17, 18, and/or 19 of STXBP1 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-GCCAGUGCCC AUAGCGGG-3′, or 5′-CUUAUGCCAG UGCCCAUA-3′ (SEQ ID NOs: 331-332) to target the STXBP1 gene (Table 2).

### VWM

Leukoencephalopathy with vanishing white matter (VWM) (OMIM 603896) can be caused by homozygous or compound heterozygous mutation in any of the 5 genes encoding subunits of the translation initiation factor EIF2B: EIF2B1 on chromosome 12q24, EIF2B2 on chromosome 14q24, EIF2B3 on chromosome 1p34, EIF2B4 on chromosome 2p23, or EIF2B5 on chromosome 3q27. VMW is an autosomal recessive neurologic disorder characterized by variable neurologic features, including progressive cerebellar ataxia, spasticity, and cognitive impairment associated with white matter lesions on brain imaging. The neurologic signs include progressive cerebellar ataxia, spasticity, inconstant optic atrophy, and relatively preserved mental abilities. Disease is chronic-progressive with, in most individuals, additional episodes of rapid deterioration following febrile infections or minor head trauma. The mode of inheritance is autosomal recessive (European Pat. Appl. No. EP4104867 A2).

In some embodiments, the methods and compositions described herein are useful for treating VWM, by delivering an antisense oligonucleotide capable of inducing skipping of an exon in EIF2B5, EIF2B2, or EIF2B1 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, or 16 target regions of the EIF2B5 gene; to at least one of exon 1, 2, 3, 4, 5, 6, 7, 8, or 9 target regions of the EIF2B2 gene, or; to at least one of exon 1, 2, 3, 4, 5, 6, 7, or 8 target regions of the EIF2B1 gene, and induce exon skipping in at least one of exon 1, 2, 3, 4, 5, 6, 7, 8, 9, 10, 11, 12, 13, 14, 15, or 16; at least one of exon 1, 2, 3, 4, 5, 6, 7, 8, or 9, or; at least one of exon 1, 2, 3, 4, 5, 6, 7, or 8, respectively. In some embodiments, the disclosure relates to antisense oligonucleotides complementary to an exon 12 target region of the EIF2B5 pre-mRNA designated as an annealing site. In some embodiments, the disclosure relates to antisense oligonucleotides complementary to an exon 13 target region of the EIF2B5 pre-mRNA designated as an annealing site. In some embodiments, the disclosure relates to antisense oligonucleotides complementary to an exon 6 target region of the EIF2B2 pre-mRNA designated as an annealing site. In some embodiments, the disclosure relates to antisense oligonucleotides complementary to an exon 1 target region of the EIF2B1 pre-mRNA designated as an annealing site (European Pat. Appl. No. EP4104867 A2).

In some embodiments, antisense oligonucleotides of the disclosure target EIF2B5 pre-mRNA and induces skipping of exon 1, 2, 3, 4, 5, 6, 7, 8, 9, 10, 11, 12, 13, 14, 15, and/or 16; target EIF2B2 pre-mRNA and induces skipping of exon 1, 2, 3, 4, 5, 6, 7, 8, or 9; or disclosure target EIF2B1 pre-mRNA and induces skipping of exon 1, 2, 3, 4, 5, 6, 7, or 8, so it is excluded or skipped from the mature, spliced mRNA transcript. By skipping exon 1, 2, 3, 4, 5, 6, 7, 8, 9, 10, 11, 12, 13, 14, 15, and/or 16, exon 1, 2, 3, 4, 5, 6, 7, 8, or 9, or exon 1, 2, 3, 4, 5, 6, 7, or 8, the disrupted reading frame is restored to an in-frame mutation. While Leukodystrophy with VWM is comprised of several genetic subtypes, antisense oligonucleotides of the disclosure were specifically designed to skip exon 12 or 13 of the EIF2B5 pre-mRNA, exon 6 of the EIF2B2 pre-mRNA, or exon 1 of the EIF2B1 pre-mRNA (European Pat. Appl. No. EP4104867 A2).

In some embodiments, the nucleobase sequence of an antisense oligonucleotides that induces exon 1, 2, 3, 4, 5, 6, 7, 8, 9, 10, 11, 12, 13, 14, 15, and/or 16 skipping is designed to be complementary to a specific target sequence within exon 1, 2, 3, 4, 5, 6, 7, 8, 9, 10, 11, 12, 13, 14, 15, and/or 16 of EIF2B5 pre-mRNA. In some embodiments, the nucleobase sequence of an antisense oligonucleotides that induces exon 11, 2, 3, 4, 5, 6, 7, 8, or 9 skipping is designed to be complementary to a specific target sequence within exon 1, 2, 3, 4, 5, 6, 7, 8, or 9 of EIF2B2 pre-mRNA. In some embodiments, the nucleobase sequence of an antisense oligonucleotides that induces exon 1, 2, 3, 4, 5, 6, 7, or 8 skipping is designed to be complementary to a specific target sequence within exon 1, 2, 3, 4, 5, 6, 7, or 8 of EIF2B1 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide is SEQ ID NOs: 325 to target the EIF2B5 gene. In some embodiments, the antisense oligonucleotide sequence is SEQ ID NOs: 326 to target the EIF2B2 gene. In some embodiments, the antisense oligonucleotide sequence is SEQ ID NOs: 327 to target the EIF2B1 gene (Table 2).

### ICCA, BFIE, and EKD1

Infantile Convulsions and paroxysmal Choreoathetosis (ICCA; OMIM 602066) syndrome is a neurological condition characterized by the occurrence of seizures during the first year of life and choreoathetotic dyskinetic attacks during childhood or adolescence. Mutations in the PRRT2 gene, located on 16p11.2, has recently been found in families affected by ICCA syndrome. Benign familial infantile epilepsy (BFIE; OMIM 607745) is a genetic epileptic syndrome characterized by the occurrence of afebrile repeated seizures in healthy infants, between the third and eighth month of life. BFIE is a genetically heterogeneous disease. In the majority of cases, mutations in the proline-rich transmembrane protein 2 (PRRT2) gene located at 16p11.2 has been found. Episodic kinesigenic dyskinesia 1 (EKD1; OMIM 128200) can be referred to as familial paroxysmal kinesigenic dyskinesia. EKD1 is a disorder characterized by episodes of abnormal movement that range from mild to severe. In some cases, a heterozygous 1-bp duplication (649dupC) in exon 2 of the PRRT2 gene in the proline-rich domain, resulting in a frameshift and introduction of a stop codon 7 amino acids downstream of the insertion (Arg217ProfsTer8) can be identified. In some cases, a heterozygous 4-bp deletion (514delTCTG) in exon 2 of the PRRT2 gene in the proline-rich domain, resulting in a frameshift and premature termination can be identified. In some cases, a heterozygous 1-bp deletion (972delA) in exon 3 of the PRRT2 gene, resulting in a frameshift and premature termination in the second transmembrane motif can be identified. (European Pat. Appl. No. EP4104867 A2).

In some embodiments, the methods and compositions described herein are useful for treating ICCA, BFIE, or EKD1, by delivering an antisense oligonucleotide capable of inducing skipping of an exon in PRRT2 pre-mRNA carrying a deleterious mutation, e.g., that causes a frameshift mutation. In certain embodiments, antisense oligonucleotides of the disclosure are complementary to at least one of exon 1, 2, or 3 target regions of the PRRT2 gene and induce exon skipping in at least one of exon 1, 2, or 3. In some embodiments, the disclosure relates to antisense oligonucleotides complementary to an exon 6 target region of the PRRT2 pre-mRNA designated as an annealing site. In some embodiments, the disclosure relates to antisense oligonucleotides complementary to an exon 1 target region of the PRRT2 pre-mRNA designated as an annealing site (European Pat. Appl. No. EP4104867 A2).

In some embodiments, antisense oligonucleotides of the disclosure target PRRT2 pre-mRNA and induces skipping of exon 1, 2, and/or 3 so it is excluded or skipped from the mature, spliced mRNA transcript. By skipping exon 1, 2, and/or 3, the disrupted reading frame is restored to an in-frame mutation. While ICCA, BFIE, or EKD1, are comprised of few genetic subtypes, antisense oligonucleotides of the disclosure were specifically designed to skip exon 1 or 2 pre-mRNA (European Pat. Appl. No. EP4104867 A2).

In some embodiments, the nucleobase sequence of an antisense oligonucleotides that induces exon 1, 2, and/or 3 skipping is designed to be complementary to a specific target sequence within exon 1, 2, and/or 3 of PRRT2 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence selected from SEQ ID NOs: 333-335 to target the PRRT2 gene (Table 2).

### ALGS

Alagille syndrome (ALGS; OMIM 118450), also known as arteriohepatic dysplasia, is a rare, debilitating, autosomal dominant, multisystem disorder (Turnpenny and Ellard, Eur. J. Hum. Gen. 2012, 20, 251-257). Patients suffer from liver damage caused by abnormalities in the bile ducts. Other effects include heart disease, vascular anomalies, skeletal anomalies, ophthalmic features, facial features, renal anomalies, growth retardation, and pancreatic insufficiency. The reported ALGS prevalence of 1:70,000 is thought to be an underestimate because of the variability and reduced penetrance of the condition. Mutations of genes involved in Notch signaling have been reported to cause ALGS. Mutations in JAG1 cause ALGS type 1, while mutations in NOTCH2 cause ALGS type 2, which is less prevalent than ALGS type 1. JAG1 encodes JAG1 protein, a cell surface ligand for the Notch transmembrane receptors. Binding of JAG1 protein to the Notch receptors triggers a signaling cascade that results in transcription of genes involved in cell fate determination and differentiation (U.S. Pat. No. 11,096,956, herein incorporated by reference in its entirety).

In some embodiments, the methods and compositions described herein are useful for treating ALGS, by delivering an antisense oligonucleotide capable of inducing skipping of an exon in JAG1 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, 19, 20, 21, 22, 23, 24, 25, or 26 target regions of the JAG1 gene and induce exon skipping in at least one of exon 1, 2, 3, 4, 5, 6, 7, 8, 9, 10, 11, 12, 13, 14, 15, 16, 17, 18, 19, 20, 21, 22, 23, 24, 25, or 26. In some embodiments, the disclosure relates to antisense oligonucleotides complementary to an exon 13 target region of the JAG1 pre-mRNA designated as an annealing site (U.S. Pat. No. 11,096,956).

In some embodiments, antisense oligonucleotides of the disclosure target JAG1 pre-mRNA and induces skipping of exon 1, 2, 3, 4, 5, 6, 7, 8, 9, 10, 11, 12, 13, 14, 15, 16, 17, 18, 19, 20, 21, 22, 23, 24, 25, and/or 26 so it is excluded or skipped from the mature, spliced mRNA transcript. By skipping exon 1, 2, 3, 4, 5, 6, 7, 8, 9, 10, 11, 12, 13, 14, 15, 16, 17, 18, 19, 20, 21, 22, 23, 24, 25, and/or 26, the disrupted reading frame is restored to an in-frame mutation. While ALGS are comprised of few genetic subtypes, antisense oligonucleotides of the disclosure were specifically designed to skip exon 13 pre-mRNA (U.S. Pat. No. 11,096,956).

In some embodiments, the nucleobase sequence of an antisense oligonucleotides that induces exon 1, 2, 3, 4, 5, 6, 7, 8, 9, 10, 11, 12, 13, 14, 15, 16, 17, 18, 19, 20, 21, 22, 23, 24, 25, and/or 26 skipping is designed to be complementary to a specific target sequence within exon 1, 2, 3, 4, 5, 6, 7, 8, 9, 10, 11, 12, 13, 14, 15, 16, 17, 18, 19, 20, 21, 22, 23, 24, 25, and/or 26 of JAG1 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence selected from SEQ ID NOs: 285-294 to target the JAG1 gene (Table 2).

### TSC

Tuberous sclerosis complex (TSC; OMIM 191092) is a disorder characterized by growth of benign tumors in multiple organ systems (Au, K., et al., J. Child Neurol., 2004, 19:699-709). Tumors of the central nervous system (CNS) are the leading io cause of morbidity and mortality, followed by renal disease. Patients can suffer from abnormalities of the brain that may include seizures, intellectual disability, and developmental delay, as well as abnormalities of the skin, lung, kidneys, and heart. The disorder affects as many as 25,000 to 40,000 15 individuals in the United States and about 1 to 2 million individuals worldwide, with an estimated prevalence of one in 6,000 newborns. TSC is a genetic disorder with an autosomal dominant inheritance pattern, caused by inherited defects or de novo 20 mutations that occur on two genes, TSC1 and TSC2. Only one of the genes needs to be affected for TSC to be present. The TSC1 gene, on chromosome 9, produces a protein called hamartin. The TSC2 gene, discovered in 1993, is on chromosome 16 and produces the protein tuberin. Scientists 25 believe these proteins act in a complex as growth suppressors by inhibiting the activation of a master, evolutionarily conserved kinase called mTOR. Loss of regulation of mTOR occurs in cells lacking either hamartin or tuberin, and this leads to abnormal differentiation and development, and to 30 the generation of enlarged cells, as are seen in TSC brain lesions (U.S. Pat. No. 11,096,956).

In some embodiments, the methods and compositions described herein are useful for treating TSC, by delivering an antisense oligonucleotide capable of inducing skipping of an exon in TSC2 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 target regions of the TSC2 gene and induce exon skipping in at least one of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In some embodiments, the disclosure relates to antisense oligonucleotides complementary to an exon 4 target region of the TSC2 pre-mRNA designated as an annealing site (U.S. Pat. No. 11,096,956).

In some embodiments, antisense oligonucleotides of the disclosure target TSC2 pre-mRNA and induces skipping of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and/or 40 so it is excluded or skipped from the mature, spliced mRNA transcript. By skipping exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and/or 40, the disrupted reading frame is restored to an in-frame mutation. While TSC are comprised of few genetic subtypes, antisense oligonucleotides of the disclosure were specifically designed to skip exon 4 pre-mRNA (U.S. Pat. No. 11,096,956).

In some embodiments, the nucleobase sequence of an antisense oligonucleotides that induces exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and/or 40 skipping is designed to be complementary to a specific target sequence within exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and/or 40 of TSC2 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence selected from SEQ ID NOs: 295-304 to target the TSC2 gene (Table 2).

### USH

Usher syndrome (USH, or just ‘Usher’; OMIM 276901) and nonsyndromic retinitis pigmentosa (NSRP) are degenerative diseases of the retina. The hearing impairment in Usher patients is mostly stable and congenital and can be partly compensated by hearing aids or cochlear implants. The degeneration of photoreceptor cells in Usher and NSRP is progressive and often leads to complete blindness between the third and fourth decade of life, thereby leaving time for therapeutic intervention. Mutations in the USH2A gene are the most frequent cause of Usher syndrome type IIa explaining up to 50% of all Usher patients worldwide (+1300 patients in the Netherlands) and, as indicated by McGee et al. (2010. J Med Genet 47 (7): 499-506), also the most prevalent cause of NSRP in the USA, likely accounting for 12-25% of all cases of retinitis pigmentosa (RP). The mutations are spread throughout the seventy-two USH2A exons and their flanking intron sequences, and consist of nonsense and missense mutations, deletions, duplications, large rearrangements, and splicing variants. Exon 13 is by far the most frequently mutated exon with two founder mutations (c.2299deIG (p.E767SfsX21) in USH2 patients and c.2276G>T (p.C759F) in NSRP patients). For exon 50, fifteen pathogenic mutations have been reported, of which at least eight are clearly protein-truncating. Also, a deep intronic mutation in intron 40 of USH2A (c.7595-2144A>G) was reported (Vache et al. 2012. Human Mutation 33 (1): 104-108), which creates a cryptic high-quality splice donor site in intron 40 resulting in the inclusion of an aberrant exon of 152 bp (Pseudo Exon 40, or PE40) in the mutant USH2A mRNA, that causes premature termination of translation (U.S. patent application No. 2022021348 A1, herein incorporated by reference in its entirety).

In some embodiments, the methods and compositions described herein are useful for treating USH, by delivering an antisense oligonucleotide capable of inducing skipping of an exon in USH2A pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 target regions of the USH2A gene and induce exon skipping in at least one of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72. In some embodiments, the disclosure relates to antisense oligonucleotides complementary to an exon 13 target region of the USH2A pre-mRNA designated as an annealing site. In some embodiments, the disclosure relates to antisense oligonucleotides complementary to an exon 50 target region of the USH2A pre-mRNA designated as an annealing site. In some embodiments, the disclosure relates to antisense oligonucleotides complementary to an exon 62 target region of the USH2A pre-mRNA designated as an annealing site (U.S. patent application No. 2022021348 A1).

In some embodiments, antisense oligonucleotides of the disclosure target USH2A pre-mRNA and induces skipping of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, and/or 72 so it is excluded or skipped from the mature, spliced mRNA transcript. By skipping exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, and/or 72, the disrupted reading frame is restored to an in-frame mutation. While USH are comprised of few genetic subtypes, antisense oligonucleotides of the disclosure were specifically designed to skip exon 13, 50, or 62 pre-mRNA (U.S. patent application No. 2022021348 A1).

In some embodiments, the nucleobase sequence of an antisense oligonucleotides that induces exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, and/or 72 skipping is designed to be complementary to a specific target sequence within exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, and/or 72 of USH2A pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence selected from SEQ ID NOs: 305-316 to target the USH2A gene (Table 2).

### PTHS

Pitt-Hopkins syndrome (PTHS; OMIM 610954) is characterized by mental retardation, wide mouth and distinctive facial features, and intermittent hyperventilation followed by apnea. PTHS is linked to haploinsufficiency of the TCF4 transcription factor gene. At least 50 mutations in the TCF4 gene have been found to cause Pitt-Hopkins syndrome. Some mutations delete a nucleotide within the TCF4 gene, while other mutations delete the TCF4 gene as well as a number of genes that surround it. Still other TCF4 gene mutations replace single nucleotides. The size of the mutation does not appear to affect the severity of the condition; individuals with large deletions and those with single nucleotide changes seem to have similar signs and symptoms. TCF4 gene mutations disrupt the protein's ability to bind to DNA and control the activity of certain genes. These gene mutations typically do not affect the TCF4 protein's ability to bind to other proteins. The TCF4 protein's inability to bind to DNA and control the activity of certain genes, particularly those genes involved in nervous system development and function, contributes to the signs and symptoms of PTHS. It is also likely that the loss of the normal proteins that are attached to the nonfunctional TCF4 proteins contribute to the features of this condition (European Pat. Appl. No. EP4104867 A2).

In some embodiments, the methods and compositions described herein are useful for treating PTHS, by delivering an antisense oligonucleotide capable of inducing skipping of an exon in TCF4 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, 19, or 20 target regions of the TCF4 gene and induce exon skipping in at least one of exon 1, 2, 3, 4, 5, 6, 7, 8, 9, 10, 11, 12, 13, 14, 15, 16, 17, 18, 19, or 20. In some embodiments, the disclosure relates to antisense oligonucleotides complementary to an exon 10 target region of the TCF4 pre-mRNA designated as an annealing site (European Pat. Appl. No. EP4104867 A2).

In some embodiments, antisense oligonucleotides of the disclosure target TCF4 pre-mRNA and induces skipping of exon 1, 2, 3, 4, 5, 6, 7, 8, 9, 10, 11, 12, 13, 14, 15, 16, 17, 18, 19, and/or 20 so it is excluded or skipped from the mature, spliced mRNA transcript. By skipping exon 1, 2, 3, 4, 5, 6, 7, 8, 9, 10, 11, 12, 13, 14, 15, 16, 17, 18, 19, and/or 20, the disrupted reading frame is restored to an in-frame mutation. While Pitt-Hopkins Syndrome are comprised of few genetic subtypes, antisense oligonucleotides of the disclosure were specifically designed to skip exon 10 pre-mRNA (European Pat. Appl. No. EP4104867 A2).

In some embodiments, the nucleobase sequence of an antisense oligonucleotides that induces exon 1, 2, 3, 4, 5, 6, 7, 8, 9, 10, 11, 12, 13, 14, 15, 16, 17, 18, 19, and/or 20 skipping is designed to be complementary to a specific target sequence within exon 1, 2, 3, 4, 5, 6, 7, 8, 9, 10, 11, 12, 13, 14, 15, 16, 17, 18, 19, and/or 20 of TCF4 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence selected from SEQ ID NOs: 338-340 to target the TCF4 gene (Table 2).

### SMS

Smith-Magenis syndrome (SMS; OMIM 182290) is caused in most cases (90%) by a 3.7-Mb interstitial deletion in chromosome 17p11.2. The disorder can also be caused by mutations in the RAI1 gene, which is within the Smith-Magenis chromosome region. Smith-Magenis syndrome is a developmental disorder that affects many parts of the body. The major features of this condition include mild to moderate intellectual disability, delayed speech and language skills, distinctive facial features, sleep disturbances, and behavioral problems. Affected individuals may have eye abnormalities that cause nearsightedness (myopia) and other vision problems. Although less common, heart and kidney defects also have been reported in people with Smith-Magenis syndrome. A small percentage of individuals with Smith-Magenis syndrome have a mutation in the RAI1 gene instead of a chromosomal deletion. Although these individuals have many of the major features of the condition, they are less likely than people with a chromosomal deletion to have short stature, hearing loss, and heart or kidney abnormalities (European Pat. Appl. No. EP4104867 A2).

In some embodiments, the methods and compositions described herein are useful for treating Smith-Magenis Syndrome, by delivering an antisense oligonucleotide capable of inducing skipping of an exon in RAI1 pre-mRNA carrying a deleterious mutation, e.g., that causes a frameshift mutation. In certain embodiments, antisense oligonucleotides of the disclosure are complementary to at least one of exon 1, 2, 3, 4, 5, or 6 target regions of the RAI1 gene and induce exon skipping in at least one of exon 1, 2, 3, 4, 5, or 6. In some embodiments, the disclosure relates to antisense oligonucleotides complementary to an exon 4 target region of the RAI1 pre-mRNA designated as an annealing site (European Pat. Appl. No. EP4104867 A2).

In some embodiments, antisense oligonucleotides of the disclosure target RAI1 pre-mRNA and induces skipping of exon 1, 2, 3, 4, 5, and/or 6 so it is excluded or skipped from the mature, spliced mRNA transcript. By skipping exon 1, 2, 3, 4, 5, and/or 6, the disrupted reading frame is restored to an in-frame mutation. While Smith-Magenis Syndrome are comprised of few genetic subtypes, antisense oligonucleotides of the disclosure were specifically designed to skip exon 4 pre-mRNA (European Pat. Appl. No. EP4104867 A2).

In some embodiments, the nucleobase sequence of an antisense oligonucleotides that induces exon 1, 2, 3, 4, 5, and/or 6 skipping is designed to be complementary to a specific target sequence within exon 1, 2, 3, 4, 5, and/or 6 of RAI1 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-UUCUUGGCAG CUGGAACA-3′ (SEQ ID NO:337) to target the RAI1 gene (Table 2).

### GEFSP9

Generalized epilepsy with febrile seizures plus-9 (OMIM 616172) is an autosomal dominant neurologic disorder characterized by onset of febrile and/or afebrile seizures in early childhood, usually before age 3 years. Seizure types are variable and include generalized tonic-clonic, atonic, myoclonic, complex partial, and absence. Most patients have remission of seizures later in childhood with no residual neurologic deficits, but rare patients may show mild developmental delay or mild intellectual disabilities. In some cases, in a patient with GEFSP9 a heterozygous c. 166C-T transition in the STX1B gene, resulting in a gln56-to-ter (Q56X) substitution can be identified (European Pat. Appl. No. EP4104867 A2).

In some embodiments, the methods and compositions described herein are useful for treating GEFSP9, by delivering an antisense oligonucleotide capable of inducing skipping of an exon in STX1B pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, or 10 target regions of the STX1B gene and induce exon skipping in at least one of exon 1, 2, 3, 4, 5, 6, 7, 8, 9, or 10. In some embodiments, the disclosure relates to antisense oligonucleotides complementary to an exon 6 target region of the STX1B pre-mRNA designated as an annealing site. In some embodiments, the disclosure relates to antisense oligonucleotides complementary to an exon 7 target region of the STX1B pre-mRNA designated as an annealing site (European Pat. Appl. No. EP4104867 A2)

In some embodiments, antisense oligonucleotides of the disclosure target STX1B pre-mRNA and induces skipping of exon 1, 2, 3, 4, 5, 6, 7, 8, 9, and/or 10 so it is excluded or skipped from the mature, spliced mRNA transcript. By skipping exon 1, 2, 3, 4, 5, 6, 7, 8, 9, and/or 10, the disrupted reading frame is restored to an in-frame mutation. While Generalized epilepsy with febrile seizures plus-9 are comprised of few genetic subtypes, antisense oligonucleotides of the disclosure were specifically designed to skip exon 6 or 7 pre-mRNA (European Pat. Appl. No. EP4104867 A2).

In some embodiments, the nucleobase sequence of an antisense oligonucleotides that induces exon 1, 2, 3, 4, 5, 6, 7, 8, 9, and/or 10 skipping is designed to be complementary to a specific target sequence within exon 1, 2, 3, 4, 5, 6, 7, 8, 9, and/or 10 of STX1B pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-CUUCCGGGAC AGUGUGGA-3′ (SEQ ID NO:336) to target the STX1B gene (Table 2).

### HGPS

Children with Hutchinson-Gilford progeria syndrome (HGPS; OMIM 176670) suffer from dramatic acceleration of some symptoms associated with normal aging, most notably cardiovascular disease that eventually leads to death from myocardial infarction and/or stroke usually in their second decade of life. For the vast majority of cases, a de novo point mutation in the lamin A (LMNA) gene is the cause of HGPS. This missense mutation creates a cryptic splice donor site that produces a mutant lamin A protein, termed “progerin,” which carries a 50-aa deletion near its C terminus (Varga et al., 2006, PNAS 103 (9): 3250-3255; U.S. Pat. No. 8,258,109 B2, herein incorporated by reference in its entirety).

In some embodiments, the methods and compositions described herein are useful for treating HGPS, by delivering an antisense oligonucleotide capable of inducing skipping of an exon in LMNA pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, or 12 target regions of the LMNA gene and induce exon skipping in at least one of exon 11, 2, 3, 4, 5, 6, 7, 8, 9, 10, 11, or 12. In some embodiments, the disclosure relates to antisense oligonucleotides complementary to an exon 6 target region of the LMNA pre-mRNA designated as an annealing site. In some embodiments, the disclosure relates to antisense oligonucleotides complementary to an exon 7 target region of the LMNA pre-mRNA designated as an annealing site (U.S. Pat. No. 8,258,109 B2).

In some embodiments, antisense oligonucleotides of the disclosure target LMNA pre-mRNA and induces skipping of exon 1, 2, 3, 4, 5, 6, 7, 8, 9, 10, 11, and/or 12 so it is excluded or skipped from the mature, spliced mRNA transcript. By skipping exon 1, 2, 3, 4, 5, 6, 7, 8, 9, 10, 11, and/or 12, the disrupted reading frame is restored to an in-frame mutation. While HGPS is comprised of few genetic subtypes, antisense oligonucleotides of the disclosure were specifically designed to skip exon 6 or 7 pre-mRNA (U.S. Pat. No. 8,258,109 B2).

In some embodiments, the nucleobase sequence of an antisense oligonucleotides that induces exon 1, 2, 3, 4, 5, 6, 7, 8, 9, 10, 11, and/or 12 skipping is designed to be complementary to a specific target sequence within exon 1, 2, 3, 4, 5, 6, 7, 8, 9, 10, 11, and/or 12 of LMNA pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises a sequence selected from SEQ ID NOs: 276-284) to target the LMNA gene (Table 2).

### FTDP-17

The MAPT (Microtubule associated protein tau) gene consists of 16 exons and its expression is regulated by complex alternative splicing. This results in the production of two types of alternatively spliced transcripts: one bearing Exon 10, also known as 4R (Four microtubule repeats) isoform and the other that lacks Exon 10 is called 3R isoform (Three microtubule repeats). Equal levels of these two isoforms are expressed in normal human adult brain. Though several mutations causing Frontotemporal dementia with parkinsonism linked to chromosome 17 (FTDP-17; OMIM 600274) are known in MAPT, a half of these affect alternative splicing of Exon 10. These include mis-sense mutations, silent mutations and point mutations which are located in Exon 10, introns 9 and 10. They are known to implicate an increase in Exon 10 causing an excessive accumulation of 4R. This leads to the formation of neurofibrillary tangles, hence resulting in neurodegeneration (U.S. patent application No. 20180066254 A1, herein incorporated by reference in its entirety).

In some embodiments, the methods and compositions described herein are useful for treating FTDP-17, by delivering an antisense oligonucleotide capable of inducing skipping of an exon in MAPT pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, or 13 target regions of the MAPT gene and induce exon skipping in at least one of exon 1, 2, 3, 4, 5, 6, 7, 8, 9, 10, 11, 12, or 13. In some embodiments, the disclosure relates to antisense oligonucleotides complementary to an exon 10 target region of the MAPT pre-mRNA designated as an annealing site (U.S. patent application No. 20180066254 A1).

In some embodiments, antisense oligonucleotides of the disclosure target MAPT pre-mRNA and induces skipping of exon 1, 2, 3, 4, 5, 6, 7, 8, 9, 10, 11, 12, and/or 13 so it is excluded or skipped from the mature, spliced mRNA transcript. By skipping exon 1, 2, 3, 4, 5, 6, 7, 8, 9, 10, 11, 12, and/or 13, the disrupted reading frame is restored to an in-frame mutation. While FTDP-17 is comprised of few genetic subtypes, antisense oligonucleotides of the disclosure were specifically designed to skip exon 10 pre-mRNA (U.S. patent application No. 20180066254 A1).

In some embodiments, the nucleobase sequence of an antisense oligonucleotides that induces exon 1, 2, 3, 4, 5, 6, 7, 8, 9, 10, 11, 12, and/or 13 skipping is designed to be complementary to a specific target sequence within exon 1, 2, 3, 4, 5, 6, 7, 8, 9, 10, 11, 12, and/or 13 of MAPT pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises a sequence selected from SEQ ID NOs: 386-388) to target the MAPT gene (Table 2).

### PHMDS, and SCZD15

Phelan-McDermid Syndrome (PHMDS; OMIM 606232) is a developmental disorder with variable features. Common features include neonatal hypotonia, global developmental delay, normal to accelerated growth, absent to severely delayed speech, autistic behavior, and minor dysmorphic features. Other less common features associated with this syndrome included increased tolerance to pain, dysplastic toenails, chewing behavior, fleshy hands, dysplastic ears, pointed chin, dolichocephaly, ptosis, tendency to overheat, and epicanthic folds. Researchers have showed that Phelan-McDermid syndrome neurons have reduced SHANK3 expression and major defects in excitatory, but not inhibitory, synaptic transmission (European Pat. Appl. No. 4104867 A2).

Susceptibility to schizophrenia 15 (SCZD15; OMIM 613950) has been associated with mutation in the SH3 and multiple ankyrin repeat domains-3 gene (SHANK3). Schizophrenia-15 is a complex, multifactorial psychotic disorder or group of disorders characterized by disturbances in the form and content of thought (e.g. delusions, hallucinations), in mood (e.g. inappropriate affect), in sense of self and relationship to the external world (e.g. loss of ego boundaries, withdrawal), and in behavior (e.g bizarre or apparently purposeless behavior). Although it affects emotions, it is distinguished from mood disorders in which such disturbances are primary. Similarly, there may be mild impairment of cognitive function, and it is distinguished from the dementias in which disturbed cognitive function is considered primary. Some patients manifest schizophrenic as well as bipolar disorder symptoms and are often given the diagnosis of schizoaffective disorder (European Pat. Appl. No. 4104867 A2).

In some embodiments, the methods and compositions described herein are useful for treating PHMDS AND/OR SCZD15, by delivering an antisense oligonucleotide capable of inducing skipping of an exon in SHANK3 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, 16, 17, 18, 19, 20, 21, or 22 target regions of the SHANK3 gene and induce exon skipping in at least one of exon 1, 2, 3, 4, 5, 6, 7, 8, 9, 10, 11, 12, 13, 14, 15, 16, 17, 18, 19, 20, 21, or 22. In some embodiments, the disclosure relates to antisense oligonucleotides complementary to an exon 10 target region of the SHANK3 pre-mRNA designated as an annealing site (U.S. patent application No. 20180066254 A1).

In some embodiments, antisense oligonucleotides of the disclosure target SHANK3 pre-mRNA and induces skipping of exon 1, 2, 3, 4, 5, 6, 7, 8, 9, 10, 11, 12, 13, 14, 15, 16, 17, 18, 19, 20, 21, and/or 22 so it is excluded or skipped from the mature, spliced mRNA transcript. By skipping exon 1, 2, 3, 4, 5, 6, 7, 8, 9, 10, 11, 12, 13, 14, 15, 16, 17, 18, 19, 20, 21, and/or 22, the disrupted reading frame is restored to an in-frame mutation. While PHMDS and/or SCZD15 is comprised of few genetic subtypes, antisense oligonucleotides of the disclosure were specifically designed to skip exon 10 pre-mRNA (European Pat. Appl. No. 4104867 A2).

In some embodiments, the nucleobase sequence of an antisense oligonucleotides that induces exon 1, 2, 3, 4, 5, 6, 7, 8, 9, 10, 11, 12, 13, 14, 15, 16, 17, 18, 19, 20, 21, and/or 22 skipping is designed to be complementary to a specific target sequence within exon 1, 2, 3, 4, 5, 6, 7, 8, 9, 10, 11, 12, 13, 14, 15, 16, 17, 18, 19, 20, 21, and/or 22 of SHANK3 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide sequence is 5′-ACCACGUUCACCCCGUUC-3′ (SEQ ID NO:358) to target the SHANK3 gene (Table 2).

### NF2

Neurofibromatosis type II (NF2; OMIM 101000) is caused by mutation in the gene encoding neurofibromin-2, which is also called merlin, on chromosome 22q12.2. Neurofibromatosis type II is an inheritable disorder with an autosomal dominant mode of transmission. Incidence of the disease is about 1 in 60,000. Through statistics, it is suspected that one-half of cases are inherited, and one-half are the result of new, de novo mutations (European Pat. Appl. No. 4104867 A2).

In some embodiments, the methods and compositions described herein are useful for treating NF2, by delivering an antisense oligonucleotide capable of inducing skipping of an exon in NF2 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 8, 9, 10, 11, 12, 13, 14, 15, or 16 target regions of the NF2 gene and induce exon skipping in at least one of exon 1, 2, 3, 4, 5, 6, 7, 8, 9, 10, 11, 12, 13, 14, 15, or 16. In some embodiments, the disclosure relates to antisense oligonucleotides complementary to an exon 10 target region of the NF2 pre-mRNA designated as an annealing site (U.S. Pat. Appl. No. 20180066254 A1).

In some embodiments, antisense oligonucleotides of the disclosure target NF2 pre-mRNA and induces skipping of exon 1, 2, 3, 4, 5, 6, 7, 8, 9, 10, 11, 12, 13, 14, 15, and/or 16 so it is excluded or skipped from the mature, spliced mRNA transcript. By skipping exon 1, 2, 3, 4, 5, 6, 7, 8, 9, 10, 11, 12, 13, 14, 15, and/or 16, the disrupted reading frame is restored to an in-frame mutation. While NF2 is comprised of few genetic subtypes, antisense oligonucleotides of the disclosure were specifically designed to skip exon 10 pre-mRNA (European Pat. Appl. No. 4104867 A2).

In some embodiments, the nucleobase sequence of an antisense oligonucleotides that induces exon 1, 2, 3, 4, 5, 6, 7, 8, 9, 10, 11, 12, 13, 14, 15, and/or 16 skipping is designed to be complementary to a specific target sequence within exon 1, 2, 3, 4, 5, 6, 7, 8, 9, 10, 11, 12, 13, 14, 15, and/or 16 of NF2 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide sequence is selected from SEQ ID NOs: 351-357 to target the NF2 gene (Table 2).

### PARK8

Parkinson's Disease 8 (PARK8; OMIM 607060) is a progressive neurological disorder estimated to affect 7-10 million people worldwide. There is no treatment available that cures or slows the progression of PD. Elevated leucine-rich repeat kinase 2 (LRRK2) activity has been associated with genetic and sporadic forms of PD and, thus, reducing LRRK2 function is a promising therapeutic strategy (Korecka et al., Mol Therap Nucleic Acid 21:623-635).

In some embodiments, the methods and compositions described herein are useful for treating PARK8, by delivering an antisense oligonucleotide capable of inducing skipping of an exon in LRRK2 pre-mRNA carrying a deleterious mutation, e.g., that causes a frameshift mutation. In certain embodiments, antisense oligonucleotides of the disclosure are complementary to at least one of exon 1, 2, 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51 target regions of the LRRK2 gene and in-duce exon skipping in at least one of exon 1, 2, 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51. In some embodiments, the disclosure relates to antisense oligonucleotides complementary to exon 31, or 41 target region of the LRRK2 pre-mRNA designated as an annealing site (U.S. Pat. No. 9,840,710, incorporated by reference herein in its entirety).

In some embodiments, antisense oligonucleotides of the disclosure target LRRK2 pre-mRNA and induces skipping of exon 1, 2, 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and/or 51 so it is excluded or skipped from the mature, spliced mRNA transcript. By skipping exon 1, 2, 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and/or 51, the disrupted reading frame is restored to an in-frame mutation. While PARK8 is comprised of few genetic subtypes, antisense oligonucleotides of the disclosure were specifically designed to skip exon 31, or 41 LRRK2 pre-mRNA (U.S. Pat. No. 9,840,710, incorporated by reference herein in its entirety).

In some embodiments, the nucleobase sequence of an antisense oligonucleotides that induces exon 1, 2, 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and/or 51 skipping is designed to be complementary to a specific target sequence within exon 1, 2, 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, and/or 51 of LRRK2 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide sequence is selected from SEQ ID NOs: 389-391 to target the LRRK2 gene (Table 2).

### Musculoskeletal Disorders

Multiple forms of muscular dystrophy are now known to be caused by defects in the O-linked glycosylation of α-dystroglycan. These dystroglycanopathies span a spectrum of phenotypes from Walker-Warburg syndrome (WWS; OMIM 236670; characterized by severe congenital muscular dystrophy, retinal and anterior chamber eye abnormalities, cobblestone lissencephaly, and hydrocephalus) to mild, adult onset LGMD. Included in this group of disorders is Fukuyama congenital muscular dystrophy (FCMD; OMIM 253800). Patients with typical FCMD display dystrophic changes in skeletal muscle, structural brain malformations, and severe ocular abnormalities. Most patients are never able to walk independently and have moderate to severe cognitive delay. The average life span is less than 20 years (Puckett et al., Further evidence of Fukutin mutations as a cause of childhood onset limb-girdle muscular dystrophy without mental retardation, Neurosmucul Disord. 2009 May; 19 (5): 352-356).

### DMD

Dystrophin-associated muscular dystrophies range from the severe Duchenne muscular dystrophy (DMD; OMIM 310200) to the milder Becker muscular dystrophy (BMD; OMIM 300376). Mapping and molecular genetic studies showed that both are the result of mutations in the huge gene that encodes dystrophin, also symbolized as DMD. Approximately two-thirds of the mutations in both forms are deletions of one or many exons in the dystrophin gene. Although there is no clear correlation found between the extent of the deletion and the severity of the disorder, DMD deletions usually result in frameshift.

In some embodiments, the methods and compositions described herein are useful for treating Duchenne muscular dystrophy (DMD), by delivering an antisense oligonucleotide capable of inducing skipping of an exon in myostatin (MSTN) pre-mRNA carrying a deleterious mutation, e.g., that causes a frameshift mutation. In certain embodiments, antisense oligonucleotides of the disclosure are complementary to at least one of exon 1, 2, or 3 target regions of the MSTN gene and induce exon skipping in at least one of exon 1, 2, or 3. In some embodiments, the disclosure relates to antisense oligonucleotides complementary to an exon 2 target region of the MSTN pre-mRNA designated as an annealing site (PCT Pub. No. WO2022212886).

In some embodiments, antisense oligonucleotides of the disclosure target MSTN pre-mRNA and induces skipping of exon 1, 2, and/or 3, so it is excluded or skipped from the mature, spliced mRNA transcript. By skipping exon 1, 2, and/or 3, the disrupted reading frame is restored to an in-frame mutation. While DMD is comprised of various genetic subtypes, antisense oligonucleotides of the disclosure were specifically designed to skip exon 1, 2, and/or 3 of MSTN pre-mRNA. In some embodiments, DMD mutations amenable to skipping exon 2 comprise a subgroup of DMD patients.

In some embodiments, the nucleobase sequence of an antisense oligonucleotides that induces exon 1, 2, and/or 3 skipping is designed to be complementary to a specific target sequence within exon 1, 2, and/or 3 of MSTN pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-AGCCCATCTTCTCCTGGTCCTGGGAAGG-3′ (SEQ ID NO:157) to target the MSTN gene (Table 2).

In some embodiments, the methods and compositions described herein are useful for treating Duchenne muscular dystrophy (DMD), by delivering an antisense oligonucleotide capable of inducing skipping of an exon in DMD pre-mRNA carrying a deleterious mutation, e.g., that causes a frameshift mutation. In certain embodiments, antisense oligonucleotides of the disclosure are complementary to at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, and 63 target regions of the dystrophin gene and induce exon skipping in at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, and 63. In some embodiments, the disclosure relates to antisense oligonucleotides complementary to an exon 23 target region of the dystrophin pre-mRNA designated as an annealing site. In some embodiments, the disclosure relates to antisense oligonucleotides complementary to an exon 43 target region of the dystrophin pre-mRNA designated as an annealing site. In some embodiments, the disclosure relates to antisense oligonucleotides complementary to an exon 43 target region of the dystrophin pre-mRNA designated as an annealing site. In some embodiments, the disclosure relates to antisense oligonucleotides complementary to an exon 44 target region of the dystrophin pre-mRNA designated as an annealing site. In some embodiments, the disclosure relates to antisense oligonucleotides complementary to an exon 45 target region of the dystrophin pre-mRNA designated as an annealing site. In some embodiments, the disclosure relates to antisense oligonucleotides complementary to an exon 50 target region of the dystrophin pre-mRNA designated as an annealing site. In some embodiments, the disclosure relates to antisense oligonucleotides complementary to an exon 51 target region of the dystrophin pre-mRNA designated as an annealing site. In some embodiments, the disclosure relates to antisense oligonucleotides complementary to an exon 52 target region of the dystrophin pre-mRNA designated as an annealing site. In some embodiments, the disclosure relates to antisense oligonucleotides complementary to an exon 53 target region of the dystrophin pre-mRNA designated as an annealing site. In some embodiments, the disclosure relates to antisense oligonucleotides complementary to an exon 55 target region of the dystrophin pre-mRNA designated as an annealing site (U.S. Pat. No. 11,193,125 B2; U.S. Pat. Appl. No. 20210147839A1; U.S. patent application No. 20210222169A1; U.S. patent application No. 20220152086 A1; U.S. patent application No. 20220333112A1; U.S. patent application No. 20210008095 A1; U.S. patent application No. 20200362336 A1; PCT Pub. No. WO2021172498 A1; U.S. patent application No. 20230038956 A1; all of which are incorporated by reference herein in their entirety).

In some embodiments, antisense oligonucleotides of the disclosure target dystrophin pre-mRNA and induces skipping of exon 23, 43, 44, 45, 50, 51, 52, 53, and/or 55, so it is excluded or skipped from the mature, spliced mRNA transcript. By skipping exon 23, 43, 44, 45, 50, 51, 52, 53, and/or 55, the disrupted reading frame is restored to an in-frame mutation. While DMD is comprised of various genetic subtypes, antisense oligonucleotides of the disclosure were specifically designed to skip exon 23, 43, 44, 45, 50, 51, 52, 53, and/or 55 of dystrophin pre-mRNA. In some embodiments, DMD mutations amenable to skipping exon 43 comprise a subgroup of DMD patients (<5%). In some embodiments, DMD mutations amenable to skipping exon 44 comprise a subgroup of DMD patients (<5%). In some embodiments, DMD mutations amenable to skipping exon 45 comprise a subgroup of DMD patients (8%). In some embodiments, DMD mutations amenable to skipping exon 50 comprise a subgroup of DMD patients (<5%). In some embodiments, DMD mutations amenable to skipping exon 51 comprise a subgroup of DMD patients (13%). In some embodiments, DMD mutations amenable to skipping exon 52 comprise a subgroup of DMD patients (<5%). In some embodiments, DMD mutations amenable to skipping exon 53 comprise a subgroup of DMD patients (10%). In some embodiments, DMD mutations amenable to skipping exon 53 comprise a subgroup of DMD patients (<5%).

In some embodiments, the nucleobase sequence of an antisense oligonucleotides that induces exon 23, 43, 44, 45, 50, 51, 52, 53, and/or 55 skipping is designed to be complementary to a specific target sequence within exon 23, 43, 44, 45, 50, 51, 52, 53, and/or 55 of dystrophin pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-GGCCAAACCTCGGCTTACCTGAAAT-3′ (SEQ ID NO: 99). In some embodiments where the antisense oligonucleotide is a peptide nucleic acid (PNA) oligonucleotide, the antisense oligonucleotide comprises the sequence 5′-KKKGGCCAAACCTCGGCTTACCTGAAATKKK-3′ (SEQ ID NO:405), where K is lysine.

In some embodiments, the antisense oligonucleotide comprises a sequence selected from SEQ ID NOs: 157-222, or SEQ ID NOs: 395-405 to target the DMD gene (Table 2).

In some embodiments, the antisense oligonucleotide composition includes any sequence disclosed in U.S. Pat. No. 9,228,187, entitled “Antisense molecules and methods for treating pathologies,” which is hereby incorporated by reference. In some embodiments, the antisense nucleotide composition includes any sequence disclosed in U.S. Pat. No. 9,447,415, entitled “Antisense oligonucleotides for inducing exon skipping and methods of use thereof,” which is hereby incorporated by reference. In some embodiments, the antisense oligonucleotide composition includes any sequence disclosed in U.S. Pat. No. 9,758,783, entitled “Antisense molecules and methods for treating pathologies,” which is hereby incorporated by reference. In some embodiments, the antisense nucleotide composition includes any sequence disclosed in U.S. Pat. No. 10,287,586, entitled “Antisense molecules and methods for treating pathologies,” which is hereby incorporated by reference. In some embodiments, the antisense oligonucleotide composition includes any sequence disclosed in U.S. Pat. No. 10,781,450, entitled “Antisense molecules and methods for treating pathologies”, which is hereby incorporated by reference.

In some embodiments, the antisense oligonucleotide is selected from the group consisting of: (i) an antisense oligonucleotide of 34 bases in length 100% complementary to a target region of exon 45 of the human dystrophin pre-mRNA, wherein the target region is annealing site H45A (−09+25), wherein the antisense oligonucleotide is a morpholino antisense oligonucleotide, and wherein the antisense oligonucleotide specifically hybridizes to the annealing site inducing exon 45 skipping; (ii) an antisense oligonucleotide of 28 bases in length 100% complementary to a target region of exon 45 of the human dystrophin pre-mRNA, wherein the target region is annealing site H45A (−03+25), wherein the antisense oligonucleotide is a morpholino antisense oligonucleotide, and wherein the antisense oligonucleotide specifically hybridizes to the annealing site inducing exon 45 skipping; (iii) an antisense oligonucleotide of 31 bases in length 100% complementary to a target region of exon 45 of the human dystrophin pre-mRNA, wherein the target region is annealing site H45A (−06+25), wherein the antisense oligonucleotide is a morpholino antisense oligonucleotide, and wherein the antisense oligonucleotide specifically hybridizes to the annealing site inducing exon 45 skipping; (iv) an antisense oligonucleotide of 31 bases in length 100% complementary to a target region of exon 45 of the human dystrophin pre-mRNA, wherein the target region is annealing site H45A (−12+19), wherein the antisense oligonucleotide is a morpholino antisense oligonucleotide, and wherein the antisense oligonucleotide specifically hybridizes to the annealing site inducing exon 45 skipping; (v) an antisense oligonucleotide of 22 bases in length 100% complementary to a target region of exon 45 of the human dystrophin pre-mRNA, wherein the target region is annealing site H45A (−03+19), wherein the antisense oligonucleotide is a morpholino antisense oligonucleotide, and wherein the antisense oligonucleotide specifically hybridizes to the annealing site inducing exon 45 skipping; (vi) an antisense oligonucleotide of 28 bases in length 100% complementary to a target region of exon 45 of the human dystrophin pre-mRNA, wherein the target region is annealing site H45A (−09+19), wherein the antisense oligonucleotide is a morpholino antisense oligonucleotide, and wherein the antisense oligonucleotide specifically hybridizes to the annealing site inducing exon 45 skipping; (vii) an antisense oligonucleotide of 28 bases in length 100% complementary to a target region of exon 45 of the human dystrophin pre-mRNA, wherein the target region is annealing site H45A (−12+16), wherein the antisense oligonucleotide is a morpholino antisense oligonucleotide, and wherein the antisense oligonucleotide specifically hybridizes to the annealing site inducing exon 45 skipping; (viii) an antisense oligonucleotide of 32 bases in length 100% complementary to a target region of exon 45 of the human dystrophin pre-mRNA, wherein the target region is annealing site H45A (−14+25), wherein the antisense oligonucleotide is a morpholino antisense oligonucleotide, and wherein the antisense oligonucleotide specifically hybridizes to the annealing site inducing exon 45 skipping; (ix) an antisense oligonucleotide of 27 bases in length 100% complementary to a target region of exon 45 of the human dystrophin pre-mRNA, wherein the target region is annealing site H45A (−08+19), wherein the antisense oligonucleotide is a morpholino antisense oligonucleotide, and wherein the antisense oligonucleotide specifically hybridizes to the annealing site inducing exon 45 skipping; (x) an antisense oligonucleotide of 32 bases in length 100% complementary to a target region of exon 45 of the human dystrophin pre-mRNA, wherein the target region is annealing site H45A (−07+25), wherein the antisense oligonucleotide is a morpholino antisense oligonucleotide, and wherein the antisense oligonucleotide specifically hybridizes to the annealing site inducing exon 45 skipping; (xi) an antisense oligonucleotide of 34 bases in length 100% complementary to a target region of exon 45 of the human dystrophin pre-mRNA, wherein the target region is annealing site H45A (−12+22), wherein the antisense oligonucleotide is a morpholino antisense oligonucleotide, and wherein the antisense oligonucleotide specifically hybridizes to the annealing site inducing exon 45 skipping; (xii) an antisense oligonucleotide of 31 bases in length 100% complementary to a target region of exon 45 of the human dystrophin pre-mRNA, wherein the target region is annealing site H45A (−09+22), wherein the antisense oligonucleotide is a morpholino antisense oligonucleotide, and wherein the antisense oligonucleotide specifically hybridizes to the annealing site inducing exon 45 skipping; (xiii) an antisense oligonucleotide of 39 bases in length 100% complementary to a target region of exon 45 of the human dystrophin pre-mRNA, wherein the target region is annealing site H45A (−09+30), wherein the antisense oligonucleotide is a morpholino antisense oligonucleotide, and wherein the antisense oligonucleotide specifically hybridizes to the annealing site inducing exon 45 skipping; (xiv) an antisense oligonucleotide of 28 bases in length 100% complementary to a target region of exon 45 of the human dystrophin pre-mRNA, wherein the target region is annealing site H45A (−06+22), wherein the antisense oligonucleotide is a morpholino antisense oligonucleotide, and wherein the antisense oligonucleotide specifically hybridizes to the annealing site inducing exon 45 skipping; (xv) an antisense oligonucleotide of 34 bases in length 100% complementary to a target region of exon 45 of the human dystrophin pre-mRNA, wherein the target region is annealing site H45A (−06+28), wherein the antisense oligonucleotide is a morpholino antisense oligonucleotide, and wherein the antisense oligonucleotide specifically hybridizes to the annealing site inducing exon 45 skipping; (xvi) an antisense oligonucleotide of 25 bases in length 100% complementary to a target region of exon 45 of the human dystrophin pre-mRNA, wherein the target region is annealing site H45A (−03+22), wherein the antisense oligonucleotide is a morpholino antisense oligonucleotide, and wherein the antisense oligonucleotide specifically hybridizes to the annealing site inducing exon 45 skipping; and (xvii) an antisense oligonucleotide of 31 bases in length 100% complementary to a target region of exon 45 of the human dystrophin pre-mRNA, wherein the target region is annealing site H45A (−03+28), wherein the antisense oligonucleotide is a morpholino antisense oligonucleotide, and wherein the antisense oligonucleotide specifically hybridizes to the annealing site inducing exon 45 skipping; or a pharmaceutically acceptable salt thereof.

In some embodiments, the antisense oligonucleotide is an antisense oligonucleotide of 22 bases comprising the base sequence CAA UGC CAU CCU GGA GUU CCU G (SEQ ID NO: 395), wherein the antisense oligonucleotide is a morpholino antisense oligonucleotide and is uniformly modified to comprise a 5-substituted pyrimidine base, or a pharmaceutically acceptable salt thereof. In other embodiments, the antisense oligonucleotide is the preceding antisense oligonucleotide, wherein the antisense oligonucleotide is chemically linked to one or more moieties or conjugates that enhance the activity, cellular distribution, or cellular uptake of the antisense oligonucleotide. In other embodiments, the antisense oligonucleotide is chemically linked to a polyethylene glycol chain. In some embodiments, the antisense oligonucleotide is an antisense oligonucleotide of 34 bases comprising the base sequence GCC CAA UGC CAU CCU GGA GUU CCU GUA AGA UAC C (SEQ ID NO:396), wherein the antisense oligonucleotide is a morpholino antisense oligonucleotide and is uniformly modified to comprise a 5-substituted pyrimidine base, or a pharmaceutically acceptable salt thereof. The antisense oligonucleotide of claim 35, wherein the antisense oligonucleotide is chemically linked to one or more moieties or conjugates that enhance the activity, cellular distribution, or cellular uptake of the antisense oligonucleotide. In other embodiments, the antisense oligonucleotide is chemically linked to a polyethylene glycol chain. In further embodiments, the antisense oligonucleotide is an antisense oligonucleotide of 31 bases comprising the base sequence GCC CAA UGC CAU CCU GGA GUU CCU GUA AGA U (SEQ ID NO:397), wherein the antisense oligonucleotide is a morpholino antisense oligonucleotide and is uniformly modified to comprise a 5-substituted pyrimidine base, or a pharmaceutically acceptable salt thereof. In other embodiments, the antisense oligonucleotide is chemically linked to one or more moieties or conjugates that enhance the activity, cellular distribution, or cellular uptake of the antisense oligonucleotide. In other embodiments, the antisense oligonucleotide is chemically linked to a polyethylene glycol chain. In other embodiments, the antisense oligonucleotide is an antisense oligonucleotide of 39 bases comprising the base sequence UUG CCG CUG CCC AAU GCC AUC CUG GAG UUC CUG UAA GAU (SEQ ID NO:398), wherein the antisense oligonucleotide is a morpholino antisense oligonucleotide and is uniformly modified to comprise a 5-substituted pyrimidine base, or a pharmaceutically acceptable salt thereof (U.S. Pat. No. 9,228,187).

In some embodiment, the antisense oligonucleotide of 20 to 31 bases comprises a base sequence that is 100% complementary to consecutive bases of exon 45 of the human dystrophin pre-mRNA, wherein the base sequence comprises at least 20 consecutive bases of CCA AUG CCA UCC UGG AGU UCC UGU AA (SEQ ID NO: 192), in which uracil bases are thymine bases, wherein the antisense oligonucleotide is a morpholino antisense oligonucleotide, and wherein the antisense oligonucleotide induces exon 45 skipping; or a pharmaceutically acceptable salt thereof.

In some embodiments, the antisense oligonucleotides are used in a method for treating a patient with Duchenne muscular dystrophy (DMD) in need thereof who has a mutation of the DMD gene that is amenable to exon 45 skipping, comprising administering to the patient an antisense oligonucleotide selected from the group consisting of: (i) an antisense oligonucleotide of 34 bases in length 100% complementary to a target region of exon 45 of the human dystrophin pre-mRNA, wherein the target region is annealing site H45A (−09+25), wherein the antisense oligonucleotide is a morpholino antisense oligonucleotide, and wherein the antisense oligonucleotide specifically hybridizes to the annealing site inducing exon 45 skipping; (ii) an antisense oligonucleotide of 28 bases in length 100% complementary to a target region of exon 45 of the human dystrophin pre-mRNA, wherein the target region is annealing site H45A (−03+25), wherein the antisense oligonucleotide is a morpholino antisense oligonucleotide, and wherein the antisense oligonucleotide specifically hybridizes to the annealing site inducing exon 45 skipping; (iii) an antisense oligonucleotide of 31 bases in length 100% complementary to a target region of exon 45 of the human dystrophin pre-mRNA, wherein the target region is annealing site H45A (−06+25), wherein the antisense oligonucleotide is a morpholino antisense oligonucleotide, and wherein the antisense oligonucleotide specifically hybridizes to the annealing site inducing exon 45 skipping; (iv) an antisense oligonucleotide of 31 bases in length 100% complementary to a target region of exon 45 of the human dystrophin pre-mRNA, wherein the target region is annealing site H45A (−12+19), wherein the antisense oligonucleotide is a morpholino antisense oligonucleotide, and wherein the antisense oligonucleotide specifically hybridizes to the annealing site inducing exon 45 skipping; (v) an antisense oligonucleotide of 22 bases in length 100% complementary to a target region of exon 45 of the human dystrophin pre-mRNA, wherein the target region is annealing site H45A (−03+19), wherein the antisense oligonucleotide is a morpholino antisense oligonucleotide, and wherein the antisense oligonucleotide specifically hybridizes to the annealing site inducing exon 45 skipping; (vi) an antisense oligonucleotide of 28 bases in length 100% complementary to a target region of exon 45 of the human dystrophin pre-mRNA, wherein the target region is annealing site H45A (−09+19), wherein the antisense oligonucleotide is a morpholino antisense oligonucleotide, and wherein the antisense oligonucleotide specifically hybridizes to the annealing site inducing exon 45 skipping; (vii) an antisense oligonucleotide of 28 bases in length 100% complementary to a target region of exon 45 of the human dystrophin pre-mRNA, wherein the target region is annealing site H45A (−12+16), wherein the antisense oligonucleotide is a morpholino antisense oligonucleotide, and wherein the antisense oligonucleotide specifically hybridizes to the annealing site inducing exon 45 skipping; (viii) an antisense oligonucleotide of 39 bases in length 100% complementary to a target region of exon 45 of the human dystrophin pre-mRNA, wherein the target region is annealing site H45A (−14+25), wherein the antisense oligonucleotide is a morpholino antisense oligonucleotide, and wherein the antisense oligonucleotide specifically hybridizes to the annealing site inducing exon 45 skipping; (ix) an antisense oligonucleotide of 27 bases in length 100% complementary to a target region of exon 45 of the human dystrophin pre-mRNA, wherein the target region is annealing site H45A (−08+19), wherein the antisense oligonucleotide is a morpholino antisense oligonucleotide, and wherein the antisense oligonucleotide specifically hybridizes to the annealing site inducing exon 45 skipping; (x) an antisense oligonucleotide of 32 bases in length 100% complementary to a target region of exon 45 of the human dystrophin pre-mRNA, wherein the target region is annealing site H45A (−07+25), wherein the antisense oligonucleotide is a morpholino antisense oligonucleotide, and wherein the antisense oligonucleotide specifically hybridizes to the annealing site inducing exon 45 skipping; (xi) an antisense oligonucleotide of 34 bases in length 100% complementary to a target region of exon 45 of the human dystrophin pre-mRNA, wherein the target region is annealing site H45A (−12+22), wherein the antisense oligonucleotide is a morpholino antisense oligonucleotide, and wherein the antisense oligonucleotide specifically hybridizes to the annealing site inducing exon 45 skipping; (xii) an antisense oligonucleotide of 31 bases in length 100% complementary to a target region of exon 45 of the human dystrophin pre-mRNA, wherein the target region is annealing site H45A (−09+22), wherein the antisense oligonucleotide is a morpholino antisense oligonucleotide, and wherein the antisense oligonucleotide specifically hybridizes to the annealing site inducing exon 45 skipping; (xiii) an antisense oligonucleotide of 39 bases in length 100% complementary to a target region of exon 45 of the human dystrophin pre-mRNA, wherein the target region is annealing site H45A (−09+30), wherein the antisense oligonucleotide is a morpholino antisense oligonucleotide, and wherein the antisense oligonucleotide specifically hybridizes to the annealing site inducing exon 45 skipping; (xiv) an antisense oligonucleotide of 28 bases in length 100% complementary to a target region of exon 45 of the human dystrophin pre-mRNA, wherein the target region is annealing site H45A (−06+22), wherein the antisense oligonucleotide is a morpholino antisense oligonucleotide, and wherein the antisense oligonucleotide specifically hybridizes to the annealing site inducing exon 45 skipping; (Xv) an antisense oligonucleotide of 34 bases in length 100% complementary to a target region of exon 45 of the human dystrophin pre-mRNA, wherein the target region is annealing site H45A (−06+28), wherein the antisense oligonucleotide is a morpholino antisense oligonucleotide, and wherein the antisense oligonucleotide specifically hybridizes to the annealing site inducing exon 45 skipping; (xvi) an antisense oligonucleotide of 25 bases in length 100% complementary to a target region of exon 45 of the human dystrophin pre-mRNA, wherein the target region is annealing site H45A (−03+22), wherein the antisense oligonucleotide is a morpholino antisense oligonucleotide, and wherein the antisense oligonucleotide specifically hybridizes to the annealing site inducing exon 45 skipping; and (xvii) an antisense oligonucleotide of 31 bases in length 100% complementary to a target region of exon 45 of the human dystrophin pre-mRNA, wherein the target region is annealing site H45A (−03+28), wherein the antisense oligonucleotide is a morpholino antisense oligonucleotide, and wherein the antisense oligonucleotide specifically hybridizes to the annealing site inducing exon 45 skipping; or a pharmaceutically acceptable salt thereof, thereby treating the patient.

In some embodiments, the antisense oligonucleotide is of 22 bases in length, wherein the antisense oligonucleotide is 100% complementary to a target region of exon 45 of the human dystrophin pre-mRNA, wherein the target region is annealing site H45A (−03+19), wherein the antisense oligonucleotide is a morpholino antisense oligonucleotide, and wherein the antisense oligonucleotide specifically hybridizes to the annealing site inducing exon 45 skipping, or a pharmaceutically acceptable salt thereof. In some embodiments, the antisense oligonucleotide has the IUPAC chemical structure P-DEOXY-P-(DIMETHYLAMINO)) (2′,3′-DIDEOXY-2′,3′-IMINO-2′,3′-SECO) (2′A-5′) (C-A-A-M5U-G-C-C-A-M5U-C-C-M5U-G-G-A-G-M5U-M5U-C-C-M5U-G), 5′-(P-(4-((2-(2-(2-HYDROXYETHOXY) ETHOXY) ETHOXY) CARBONYL)-1-PIPERAZINYL)-N,N-DIMETHYLPHOSPHONAMIDATE (SEQ ID NO:399)

In some embodiments, the antisense oligonucleotides are used in a method for restoring an mRNA reading frame to induce dystrophin protein production in a patient with Duchenne muscular dystrophy (DMD) in need thereof who has a mutation of the DMD gene that is amenable to exon 45 skipping, comprising administering to the patient an antisense oligonucleotide of 22 bases in length, wherein the antisense oligonucleotide is 100% complementary to a target region of exon 45 of the human dystrophin pre-mRNA, wherein the target region is annealing site H45A (−03+19), wherein the antisense oligonucleotide is a morpholino antisense oligonucleotide, and wherein the antisense oligonucleotide specifically hybridizes to the annealing site inducing exon 45 skipping, or a pharmaceutically acceptable salt thereof, thereby restoring the mRNA reading frame to induce dystrophin protein production in the patient.

In some embodiments, the antisense oligonucleotide composition includes any sequence disclosed in U.S. Pat. No. 9,018,368, entitled “Antisense oligonucleotides for inducing exon skipping and methods of use thereof,” which is hereby incorporated by reference.

In some embodiments, the antisense oligonucleotide composition includes any sequence disclosed in U.S. Pat. No. 9,243,245, entitled “Means and methods for counteracting muscle disorders,” which is hereby incorporated by reference. In some embodiments, the antisense oligonucleotide composition includes any sequence disclosed in U.S. Pat. No. 9,506,058, entitled “Compositions for treating muscular dystrophy,” which is hereby incorporated by reference. In some embodiments, the antisense oligonucleotide composition includes any sequence disclosed in U.S. Pat. No. 10,337,003, entitled “Compositions for treating muscular dystrophy,” which is hereby incorporated by reference. In some embodiments, the antisense nucleotide composition includes any sequence disclosed in U.S. Pat. No. 10,364,431, entitled “Compositions for treating muscular dystrophy”, which is hereby incorporated by reference. In some embodiments, the antisense oligonucleotide composition includes any sequence disclosed in U.S. Pat. No. 10,781,451, entitled “Antisense oligonucleotides for inducing exon skipping and methods of use thereof,” which is hereby incorporated by reference.

In some embodiments, the antisense oligonucleotide of 30 bases comprises the base sequence CUC CAA CAU CAA GGA AGA UGG CAU UUC UAG (SEQ ID NO:207), in which the uracil bases are thymine bases (CTC CAA CAT CAA GGA AGA TGG CAT TTC TAG, SEQ ID NO:208), wherein the antisense oligonucleotide is a morpholino antisense oligonucleotide, and wherein the antisense oligonucleotide is chemically linked to a polyethylene glycol chain. In a further embodiment, the antisense oligonucleotide is included in a pharmaceutical composition comprising an antisense oligonucleotide of 30 bases comprising the base sequence CUC CAA CAU CAA GGA AGA UGG CAU UUC UAG (SEQ ID NO:207), in which the uracil bases are thymine bases (CTC CAA CAT CAA GGA AGA TGG CAT TTC TAG, SEQ ID NO:208), wherein the antisense oligonucleotide is a morpholino antisense oligonucleotide, and wherein the antisense oligonucleotide is chemically linked to a polyethylene glycol chain, and a pharmaceutically acceptable carrier. In other embodiments, the antisense oligonucleotide is in a composition comprising: a first compound that increases the level of a functional dystrophin protein produced in a muscle cell of a Duchenne Muscular Dystrophy (DMD) or Becker Muscular Dystrophy (BMD) individual, wherein said first compound is an antisense oligonucleotide that induces skipping of exon 51 of human dystrophin pre-mRNA of said individual; and a second compound comprising a steroid; wherein, upon administration to a DMD or BMD patient, the composition increases the ratio of said dystrophin to laminin-α2 in muscle tissue of said patient as compared to the ratio of said dystrophin to laminin-α2 in muscle tissue of a patient administered with said first compound and not said second compound; and wherein said antisense oligonucleotide is 100% complementary to a portion of exon 51 that is 13 to 50 nucleotides in length and wherein said oligonucleotide comprises a non naturally-occurring modification. In other embodiments, the antisense oligonucleotides are used in a method for treating a patient with Duchenne muscular dystrophy (DMD) in need thereof who has a mutation of the DMD gene that is amenable to exon 51 skipping, comprising intravenously administering to the patient eteplirsen at a dose of about 30 mg/kg weekly for more than 120 weeks, such that disease progression in the patient is delayed, thereby treating the patient. In some embodiments, the antisense oligonucleotide is used in a method of treating Duchenne muscular dystrophy (DMD) in a human subject who has a mutation of the DMD gene that is amenable to exon 51 skipping, comprising administering to the human subject a composition comprising eteplirsen and a phosphate-buffered saline at a dose of eteplirsen of about 30 mg/kg to about 50 mg/kg for a period of time sufficient to increase the number of dystrophin-positive fibers in a subject to at least 20% of normal. In some embodiments, the antisense oligonucleotide is used in a method for treating Duchenne muscular dystrophy (DMD) in a patient in need thereof who has a mutation of the DMD gene that is amenable to exon 51 skipping, comprising intravenously administering to the patient a composition comprising eteplirsen, or a pharmaceutically acceptable salt thereof, and a pharmaceutically acceptable carrier, wherein eteplirsen, or a pharmaceutically acceptable salt thereof, is administered at a dose of about 30 mg/kg once a week for more than 120 weeks, such that disease progression in the patient is delayed, thereby treating the patient. In some embodiments, the antisense oligonucleotide is an antisense oligonucleotide of 30 bases comprising the base sequence CUC CAA CAU CAA GGA AGA UGG CAU UUC UAG (SEQ ID NO: 207), in which the uracil bases are thymine bases (CTC CAA CAT CAA GGA AGA TGG CAT TTC TAG, SEQ ID NO:208), wherein the antisense oligonucleotide is a morpholino antisense oligonucleotide.

In some embodiments, the antisense oligonucleotide used to treat DMD has the IUPAC chemical structure P-DEOXY-P-(DIMETHYLAMINO)) (2′,3′-DIDEOXY-2′,3′-IMINO-2′,3′-SECO) (2′a→5′) (C-m5U-C-C-A-A-C-A-m5U-C-A-A-G-G-A-A-G-A-m5U-G-G-C-A-m5U-m5U-m5U-C-m5U-A-G),5′-(P-(4-((2-(2-(2-HYDROXYETHOXY)-ETHOXY)-ETHOXY)-CARBONYL)-1-PIPERAZINYL)-N,N-DIMETHYLPHOSPHONAMIDATE) (SEQ ID NO: 400)

In some embodiments, the antisense oligonucleotide composition includes any sequence dis-closed in U.S. Pat. No. 9,024,007, entitled “Antisense oligonucleotides for inducing exon skipping and methods of use thereof,” which is hereby incorporated by reference. In some embodiments, the antisense oligonucleotide composition includes any sequence disclosed in U.S. Pat. No. 9,994,851, entitled “Antisense oligonucleotides for inducing exon skipping and methods of use thereof,” which is hereby incorporated by reference. In some embodiments, the antisense oligonucleotide composition includes any sequence disclosed in U.S. Pat. No. 10,227,590, entitled “Antisense oligonucleotides for inducing exon skipping and methods of use thereof,” which is hereby incorporated by reference. In some embodiments, the antisense oligonucleotide composition includes any sequence disclosed in U.S. Pat. No. 10,266,827, entitled “Antisense oligonucleotides for inducing exon skipping and methods of use thereof,” which is hereby incorporated by reference. In some embodiments, the antisense oligonucleotide composition includes any sequence disclosed in U.S. Pat. No. 10,421,966, entitled “Antisense oligonucleotides for inducing exon skipping and methods of use thereof”, which is hereby incorporated by reference. In some embodiments, the antisense oligonucleotide composition includes any sequence disclosed in U.S. Pat. No. 10,968,450, entitled “Antisense oligonucleotides for inducing exon skipping and methods of use thereof”, which is hereby incorporated by reference. In some embodiments, the antisense oligonucleotide composition includes any sequence disclosed in U.S. Pat. No. 10,995,337, entitled “Antisense oligonucleotides for inducing exon skipping and methods of use thereof”, which is hereby incorporated by reference.

In some embodiments, the antisense oligonucleotide is an antisense oligonucleotide of 25 bases comprising a base sequence that is 100% complementary to 25 consecutive nucleotides of a target region of exon 53 of the human dystrophin pre-mRNA, wherein the target region is within annealing site H53A (+23+47) and annealing site H53A (+39+69), wherein the antisense oligonucleotide base sequence comprises at least 20 consecutive bases of CAU UCA ACU GUU GCC UCC GGU UCU GAA GGU G (SEQ ID NO:212), in which uracil bases are thymine bases, wherein the antisense oligonucleotide is a morpholino antisense oligonucleotide, wherein the antisense oligonucleotide is chemically linked to a polyethylene glycol chain, and wherein the antisense oligonucleotide specifically hybridizes to the target region to induce exon 53 skipping. In a further embodiment, the antisense oligonucleotide is included in a pharmaceutical composition comprising an antisense oligonucleotide of 25 bases comprising a base sequence that is 100% complementary to 25 consecutive nucleotides of a target region of exon 53 of the human dystrophin pre-mRNA, wherein the target region is within annealing site H53A (+23+47) and annealing site H53A (+39+69), wherein the antisense oligonucleotide base sequence comprises at least 20 consecutive bases of CAU UCA ACU GUU GCC UCC GGU UCU GAA GGU G (SEQ ID NO: 212), in which uracil bases are thymine bases, wherein the antisense oligonucleotide is a morpholino antisense oligonucleotide, wherein the antisense oligonucleotide is chemically linked to a polyethylene glycol chain, and wherein the antisense oligonucleotide specifically hybridizes to the target region to induce exon 53 skipping, and a pharmaceutically acceptable carrier. In other embodiments, the antisense oligonucleotide is an antisense oligonucleotide of 20 to 31 bases comprising a base sequence that is 100% complementary to consecutive bases of a target region of exon 53 of the human dystrophin pre-mRNA, wherein the target region is within annealing site H53A (+23+47) and annealing site H53A (+39+69), wherein the base sequence comprises at least 12 consecutive bases of CUG AAG GUG UUC UUG UAC UUC AUC C (SEQ ID NO: 211), in which uracil bases are thymine bases, wherein the antisense oligonucleotide is a morpholino antisense oligonucleotide, and wherein the antisense oligonucleotide induces exon 53 skipping; or a pharmaceutically acceptable salt thereof. In some embodiments, the antisense oligonucleotide is an antisense oligonucleotide of 20 to 31 bases comprising a base sequence that is 100% complementary to consecutive bases of a target region of exon 53 of the human dystrophin pre-mRNA, wherein the base sequence comprises at least 12 consecutive bases of CUG AAG GUG UUC UUG UAC UUC AUC C (SEQ ID NO: 211), in which uracil bases are thymine bases, wherein the antisense oligonucleotide is a morpholino antisense oligonucleotide, and wherein the antisense oligonucleotide induces exon 53 skipping; or a pharmaceutically acceptable salt thereof. In other embodiments, the antisense oligonucleotide is used in a method for treating a patient with Duchenne muscular dystrophy (DMD) in need thereof who has a mutation of the DMD gene that is amenable to exon 53 skipping, comprising administering to the patient an antisense oligonucleotide of 20 to 31 bases comprising a base sequence that is 100% complementary to consecutive bases of a target region of exon 53 of the human dystrophin pre-mRNA, wherein the base sequence comprises at least 12 consecutive bases of CUG AAG GUG UUC UUG UAC UUC AUC C (SEQ ID NO:211), in which uracil bases are thymine bases, wherein the antisense oligonucleotide is a morpholino antisense oligonucleotide, and wherein the antisense oligonucleotide induces exon 53 skipping; or a pharmaceutically acceptable salt thereof. In some embodiments, the antisense oligonucleotide is an antisense oligonucleotide of 25 bases comprising a base sequence that is 100% complementary to consecutive bases of a target region of exon 53 of the human dystrophin pre-mRNA, wherein the base sequence comprises at least 12 consecutive bases of CUG AAG GUG UUC UUG UAC UUC AUC C (SEQ ID NO:211), in which uracil bases are thymine bases, wherein the antisense oligonucleotide is a morpholino antisense oligonucleotide, wherein the antisense oligonucleotide is chemically linked to a polyethylene glycol chain, and wherein the antisense oligonucleotide induces exon 53 skipping; or a pharmaceutically acceptable salt thereof. In other embodiments, the antisense oligonucleotide is an antisense oligonucleotide comprising a base sequence 25 bases in length that is 100% complementary to 25 consecutive nucleotide bases of a target region of exon 53 of the human dystrophin pre-mRNA, wherein the antisense oligonucleotide base sequence comprises at least 20 consecutive bases of CAU UCA ACU GUU GCC UCC GGU UCU GAA GGU G (SEQ ID NO:212), in which the uracil bases are thymine bases, wherein the antisense oligonucleotide is a morpholino antisense oligonucleotide, and wherein the antisense oligonucleotide specifically hybridizes to the target region and induces exon 53 skipping, or a pharmaceutically acceptable salt thereof. In some embodiments, the antisense oligonucleotide is used in a method of treating Duchenne muscular dystrophy, comprising administering an effective amount of an antisense oligonucleotide comprising a base sequence 25 bases in length that is 100% complementary to 25 consecutive bases of a target region of exon 53 of the human dystrophin pre-mRNA, said morpholino antisense oligonucleotide is chemically linked to a polyethylene glycol chain; wherein the antisense oligonucleotide base sequence comprises at least 20 consecutive bases of CAU UCA ACU GUU GCC UCC GGU UCU GAA GGU G (SEQ ID NO: 212), in which the uracil bases are thymine bases, wherein the antisense oligonucleotide is a morpholino antisense oligonucleotide, and wherein the antisense oligonucleotide specifically hybridizes to the target region and induces exon 53 skipping.

In some embodiments, the antisense oligonucleotide has the sequence GUU GCC UCC GGU UCU GAA GGU GUU C (SEQ ID NO:401). In further embodiments, the antisense oligonucleotide has the IUPAC chemical structure P-DEOXY-P-(DIMETHYLAMINO)) (2′,3′-DIDEOXY-2′,3′-IMINO-2′,3′-SECO) (2′A→5′) (G-m5U-m5U-G-C-C-m5U-C-C-G-G-m5U-M5U-C-m5U-G-A-A-G-G-m5U-G-m5U-m5U-C), 5′-(P-(4-((2-(2-(2-HYDROXYETHOXY)-ETHOXY-) ETHOXY)-CARBONYL)-1-PIPERAZINYL)-N,N-DIMETHYLPHOSPHONAMIDATE (SEQ ID NO:403).

In some embodiments, the antisense oligonucleotide composition includes any sequence dis-closed in U.S. Pat. No. 9,079,934, entitled “Antisense nucleic acids,” which is hereby incorporated by reference. In some embodiments, the antisense oligonucleotide composition includes any sequence disclosed in U.S. Pat. No. 10,870,676, entitled “Antisense nucleic acids,” which is hereby incorporated by reference.

In some embodiments, the antisense oligonucleotide is an antisense oligomer which causes skipping of the 53rd exon in the human dystrophin gene, consisting of the nucleotide sequence of 5′-CCTCCGGTTCTGAAGGTGTTC-3′ (SEQ ID NO:213), wherein the antisense oligomer is an oligonucleotide having the sugar moiety and/or the phosphate-binding region of at least one nucleotide constituting the oligonucleotide modified, or a morpholino oligomer. In other embodiments, the antisense oligonucleotide comprises the sugar moiety of at least one nucleotide constituting the oligonucleotide is a ribose in which the 2′—OH group is replaced by any one selected from the group consisting of OR, R, R′OR, SH, SR, NH2, NHR, NR2, N3, CN, F, Cl, Br and I (wherein R is an alkyl or an aryl and R′ is an alkylene). In other embodiments, the antisense oligomer comprises the phosphate-binding region of at least one nucleotide constituting the oligonucleotide is any one selected from the group consisting of a phosphorothioate bond, a phosphorodithioate bond, an alkylphosphonate bond, a phosphoramidate bond and a boranophosphate bond. In other embodiments, the antisense oligonucleotide is included in a pharmaceutical composition for the treatment of muscular dystrophy, comprising as an active, ingredient the antisense oligonucleotide as defined above, or a pharmaceutically acceptable salt or hydrate thereof. In other embodiments, the antisense oligonucleotide is used in a method of treating Duchenne muscular dystrophy (DMD) in a patient in need thereof comprising administering an antisense oligonucleotide, or a pharmaceutically acceptable salt or hydrate thereof, wherein the antisense oligonucleotide consists of a nucleotide sequence complementary to the sequence consisting of the 36th to the 56th nucleotides from the 5′ end of the 53rd exon in a human dystrophin pre-mRNA.

In some embodiments, the antisense oligonucleotide has the sequence CCU CCG GUU CUG AAG GUG UUC (SEQ ID NO:402). In other embodiments, the antisense oligonucleotide has the IUPAC chemical structure P-DEOXY-P-(DIMETHYLAMINO)) (2′,3′-DIDEOXY-2′,3′-IMINO-2′,3′-SECO) (2′A→5′) (C-C-m5U-C-C-G-G-m5U-m5U-C-m5U-G-A-A-G-G-m5U-G-m5U-m5U-C) (SEQ ID NO:404).

### DM1 and DM2

Myotonic dystrophy type 1 (DM1; OMIM 160900) and type 2 (DM2; OMIM 602668) are associated with long polyCUG and polyCCUG repeats in the 3′-UTR and intron 1 regions of the transcript dystrophia myotonica protein kinase (DMPK) and zinc finger protein 9 (ZNF9), respectively. While normal individuals have as many as 30 CTG repeats, DM1 patients carry a larger number of repeats ranging from 50 to thousands. The severity of the disease and the age of onset correlates with the number of repeats. Patients with adult onsets show milder symptoms and have less than 100 repeats, juvenile onset DM1 patients carry as many as 500 repeats and congenital cases usually have around a thousand CTG repeats. The expanded transcripts containing CUG repeats form a secondary structure, accumulate in the nucleus in the form of nuclear foci and sequester RNAbinding proteins (RNA-BP) (U.S. Pat. No. 10,106,796, herein incorporated by reference in its entirety).

In some embodiments, the methods and compositions described herein are useful for treating myotonic dystrophy type 1 (DM1) or 2 (DM2), by delivering an antisense oligonucleotide capable of inducing skipping of an exon in DMPK or ZNF9 pre-mRNA carrying a deleterious mutation, e.g., that causes a frameshift mutation. In certain embodiments, antisense oligonucleotides of the disclosure are complementary to the 3′-UTR polyGUC repeats target region of the DMPK gene; or to the intron 1 polyCCUG repeats target region of the ZNF9 gene, and induce exon skipping in at least one of exon 1, 2, 3, 4, 5, 6, 7, 8, 9, 10, 11, 12, 13, 14, or 15 of the DMPK gene; or in at least one of exon 1, 2, 3, 4, or 5 of the ZNF9 gene.

In some embodiments, antisense oligonucleotides of the disclosure target DMPK pre-mRNA and induces skipping of exon 1, 2, 3, 4, 5, 6, 7, 8, 9, 10, 11, 12, 13, 14, or 15 of the DMPK gene, so it is excluded or skipped from the mature, spliced mRNA transcript. By skipping exon 1, 2, 3, 4, 5, 6, 7, 8, 9, 10, 11, 12, 13, 14, or 15, the disrupted reading frame is restored to an in-frame mutation. In some embodiments, antisense oligonucleotides of the disclosure target ZNF9 pre-mRNA and induces skipping of exon 1, 2, 3, 4, or 5 of the ZNF9 gene, so it is excluded or skipped from the mature, spliced mRNA transcript. By skipping exon 1, 2, 3, 4, or 5, the disrupted reading frame is restored to an in-frame mutation.

In some embodiments, the nucleobase sequence of an antisense oligonucleotides that induces exon 1, 2, 3, 4, 5, 6, 7, 8, 9, 10, 11, 12, 13, 14, or 15 skipping is designed to be complementary to a specific target sequence within exon 1, 2, 3, 4, 5, 6, 7, 8, 9, 10, 11, 12, 13, 14, or 15 of DMPK pre-mRNA. In some embodiments, the nucleobase sequence of an antisense oligonucleotides that induces exon 1, 2, 3, 4, or 5 skipping is designed to be complementary to a specific target sequence within exon 1, 2, 3, 4, or 5 of ZNF9 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises one of the sequence SEQ ID NOs: 223-226 to target the DMPK gene. In some embodiments, the antisense oligonucleotide comprises one of the sequence SEQ ID NOs: 227-232 to target the ZNF9 gene (Table 2).

### SMA

Spinal muscular atrophy (SMA; OMIM 253400) is a neuromuscular disease caused by mutations in telomeric SMN1, a gene encoding a ubiquitously expressed protein (survival of motor neuron SMN) involved in spliceosome biogenesis. The SMN gene product is intracellular and SMN deficiency results in selective toxicity to lower motor neurons, resulting in progressive neuron loss and muscle weakness. The severity of the disease is modified by the copy number of a centromeric duplication of the homologous gene (SMN2), which carries a splice site mutation that results in production of only small amounts of the full length SMN transcript. Patients who carry one to two copies of SMN2 present with the severe form of SMA, characterized by onset in the first few months of life and rapid progression to respiratory failure. Patients with three copies of SMN2 generally exhibit an attenuated form of the disease, typically presenting after six months of age. Though many never gain the ability to walk, they rarely progress to respiratory failure, and often live into adulthood. Patients with four SMN2 copies may not present until adulthood with gradual onset of muscle weakness.

Accordingly, in some aspects a method of treating SMA in a subject (e.g., a human subject) having SMA involves administering to the subject a recombinant nucleic acid that encodes SMN1 (also referred to as a recombinant SMN1 gene), and an ASO that increases full-length SMN2 mRNA in a subject (also referred to as an SMN2 ASO) (U.S. patent application No. 20210308281 A1, herein incorporated by reference in its entirety). In some embodiments, the methods and compositions described herein are useful for treating Spinal muscular atrophy type 3, by delivering an antisense oligonucleotide capable of inducing skipping of an exon in SMN2 pre-mRNA carrying a deleterious mutation, e.g., that causes a frameshift mutation. In certain embodiments, antisense oligonucleotides of the disclosure are complementary to exon 7 target regions of the SMN2 gene, and induce exon skipping in at least one of exon 7 the SMN2 gene.

In some embodiments, antisense oligonucleotides of the disclosure target SMN2 pre-mRNA and induces skipping of exon 7 of the SMN2 gene, so it is excluded or skipped from the mature, spliced mRNA transcript. By skipping exon 7, the disrupted reading frame is restored to an in-frame mutation.

In some embodiments, the nucleobase sequence of an antisense oligonucleotides that induces exon 7 skipping is designed to be complementary to a specific target sequence within exon 7 of SMN2 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises one of the sequence 5′-tcactttcataatgctgg-3′ (SEQ ID NO:233) to target the SMN2 gene.

### IBMPFD1

In some embodiments, the methods and compositions described herein are useful for treating Inclusion body myopathy with early-onset Paget disease and frontotemporal dementia 1 (IBMPFD1; OMIM 167320), by delivering an antisense oligonucleotide capable of inducing skipping of an exon in VCP pre-mRNA carrying a deleterious mutation, e.g., that causes a frameshift mutation. In certain embodiments, antisense oligonucleotides of the disclosure are complementary to exon 5 target regions of the VCP gene, and induce exon skipping in at least one of exon 5 the VCP gene.

In some embodiments, antisense oligonucleotides of the disclosure target VPC pre-mRNA and induces skipping of exon 5 of the VCP gene, so it is excluded or skipped from the mature, spliced mRNA transcript. By skipping exon 5, the disrupted reading frame is restored to an in-frame mutation.

In some embodiments, the nucleobase sequence of an antisense oligonucleotides that induces exon 5 skipping is designed to be complementary to a specific target sequence within exon 5 of VCP pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises one of the sequence SEQ ID NOs: 265-270 to target the VCP gene.

### Cardiovascular Diseases

### FH

Familial hypercholesterolemia (FH; OMIM 144010) is a condition characterized by extremely high concentrations of low-density lipoprotein (LDL) cholesterol, most commonly due to genetic defects in the hepatic LDL receptor. Patients with the most severe form, homozygous FH, develop life-threatening cardiovascular disease (CVD) in early adulthood. Lowering LDL cholesterol is known to reduce mortality and morbidity, delaying the onset of CVD (Disterer et al., Mol Therap. 2013 21 (3); 602-609; PCT Publication No. WO2013057485 A1).

In some embodiments, the methods and compositions described herein are useful for treating FH, by delivering an antisense oligonucleotide capable of inducing skipping of an exon in APOB pre-mRNA carrying a deleterious mutation, e.g., that causes a frameshift mutation. In certain embodiments, antisense oligonucleotides of the disclosure are complementary to exon 27 target regions of the APOB gene, and induce exon skipping in at least one of exon 27 the APOB gene.

In some embodiments, antisense oligonucleotides of the disclosure target APOB pre-mRNA and induces skipping of exon 27 of the APOB gene, so it is excluded or skipped from the mature, spliced mRNA transcript. By skipping exon 27, the disrupted reading frame is restored to an in-frame mutation.

In some embodiments, the nucleobase sequence of an antisense oligonucleotides that induces exon 27 skipping is designed to be complementary to a specific target sequence within exon 27 of APOB pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises a sequence selected from SEQ ID NOs: 368-370 to target the APOB gene (Table 2).

### Metabolic Diseases

### ACADMD

Medium-chain Acyl-CoA dehydrogenase (MCAD) deficiency (ACADMD; OMIM 201450) is the most commonly recognized defect of the mitochondrial P-oxidation in humans. It is a potentially fatal autosomal recessive inherited defect, which may present in the first 2 years of life if patients experience periods of metabolic stress to the P-oxidation system (Vianey-Liaud et al. 1987; Roe and Coates 1989). The clinical picture is heterogeneous, ranging from severe episodes of hypoglycemia and coma to years of remaining without symptoms (Roe and Coates 1989). Occasionally, patients with ACADMD die suddenly and unexpectedly (Roe and Coates 1989). Approximately 80% of patients with ACADMD are homozygous for the G985 mutation and can thus be easily diagnosed by a simple PCR-based assay (Gregersen et al. 1991b; Yokota et al. 1991). The majority of the remaining patients are compound heterozygotes with G985 as one of the disease alleles (Andresen et al., Disease-causing mutations in exon 11 of the medium chain Acyl-CoA dehydrogenase gene, Am. J. Hum. Genet 54:975-988, 1994; Holm et al., 2022, Hum Mutat 43 (2): 253-265).

In some embodiments, the methods and compositions described herein are useful for treating ACADMD, by delivering an antisense oligonucleotide capable of inducing skipping of an exon in MCAD pre-mRNA carrying a deleterious mutation, e.g., that causes a frameshift mutation. In certain embodiments, antisense oligonucleotides of the disclosure are complementary to exon 5 target regions of the MCAD gene, and induce exon skipping in at least one of exon 5 the MCAD gene. In certain embodiments, antisense oligonucleotides of the disclosure are complementary to exon 11 target regions of the MCAD gene, and induce exon skipping in at least one of exon 11 the MCAD gene.

In some embodiments, antisense oligonucleotides of the disclosure target MCAD pre-mRNA and induces skipping of exon 5 of the MCAD gene, so it is excluded or skipped from the mature, spliced mRNA transcript. By skipping exon 5, the disrupted reading frame is restored to an in-frame mutation. In some embodiments, antisense oligonucleotides of the disclosure target MCAD pre-mRNA and induces skipping of exon 11 of the MCAD gene, so it is excluded or skipped from the mature, spliced mRNA transcript. By skipping exon 11, the disrupted reading frame is restored to an in-frame mutation.

In some embodiments, the nucleobase sequence of an antisense oligonucleotides that induces exon 5 skipping is designed to be complementary to a specific target sequence within exon 5 of MCAD pre-mRNA. In some embodiments, the nucleobase sequence of an antisense oligonucleotides that induces exon 11 skipping is designed to be complementary to a specific target sequence within exon 11 of MCAD pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

### PKU

Deficiency of phenylalanine hydroxylase (PAH, EC 1.14.16.1) is causing phenylketonuria (PKU, OMIM 261600), an autosomal recessively inherited disease presenting with elevated blood phenylalanine (Phe) levels. The phenotypic severity of PKU is characterized by the type of mutation, and thus by residual PAH enzyme activity. The fully functional homotetrameric PAH catalyzes hydroxylation of Phe to tyrosine (Tyr) in the presence of cofactor (6R)-L-erythro-5,6,7,8-tetrahydrobiopterin (BH4) and molecular oxygen. According to the Locus Knowledgebase (PAHdb, www.pahdb.mcgill.ca), about 60% of mutations in the PAH gene are missense mutations, which may lead to a misfolding of the protein, disturbing the complex enzyme regulation and changes in kinetics, due to altered affinities for the Phe substrate and the BH4 cofactor (Heintz et al., Quantification of phenylalanine hydroxylase activity by isotope-dilution liquid chromatography-electrospray ionization tandem mass spectrometry, Mol Genet and Metab 2012 105 (4); 559-565).

In some embodiments, the methods and compositions described herein are useful for treating phenylketonuria, by delivering an antisense oligonucleotide capable of inducing skipping of an exon in PAH pre-mRNA carrying a deleterious mutation, e.g., that causes a frameshift mutation. In certain embodiments, antisense oligonucleotides of the disclosure are complementary to exon 27 target regions of the PAH gene, and induce exon skipping in at least one of exon 11 the PAH gene.

In some embodiments, antisense oligonucleotides of the disclosure target PAH pre-mRNA and induces skipping of exon 11 of the PAH gene, so it is excluded or skipped from the mature, spliced mRNA transcript. By skipping exon 11, the disrupted reading frame is restored to an in-frame mutation.

In some embodiments, the nucleobase sequence of an antisense oligonucleotides that induces exon 11 skipping is designed to be complementary to a specific target sequence within exon 11 of PAH pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-ATCCTCTTTGGTAACCTCACCTCAC-3′ (SEQ ID NO:371) to target the PAH gene (Table 2).

### NPC1

Niemann-Pick Disease, type IC (NPC1; OMIM 257220) is an autosomal recessive lipid storage disorder characterized by progressive neurodegeneration. Approximately 95% of cases are caused by mutations in the NPC1 gene, referred to as type C1; 5% are caused by mutations in the NPC2 gene, referred to as type C2. The clinical manifestations of types C1 and C2 are similar because the respective genes are both involved in egress of lipids, particularly cholesterol, from late endosomes or lysosomes. Patients usually develop difficulty coordinating movements (ataxia), an inability to move the eyes vertically (vertical supranuclear gaze palsy), poor muscle tone (dystonia), severe liver disease, and interstitial lung disease. Individuals with NPC1 have problems with speech and swallowing that worsen over time, eventually interfering with feeding. Affected individuals often experience progressive decline in intellectual function and about one-third have seizures.

In some embodiments, the methods and compositions described herein are useful for treating NPC1, by delivering an antisense oligonucleotide capable of inducing skipping of an exon in NPC1 pre-mRNA carrying a deleterious mutation, e.g., that causes a frameshift mutation. In certain embodiments, antisense oligonucleotides of the disclosure are complementary to exon 27 target regions of the NPC1 gene, and induce exon skipping in at least one of exon 15 the NPC1 gene.

In some embodiments, antisense oligonucleotides of the disclosure target NPC1 pre-mRNA and induces skipping of exon 15 of the NPC1 gene, so it is excluded or skipped from the mature, spliced mRNA transcript. By skipping exon 15, the disrupted reading frame is restored to an in-frame mutation.

In some embodiments, the nucleobase sequence of an antisense oligonucleotides that induces exon 15 skipping is designed to be complementary to a specific target sequence within exon 15 of NPC1 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises the sequence 5′-UGGCAUCACGGACAAUGC-3′ (SEQ ID NO:341) to target the NPC1 gene (Table 2).

### Cancers

### RAS-Associated Cancers

The three closely related human RAS genes, HRAS, KRAS, and NRAS, are all widely expressed and are important for regulation of numerous cellular processes through the RAS-MAP-kinase and PI3K/Akt pathways. They each exhibit oncogenic activity and more than 30% of all human tumors have mutations leading to constitutively active RAS proteins. Different RAS oncogenes are preferentially associated with different types of human cancers. Therefore, the RAS oncogenes are already targets for numerous different anticancer treatments (U.S. Pat. No. 10,266,828, incorporated herein by reference in its entirety).

In some embodiments, the methods and compositions described herein are useful for treating various RAS-associated cancers including, but not limited to, bladder cancer (OMIM 109800), breast cancer (OMIM 114480), gastric cancer (OMIM 613659), acute myeloid leukemia (OMIM 601626), lung cancer (OMIM 211980), pancreatic carcinoma (OMIM 260350), RAS-associated autoimmune leukoproliferative disorder type IV (OMIM 614470), colorectal cancer (OMIM 114500), and follicular thyroid carcinoma (OMIM 188470), by delivering an antisense oligonucleotide capable of inducing skipping of an exon in HRAS, KRAS, or NRAS pre-mRNA carrying a deleterious mutation, e.g. that causes a frameshift mutation. In some embodiments, antisense nucleotides of the disclosure are complementary to at least one of exon 1, 2, 3, 4, 5, or 6 target regions of the HRAS gene; at least one of exon 1, 2, 3, 4, or 5 target regions of the KRAS gene; at least one of exon 1, 2, 3, 4, 5, 6, or 7 target regions of the NRAS gene; and induce skipping in at least one of exon 1, 2, 3, 4, 5, or 6 of the HRAS gene; 1, 2, 3, 4, or 5 of the KRAS gene; and 1, 2, 3, 4, 5, 6, or 7 of the NRAS gene, respectively. In some embodiments, the disclosure relates to antisense oligonucleotides complementary to an exon 2 target region of the HRAS pre-mRNA designated as an annealing site. In some embodiments, the disclosure relates to antisense oligonucleotides complementary to an exon 2 target region of the KRAS pre-mRNA designated as an annealing site. In some embodiments, the disclosure relates to antisense oligonucleotides complementary to an exon 2 target region of the NRAS pre-mRNA designated as an annealing site.

In some embodiments, antisense oligonucleotides of the disclosure target HRAS pre-mRNA and induces skipping of exon 2, so it is excluded or skipped from the mature, spliced mRNA transcript. By skipping exon 2, the disrupted reading frame is restored to an in-frame mutation. In some embodiments, antisense oligonucleotides of the disclosure target KRAS pre-mRNA and induces skipping of exon 2, so it is excluded or skipped from the mature, spliced mRNA transcript. By skipping exon 2, the disrupted reading frame is restored to an in-frame mutation. In some embodiments, antisense oligonucleotides of the disclosure target NRAS pre-mRNA and induces skipping of exon 2, so it is excluded or skipped from the mature, spliced mRNA transcript. By skipping exon 2, the disrupted reading frame is restored to an in-frame mutation.

In some embodiments, the nucleobase sequence of an antisense oligonucleotides that induces exon 2 skipping is designed to be complementary to a specific target sequence within exon 2 of HRAS, KRAS, or NRAS pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises a sequence selected from SEQ ID NOs: 234-244 to target the HRAS gene. In some embodiments, the antisense oligonucleotide comprises a sequence selected from SEQ ID NOs: 245-255 to target the KRAS gene. In some embodiments, the antisense oligonucleotide comprises a sequence selected from SEQ ID NOs: 256-264 to target the NRAS gene.

### MLD

Metachromatic leukodystrophy (MLD; OMIM 250100) is a lysosomal storage disease caused by an arylsulfatase A (ARSA) deficiency and characterized by severe neurological symptoms resulting from demyelination within the central and peripheral nervous systems. This disease is characterized pathologically by myelin degeneration in both the central and peripheral nervous systems (CNS and PNS). Although enzyme replacement therapy using human ARSA has been tried, this approach does not effectively relieve the neurological symptoms. Gene therapy is one of the potentially effective strategies under consideration for use in the treatment of CNS disorders, and several gene therapy protocols for treating MLD have been proposed (Miyake et al., 2021, Sci Rep 11:20513).

In some embodiments, the methods and compositions described herein are useful for treating MLD, by delivering an antisense oligonucleotide capable of inducing skipping of an exon in ARSA pre-mRNA carrying a deleterious mutation, e.g., that causes a frameshift mutation. In certain embodiments, antisense oligonucleotides of the disclosure are complementary to exon 2 target regions of the ARSA gene, and induce exon skipping in at least one of exon 2 the ARSA gene. In certain embodiments, antisense oligonucleotides of the disclosure are complementary to exon 3 target regions of the ARSA gene, and induce exon skipping in at least one of exon 3 the ARSA gene. In certain embodiments, antisense oligonucleotides of the disclosure are complementary to exon 4 target regions of the ARSA gene, and induce exon skipping in at least one of exon 4 the ARSA gene (European Pat. Appl. EP4104867 A2).

In some embodiments, antisense oligonucleotides of the disclosure target ARSA pre-mRNA and induces skipping of exon 2, 3, and/or 4 of the ARSA gene, so it is excluded or skipped from the mature, spliced mRNA transcript. By skipping exon 2, 3, and/or 4, the disrupted reading frame is restored to an in-frame mutation.

In some embodiments, the nucleobase sequence of an antisense oligonucleotides that induces exon 2, 3, and/or 4 skipping is designed to be complementary to a specific target sequence within exon 2, 3, and/or 4 of ARSA pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises a sequence selected from SEQ ID NOs: 359-363 to target the ARSA gene (Table 2).

### Lung Disorders

### CF

Cystic fibrosis (CF; 219700) is a common, severe autosomal recessive disease caused by mutations in the CFTR gene. The CFTR gene encodes for a chloride channel responsible for chloride transport in epithelial cells. The major manifestations of CF are in the lungs, with more than 90% mortality related to the respiratory disease. The disease in the respiratory tract is linked to the insufficient CFTR function in the airway epithelium (PCT Publication No. WO2021199029 A1, incorporated herein by reference in its entirety).

In some embodiments, the methods and compositions described herein are useful for treating cystic fibrosis, by delivering an antisense oligonucleotide capable of inducing skipping of an exon in CTFR pre-mRNA carrying a deleterious mutation, e.g., that causes a frameshift mutation. In certain embodiments, antisense oligonucleotides of the disclosure are complementary to exon 24 target regions of the CTFR gene, and induce exon skipping in at least one of exon 24 the CTFR gene. (PCT Publication No. WO2021199029 A1).

In some embodiments, antisense oligonucleotides of the disclosure target CTFR pre-mRNA and induces skipping of exon 24 of the CTFR gene, so it is excluded or skipped from the mature, spliced mRNA transcript. By skipping exon 24, the disrupted reading frame is restored to an in-frame mutation.

In some embodiments, the nucleobase sequence of an antisense oligonucleotides that induces exon 24 skipping is designed to be complementary to a specific target sequence within exon 24 of CTFR pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide. In some embodiments, the antisense oligonucleotide comprises a sequence selected from SEQ ID NOs: 364-367 to target the CTFR gene (Table 2).

### Other Diseases

### IBS

The term “inflammatory bowel disease” (IBS17; OMIM 612261) means an inflammatory disease in bowel that involves Th17 cells. Crohn's disease and Ulcerative colitis represent exemplary diseases of the inflammatory bowel disease.

In some embodiments, the methods and compositions described herein are useful for treating IBS17, by delivering an antisense oligonucleotide capable of inducing skipping of an exon in IL23R pre-mRNA carrying a deleterious mutation, e.g., that causes a frameshift mutation. In certain embodiments, antisense oligonucleotides of the disclosure are complementary to exon 9 target regions of the IL23R gene, and induce exon skipping in at least one of exon 9 the IL23R gene. (U.S. Pat. No. 9,868,776, incorporated by reference herein in its entirety).

In some embodiments, antisense oligonucleotides of the disclosure target IL23R pre-mRNA and induces skipping of exon 9 of the IL23R gene, so it is excluded or skipped from the mature, spliced mRNA transcript. By skipping exon 9, the disrupted reading frame is restored to an in-frame mutation.

In some embodiments, the nucleobase sequence of an antisense oligonucleotides that induces ex-on 9 skipping is designed to be complementary to a specific target sequence within exon 9 of IL23R pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises a sequence selected from SEQ ID NOs: 379-385 to target the IL23R gene (Table 2).

### DEB

Epidermolysis bullosa is a group of inherited mechanobullous disorders characterized by fragility of the skin within the cutaneous basement membrane zone, with considerable clinical and genetic heterogeneity, inherited either in an autosomal dominant or autosomal recessive fashion. Traditionally, EB has been divided into three broad categories based on the level of tissue separation, determined by diagnostic electron microscopy and/or immunoepitope mapping: the simplex forms of EB (EBS) demonstrate tissue separation within the basal keratinocytes at the bottom layer of epidermis; the junctional forms of EB (JEB) display cleavage within the lamina lucida in the dermoepidermal basement membrane; and in the dystrophic forms (DEB; OMIM 131750), tissue separation occurs below the lamina densa within the upper papillary dermis. DEB is caused by mutations in COL7A1, on chromosomal region 3p21, encoding type VII collagen. (U.S. Pat. No. 9,340,783, incorporated herein by reference in its entirety).

In some embodiments, the methods and compositions described herein are useful for treating DEB, by delivering an antisense oligonucleotide capable of inducing skipping of an exon in COL7A1 pre-mRNA carrying a deleterious mutation, e.g., that causes a frameshift mutation. In certain embodiments, antisense oligonucleotides of the disclosure are complementary to exon 73, 74, or 80 target regions of the COL7A1 gene, and induce exon skipping in at least one of exon 73, 74, or 80 the COL7A1 gene.

In some embodiments, antisense oligonucleotides of the disclosure target COL7A1 pre-mRNA and induces skipping of exon 73, 74, and/or 80 of the COL7A1 gene, so it is excluded or skipped from the mature, spliced mRNA transcript. By skipping exon 73, 74, and/or 80, the disrupted reading frame is restored to an in-frame mutation.

In some embodiments, the nucleobase sequence of an antisense oligonucleotides that induces exon 73, 74, and/or 80 skipping is designed to be complementary to a specific target sequence within exon 73, 74, and/or 80 of COL7A1 pre-mRNA. In some embodiments, the antisense oligomer is a phosphorodiamidate morpholino oligomer (PMO) wherein each morpholino ring of the PMO is linked to a nucleobase including, for example, nucleobases found in DNA (adenine, cytosine, guanine, and thymine). In some embodiments, the antisense oligomer is a PNA oligonucleotide.

In some embodiments, the antisense oligonucleotide comprises a sequence selected from SEQ ID NOs: 372-378 to target the COL7A1 gene (Table 2).

TABLE 1
Example genes targeted by exon skipping oligos.

| Neurogenetic Diseases | Mutant Protein (Genes) | Exon(s) or intronic |
|---|---|---|
| Ataxia telangiectasia | Ataxia telangiectasia | Exons 8 and 55 |
| | mutated (ATM) | (Siva et al. Nucleic Acid |
| | | Ther. 2014 February; 24(1): |
| | | 69-86.) |
| Congenital disorder of | phosphomannomutase 2 | Intronic 5′ and 3′ |
| glycosylation | (PMM2) | cryptic splice sites etween |
| | | exon 7 and 8 |
| | | (Siva et al. Nucleic Acid |
| | | Ther. 2014 February; 24(1): |
| | | 69-86.) |
| Frontotemporal | Microtubule-associated | Exon 10 |
| dementia and | protein tau (MAPT) | (Siva et al. Nucleic Acid |
| Parkinsonism linked | | Ther. 2014 February; 24(1): |
| to chromosome 17 | | 69-86.) |
| Niemann Pick disease | Niemann-Pick C1 (NPC1) | Intronic cryptic 5′ |
| type C | | splice site between exon 9 |
| | | and 10 |
| | | (Siva et al. Nucleic Acid |
| | | Ther. 2014 February; 24(1): |
| | | 69-86. |
| Neurofibromatosis | Neurofibromin (NF1) | Intronic cryptic 5′ |
| type 1 | | splice site between exon 4 |
| | | and 4a; exon 30 and 31; |
| | | exon 45 and 46; and exon 9a |
| | | and 9b |
| | | (Siva et al. Nucleic Acid |
| | | Ther. 2014 February; 24(1): |
| | | 69-86.) |
| Neurofibromatosis | Merlin (NF2) | Intronic cryptic 5′ |
| type 2 | | splice site between exon 13 |
| | | and 14 |
| | | (Siva et al. Nucleic Acid |
| | | Ther. 2014 February; 24(1): |
| | | 69-86.) |
| Megalencephalic | Oligomeric plasma | Intronic cryptic 5′ |
| leukoencephalopathy | membrane (MLC1) | splice site between exon 10 |
| with subcortical cysts | | and 11 |
| type 1 | | (Silva et al. 2014) |
| Pelizaeus-Merzbacher | Proteolipid protein (PLP1) | Exon 3A/3B |
| disease | | (Silva et al. 2014) |
| Familial dysautonomia | Inhibitor kinase complex- | Exon 20 |
| | associated protein | (U.S. Pat. No. 11,179,472) |
| | (IKBKAP) | |
| Spinal muscular atrophy | Survival of motor neuron | Exon 7 |
| | (SMN2) | (U.S. Pat. No. 11,179,472) |
| Myotonic dystrophy (DM) Type I | Dystrophia myotonica | Exon 15 |
| | protein kinase (DMPK) | (U.S. Pat. No. 11,179,472) |
| Myotonic dystrophy (DM) Type II | Cellular nucleic acid- | Between exon 1 and 2 |
| | binding protein | (U.S. Pat. No. 11,179,472) |
| | (CNBP/ZNF9) | |
| Frontotemporal lobar | TAR DNA-binding protein | Exon 6 |
| degeneration (FTLD), | 43 (TARDBP) | (U.S. Pat. No. 11,179,472) |
| amyotrophic lateral sclerosis | | |
| (ALS) | | |
| Hutchinson-Gilford syndrome | Prelamin A (LMNA) | Exons 4 and 5 |
| | | (U.S. Pat. No. 11,179,472) |
| Fukuyama congenital muscular | Fukutin (FKTN) | Exons 4 and 5 |
| dystrophy (FCMD), Walker- | | (U.S. Pat. No. 11,179,472) |
| Warburg syndrome (WWS), limb- | | |
| girdle muscular dystrophy type | | |
| 2M (LGMD2M), and dilated | | |
| cardiomyopathy type 1X | | |
| (CMD1X) | | |
| Parkinson's Disease PD | LRRK2 | Exon 41 |
| | | (Korecka et al., Mol Ther |
| | | Nucleic Acids. 2020 Sep. 4; |
| | | 21: 623-635) |
| Cardiovascular Diseases | Mutant Protein (Genes) | Exon(s) or intronic |
| Familial hypercholesterolemia | LDL receptor protein | Exon 12 |
| (FH) | (LDLR) | (U.S. Pat. No. 11,179,472) |
| | APOB | Exon 27 |
| | | (Disterer et al. Mol Therap |
| | | 2013 21(3); 602-609) |

| Metabolic Diseases | Mutant Protein (Genes) | Exon(s) or intronic |
|---|---|---|
| Medium-chain acyl-coenzyme A | Medium-chain acyl-CoA | Exon 11 |
| dehydrogenase deficiency | dehydrogenase (ACADM) | (Bruun et al. Molecular |
| | | Genetics and Metabolism |
| | | Volume 110, Issues 1-2, |
| | | Pages 122) |
| Phenylketonuria | phenylalanine hydroxylase | Exon 11 |
| | (PAH) | (Heintz et al. Mol Genet |
| | | Metab. 2012 |
| | | Aug; 105(4): 559-565) |

| Cancers | Mutation Protein (Genes) | Exon(s) or intronic |
|---|---|---|
| Colorectal cancer, lung cancer | K-Ras (KRAS) | Exon 2 |
| | | (WO/2015/091525) |

| Skeletal Muscle Disorders | Mutation Protein (Genes) | Exon(s) or intronic |
|---|---|---|
| Duchenne muscular dystrophy | Myostatin (MSTN) | Exon 2 |
| (DMD) | | (Kemaladewi, DU et al. |
| | | BMC Med Genomics 4, 36 |
| | | (2011)) |
| Duchenne muscular dystrophy | Dystrophin (DMD) | Exons 45, 51, 53 |
| (DMD) | | (Kemaladewi, DU et al. |
| | | BMC Med Genomics 4, 36 |
| | | (2011)) |
| Becker muscular dystrophy | Dystrophin (DMD) | Exons 9, 25, 27, 31, 37, 38, |
| | | 41, 72 and 74 |
| | | (Okubo et al. Hum Genet. |
| | | 2020; 139(2): 247-255) |

| Lung Disorders | Mutation Protein (Genes) | Exon(s) or intronic |
|---|---|---|
| Cystic fibrosis | Cystic fibrosis | Exon 23 |
| | transmembrane | (Kim et al., 2022) |
| | conductance regulator | |
| | (CFTR) | |

| Other diseases | Mutation protein (Genes) | Exon(s) or intronic |
|---|---|---|
| Irritable Bowel Syndrome | IL23R | Exon 9 |
| | | (U.S. Pat. No. 9,868,776B2) |
| Dystrophic Epidermolysis Bullosa | COL7A1 | Exons 73, 74, and 80 |
| | | (U.S. Pat. No. 9,340,783B2) |

TABLE 2
Example sequences of oligos for use in treating various disorders.

| | | | | SEQ |
|---|---|---|---|---|
| | | Target | | ID |
| Disease | Target Gene | exon | Sequence | NO |
| Congenital | PMM2 | 7 | TAGCTGCAAAGCAAGTGAAGCGGAC | 150 |
| disorder of | (NM_000303.3) | | ATCACAAACACAACCTACCTCAGGC | 151 |
| glycosylation | | | | |
| Familial | IKBKAP | 20 | TCGCAAACAGTACAATGGCG | 152 |
| dysautonomia | (NM_003640.5) | | AACAGTACAATGGCG | 153 |
| | | | TCGCAAACAGTACAA | 154 |
| | | | ACTAGTCGCAAACAG | 155 |
| | | | AGCTAACTAGTCGCA | 156 |
| Duchenne | MSTN | 2 | AGCCCATCTTCTCCTGGTCCTGGGAAGG | 157 |
| Muscular | (NM_005259.3) | | | |
| Dystrophy | DMD | 44 | GTGTCTTTCT GAGCCGCCAT TTCTCA | 158 |
| | (NM_004006.3) | | TCTGTTAGCC ACGCCGCCAT TT | 159 |
| | | | CTGTTAGCCA CTCGCCGCCA TTTC | 160 |
| | | | TTGTGTCTTT CTTCTGTTAG CCAC | 161 |
| | | | TGTTAGCCAC TGATCTCAAC AGATCT | 162 |
| | | | GTTCAGCTTC TGTTCTCAAC AGATCT | 163 |
| | | | TTGTGTCTTT CTGCGCCGCC ATTTCT | 164 |
| | | | CTGAGAAACT GTTCGCCGCC ATTTCT | 165 |
| | | | TCTCAGGAAT TTGCGCCGCC ATTTCT | 166 |
| | | | TGTTAGCCAC TGACGCCGCC ATTTCT | 167 |
| | | | GTTCAGCTTC TGTCGCCGCC ATTTCT | 168 |
| | | | ATTCTCAGGA ATTTGAACGC | |
| | | | CGCCATTTCT | 169 |
| | | | ATTTGTGTCT TTCTGTTTCT | 170 |
| | | | CAACAGATCT | |
| | | | ATTCTCAGGA ATTTGTTTCT CAACA | 171 |
| | | | ATTTGTGTCT TTCTGAACGC | 172 |
| | | | CGCCATTTCT | |
| | | | TCTCAGGAAT TTGTCTCAAC AGATCT | 173 |
| | | 45 | GTTTGCCGCTGCCTCCTGGAGTTCCT | 174 |
| | | | GTTTGCCGCTGCCCTGGAGTTCCT | 175 |
| | | | CAGTTTGCCGCTGCCCATCCTGGAGTTCCT | 176 |
| | | | CAGTTTGCCGCTGCCCTGGAGTTCCT | 177 |
| | | | TTCTTCCCCAGTTGCGCCATCCTGGAGTTC | 178 |
| | | | CAGACCTCCTGCCACGCCATCCTGGAGTT | 179 |
| | | | C | |
| | | | TTGCCGCTGCCCACATCCTGGAGTTC | 180 |
| | | | GCCGCTGCCCACATCCTGGAGTTCCT | 181 |
| | | | CCGCTGCCCAATGTCCTGGAGTTCCT | 182 |
| | | | GCUGCCCAAUGCCAUCCUGGAGUUC | 183 |
| | | | ″GCUGCCCAAUGCCAUCCUGGAGUUC | 184 |
| | | | UUGCCGCUGCCCAAUGCCAUCCUGG″ | 185 |
| | | | ACAGUUUGCCGCUGCCCAAUGCCAU | 186 |
| | | | UGACAACAGUUUGCCGCUGCCCAAU | 187 |
| | | | UGUUCUGACAACAGUUUGCCGCUGC | 188 |
| | | | UUCAAUGUUCUGACAACAGUUUGCC | 189 |
| | | | UUGCAUUCAAUGUUCUGACAACAGU | 190 |
| | | | CCCAGUUGCAUUCAAUGUUCUGACA | 191 |
| | | | CCAAUGCCAUCCUGGAGUUCCUGUAA | 192 |
| | | | CAA UGC CAU CCU GGA GUU CCU G | 395 |
| | | | GCC CAA UGC CAU CCU GGA GUU CCU | 396 |
| | | | GUA AGA UAC C | |
| | | | GCC CAA UGC CAU CCU GGA GUU CCU | 397 |
| | | | GUA AGA U | |
| | | | UUG CCG CUG CCC AAU GCC AUC CUG | 398 |
| | | | GAG UUC CUG UAA GAU | |
| | | 50 | CAATGCCATCCTGGAGTTCCTG | 193 |
| | | | ATGGGATCCAGTATACTTACA | 194 |
| | | | ATGGGATCCAGTATACTTAC | 195 |
| | | | TGGGATCCAGTATACTTAC | 196 |
| | | | ATGGGATCCAGTATACTTA | 197 |
| | | | GATCCAGTATACTTACAGG | 198 |
| | | 51 | AATGGGATCCAGTATACTT | 199 |
| | | | CTCCAACATC AAGGAAGATG | 200 |
| | | | GCATTTCTAG | |
| | | | TCAAGGAAGATGGCATTTCT | 201 |
| | | | GTGTCACCAGAGTAACAGTCTGAGTAGGA | 202 |
| | | | G | |
| | | | AGGTTGTGTCACCAGAGTAACAGTCTGAG | 203 |
| | | | T | |
| | | | GGCAGTTTCCTTAGTAACCACAGGTTGTG | 204 |
| | | | T | |
| | | | AGATGGCAGTTTCCTTAGTAACCACAGGT | 205 |
| | | | T | |
| | | | ATGGCATTTCTAGTTTGGAGATGGCAGTT | 206 |
| | | | T | |
| | | | CUC CAA CAU CAA GGA AGA UGG CAU | 207 |
| | | | UUC UAG | |
| | | | CTCCAACATCAAGGAAGATGGCATTTCTA | 208 |
| | | | G | |
| | | 53 | CCTCCGGTTC TGAAGGTGTT C | 209 |
| | | | AUU CAA CUG UUG CCU CCG GUU CUG | |
| | | | AAG GUG | 210 |
| | | | CUG AAG GUG UUC UUG UAC UUC AUC C | 211 |
| | | | CAU UCA ACU GUU GCC UCC GGU UCU | |
| | | | GAA GGU G | 212 |
| | | | CCTCCGGTTCTGAAGGTGTTC | 213 |
| | | | GTTGCCTCCGGTTCTGAAGGTGTTC | 214 |
| | | | GUUGCCUCCGGUUCUGAAGGUGUUC | 401 |
| | | | CCUCCGGUUCUGAAGGUGUUC | 402 |
| | | | GTTGCCTCCGGTTCTGAAGGTGTTC | 214 |
| | | 55 | GCAGCCUCUCGCUCACUCACC | 215 |
| | | | CCAAAGCAGCCUCUCGCUCAC | 216 |
| | | | UUCUUCCAAAGCAGCCUCUCG | 217 |
| | | | AUCUAUGAGUUUCUUCCAAAG | 218 |
| | | | UGUUGCAGUAAUCUAUGAGUU | 219 |
| | | | CAGGGGGAACUGUUGCAGUAA | 220 |
| | | | UUUCCAGGUCCAGGGGGAACU | 221 |
| | | | GCAAGAAACUUUUCCAGGUCC | 222 |
| | | | CAG CAG CAG | 223 |
| Myotonic | DMPK | 3′-UTR | CAG CAG CAG CAG | 224 |
| Dystrophy 1 | (NM_004409.5) | | CAG CAG CAG CAG CAG | 225 |
| (DM1) | | | CAG CAG CAG CAG CAG CAG | 226 |
| | | | CAG GCA GGC | 227 |
| Myotonic | ZNF9 | Intron 1 | CAG GCA GGC AGG | 228 |
| Dystrophy 2 | (NM_001127192. | | AGG CAG GCA | 229 |
| (DM2) | 2) | | AGG CAG GCA GGC | 230 |
| | | | GGC AGG CAG | 231 |
| | | | GGC AGG CAG GCA | 232 |
| | | | TCACTTTCAT AATGCTGG | 233 |
| Spinal Muscular | SMN2 | 7 | CUGCCAAGGA GGGCCCUGCU CAGCC | 234 |
| Atrophy, type 3 | (NM_017411.4) | | | |
| Bladder cancer | HRAS | 2 | CGCACUCUUG CCCACACCGC CGGCG | 235 |
| | (NM_005343.4) | | CUCACCUCUA UAGUGGGGUC GUAUU | 236 |
| | | | UUGCCCACAC CGCCGGCGCC C | 237 |
| | | | UUGCCCACAC CGACGGCGCC C | 238 |
| | | | UAUAGUGGGG UCGUAUUCGU CCACA | 239 |
| | | | UGGUUCUGGA UCAGCUGGAU GGUCA | 240 |
| | | | CGGGGUCCUC CUACAGGGUC UCCUG | 241 |
| | | | GUCAUCGCUC CUCAGGGGCC UGCGG | 242 |
| | | | CCCACCACCA CCAGCUUAUA UUCCG | 243 |
| | | | GGGUCUCCUG CCCCACCUGC CAAGG | 244 |
| | | | UUCAGUCAUU UUCAGCAGGC CUUAU | 245 |
| Bladder cancer; | KRAS | 2 | UUUACCUCUA UUGUUGGAUC AUAUU | 246 |
| Breast cancer; | (NM_004985.5) | | AUGAAAAUGG UCAGAGAAAC CUUUA | 247 |
| Gastric cancer; | | | UCUGUAUCAA AGAAUGGUCC UGCAC | 248 |
| Acute Myeloid | | | CAGUAAUAUG CAUAUUAAAA CAAGA | 249 |
| Leukemia; | | | AAUAAAAAUA AUGAAAAUGU GACUA | 250 |
| Lung cancer; | | | UAUUAGAACA UGUCACACAU AAGGU | 251 |
| Pancreatic | | | AGUUUAUAUU CAGUCAUUUU CAGCA | 252 |
| carcinoma; | | | UACGCCACCA GCUCCAACUA CCACA | 253 |
| RAS-associated | | | GCUGUAUCGU CAAGGCACUC UUGCC | 254 |
| autoimmune | | | UCGUCCACAA AAUGAUUCUG AAUUA | 255 |
| leukoproliferative | | | UUUCACACCA GCAAGAACCU GUUGG | 256 |
| disorder type | | | | |
| IV | | | | |
| RAS-associated | NRAS | 2 | CCUCACCUCU AUGGUGGGAU CAUAU | 257 |
| autoimmune | (NM_002524.5) | | ACCACCAGUU UGUACUCAGU CAUUU | 258 |
| lymphoproliferative | | | CAACACCACC UGCUCCAACC ACCAC | 259 |
| syndrome | | | GUGCGCUUUU CCCAACACCA CCUGC | 260 |
| type IV; | | | UGGUUCUGGA UUAGCUGGAU UGUCA | 261 |
| Colorectal; | | | GGUGGGAUCA UAUUCAUCUA CAAAG | 262 |
| Thyroid | | | AAUCAGGGUU AAUUGGCGAG CCACA | 263 |
| carcinoma, | | | CAGGAUCAGG UCAGCGGGCU ACCAC | 264 |
| follicular | | | AAA AAU GUC UCC UGC GAG AGC AAA C | 265 |
| Valosine- | VCP | 5 | CCA CCA UGG ACA AGA AAA AUG UCU C | 266 |
| containing | (NM_007126.5) | | CAG CAC GCA UCC CAC CAU GGA C | 267 |
| protein (VCP)- | | | AUC ACU GUG UCU GGA GCA ACA AUG C | 268 |
| associated | | | UCG UUU GAU AGG CUC CCC UUC GCA G | 269 |
| inclusion body | | | AUC AGG GAG AAA ACU CAC CUC UCG U | 270 |
| myopathy (IBM) | | | ACCUACCCAG UUCGGAAUGA UC | 271 |
| Inflammatory | IL23R | 9 | CUACCCAGUU CGGAAUGA | 272 |
| Bowel diseases | (NM_144701.3) | | UACCCAGUUC GGAAU | 273 |
| (IBS) | | | ATGTCTCCTC TGTTGTCTAA TG | 274 |
| | | | TAACAGCAAA GACGATCATT CC | 275 |
| | | | GGGCTCTGGGCTCCTGAGCC | 276 |
| Hutchison- | LMNA | 11-12 | CCGAGCTGCTGCAGTGGGAG | 277 |
| Gilford Progeria | (NM_ | | GCAGGTCCCGCACAGCACGG | 278 |
| Syndrome | 001406990.1) | | TGGCAGATGCCTTGTCGGCA | 279 |
| (HGPS) | | | GAGCCGCTGGCAGATGCCTT | 280 |
| | | | CTGAGCCGCTGGCAGATGCC | 281 |
| | | | AGGAGATGGGTCCGCCCACC | 282 |
| | | | CAGAGGAGATGGGTCCGCCC | 283 |
| | | | GCCAGAGGAGATGGGTCCGC | 284 |
| | | | AGGUCCCGGGAGAAGGGA | 285 |
| Alagille | JAG1 | 13 | CAGCCAGGUCCCGGGAGA | 286 |
| syndrome | (NM_000214.3) | | GGAGACAGCCAGGUCCCG | 287 |
| | | | AGUCUGGAGACAGCCAGG | 288 |
| | | | GAGCAAGUCUGGAGACAG | 289 |
| | | | AAAAGGAGCAAGUCUGGA | 290 |
| | | | GGGACAAAAGGAGCAAGU | 291 |
| | | | GCAGUGGUAGUAAGUGGG | 292 |
| | | | GGGAUCAGAUCACAGCCA | 293 |
| | | | CAGAUCACAGCCAUGCAC | 294 |
| | | | CCCAGCGUCGCCCUGGGC | 295 |
| Tuberous | TSC2 | 4 | CCCAUCCCAGCGUCGCCC | 296 |
| Sclerosis | (NM_000548.5) | | CGUCACCCAUCCCAGCGU | 297 |
| Complex | | | CCUGACGUCACCCAUCCC | 298 |
| | | | GGCAGCCUGACGUCACCC | 299 |
| | | | CAGUGGGCAGCCUGACGU | 300 |
| | | | ACAGUCAGUGGGCAGCCU | 301 |
| | | | ACAGGACAGUCAGUGGGC | 302 |
| | | | ACAGGAUCAGCAGAGCCU | 303 |
| | | | AUCAGCAGAGCCUGCCAG | 304 |
| | | | AGGGCUUAAA AUUUCUCCUG C | 305 |
| Usher Syndrome | USH2A | 62 | CCAAGGGCUU AAAAUUUCUC C | 306 |
| | (NM_206933.4) | | UUUGAAUCAG AGUCCAAGGG C | 307 |
| | | | AAGUUUCUCA GUCCACUUGG G | 308 |
| | | | CCGGCCAUUC UCUUUCUGUU C | 309 |
| | | | AUGCCCGGCC AUUCUCUUUC | 310 |
| | | | UGACCACUGU AGUAGCAAUG | 311 |
| | | | ACACCAUUGG UUCUCAUAGG | 312 |
| | | | AUGGUUUGCA GCCACAACAC | 313 |
| | | | CCACAACACC AAUGCGAUAU | 314 |
| | | | GCAUGGUUUG CAGCCACAAC | 315 |
| | | | CUUGGGGAAG AUUCUAAGGU | 316 |
| | | | GUAUCCGCUG AGGGAAUA | 317 |
| Aicardi- | ADAR | 2 or 3 | GUGUAGUAUC CGCUGAGG | 318 |
| Goutieres | (NM_001193495) | | GAUGGGUGUA GUAUCCGC | 319 |
| syndromc-6 | | | AAAUGGAUGG GUGUAGUA | 320 |
| (AGS6) | | | CCUUGAAAUG GAUGGGUG | 321 |
| | | | GGCGCAGAAC CACCAGGU | 322 |
| Familial | ATP1A2 | 22 or 23 | AGGGAGUUGC AAUAUCGA | |
| hemiplegic | (NM_000702) | | | |
| migraine-2; | | | | |
| Familiar Basilar | | | | |
| Migraine; | | | | 323 |
| Alternating | | | | |
| hemiplegia of | | | | |
| childhood | | | | |
| Progressive | PRICKLE2 | 4 or 5 | GCCUCCACAG AUUCAGGG | 324 |
| Myoclonic | (NM_198859) | | | |
| Epilepsy 5 | | | | |
| Mental | SETD5 | 4 or 5 | AAACUUCAUU CUGGAACA | 325 |
| Retardation-23 | (NM_001080517) | | | |
| (MRD23); | | | | |
| 3p25 | | | | |
| Microdeletion | | | | |
| Syndrome | | | | |
| Leukoencephalopathy | EIF2B5 | 12 or 13 | AGGGCUUUGG CCAUUUUC | 326 |
| with | (NM_003907) | | | |
| vanishing white | EIF2B1 | 6 | ACAGUUUGCA CUUCCGGG | 327 |
| matter | (NM_001414) | | | |
| | EIF2B2 | 1 | CGCCUAAGGA AUUCACUC | 328 |
| | (NM_014239) | | | |
| Zellweger | PEX1 | 10, 19, 21, | CGCCUAAGGA AUUCACUC | 329 |
| Syndrome; | (NM_000466) | or 14 | | |
| Heimler | PEX1 | 10, 18, 20, | GGAUACGCCU AAGGAAUU | 330 |
| Syndrome-1 | (NM_001282677) | or 13 | | |
| | PEX1 | 10, 19, 21, | GCCAGUGCCC AUAGCGGG | 331 |
| | (NM_001282678) | or 14 | | |
| Early Infantile | STXBP1 | 18, or 19 | CUUAUGCCAG UGCCCAUA | 332 |
| Epileptic | (NM_003165) | | | |
| encephalopathy- | STXBP1 | 18 | CCCAAGGGAG CCACGCGU | 333 |
| 4 | (NM_001032221) | | | |
| Familial Infantile | PRRT2 | 1 | AAGGGAGCCA CGCGUUAC | 334 |
| convulsion with | (NM_001256443) | | | |
| paroxysmal | PRRT2 | 1 | AGCCCAAGGG AGCCACGC | 335 |
| choreoathetosis; | (NM_145239) | | | |
| Episodic | PRRT2 | 1 | CUUCCGGGAC AGUGUGGA | |
| kinesigenic | (NM_001256442) | | | |
| dyskenisia 1; | | | | |
| Benign familial | | | | 336 |
| infantile | | | | |
| seizures-2 | | | | |
| Generalized | STX1B | 6, or 7 | UUCUUGGCAG CUGGAACA | 337 |
| epilepsy with | (NM_0582874) | | | |
| febrile seizure | | | | |
| plus type 9 | | | | |
| Smith-Magenis | RAI1 | 4 | GUAGUCCUGG UGGCAUGC | 338 |
| syndrome | (NM_030665) | | | |
| Pitt-Hopkins | TCF4 | 10 | CCCCUGUAGU CCUGGUGG | 339 |
| syndrome | (NM_001243236) | | | |
| | TCF4 | 10 | CUGUAGUCCU GGUGGCAU | 340 |
| | (NM_001243235) | | | |
| | TCF4 | 10 | UGGCAUCACG GACAAUGC | 341 |
| | (NM_001243234) | | | |
| Nicmann-Pick | NPC1 | 15 | CGACAAAGAG AUUCAGCA | 342 |
| disease type C1 | (NM_000271) | | | |
| Episodic Ataxia | CACNAIA | 36, or 37 | GACGGCGACA AAGAGAUU | 343 |
| type 2; | (NM_000068) | | | |
| Familial | CACNA1A | 36, or 37 | AUGAUGACGG CGACAAAG | 344 |
| Hemiplagic | (NM_023035) | | | |
| Migraine; | CACNAIA | 36, or 37 | UGUCCAUGAU GACGGCGA | 345 |
| SpinoCerebellar | (NM_001174080) | | | |
| Ataxia type 6 | CACNAIA | 36, or 37 | AAAGUUGUCC AUGAUGAC | 346 |
| | (NM_001127222) | | | |
| | CACNA1A | 36, or 37 | GACUUGUGGG UGUUCUCA | 347 |
| | (NM_001127221) | | | |
| Hereditary | DNMT1 | 29, or 14 | GAGUGGACUU GUGGGUGU | 348 |
| Sensory | (NM_001379) | | | |
| Neuropathy type | DNMT1 | 30, or 15 | CGCUGGAGUG GACUUGUG | 349 |
| IE; | (NM_001130823) | | | |
| Autosomal | DNMT1 | 29, or 14 | UAGCUCGCUG GAGUGGAC | 350 |
| dominant | (NM_001318730) | | | |
| cerebellar ataxia, | DNMT1 | 30, or 15 | GUCUUCUGCC UUGGGCUU | 351 |
| deafness and | (NM_001318731) | | | |
| narcolepsy | | | | |
| Neurofibromatosis | NF2 | 14 | GAUAGGUCUU CUGCCUUG | 352 |
| type 2; | (NM_181830) | | | |
| NF2-related | NF2 | 15 | AUCUAAAAUC UGCUUCUU | 353 |
| meningioma; | (NM_181829) | | | |
| Shwannomatosis | NF2 | 4 | AUGCAGAUAG GUCUUCUG | 354 |
| 1 | (NM_181833) | | | |
| | NF2 | 16 | GUCUUCUGCC UUGGGCUU | 355 |
| | (NM_181832) | | | |
| | NF2 | 15 | CUCAAAUGCA GAUAGGUC | 356 |
| | (NM_181828) | | | |
| | NF2 | 16 | UCUUUUCCAU GUAUUCCA | 357 |
| | (NM_016418) | | | |
| | NF2 | 15 | ACCACGUUCA CCCCGUUC | 358 |
| | (NM_000268) | | | |
| Phelan- | SHANK3 | 16 | GGCCGGUCAG GAGGGCGG | 359 |
| McDermid | (NM_033517) | | | |
| syndrome | | | | |
| (PHMDS); | | | | |
| Schizophrenia- | | | | |
| 15 (SCZD15)″ | | | | |
| Metachromatic | ARSA | 2, or 3 | GAGCCGGCCG GUCAGGAG | 360 |
| Leukodystrophy | (NM_000487) | | | |
| | ARSA | 3, or 4 | ACCGGGAGCC GGCCGGUC | 361 |
| | (NM_001085425) | | | |
| | ARSA | 3, or 4 | UCCGAACCGG GAGCCGGC | 362 |
| | (NM_001085426) | | | |
| | ARSA | 3, or 4 | UCCGAACCGG GAGCCGGC | 363 |
| | (NM_001085427) | | | |
| | ARSA | 2, or 3 | CCAGAAAAAAUAAAUACUUUC | 364 |
| | (NM_001085428) | | | |
| Cystic Fibrosis | CTFR | 24 | AAUGUUCCAGAAAAAAUA | 365 |
| | (EF466097.1) | | UCUAAAUGUUCCAGAAAA | 366 |
| | | | CCACUGUUCAUAGGGAUC | 367 |
| | | | CCAUACCUGUAUAGGAGAGAUUCAUA | 368 |
| Familial | APOB | 27 | CCAUACCUGUAUAG GAGAGAAU-UAGA | 369 |
| Hyper- | (NM_000384.3) | 27 | CCAUACCUGUAUAGGAGAGAAUUAGAUU | 370 |
| cholesterolemia | | | CAUA | |
| | | 27 | ATCCTCTTTGGTAACCTCACCTCAC | 371 |
| Phenylketonuria | PAH | 11 | UCUCCACGGUCGCCCUUCAGCCCGCGUU | 372 |
| | (NM_001354304. | | CU | |
| | 2) | | | |
| Dystrophic | COL7A1 | 73, 74, or | UCUCCACGGUCGCCCUUCAGCCCGC | 373 |
| Epidermolysis | (NM_000094.4) | 80 | CCUUUCUCUCCCCGUUCUCCCUGAA | 374 |
| Bullosa, | | | CCUUUCUCUCCCCAUUCUCCCUGAA | 375 |
| autosomal | | | CCACCUGUUCUCCACGUUCUCCUUUC | 376 |
| dominant | | | GGCCUCUUGGACCCUGCAGACCCU | 377 |
| | | | GGCCUCUUGGACCCUACAGACCCU | 378 |
| | | | ACCUACCCAG UUCGGAAUGA UC | 379 |
| Inflammatory | ILR23 | 9 | CUACCCAGUU CGGAAUGA | 380 |
| Bowel Disease | (NM_144701.3) | | UACCCAGUUC GGAAU | 381 |
| | | | ATGTCTCCTC TGTTGTCTAA TG | 382 |
| | | | TAACAGCAAA GACGATCATT CC | 383 |
| | | | GAAACAACTTTTTGTTTGAGAA | 384 |
| | | | GTTTTTCCAGAGTGCACAACTA | 385 |
| | | | F: AGUCCAAGUGUGGCUCAAA | 386 |
| | | | R: UUUGAGCCACACUUGGACU | |
| Frontotemporal | MAPT | 10 | F: GGCUCAAAGGAUAAUAUCA | 387 |
| dementia with | (NM_ | | | |
| parkinsonism | 001377265.1) | | R: UGAUAUUAUCCUUUGAGCC | |
| linked to | | | F: GCAACGUCCAGUCCAAGUG | 388 |
| chromosome 17 | | | R: CACUUGGACUGGACGUUGC | |
| Parkinson's | LRRK2 | 41 | AGACAGACCTGATCACCTACCTGGT | 389 |
| Disease | (NM_198578.4) | | GGTATCTGCCAGAAAATGCACAGGA | 390 |
| | | | CTACCAGCCTACCATGTTACCTTGA | 391 |
| | | 31 | | |

### Kits

In some aspects, the disclosure encompasses kits comprising one or more containers and comprising one or more doses of a complex formed between a therapeutic mRNA molecule and a 3E10 antibody or antigen binding fragment thereof disclosed herein. In certain embodiments, a unit dosage is provided wherein the unit dosage contains a predetermined amount of a composition comprising, for example, a complex formed between a therapeutic mRNA molecule and a 3E10 antibody or antigen binding fragment thereof disclosed herein, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the composition may be provided as a lyophilized powder that may be reconstituted upon addition of an appropriate liquid, for example, sterile water. In certain preferred embodiments, the composition comprises one or more substances that inhibit protein aggregation, including, but not limited to, sucrose and arginine. Any label on, or associated with, the container(s) indicates that the enclosed composition is used for diagnosis or treatment.

The present invention also provides kits for producing single-dose or multi-dose administration units of a complex formed between a therapeutic mRNA molecule and a 3E10 antibody or antigen binding fragment thereof disclosed herein and, optionally, one or more other diagnostic or therapeutic agents. The kit comprises a container and a label or package insert on or associated with the container. Suitable containers include, for example, bottles, vials, syringes, etc. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). Such kits will generally contain a pharmaceutically acceptable formulation of a complex formed between a therapeutic mRNA molecule and a 3E10 antibody or antigen binding fragment thereof and, optionally, one or more other therapeutic agents in the same or different suitable containers. The kits may also contain other pharmaceutically acceptable formulations, for combination therapy.

More specifically the kits may have a single container that contains the complex formed between a therapeutic mRNA molecule and a 3E10 antibody or antigen binding fragment thereof), with or without additional components, or they may have distinct containers for each desired agent. Alternatively, the complex formed between a therapeutic mRNA molecule and a 3E10 antibody or antigen binding fragment thereof and any optional therapeutic agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the complex formed between a therapeutic mRNA molecule and a 3E10 antibody or antigen binding fragment thereof and any optional components to the patient, e.g., one or more needles or syringes, from which the formulation may be injected or introduced into the patient.

### EXAMPLES

With respect to the experiments below, standard 3E10 sequence was used except wherein noted to be the D31N variant. Both standard 3E10 and the D31N variant were used as full-length antibodies.

### Example 1—Carrier DNA Enhances mRNA to Non-Tumor Tissue

2 μg of fluorescently labeled mRNA was mixed with 20 μg of 3E10-D31N with or without carrier DNA (5 ug) for 15 minutes at room temperature. mRNA complexed to 3E10 was injected to fetuses at E15.5. 24-48 hours after treatment, fetuses were harvested and analyzed for mRNA delivery using IVIS imaging.

Without carrier DNA, 3E10-D31N complexed to mRNA was rapidly cleared from fetuses at 24 hours. The addition of carrier DNA, however, resulted in detectable mRNA signal in multiple tissues of the fetus at 48 hours.

### Example 2—3E10 (D31N) Complexed with mRNA and Carrier DNA Results in Sustained Levels Protein Expression

10 μg of luciferase mRNA and 10 μg of single stranded carrier DNA (60 nts) was mixed with 100 μg of 3E10 (WT) or 3E10 (D31N) for 15 minutes at room temperature. mRNA complexed to 3E10 was injected intramuscularly (IM) in the right quadricep of each mouse. Luciferase expression was monitored over 6 days.

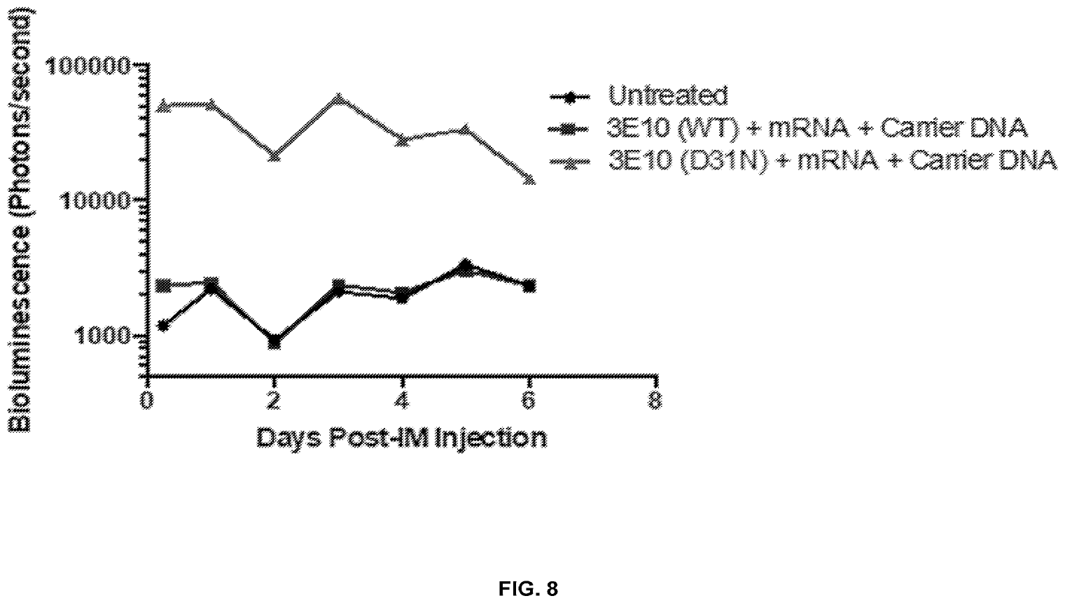

As seen in FIG. 8, administration of 3E10 (D31N) complexed with mRNA and carrier DNA resulted in sustained levels of luciferase expression, while 3E10 (WT) complexed to mRNA and carrier DNA failed to produce any appreciable signal above background.

### Example 3—Distribution of IV Injected 3E10 In Vivo

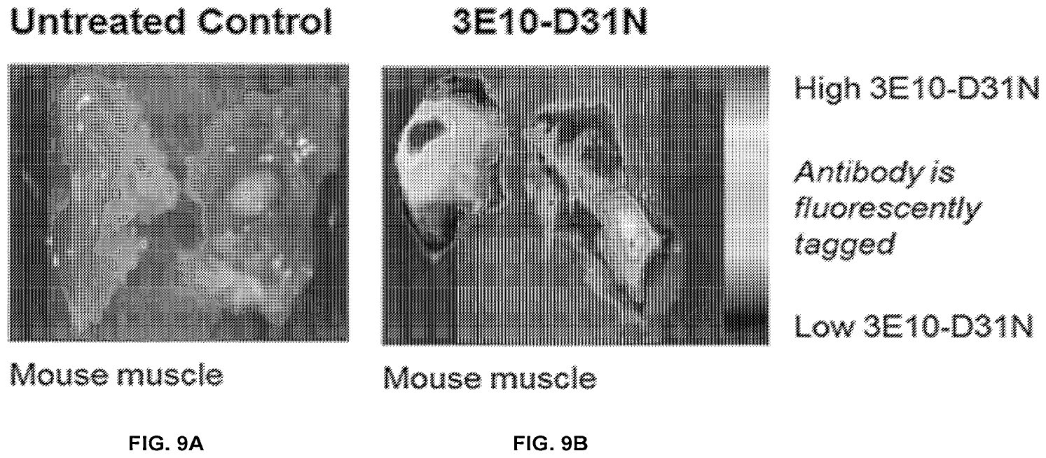

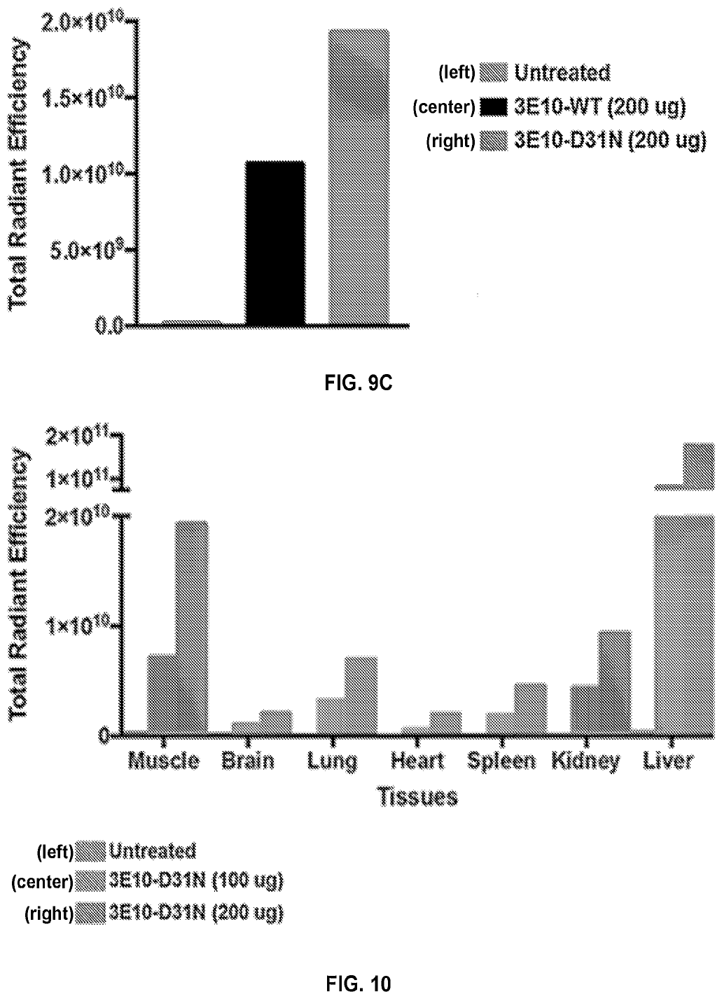

Distribution of IV injected 3E10 to muscle was investigated. Mice were injected intravenously with 200 μg of 3E10, WT or D31N, labeled with VivoTag680 (Perkin Elmer). Four hours after injection, muscle was harvested and imaged by IVIS (Perkin Elmer) (FIGS. 9A and 9B). Quantification of IVIS image demonstrates that 3E10-D31N achieves higher distribution to muscle when compared to 3E10-WT (FIG. 9C).

Dose-dependent biodistribution of 3E10-D31N to tissues was investigated. Mice were injected intravenously with 100 μg or 200 μg of 3E10-D31N labeled with VivoTag680 (Perkin Elmer). 24 hours after injection, tissues were harvested and imaged by IVIS (Perkin Elmer). Quantification of tissue distribution demonstrated a dose-dependent, two-fold increase in muscle accumulation without a commensurate increase in multiple tissues including liver (FIG. 10).

### Example 4-Molecular Modeling of 3E10 and Engineered Variants Thereof

WT HEAVY CHAIN scFv SEQUENCE
(SEQ ID NO: 392)
E VOLVESGGGL VKPGGSRKLS CAASGFTFSD
YGMHWVRQAP EKGLEWVAYI SSGSSTIYYA
DTVKGRFTIS RDNAKNTLFL QMTSLRSEDT
AMYYCARRGL LLDYWGQGTT LTVS
LIGHT CHAIN scFv SEQUENCE
(SEQ ID NO: 393)
D IVLTQSPASL AVSLGQRATI SCRASKSVST
SSYSYMHWYQ QKPGQPPKLL IKYASYLESG
VPARFSGSGS GTDFTLNIHP VEEEDAATYY
CQHSREFPWT FGGGTKLEIK RADAAPGGGG
SGGGGSGGGGS

Molecular modeling of 3E10 (Pymol) revealed a putative Nucleic Acid Binding pocket (NAB1) (FIGS. 11A-11B). Mutation of aspartic acid at residue 31 of CDR1 to asparagine increased the cationic charge of this residue and enhanced nucleic acid binding and delivery in vivo (3E10-D31N).

Mutation of aspartic acid at residue 31 of CDR1 to arginine (3E10-D31R), further expanded the cationic charge while mutation to lysine (3E10-D31K) changed charge orientation (FIG. 11A).

NAB1 amino acids predicted from molecular modeling have been underlined in the heavy and light chain sequences above. FIG. 11B is an illustration showing molecular modeling of 3E10-scFv (Pymol) with NAB1 amino acid residues illustrated with punctate dots.

### Example 5—Intermuscular Injection of 3E10 (D31N) Complexed with mRNA Results in Sustained Protein Expression in Skeletal Muscle

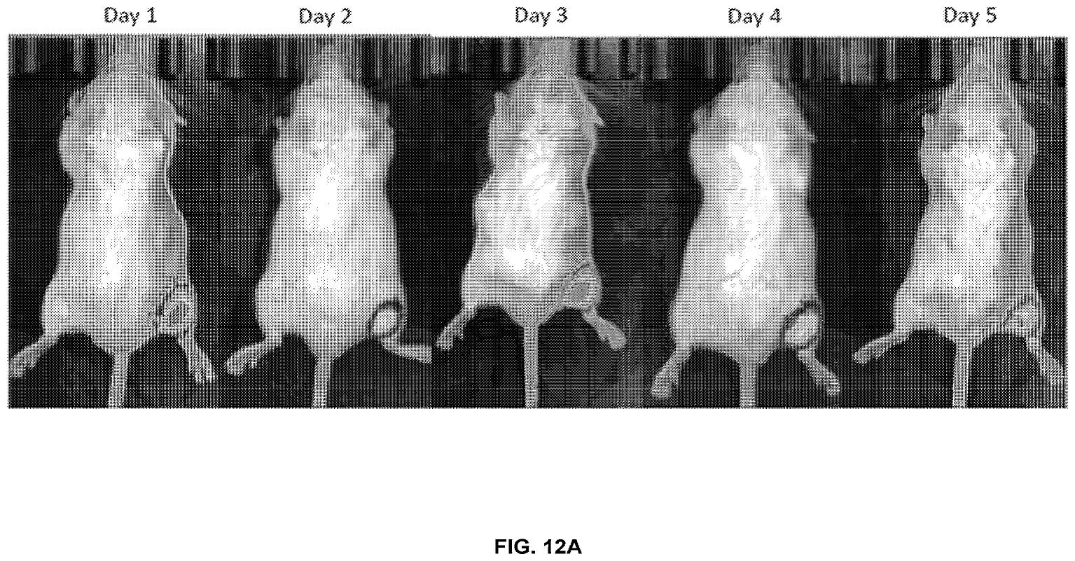

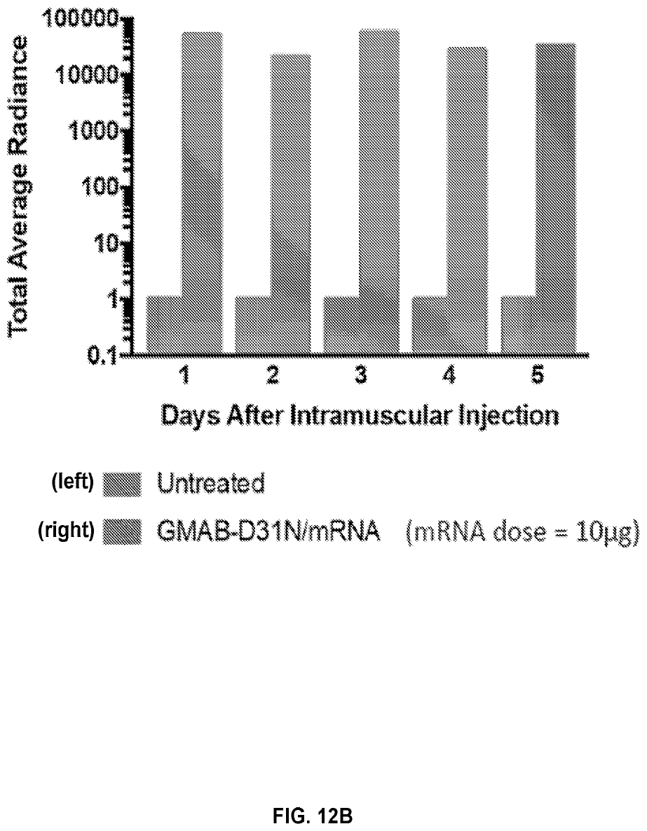

It was next investigated whether intramuscular administration of a 3E10 (D31N)-mRNA complex would result in sustained expression of the mRNA in skeletal muscle. Briefly, complexes of 3E10 (D31N) and mRNA encoding green fluorescent protein, a luciferase, having the sequence GFP_mRNA shown below as (SEQ ID NO:394), were formed by mixing 3E10 (D31N) and mRNA at a 20:1 molar ratio. The resulting complex was administered by intermuscular injection into the hind-leg skeletal muscle of a mouse. Bioluminescence in the skeletal muscle, indicating expression of the luciferase from the injected mRNA, was imaged (FIG. 12A) and quantified (FIG. 12B) over five days. As shown in FIG. 12B, expression levels of the mRNA-encoded luciferase were sustained for at least five days.

### Example 6-3E10 (D31N) Protects mRNA Against RNA Degradation

It was next investigated whether complexing mRNA with 3E10 (D31N) would protect the mRNA from degradation. Briefly, complexes of 3E10 (D31N) and mRNA encoding green fluorescent protein, a luciferase, having the sequence GFP_mRNA shown below as (SEQ ID NO: 394), were formed by mixing 3E10 (D31N) and mRNA at a 20:1 molar ratio. The free mRNA and the 3E10-mRNA complex were then incubated with 1% serum, 10% serum, or 16 μg/mL RNAse A. Gel electrophoresis analysis of the reactions was performed (FIG. 13A). As shown in FIG. 13A, free mRNA was degraded by incubation with each of 1% serum, 10% serum, and RNAse A. However, no apparent RNA degradation was observed when the complexed mRNA was incubated with any of 1% serum, 10% serum, or RNAse A, suggesting that 3E10 (D31N) protects mRNA from degradation.

>GFP_mRNA
(SEQ ID NO: 394)
AUGGUGAGCAAGGGCGAGGAGCUGUUCACCGGGGUGGUGCCCAUCC
UGGUCGAGCUGGACGGCGACGUAAACGGCCACAAGUUCAGCGUGU
CCGGCGAGGGCGAGGGCGAUGCCACCUACGGCAAGCUGACCCUGA
AGUUCAUCUGCACCACCGGCAAGCUGCCCGUGCCCUGGCCCACCC
UCGUGACCACCCUGACCUACGGCGUGCAGUGCUUCAGCCGCUACC
CCGACCACAUGAAGCAGCACGACUUCUUCAAGUCCGCCAUGCCCG
AAGGCUACGUCCAGGAGCGCACCAUCUUCUUCAAGGACGACGGCA
ACUACAAGACCCGCGCCGAGGUGAAGUUCGAGGGCGACACCCUGG
UGAACCGCAUCGAGCUGAAGGGCAUCGACUUCAAGGAGGACGGCA
ACAUCCUGGGGCACAAGCUGGAGUACAACUACAACAGCCACAACG
UCUAUAUCAUGGCCGACAAGCAGAAGAACGGCAUCAAGGUGAACU
UCAAGAUCCGCCACAACAUCGAGGACGGCAGCGUGCAGCUCGCCG
ACCACUACCAGCAGAACACCCCCAUCGGCGACGGCCCCGUGCUGC
UGCCCGACAACCACUACCUGAGCACCCAGUCCGCCCUGAGCAAAG
ACCCCAACGAGAAGCGCGAUCACAUGGUCCUGCUGGAGUUCGUGA
CCGCCGCCGGGAUCACUCUCGGCAUGGACGAGCUGUACAAGUAA.

Next, it was investigated whether mRNA complexed at lower molar ratios were also protected against RNA degradation. Briefly, complexes of 3E10 (D31N) and mRNA encoding green fluorescent protein (GFP_mRNA; SEQ ID NO:394) were formed by mixing 3E10 (D31N) and mRNA at a 2:1 molar ratio. The free mRNA and the 3E10-mRNA complex were then incubated with RNAse A under the conditions described above. Gel electrophoresis analysis of the reactions was performed (FIG. 13B). As shown in FIG. 13B, free mRNA was completely degraded by incubation with RNAse A. However, complexing the mRNA with 3E10 (D31N) at a 2:1 molar ratio resulted in some protection of the mRNA against degradation, as indicated by the presence of an RNA signal in the well, indicating the presence of intact 3E10 (D31N)-mRNA complex. The protection provided at a 2:1 molar ratio appears to be less than the protection afforded the mRNA when complexed at a 20:1 molar ratio.

### Example 7—Cellular Uptake of 5-TAMRA Labeled Exon Skipping Peptide Nucleic Acids (PNAs) by WT-3E10

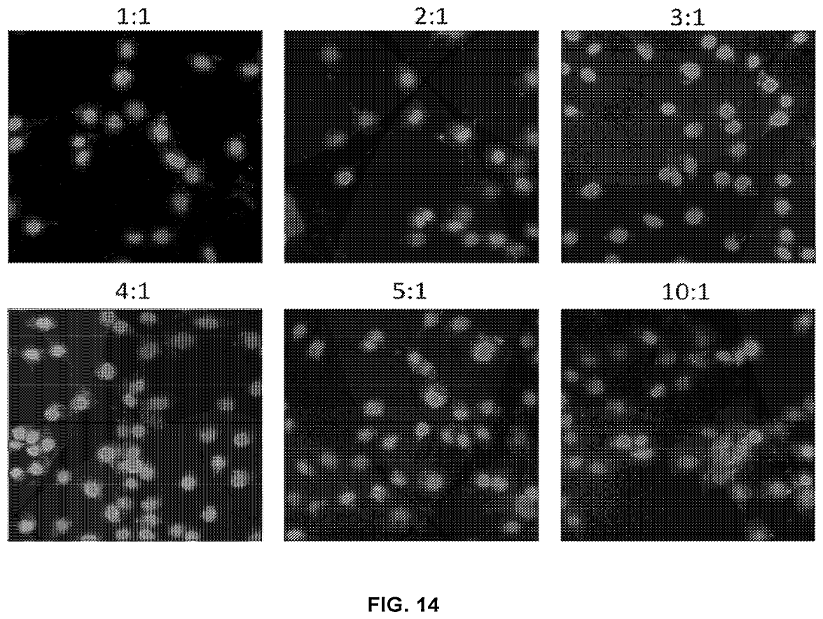

The cellular uptake of a 5-TAMA-labeled exon skipping PNA by 3E10 was investigated. As shown in FIG. 14, complexing WT-3E10 with PNA significantly enhanced PNA delivery. The best delivery of the exon skipping PNA was observed at molar ratios (WT-3E10: PNA) of 3:1, 4:1, 5:1, and 10:1, respectively.

### Example 8—An Exon skipping Antisense Oligonucleotide Targets Exon 23 in murine Dystrophin Using WT-3E10 after 48 and 72 Hour Incubation with Murine Myoblast Cells

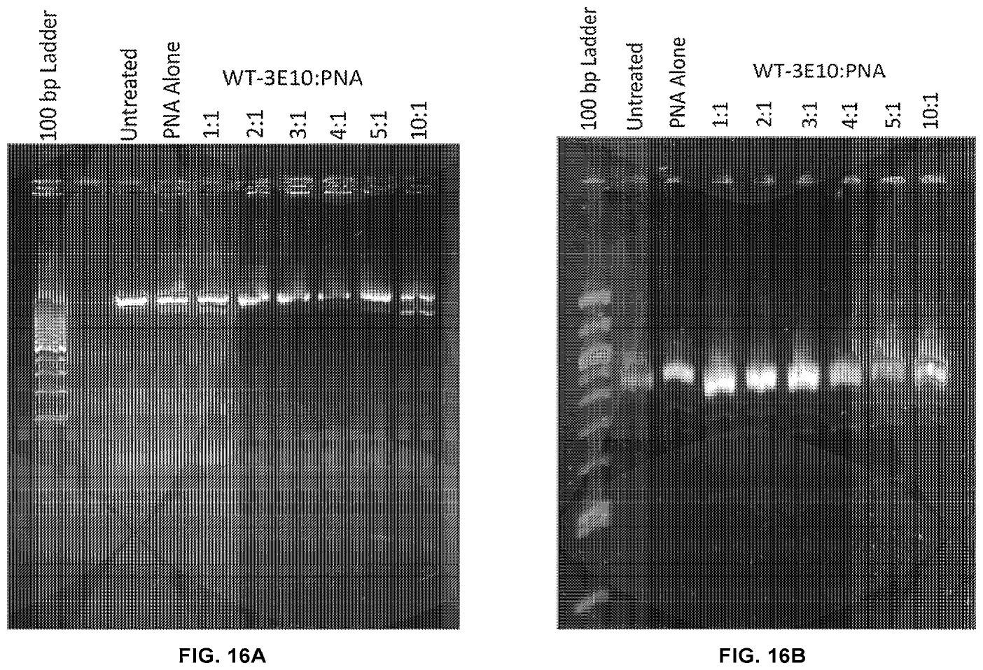

Exon skipping was investigated using WT-3E10 and a peptide nucleic acid targeting exon 23, DMD-TAM-PNA (SEQ ID NO:405, FIG. 15), 1 nmol per well (1 μM) at various molar ratios (WT-3E10: PNA) 1:1, 2:1, 3:1, 4:1, and 10:1, respectively. These complexes were incubated for 48 and 72 hours with murine myoblasts (100,000 cells/1 mL). Murine myoblast cells for all samples were assayed by RT-PCR and nested PCR to determine the occurrence of exon 23 skipping. As shown in FIG. 16A-16B, exon 23 skipping was observed only for WT-3E10: PNA ratios of 5:1 and 10:1 demonstrated by the DNA bands of 901 bp and 688 bp.

### Example 9—an Exon Skipping Antisense Oligonucleotide Targets Exon 23 in Murine Dystrophin Using 3E10 (D31N) after 48 and 72 Hour Incubation with Murine Myoblast Cells

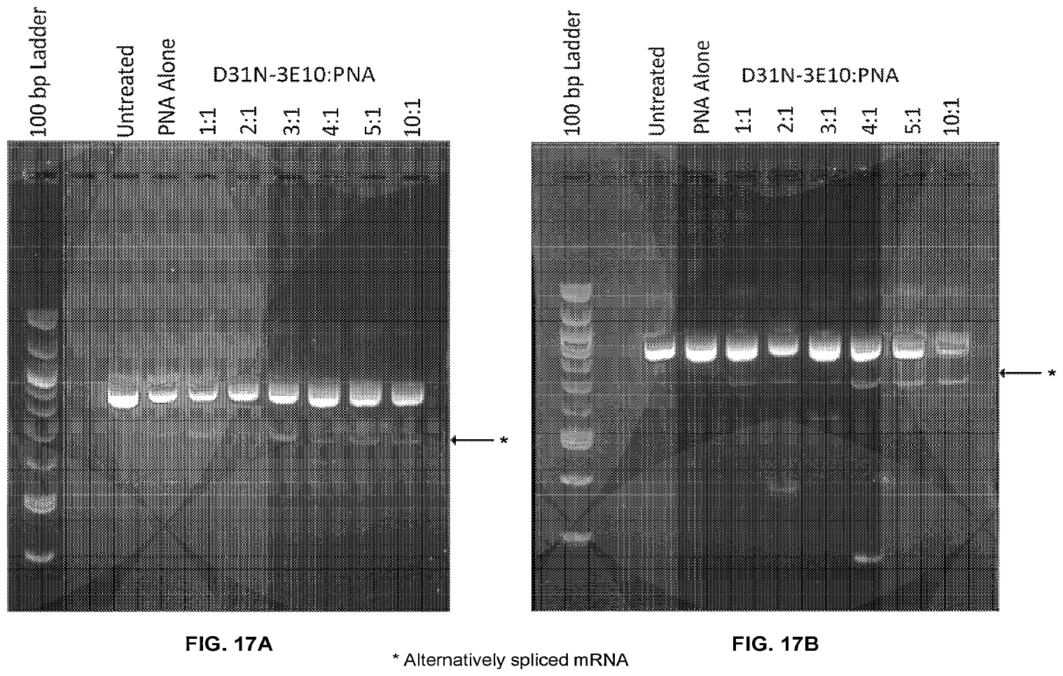

Exon skipping was investigated using 3E10 (D31N) and a peptide nucleic acid targeting exon 23, DMD-TAM-PNA (SEQ ID NO:405), 1 nmol per well (1 μM) at various molar ratios 3E10 (D31N): PNA 1:1, 2:1, 3:1, 4:1, and 10:1, respectively. These complexes were incubated for 48 and 72 hours with murine myoblasts (100,000 cells/1 mL). Murine myoblast cells for all samples were assayed by RT-PCR and nested PCR to determine the occurrence of exon 23 skipping. As shown in FIG. 17A-17B, exon 23 skipping was observed for all 3E10 (D31N): PNA ratios of 1:1, 2:1, 3:1, 4:1, 5:1, and 10:1 evidenced by the DNA bands of 901 bp and 688 bp.

### Example 10—Exon Skipping Negatively Charged Antisense Oligonucleotides Target Exon 23 in Murine Dystrophin Using 3E10 (D31N) after 48 Hour Incubation with Murine Myoblast Cells

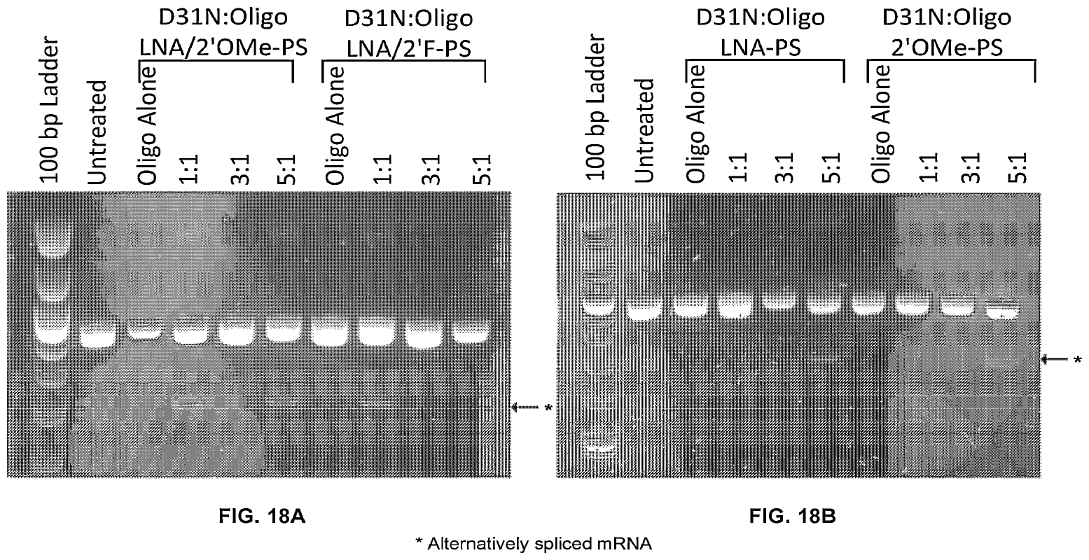

Exon skipping was investigated using 3E10 (D31N) and four negatively charged targeting exon 23: A) LNA/2′-OMe-PS or LNA-2′F-PS (SEQ ID NOs: 100 and 101, FIG. 15), or B) LNA-PS or 2′OMe-PS (SEQ ID NOs: 102 and 103, FIG. 15), 1 nmol per well (1 μM at various molar ratios 3E10 (D31N): PNA 1:1, 3:1, and 5:1, respectively. These complexes were incubated for 48 with murine myoblasts (100,000 cells/1 mL). Murine myoblast cells for all samples were assayed by RT-PCR and nested PCR to determine the occurrence of exon 23 skipping. As shown in FIG. 18A-18B, exon 23 skipping was observed for 3E10 (D31N): Oligo ratios of A) LNA/2′OMe-PS 1:1, 3:1, and 5:1, and LNA/2′F-PS 1:1, and B) LNA-PS 5:1, and 2′OMe-PS 5:1, evidenced by the DNA bands of 901 bp and 688 bp.

What is claimed is:

1. A method for treating a genetic disease in a subject in need thereof, the method comprising:
parenterally administering a therapeutically effective amount of a composition comprising a complex formed between (i) a 3E10 antibody or antigen-binding fragment thereof, and (ii) an antisense oligonucleotide complementary to a respective exon, splice site for a respective exon, or a cryptic splice site for a respective exon in a pre-mRNA for a gene that is mutated in the genetic disease, wherein the antisense oligonucleotide is substantially resistant to degradation by RNase-H.

2. The method of claim 1, wherein the 3E10 antibody or antigen-binding fragment thereof comprises:
(a) a light chain variable region (VL) complementarity determining region (CDR) 1 comprising an amino acid sequence having no more than two amino acid substitutions relative to 3E10-VL-CDR1 (SEQ ID NO:9),
(b) a VL CDR2 comprising an amino acid sequence having no more than two amino acid substitutions relative to 3E10-VL-CDR2 (SEQ ID NO:10),
(c) a VL CDR3 comprising an amino acid sequence having no more than two amino acid substitutions relative to 3E10-VL-CDR3 (SEQ ID NO:11),
(d) a heavy chain variable region (VH) CDR1 comprising an amino acid sequence having no more than two amino acid substitutions relative to 3E10-VH-CDR1a (SEQ ID NO:16),
(e) a VH CDR2 comprising an amino acid sequence having no more than two amino acid substitutions relative to 3E10-VH-CDR2 (SEQ ID NO:4), and
(f) a VH CDR3 comprising an amino acid sequence having no more than two amino acid substitutions relative to 3E10-VH-CDR3 (SEQ ID NO:5).

3. The method of claim 2, wherein the VL CDR1 has an amino acid sequence of 3E10-VL-CDR1m (SEQ ID NO:61).

4. The method of claim 2, wherein the VL CDR1 has the amino acid sequence of 3E10-VL-CDR1 (SEQ ID NO:9).

5. The method of any one of claims 2-4, wherein the VL CDR2 has an amino acid sequence of 3E10-VL-CDR2m (SEQ ID NO:62).

6. The method of any one of claims 2-4, wherein the VL CDR2 has the amino acid sequence of 3E10-VL-CDR2 (SEQ ID NO:10).

7. The method of any one of claims 2-6, wherein the VL CDR3 has an amino acid sequence of 3E10-VL-CDR3m (SEQ ID NO:63).

8. The method of any one of claims 2-6, wherein the VL CDR3 has the amino acid sequence of 3E10-VL-CDR3 (SEQ ID NO:11).

9. The method of any one of claims 2-8, wherein the VH CDR1 has an amino acid sequence of 3E10-VH-CDR1m (SEQ ID NO:58).

10. The method of any one of claims 2-8, wherein the VH CDR1 has the amino acid sequence of 3E10-VH-CDR1a (SEQ ID NO:16).

11. The method of any one of claims 2-10, wherein the VH CDR2 has an amino acid sequence selected of 3E10-VH-CDR2m (SEQ ID NO:59).

12. The method of any one of claims 2-10, wherein the VH CDR2 has the amino acid sequence of 3E10-VH-CDR2 (SEQ ID NO:4).

13. The method of any one of claims 2-12, wherein the VH CDR3 has an amino acid sequence of 3E10-VH-CDR3m (SEQ ID NO:60).

14. The method of any one of claims 2-12, wherein the VH CDR3 has the amino acid sequence of 3E10-VH-CDR3 (SEQ ID NO:5).

15. The method of claim 1, wherein the 3E10 antibody or antigen-binding fragment thereof comprises:
(a) a light chain variable region (VL) complementarity determining region (CDR) 1 comprising the amino acid sequence of 3E10-VL-CDR1 (SEQ ID NO:9),
(b) a VL CDR2 comprising the amino acid sequence of 3E10-VL-CDR2 (SEQ ID NO: 10),
(c) a VL CDR3 comprising the amino acid sequence of 3E10-VL-CDR3 (SEQ ID NO: 11),
(d) a heavy chain variable region (VH) CDR1 comprising the amino acid sequence of 3E10-VH-CDR1a (SEQ ID NO:16),
(e) a VH CDR2 comprising the amino acid sequence of 3E10-VH-CDR2 (SEQ ID NO: 4), and
(f) a VH CDR3 comprising the amino acid sequence of 3E10-VH-CDR3 (SEQ ID NO:5).

16. The method of any one of claims 1-15, wherein the 3E10 antibody or antigen binding fragment thereof comprises a VL comprising an amino acid sequence that is at least 85% identical to 3E10-VL (SEQ ID NO:8).

17. The method of claim 16, wherein the 3E10 antibody or antigen binding fragment thereof comprises a VL comprising an amino acid sequence that is at least 95% identical to 3E10-VL (SEQ ID NO:8).

18. The method of any one of claims 1-17, wherein the 3E10 antibody or antigen binding fragment thereof comprises a VH comprising an amino acid sequence that is at least 85% identical to 3E10-VH (SEQ ID NO:2).

19. The method of claim 18, wherein the 3E10 antibody or antigen binding fragment thereof comprises a VH comprising an amino acid sequence that is at least 95% identical to 3E10-VH (SEQ ID NO:2).

20. The method of any one of claims 1-15, wherein the 3E10 antibody or antigen binding fragment thereof is a humanized 3E10 antibody or antigen-binding fragment thereof.

21. The method of claim 20, wherein the humanized 3E10 antibody or antigen-binding fragment thereof comprises a light chain variable domain (3E10-VL) and a heavy chain variable domain (3E10-VH), wherein:
the 3E10-VL comprises an amino acid sequence that is at least 97% identical to an amino acid sequence selected from the group consisting of 3E10-VL-h1 (SEQ ID NO:125), 3E10-VL-h2 (SEQ ID NO:126), 3E10-VL-h3 (SEQ ID NO:127), 3E10-VL-h4 (SEQ ID NO:128), 3E10-VL-h5 (SEQ ID NO:129), and 3E10-VL-h6 (SEQ ID NO:130), and
the 3E10-VH comprises an amino acid sequence that is at least 95% identical to an amino acid sequence selected from the group consisting of 3E10-VH-h1 (SEQ ID NO:104), 3E10-VH-h2 (SEQ ID NO:105), 3E10-VH-h3 (SEQ ID NO:106), 3E10-VH-h4 (SEQ ID NO:107), 3E10-VH-h5 (SEQ ID NO:108), 3E10-VH-h6 (SEQ ID NO:109), and 3E10-VH-h7 (SEQ ID NO:110).

22. The method of claim 21, wherein the 3E10-VH comprises an amino acid sequence that is at least 95%, 96%, 97%, 98%, 99%, or 100% identical to 3E10-VH-h6 (SEQ ID NO:109) and the 3E10-VL comprises an amino acid sequence that is at least 97%, 98%, 99%, or 100% identical to 3E10-VL-h6 (SEQ ID NO:130).

23. The method of claim 21, wherein the 3E10-VH comprises an amino acid sequence of 3E10-VH-h6 (SEQ ID NO:109) and the 3E10-VL comprises an amino acid sequence of 3E10-VL-h6 (SEQ ID NO:130).

24. The method of claim 20, wherein the humanized 3E10 antibody or antigen-binding fragment thereof comprises a light chain (3E10-LC) and a heavy chain (3E10-HC), wherein:
the 3E10-LC comprises an amino acid sequence that is at least 97% identical to an amino acid sequence selected from the group consisting of 3E10-LC-h1m (SEQ ID NO:131), 3E10-LC-h2m (SEQ ID NO:132), 3E10-LC-h3m (SEQ ID NO:133), 3E10-LC-h4m (SEQ ID NO:134), 3E10-LC-h5m (SEQ ID NO:135), and 3E10-LC-h6m (SEQ ID NO:136), and
the 3E10-HC comprises an amino acid sequence that is at least 95% identical to an amino acid sequence selected from the group consisting of 3E10-HC-h1m (SEQ ID NO:111), 3E10-HC-h2m (SEQ ID NO:112), 3E10-HC-h3m (SEQ ID NO:113), 3E10-HC-h4m (SEQ ID NO: 114), 3E10-HC-h5m (SEQ ID NO:115), 3E10-HC-h6m (SEQ ID NO:116), and 3E10-HC-h7m (SEQ ID NO:117).

25. The method of claim 24, wherein the 3E10-HC comprises the amino acid sequence of 3E10-HC-h6m (SEQ ID NO:116) and the 3E10-LC comprises the amino acid sequence of 3E10-LC-h6m (SEQ ID NO:136).

26. The method of claim 24, wherein the 3E10-HC comprises the amino acid sequence of 3E10-HC-h6 (SEQ ID NO:123) and the 3E10-LC comprises the amino acid sequence of 3E10-LC-h6 (SEQ ID NO:142).

27. The method of any one of claims 1-26, wherein the 3E10 antibody or antigen binding fragment thereof comprises:
a heavy chain comprising, from N- to C-terminal, VH-CH1-hinge-CH2-CH3, and a light chain comprising, from N- to C-terminal, VL-CL.

28. The method of claim 27, wherein the hinge-CH2-CH3 is an Fc domain selected from the group consisting of the Fc domain from human IgG1, IgG2, IgG3 and IgG4.

29. The method of any one of claims 1-28, wherein the genetic disease is selected from those genetic diseases listed in Table 1 and the target gene is a gene corresponding to the genetic disease, as listed in Table 1.

30. The method of claim 29, wherein the respective exon is an exon corresponding to the genetic disease, as listed in Table 1.

31. The method of claim 29, wherein the genetic disease is Duchenne muscular dystrophy and the gene is a Dystrophin (DMD) gene.

32. The method of claim 31, wherein the splice site or cryptic splice site is for a DMD exon selected from the group consisting of exon 23, 43, 44, 45, 50, 51, 52, 53, and 55.

33. The method of claim 31, wherein the antisense oligonucleotide comprises a sequence selected from the group consisting of SEQ ID NO:99, SEQ ID NOs: 158-222, and SEQ ID NO: 395-405.

34. The method of claim 31, wherein the antisense oligonucleotide comprises a sequence of CAAUGCCAUCCUGGAGUUCCUG (SEQ ID NO:395).

35. The method of claim 34, wherein the antisense oligonucleotide is P-DEOXY-P-(DIMETHYLAMINO)) (2′,3′-DIDEOXY-2′,3′-IMINO-2′,3′-SECO) (2′A-5′) (C-A-A-M5U-G-C-C-A-M5U-C-C-M5U-G-G-A-G-M5U-M5U-C-C-M5U-G), 5′-(P-(4-((2-(2-(2-HYDROXYETHOXY) ETHOXY) ETHOXY) CARBONYL)-1-PIPERAZINYL)-N,N-DIMETHYLPHOSPHONAMIDATE (SEQ ID NO:399).

36. The method of claim 31, wherein the antisense oligonucleotide comprises a sequence of CTCCAACATCAAGGAAGATGGCATTTCTAG (SEQ ID NO:208).

37. The method of claim 36, wherein the antisense oligonucleotide is P-deoxy-P-(DIMETHYLAMINO)) (2′,3′-DIDEOXY-2′,3′-IMINO-2′,3′-SECO) (2′a→5′) (C-m5U-C-C-A-A-C-A-m5U-C-A-A-G-G-A-A-G-A-m5U-G-G-C-A-m5U-m5U-m5U-C-m5U-A-G),5′-(P-(4-((2-(2-(2-HYDROXYETHOXY) ETHOXY) ETHOXY) CARBONYL)-1-PIPERAZINYL)-N,N-DIMETHYLPHOSPHONAMIDATE) (SEQ ID NO:400).

38. The method of claim 31, wherein the antisense oligonucleotide comprises a sequence of GUUGCCUCCGGUUCUGAAGGUGUUC (SEQ ID NO:401).

39. The method of claim 38, wherein the antisense oligonucleotide is P-DEOXY-P-(DIMETHYLAMINO)) (2′,3′-DIDEOXY-2′,3′-IMINO-2′,3′-SECO) (2′A→5′) (G-M5U-M5U-G-C-C-M5U-C-C-G-G-M5U-M5U-C-M5U-G-A-A-G-G-M5U-G-M5U-M5U-C), 5′-(P-(4-((2-(2-(2-HYDROXYETHOXY) ETHOXY) ETHOXY) CARBONYL)-1-PIPERAZINYL)-N,N-DIMETHYLPHOSPHONAMIDATE (SEQ ID NO:403).

40. The method of claim 31, wherein the antisense oligonucleotide comprises a sequence of CCUCCGGUUCUGAAGGUGUUC (SEQ ID NO:402).

41. The method of claim 40, wherein the antisense oligonucleotide is P-DEOXY-P-(DIMETHYLAMINO)) (2′,3′-DIDEOXY-2′,3′-IMINO-2′,3′-SECO) (2′A→5′) (C-C-M5U-C-C-G-G-M5U-M5U-C-M5U-G-A-A-G-G-M5U-G-M5U-M5U-C) (SEQ ID NO:404).

42. The method of any one of claims 1-28, wherein the target gene is a collagen alpha-1 (VII) chain (COL7A1) gene.

43. The method of claim 42, wherein the splice site or cryptic splice site is for exon 73 of the COL7A1 gene.

44. The method of any one of claims 1-28, wherein the target gene is a Usherin (USH2A) gene.

45. The method of claim 44, wherein the splice site or cryptic splice site is for exon 13 of the USH2A gene.

46. The method of any one of claims 1-28, wherein the genetic disease is selected from the group consisting of a congenital disorder of glycosylation, familial dysautonomia, a dystrophy, Duchenne muscular dystrophy, myotonic dystrophy 1 (DM1), myotonic dystrophy 2 (DM2), spinal muscular atrophy, a cancer, bladder cancer, breast cancer, gastric cancer, acute myeloid leukemia, lung cancer, pancreatic carcinoma, RAS-associated autoimmune leukoproliferative disorder type IV, colorectal cancer, follicular thyroid carcinoma, Valosine-containing protein (VCP)-associated inclusion body myopathy (IBM), inflammatory bowel diseases (IBS), Hutchison-Gilford progeria syndrome (HGPS), Alagille syndrome, tuberous sclerosis, Usher syndrome, Aicardi-Goutieres syndrome-6 (AGS6), familial hemiplegic migraine, familiar basilar migraine, alternating hemiplegia, progressive myoclonic epilepsy, mental retardation-23 (MRD23), 3p25 microdeletion syndrome, leukoencephalopathy, Zellweger syndrome, heimler syndrome-1, early infantile epileptic encephalopathy, familial infantile convulsion with paroxysmal choreoathetosis, episodic kinesigenic dyskenisia 1, benign familial infantile seizures, epilepsy, Smith-Magenis syndrome, Pitt-Hopkins syndrome, Niemann-Pick disease, episodic ataxia type 2, familial hemiplagic migraine, SpinoCerebellar Ataxia type 6, hereditary sensory neuropathy type IE, autosomal dominant cerebellar ataxia, deafness, narcolepsy, neurofibromatosis type 2, NF2-related meningioma, shwannomatosis, Phelan-McDermid syndrome (PHMDS), schizophrenia-15 (SCZD15), metachromatic leukodystrophy, cystic fibrosis, familial hypercholesterolemia, phenylketonuria, Hutchinson-Gilford progeria syndrome, inflammatory bowel disease, frontotemporal dementia with parkinsonism, dystrophic epidermolysis bullosa, and Parkinson's disease.

47. The method of any one of claims 1-45, wherein the antisense oligonucleotide is a PNA oligonucleotide.

48. The method of claim 47, wherein the antisense oligonucleotide is selected from the group consisting of a phosphorodiamidate morpholino oligonucleotide (PMO), a 2′-O-methyl phosphorothioate oligonucleotide, a locked nucleic acid (LNA) oligonucleotide, and a bridged nucleic acid (BNA) oligonucleotide.

49. The method of any one of claims 1-45, wherein the antisense oligonucleotide comprises at least one 2′ ribose substitution.

50. The method of claim 49, wherein the 2′ ribose substitution is selected from the group consisting of 2′-O-methyl, 2′-O-methoxyethyl (2′-O-MOE), 2′-O-aminopropyl, 2′-deoxy, 2′-deoxy-2′-fluoro, 2′-deoxy-2′,4′-difluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide; an ethylene nucleic acid (ENA); or a combination thereof.

51. The method of any one of claims 1-48, wherein the composition comprises a molar ratio of (i) 3E10 antibody or antigen-binding fragment thereof to (ii) therapeutic polynucleotide of no more than 10:1.

52. The method of claim 51, wherein the composition comprises a molar ratio of (i) 3E10 antibody or antigen-binding fragment thereof to (ii) therapeutic polynucleotide of no more than 1:1.

53. The method of claim 51, wherein the composition comprises a molar ratio of (i) 3E10 antibody or antigen-binding fragment thereof to (ii) therapeutic polynucleotide of no more than 1:3.

54. The method of claim 51, wherein the composition comprises a molar ratio of (i) 3E10 antibody or antigen-binding fragment thereof to (ii) therapeutic polynucleotide of no more than 1:5.

55. The method of claim 51, wherein the composition comprises a molar ratio of (i) 3E10 antibody or antigen-binding fragment thereof to (ii) therapeutic polynucleotide of no more than 1:10.

56. The method of any one of claims 51-55, wherein the composition comprises a molar ratio of (i) 3E10 antibody or antigen-binding fragment thereof to (ii) therapeutic polynucleotide of at least 1:50.

57. The method of claim 56, wherein the composition comprises a molar ratio of (i) 3E10 antibody or antigen-binding fragment thereof to (ii) therapeutic polynucleotide of at least 1:25.

58. The method of any one of claims 1-57, wherein the parenteral administration is intramuscular administration, intravenous administration, or subcutaneous administration.

59. A pharmaceutical composition comprising a therapeutically effective amount of a complex formed between (i) a 3E10 antibody or antigen-binding fragment thereof, and (ii) an antisense oligonucleotide complementary to a respective exon, splice site for a respective exon, or a cryptic splice site for a respective exon in a pre-mRNA for a gene that is mutated in the genetic disease, wherein the antisense oligonucleotide is substantially resistant to degradation by RNase-H.

60. The pharmaceutical composition of claim 59, wherein the 3E10 antibody or antigen-binding fragment thereof comprises:
(a) a light chain variable region (VL) complementarity determining region (CDR) 1 comprising an amino acid sequence having no more than two amino acid substitutions relative to 3E10-VL-CDR1 (SEQ ID NO:9),
(b) a VL CDR2 comprising an amino acid sequence having no more than two amino acid substitutions relative to 3E10-VL-CDR2 (SEQ ID NO:10),
(c) a VL CDR3 comprising an amino acid sequence having no more than two amino acid substitutions relative to 3E10-VL-CDR3 (SEQ ID NO:11),
(d) a heavy chain variable region (VH) CDR1 comprising an amino acid sequence having no more than two amino acid substitutions relative to 3E10-VH-CDR1a (SEQ ID NO:16),
(e) a VH CDR2 comprising an amino acid sequence having no more than two amino acid substitutions relative to 3E10-VH-CDR2 (SEQ ID NO:4), and
(f) a VH CDR3 comprising an amino acid sequence having no more than two amino acid substitutions relative to 3E10-VH-CDR3 (SEQ ID NO:5).

61. The pharmaceutical composition of claim 60, wherein the VL CDR1 has an amino acid sequence of 3E10-VL-CDR1m (SEQ ID NO:61).

62. The pharmaceutical composition of claim 60, wherein the VL CDR1 has the amino acid sequence of 3E10-VL-CDR1 (SEQ ID NO:9).

63. The pharmaceutical composition of any one of claims 60-62, wherein the VL CDR2 has an amino acid sequence of 3E10-VL-CDR2m (SEQ ID NO:62).

64. The pharmaceutical composition of any one of claims 60-62, wherein the VL CDR2 has the amino acid sequence of 3E10-VL-CDR2 (SEQ ID NO:10).

65. The pharmaceutical composition of any one of claims 60-64, wherein the VL CDR3 has an amino acid sequence of 3E10-VL-CDR3m (SEQ ID NO:63).

66. The pharmaceutical composition of any one of claims 60-64, wherein the VL CDR3 has the amino acid sequence of 3E10-VL-CDR3 (SEQ ID NO:11).

67. The pharmaceutical composition of any one of claims 60-66, wherein the VH CDR1 has an amino acid sequence of 3E10-VH-CDR1m (SEQ ID NO:58).

68. The pharmaceutical composition of any one of claims 60-66, wherein the VH CDR1 has the amino acid sequence of 3E10-VH-CDR1a (SEQ ID NO:16).

69. The pharmaceutical composition of any one of claims 60-68, wherein the VH CDR2 has an amino acid sequence selected of 3E10-VH-CDR2m (SEQ ID NO:59).

70. The pharmaceutical composition of any one of claims 60-68, wherein the VH CDR2 has the amino acid sequence of 3E10-VH-CDR2 (SEQ ID NO:4).

71. The pharmaceutical composition of any one of claims 60-70, wherein the VH CDR3 has an amino acid sequence of 3E10-VH-CDR3m (SEQ ID NO:60).

72. The pharmaceutical composition of any one of claims 60-70, wherein the VH CDR3 has the amino acid sequence of 3E10-VH-CDR3 (SEQ ID NO:5).

73. The pharmaceutical composition of claim 59, wherein the 3E10 antibody or antigen-binding fragment thereof comprises:
(a) a light chain variable region (VL) complementarity determining region (CDR) 1 comprising the amino acid sequence of 3E10-VL-CDR1 (SEQ ID NO:9),
(b) a VL CDR2 comprising the amino acid sequence of 3E10-VL-CDR2 (SEQ ID NO: 10),
(c) a VL CDR3 comprising the amino acid sequence of 3E10-VL-CDR3 (SEQ ID NO: 11),
(d) a heavy chain variable region (VH) CDR1 comprising the amino acid sequence of 3E10-VH-CDR1a (SEQ ID NO:16),
(e) a VH CDR2 comprising the amino acid sequence of 3E10-VH-CDR2 (SEQ ID NO: 4), and
(f) a VH CDR3 comprising the amino acid sequence of 3E10-VH-CDR3 (SEQ ID NO:5).

74. The pharmaceutical composition of any one of claims 59-73, wherein the 3E10 antibody or antigen binding fragment thereof comprises a VL comprising an amino acid sequence that is at least 85% identical to 3E10-VL (SEQ ID NO:8).

75. The pharmaceutical composition of claim 74, wherein the 3E10 antibody or antigen binding fragment thereof comprises a VL comprising an amino acid sequence that is at least 95% identical to 3E10-VL (SEQ ID NO:8).

76. The pharmaceutical composition of any one of claims 59-75, wherein the 3E10 antibody or antigen binding fragment thereof comprises a VH comprising an amino acid sequence that is at least 85% identical to 3E10-VH (SEQ ID NO:2).

77. The pharmaceutical composition of claim 76, wherein the 3E10 antibody or antigen binding fragment thereof comprises a VH comprising an amino acid sequence that is at least 95% identical to 3E10-VH (SEQ ID NO:2).

78. The pharmaceutical composition of any one of claims 59-77, wherein the 3E10 antibody or antigen binding fragment thereof is a humanized 3E10 antibody or antigen-binding fragment thereof.

79. The pharmaceutical composition of claim 78, wherein the humanized 3E10 antibody or antigen-binding fragment thereof comprises a light chain variable domain (3E10-VL) and a heavy chain variable domain (3E10-VH), wherein:
the 3E10-VL comprises an amino acid sequence that is at least 97% identical to an amino acid sequence selected from the group consisting of 3E10-VL-h1 (SEQ ID NO:125), 3E10-VL-h2 (SEQ ID NO:126), 3E10-VL-h3 (SEQ ID NO:127), 3E10-VL-h4 (SEQ ID NO:128), 3E10-VL-h5 (SEQ ID NO:129), and 3E10-VL-h6 (SEQ ID NO:130), and
the 3E10-VH comprises an amino acid sequence that is at least 95% identical to an amino acid sequence selected from the group consisting of 3E10-VH-h1 (SEQ ID NO:104), 3E10-VH-h2 (SEQ ID NO:105), 3E10-VH-h3 (SEQ ID NO:106), 3E10-VH-h4 (SEQ ID NO:107), 3E10-VH-h5 (SEQ ID NO:108), 3E10-VH-h6 (SEQ ID NO:109), and 3E10-VH-h7 (SEQ ID NO:120).

80. The pharmaceutical composition of claim 79, wherein the 3E10-VH comprises an amino acid sequence that is at least 95%, 96%, 97%, 98%, 99%, or 100% identical to 3E10-VH-h6 (SEQ ID NO:109) and the 3E10-VL comprises an amino acid sequence that is at least 97%, 98%, 99%, or 100% identical to 3E10-VL-h6 (SEQ ID NO:130).

81. The pharmaceutical composition of claim 79, wherein the 3E10-VH comprises an amino acid sequence of 3E10-VH-h6 (SEQ ID NO:69) and the 3E10-VL comprises an amino acid sequence of 3E10-VL-h6 (SEQ ID NO:90).

82. The pharmaceutical composition of claim 79, wherein the humanized 3E10 antibody or antigen-binding fragment thereof comprises a light chain (3E10-LC) and a heavy chain (3E10-HC), wherein:
the 3E10-LC comprises an amino acid sequence that is at least 97% identical to an amino acid sequence selected from the group consisting of 3E10-LC-h1m (SEQ ID NO:131), 3E10-LC-h2m (SEQ ID NO:132), 3E10-LC-h3m (SEQ ID NO:133), 3E10-LC-h4m (SEQ ID NO:134), 3E10-LC-h5m (SEQ ID NO:135), and 3E10-LC-h6m (SEQ ID NO:136), and
the 3E10-HC comprises an amino acid sequence that is at least 95% identical to an amino acid sequence selected from the group consisting of 3E10-HC-h1m (SEQ ID NO:111), 3E10-HC-h2m (SEQ ID NO:112), 3E10-HC-h3m (SEQ ID NO:113), 3E10-HC-h4m (SEQ ID NO: 114), 3E10-HC-h5m (SEQ ID NO:115), 3E10-HC-h6m (SEQ ID NO:116), and 3E10-HC-h7m (SEQ ID NO:117).

83. The pharmaceutical composition of claim 82, wherein the 3E10-HC comprises the amino acid sequence of 3E10-HC-h6m (SEQ ID NO:116) and the 3E10-LC comprises the amino acid sequence of 3E10-LC-h6m (SEQ ID NO:136).

84. The pharmaceutical composition of claim 82, wherein the 3E10-HC comprises the amino acid sequence of 3E10-HC-h6 (SEQ ID NO:123) and the 3E10-LC comprises the amino acid sequence of 3E10-LC-h6 (SEQ ID NO:142).

85. The pharmaceutical composition of any one of claims 59-84, wherein the 3E10 antibody or antigen binding fragment thereof comprises:
a heavy chain comprising, from N- to C-terminal, VH-CH1-hinge-CH2-CH3, and a light chain comprising, from N- to C-terminal, VL-CL.

86. The pharmaceutical composition of claim 85, wherein the hinge-CH2-CH3 is an Fc domain selected from the group consisting of the Fc domain from human IgG1, IgG2, IgG3 and IgG4.

87. The pharmaceutical composition of any one of claims 59-86, wherein the genetic disease is selected from those genetic diseases listed in Table 1 and the target gene is a gene corresponding to the genetic disease, as listed in Table 1.

88. The pharmaceutical composition of claim 87, wherein the respective exon is an exon corresponding to the genetic disease, as listed in Table 1.

89. The pharmaceutical composition of claim 87, wherein the genetic disease is Duchenne muscular dystrophy and the gene is a Dystrophin (DMD) gene.

90. The pharmaceutical composition of claim 89, wherein the splice site or cryptic splice site is for a DMD exon selected from the group consisting of exon 23, 43, 44, 45, 50, 51, 52, 53, and 55.

91. The pharmaceutical composition of claim 89, wherein the antisense oligonucleotide comprises a sequence selected from the group consisting of SEQ ID NO:99, SEQ ID NOs: 158-222, and SEQ ID NO:395-405.

92. The pharmaceutical composition of claim 89, wherein the antisense oligonucleotide comprises a sequence of CAAUGCCAUCCUGGAGUUCCUG (SEQ ID NO:395).

93. The pharmaceutical composition of claim 89, wherein the antisense oligonucleotide is P-DEOXY-P-(DIMETHYLAMINO)) (2′,3′-DIDEOXY-2′,3′-IMINO-2′,3′-SECO) (2′A-5′) (C-A-A-M5U-G-C-C-A-M5U-C-C-M5U-G-G-A-G-M5U-M5U-C-C-M5U-G), 5′-(P-(4-((2-(2-(2-HYDROXYETHOXY) ETHOXY) ETHOXY) CARBONYL)-1-PIPERAZINYL)-N,N-DIMETHYLPHOSPHONAMIDATE (SEQ ID NO:399).

94. The pharmaceutical composition of claim 89, wherein the antisense oligonucleotide comprises a sequence of CTCCAACATCAAGGAAGATGGCATTTCTAG (SEQ ID NO:208).

95. The pharmaceutical composition of claim 89, wherein the antisense oligonucleotide is P-DEOXY-P-(DIMETHYLAMINO)) (2′,3′-DIDEOXY-2′,3′-IMINO-2′,3′-SECO) (2′a→5′) (C-m5U-C-C-A-A-C-A-m5U-C-A-A-G-G-A-A-G-A-m5U-G-G-C-A-m5U-m5U-m5U-C-m5U-A-G),5′-(P-(4-((2-(2-(2-HYDROXYETHOXY) ETHOXY) ETHOXY) CARBONYL)-1-PIPERAZINYL)-N,N-DIMETHYLPHOSPHONAMIDATE) (SEQ ID NO:400).

96. The pharmaceutical composition of claim 89, wherein the antisense oligonucleotide comprises a sequence of GUUGCCUCCGGUUCUGAAGGUGUUC (SEQ ID NO:401).

97. The pharmaceutical composition of claim 89, wherein the antisense oligonucleotide is P-DEOXY-P-(DIMETHYLAMINO)) (2′,3′-DIDEOXY-2′,3′-IMINO-2′,3′-SECO) (2′A→5′) (G-M5U-M5U-G-C-C-M5U-C-C-G-G-M5U-M5U-C-M5U-G-A-A-G-G-M5U-G-M5U-M5U-C), 5′-(P-(4-((2-(2-(2-HYDROXYETHOXY) ETHOXY) ETHOXY) CARBONYL)-1-PIPERAZINYL)-N,N-DIMETHYLPHOSPHONAMIDATE (SEQ ID NO:403).

98. The pharmaceutical composition of claim 89, wherein the antisense oligonucleotide comprises a sequence of CCUCCGGUUCUGAAGGUGUUC (SEQ ID NO:402).

99. The pharmaceutical composition of claim 89, wherein the antisense oligonucleotide is P-DEOXY-P-(DIMETHYLAMINO)) (2′,3′-DIDEOXY-2′,3′-IMINO-2′,3′-SECO) (2′A→5′) (C-C-M5U-C-C-G-G-M5U-M5U-C-M5U-G-A-A-G-G-M5U-G-M5U-M5U-C) (SEQ ID NO:404).

100. The pharmaceutical composition of any one of claims 59-86, wherein the target gene is a collagen alpha-1 (VII) chain (COL7A1) gene.

101. The pharmaceutical composition of claim 100, wherein the splice site or cryptic splice site is for exon 73 of the COL7A1 gene.

102. The pharmaceutical composition of any one of claims 59-86, wherein the target gene is a Usherin (USH2A) gene.

103. The pharmaceutical composition of claim 102, wherein the splice site or cryptic splice site is for exon 13 of the USH2A gene.

104. The pharmaceutical composition of any one of claims 59-86, wherein the genetic disease is selected from the group consisting of a congenital disorder of glycosylation, familial dysautonomia, a dystrophy, Duchenne muscular dystrophy, myotonic dystrophy 1 (DM1), myotonic dystrophy 2 (DM2), spinal muscular atrophy, a cancer, bladder cancer, breast cancer, gastric cancer, acute myeloid leukemia, lung cancer, pancreatic carcinoma, RAS-associated autoimmune leukoproliferative disorder type IV, colorectal cancer, follicular thyroid carcinoma, Valosine-containing protein (VCP)-associated inclusion body myopathy (IBM), inflammatory bowel diseases (IBS), Hutchison-Gilford progeria syndrome (HGPS), Alagille syndrome, tuberous sclerosis, Usher syndrome, Aicardi-Goutieres syndrome-6 (AGS6), familial hemiplegic migraine, familiar basilar migraine, alternating hemiplegia, progressive myoclonic epilepsy, mental retardation-23 (MRD23), 3p25 microdeletion syndrome, leukoencephalopathy, Zellweger syndrome, heimler syndrome-1, early infantile epileptic encephalopathy, familial infantile convulsion with paroxysmal choreoathetosis, episodic kinesigenic dyskenisia 1, benign familial infantile seizures, epilepsy, Smith-Magenis syndrome, Pitt-Hopkins syndrome, Niemann-Pick disease, episodic ataxia type 2, familial hemiplagic migraine, SpinoCerebellar Ataxia type 6, hereditary sensory neuropathy type IE, autosomal dominant cerebellar ataxia, deafness, narcolepsy, neurofibromatosis type 2, NF2-related meningioma, shwannomatosis, Phelan-McDermid syndrome (PHMDS), schizophrenia-15 (SCZD15), metachromatic leukodystrophy, cystic fibrosis, familial hypercholesterolemia, phenylketonuria, Hutchinson-Gilford progeria syndrome, inflammatory bowel disease, frontotemporal dementia with parkinsonism, and Parkinson's disease.

105. The pharmaceutical composition of any one of claims 59-104, wherein the antisense oligonucleotide is a PNA oligonucleotide.

106. The pharmaceutical composition of claim 105, wherein the antisense oligonucleotide is selected from the group consisting of a phosphorodiamidate morpholino oligonucleotide (PMO), a 2′-O-methyl phosphorothioate oligonucleotide, a locked nucleic acid (LNA) oligonucleotide, and a bridged nucleic acid (BNA) oligonucleotide.

107. The pharmaceutical composition of any one of claims 59-104, wherein the antisense oligonucleotide comprises at least one 2′ ribose substitution.

108. The pharmaceutical composition of claim 107, wherein the 2′ ribose substitution is selected from the group consisting of 2′-O-methyl, 2′-O-methoxyethyl (2′-O-MOE), 2′-O-aminopropyl, 2′-deoxy, 2′-deoxy-2′-fluoro, 2′-deoxy-2′,4′-difluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide; an ethylene nucleic acid (ENA); or a combination thereof.

109. The pharmaceutical composition of any one of claims 59-107, wherein the composition comprises a molar ratio of (i) 3E10 antibody or antigen-binding fragment thereof to (ii) therapeutic polynucleotide of no more than 10:1.

110. The pharmaceutical composition of claim 109, wherein the composition comprises a molar ratio of (i) 3E10 antibody or antigen-binding fragment thereof to (ii) therapeutic polynucleotide of no more than 1:1.

111. The pharmaceutical composition of claim 109, wherein the composition comprises a molar ratio of (i) 3E10 antibody or antigen-binding fragment thereof to (ii) therapeutic polynucleotide of no more than 1:3.

112. The pharmaceutical composition of claim 109, wherein the composition comprises a molar ratio of (i) 3E10 antibody or antigen-binding fragment thereof to (ii) therapeutic polynucleotide of no more than 1:5.

113. The pharmaceutical composition of claim 109, wherein the composition comprises a molar ratio of (i) 3E10 antibody or antigen-binding fragment thereof to (ii) therapeutic polynucleotide of no more than 1:10.

114. The pharmaceutical composition of any one of claims 109-113, wherein the composition comprises a molar ratio of (i) 3E10 antibody or antigen-binding fragment thereof to (ii) therapeutic polynucleotide of at least 1:50.

115. The pharmaceutical composition of claim 114, wherein the composition comprises a molar ratio of (i) 3E10 antibody or antigen-binding fragment thereof to (ii) therapeutic polynucleotide of at least 1:25.

116. The pharmaceutical composition of any one of claims 59-115, wherein the pharmaceutical composition is formulated for parenteral administration.

117. The pharmaceutical composition of claim 116, wherein the parenteral administration is intramuscular administration, intravenous administration, or subcutaneous administration.


SEQUENCE LISTING (408 sequences)

SEQ ID NO: 1 - protein, 446 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 116 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 5 aa, synthetic construct
DYGMH

SEQ ID NO: 4 - protein, 17 aa, synthetic construct
YISSGSSTIYYADTVKG

SEQ ID NO: 5 - protein, 7 aa, synthetic construct
RGLLLDY

SEQ ID NO: 6 - protein, 19 aa, synthetic construct
MGWSCIILFLVATATGVHS

SEQ ID NO: 7 - protein, 218 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 111 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 9 - protein, 15 aa, synthetic construct
RASKSVSTSSYSYMH

SEQ ID NO: 10 - protein, 7 aa, synthetic construct
YASYLES

SEQ ID NO: 11 - protein, 9 aa, synthetic construct
QHSREFPWT

SEQ ID NO: 12 - protein, 19 aa, synthetic construct
MGWSCIILFLVATATGVHS

SEQ ID NO: 13 - protein, 446 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 116 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 15 - protein, 5 aa, synthetic construct
NYGMH

SEQ ID NO: 16 - protein, 5 aa, synthetic construct
BYGMH

SEQ ID NO: 17 - protein, 17 aa, synthetic construct
YISSGSSTIYYADTVKG

SEQ ID NO: 18 - protein, 7 aa, synthetic construct
RGLLLDY

SEQ ID NO: 19 - protein, 19 aa, synthetic construct
MGWSCIILFLVATATGVHS

SEQ ID NO: 20 - protein, 218 aa, synthetic construct; identical to SEQ ID NO: 7

SEQ ID NO: 21 - protein, 111 aa, synthetic construct; identical to SEQ ID NO: 8

SEQ ID NO: 22 - protein, 15 aa, synthetic construct
RASKSVSTSSYSYMH

SEQ ID NO: 23 - protein, 7 aa, synthetic construct
YASYLES

SEQ ID NO: 24 - protein, 9 aa, synthetic construct
QHSREFPWT

SEQ ID NO: 25 - protein, 19 aa, synthetic construct
MGWSCIILFLVATATGVHS

SEQ ID NO: 26 - protein, 17 aa, synthetic construct
YISSGSSTIYYADSVKG

SEQ ID NO: 27 - protein, 17 aa, synthetic construct
YISSSSSTIYYADSVKG

SEQ ID NO: 28 - protein, 15 aa, synthetic construct
RASKSVSTSSYSYLA

SEQ ID NO: 29 - protein, 15 aa, synthetic construct
RASKTVSTSSYSYMH

SEQ ID NO: 30 - protein, 7 aa, synthetic construct
YASYLQS

SEQ ID NO: 31 - protein, 17 aa, synthetic construct
  VARIANT 5: note = X is separately any amino acid
  VARIANT 14: note = X is separately any amino acid
YISSXSSTIYYADXVKG

SEQ ID NO: 32 - protein, 15 aa, synthetic construct
  VARIANT 5: note = X is separately any amino acid
  VARIANT 14..15: note = X are separately any amino acids
RASKXVSTSSYSYXX

SEQ ID NO: 33 - protein, 7 aa, synthetic construct
  VARIANT 6: note = X is separately any amino acid
YASYLXS

SEQ ID NO: 34 - protein, 5 aa, synthetic construct
QYGMH

SEQ ID NO: 35 - protein, 5 aa, synthetic construct
EYGMH

SEQ ID NO: 36 - skipped

SEQ ID NO: 37 - protein, 5 aa, synthetic construct
  VARIANT 5: note = X is K or R
QYGMX

SEQ ID NO: 38 - protein, 5 aa, synthetic construct
  VARIANT 5: note = X is K or R
EYGMX

SEQ ID NO: 39 - protein, 17 aa, synthetic construct
YISSGSSTIYYAETVKG

SEQ ID NO: 40 - protein, 17 aa, synthetic construct
  VARIANT 16: note = X is R or H
YISSGSSTIYYADTVXG

SEQ ID NO: 41 - protein, 17 aa, synthetic construct
  VARIANT 16: note = X is R or H
YISSGSSTIYYAETVXG

SEQ ID NO: 42 - protein, 7 aa, synthetic construct
  VARIANT 1: note = X is K or H
XGLLLDY

SEQ ID NO: 43 - protein, 8 aa, synthetic construct
RGLLLBEY

SEQ ID NO: 44 - protein, 7 aa, synthetic construct
  VARIANT 1: note = X is K or H
XGLLLEY

SEQ ID NO: 45 - protein, 15 aa, synthetic construct
  VARIANT 1: note = X is K or H
XASKSVSTSSYSYMH

SEQ ID NO: 46 - protein, 15 aa, synthetic construct
  VARIANT 4: note = X is R or H
RASXSVSTSSYSYMH

SEQ ID NO: 47 - protein, 15 aa, synthetic construct
  VARIANT 15: note = X is K or R
RASKSVSTSSYSYMX

SEQ ID NO: 48 - protein, 15 aa, synthetic construct
  VARIANT 1: note = X is K or H
  VARIANT 4: note = X is R or H
XASXSVSTSSYSYMH

SEQ ID NO: 49 - protein, 15 aa, synthetic construct
  VARIANT 1: note = X is K or H
  VARIANT 15: note = X is K or R
XASKSVSTSSYSYMX

SEQ ID NO: 50 - protein, 15 aa, synthetic construct
  VARIANT 4: note = X is R or H
  VARIANT 15: note = X is K or R
RASXSVSTSSYSYMX

SEQ ID NO: 51 - protein, 7 aa, synthetic construct
YASYLDS

SEQ ID NO: 52 - protein, 9 aa, synthetic construct
  VARIANT 2: note = X is K or R
QXSREFPWT

SEQ ID NO: 53 - protein, 9 aa, synthetic construct
  VARIANT 4: note = X is K or H
QHSXEFPWT

SEQ ID NO: 54 - protein, 9 aa, synthetic construct
QHSRDFPWT

SEQ ID NO: 55 - protein, 9 aa, synthetic construct
  VARIANT 2: note = X is K or R
  VARIANT 4: note = X is K or H
QXSXEFPWT

SEQ ID NO: 56 - protein, 9 aa, synthetic construct
  VARIANT 2: note = X is K or R
QXSRDFPWT

SEQ ID NO: 57 - protein, 9 aa, synthetic construct
  VARIANT 4: note = X is K or H
QHSXDFPWT

SEQ ID NO: 58 - skipped

SEQ ID NO: 59 - protein, 17 aa, synthetic construct
  VARIANT 5: note = X is G or S
  VARIANT 13: note = X is D or E
  VARIANT 14: note = X is T or S
  VARIANT 16: note = X is K, R, or H
YISSXSSTIYYAXXVXG

SEQ ID NO: 60 - protein, 7 aa, synthetic construct
  VARIANT 1: note = X is K, R, or H
  VARIANT 6: note = X is D or E
XGLLLXY

SEQ ID NO: 61 - protein, 15 aa, synthetic construct
  VARIANT 1: note = X is K, R, or H
  VARIANT 4: note = X is K, R, or H
  VARIANT 5: note = X is T or S
  VARIANT 14: note = X is M or L
  VARIANT 15: note = X is K, R, H, or A
XASXXVSTSSYSYXX

SEQ ID NO: 62 - protein, 7 aa, synthetic construct
  VARIANT 6: note = X is D, E, N, or Q
YASYLXS

SEQ ID NO: 63 - protein, 9 aa, synthetic construct
  VARIANT 2: note = X is K, R, or H
  VARIANT 4: note = X is K, R, or H
  VARIANT 5: note = X is D or E
QXSXXFPWT

SEQ ID NO: 64 - protein, 116 aa, synthetic construct
EVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 65 - protein, 116 aa, synthetic construct
EVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 66 - protein, 116 aa, synthetic construct
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 67 - protein, 116 aa, synthetic construct
EVQLVQSGGGLIQPGGSLRLSCAASGFTFSNYGMHWVRQAPGKGLEWVSYISSGSSTIYYADSVKGRFTISRDNSKNTLYLQMNSLRAEDTAVYYCARRGLLLDYWGQGTTVTVSS

SEQ ID NO: 68 - protein, 116 aa, synthetic construct
EVQLVESGGGLIQPGGSLRLSCAASGFTFSNYGMHWVRQAPGKGLEWVSYISSGSSTIYYADSVKGRFTISRDNSKNTLYLQMTSLRAEDTAVYYCARRGLLLDYWGQGTTLTVSS

SEQ ID NO: 69 - protein, 116 aa, synthetic construct
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 70 - protein, 116 aa, synthetic construct
EVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 71 - protein, 116 aa, synthetic construct
EVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 72 - protein, 116 aa, synthetic construct
EVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 73 - protein, 116 aa, synthetic construct
EVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 74 - protein, 111 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIK

SEQ ID NO: 75 - protein, 111 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIK

SEQ ID NO: 76 - protein, 111 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIK

SEQ ID NO: 77 - protein, 111 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIK

SEQ ID NO: 78 - protein, 111 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 79 - protein, 111 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIK

SEQ ID NO: 80 - protein, 111 aa, synthetic construct
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIK

SEQ ID NO: 81 - protein, 111 aa, synthetic construct
DIQMTQSPSSLSASVGDRVTITCRASKSVSTSSYSYLAWYQQKPEKAPKLLIKYASYLQSGVPSRFSGSGSGTDFTLTISSLQPEDFATYYCQHSREFPWTFGAGTKLELK

SEQ ID NO: 82 - protein, 111 aa, synthetic construct
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 83 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 84 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 85 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 86 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 87 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 88 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 89 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 90 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 91 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 92 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 93 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 94 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 95 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 96 - protein, 515 aa, synthetic construct
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 97 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 98 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 99 - RNA, 25 nt, synthetic construct
  source 1..25: mol_type = other RNA
ggccaaacctcggcttacctgaaat

SEQ ID NO: 100 - DNA, 18 bp, synthetic construct
  source 1..18: mol_type = other DNA
  modified_base order(1..6,8..13,15,17): mod_base = OTHER; note = 2-Prime-OMe nucleotides
  modified_base order(7,14,16,18): mod_base = OTHER; note = Locked nucleic acids
  misc_feature order(1..13,15..18): note = RNA
  misc_feature 14: note = DNA
gccaaacctcggcttacc

SEQ ID NO: 101 - DNA, 18 bp, synthetic construct
  source 1..18: mol_type = other DNA
  modified_base order(1..8,10,11,13,14,16,17): mod_base = OTHER; note = 2-Prime-Fluoro nucleotides
  modified_base order(9,12,15,18): mod_base = OTHER; note = Locked nucleic acids
  misc_feature order(1..8,10..14,16..18): note = RNA
  misc_feature order(9,15): note = DNA
gccaaacctcggcttacc

SEQ ID NO: 102 - DNA, 18 bp, synthetic construct
  source 1..18: mol_type = other DNA
  misc_feature order(1..8,10..14,16..18): note = RNA
  misc_feature order(9,15): note = DNA
  modified_base 1..18: mod_base = OTHER; note = Locked nucleic acids
gccaaacctcggcttacc

SEQ ID NO: 103 - DNA, 18 bp, synthetic construct
  source 1..18: mol_type = other DNA
  misc_feature order(1..13,15..18): note = RNA
  misc_feature 14: note = DNA
  modified_base 1..18: mod_base = OTHER; note = 2-Prime-OMe nucleotides
gccaaacctcggcttacc

SEQ ID NO: 104 - protein, 116 aa, synthetic construct
EVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSS

SEQ ID NO: 105 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSS

SEQ ID NO: 106 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSS

SEQ ID NO: 107 - protein, 116 aa, synthetic construct
EVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSS

SEQ ID NO: 108 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSS

SEQ ID NO: 109 - protein, 116 aa, synthetic construct
EVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSS

SEQ ID NO: 110 - protein, 116 aa, synthetic construct
EVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSS

SEQ ID NO: 111 - protein, 446 aa, synthetic construct
EVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 446 aa, synthetic construct
EVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 446 aa, synthetic construct
EVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 446 aa, synthetic construct
EVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 465 aa, synthetic construct
MGWSCIILFLVATATGVHSEVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 465 aa, synthetic construct
MGWSCIILFLVATATGVHSEVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 465 aa, synthetic construct
MGWSCIILFLVATATGVHSEVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 465 aa, synthetic construct
MGWSCIILFLVATATGVHSEVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 111 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIK

SEQ ID NO: 126 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIK

SEQ ID NO: 127 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIK

SEQ ID NO: 128 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIK

SEQ ID NO: 129 - protein, 111 aa, synthetic construct
DIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIK

SEQ ID NO: 130 - protein, 111 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIK

SEQ ID NO: 131 - protein, 218 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 218 aa, synthetic construct
DIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 218 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 237 aa, synthetic construct
MGWSCIILFLVATATGVHSDIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 237 aa, synthetic construct
MGWSCIILFLVATATGVHSDIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 31 aa, synthetic construct
RASKSVSTSSYSYMHWYQQKPGQPPKLLIKY

SEQ ID NO: 144 - protein, 31 aa, synthetic construct
RASKTVSTSSYSYMHWYQQKPGQPPKLLIKY

SEQ ID NO: 145 - protein, 33 aa, synthetic construct
RVTITCRASKSVSTSSYSYMHWYQQKPGKAPKL

SEQ ID NO: 146 - protein, 32 aa, synthetic construct
  VARIANT 1: note = X can be any residue, but preferentially a basic residue R or K
  VARIANT 30: note = X can be any residue, but preferentially a basic residue R or K
XRASKTVSTSSYSYMHWYQQKPGQPPKLLXKY

SEQ ID NO: 147 - skipped

SEQ ID NO: 148 - protein, 115 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVS

SEQ ID NO: 149 - protein, 132 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGS

SEQ ID NO: 150 - DNA, 25 bp, synthetic construct
  source 1..25: mol_type = other DNA
tagctgcaaagcaagtgaagcggac

SEQ ID NO: 151 - DNA, 25 bp, synthetic construct
  source 1..25: mol_type = other DNA
atcacaaacacaacctacctcaggc

SEQ ID NO: 152 - DNA, 20 bp, synthetic construct
  source 1..20: mol_type = other DNA
tcgcaaacagtacaatggcg

SEQ ID NO: 153 - DNA, 15 bp, synthetic construct
  source 1..15: mol_type = other DNA
aacagtacaatggcg

SEQ ID NO: 154 - DNA, 15 bp, synthetic construct
  source 1..15: mol_type = other DNA
tcgcaaacagtacaa

SEQ ID NO: 155 - DNA, 15 bp, synthetic construct
  source 1..15: mol_type = other DNA
actagtcgcaaacag

SEQ ID NO: 156 - DNA, 15 bp, synthetic construct
  source 1..15: mol_type = other DNA
agctaactagtcgca

SEQ ID NO: 157 - DNA, 28 bp, synthetic construct
  source 1..28: mol_type = other DNA
agcccatcttctcctggtcctgggaagg

SEQ ID NO: 158 - DNA, 26 bp, synthetic construct
  source 1..26: mol_type = other DNA
gtgtctttctgagccgccatttctca

SEQ ID NO: 159 - DNA, 22 bp, synthetic construct
  source 1..22: mol_type = other DNA
tctgttagccacgccgccattt

SEQ ID NO: 160 - DNA, 24 bp, synthetic construct
  source 1..24: mol_type = other DNA
ctgttagccactcgccgccatttc

SEQ ID NO: 161 - DNA, 24 bp, synthetic construct
  source 1..24: mol_type = other DNA
ttgtgtctttcttctgttagccac

SEQ ID NO: 162 - DNA, 26 bp, synthetic construct
  source 1..26: mol_type = other DNA
tgttagccactgatctcaacagatct

SEQ ID NO: 163 - DNA, 26 bp, synthetic construct
  source 1..26: mol_type = other DNA
gttcagcttctgttctcaacagatct

SEQ ID NO: 164 - DNA, 26 bp, synthetic construct
  source 1..26: mol_type = other DNA
ttgtgtctttctgcgccgccatttct

SEQ ID NO: 165 - DNA, 26 bp, synthetic construct
  source 1..26: mol_type = other DNA
ctgagaaactgttcgccgccatttct

SEQ ID NO: 166 - DNA, 26 bp, synthetic construct
  source 1..26: mol_type = other DNA
tctcaggaatttgcgccgccatttct

SEQ ID NO: 167 - DNA, 26 bp, synthetic construct
  source 1..26: mol_type = other DNA
tgttagccactgacgccgccatttct

SEQ ID NO: 168 - DNA, 26 bp, synthetic construct
  source 1..26: mol_type = other DNA
gttcagcttctgtcgccgccatttct

SEQ ID NO: 169 - DNA, 30 bp, synthetic construct
  source 1..30: mol_type = other DNA
attctcaggaatttgaacgccgccatttct

SEQ ID NO: 170 - DNA, 30 bp, synthetic construct
  source 1..30: mol_type = other DNA
atttgtgtctttctgtttctcaacagatct

SEQ ID NO: 171 - DNA, 25 bp, synthetic construct
  source 1..25: mol_type = other DNA
attctcaggaatttgtttctcaaca

SEQ ID NO: 172 - DNA, 30 bp, synthetic construct
  source 1..30: mol_type = other DNA
atttgtgtctttctgaacgccgccatttct

SEQ ID NO: 173 - DNA, 26 bp, synthetic construct
  source 1..26: mol_type = other DNA
tctcaggaatttgtctcaacagatct

SEQ ID NO: 174 - DNA, 26 bp, synthetic construct
  source 1..26: mol_type = other DNA
gtttgccgctgcctcctggagttcct

SEQ ID NO: 175 - DNA, 24 bp, synthetic construct
  source 1..24: mol_type = other DNA
gtttgccgctgccctggagttcct

SEQ ID NO: 176 - DNA, 30 bp, synthetic construct
  source 1..30: mol_type = other DNA
cagtttgccgctgcccatcctggagttcct

SEQ ID NO: 177 - DNA, 26 bp, synthetic construct
  source 1..26: mol_type = other DNA
cagtttgccgctgccctggagttcct

SEQ ID NO: 178 - DNA, 30 bp, synthetic construct
  source 1..30: mol_type = other DNA
ttcttccccagttgcgccatcctggagttc

SEQ ID NO: 179 - DNA, 30 bp, synthetic construct
  source 1..30: mol_type = other DNA
cagacctcctgccacgccatcctggagttc

SEQ ID NO: 180 - DNA, 26 bp, synthetic construct
  source 1..26: mol_type = other DNA
ttgccgctgcccacatcctggagttc

SEQ ID NO: 181 - DNA, 26 bp, synthetic construct
  source 1..26: mol_type = other DNA
gccgctgcccacatcctggagttcct

SEQ ID NO: 182 - DNA, 26 bp, synthetic construct
  source 1..26: mol_type = other DNA
ccgctgcccaatgtcctggagttcct

SEQ ID NO: 183 - RNA, 25 nt, synthetic construct
  source 1..25: mol_type = other RNA
gctgcccaatgccatcctggagttc

SEQ ID NO: 184 - RNA, 25 nt, synthetic construct
  source 1..25: mol_type = other RNA
gctgcccaatgccatcctggagttc

SEQ ID NO: 185 - RNA, 25 nt, synthetic construct
  source 1..25: mol_type = other RNA
ttgccgctgcccaatgccatcctgg

SEQ ID NO: 186 - RNA, 25 nt, synthetic construct
  source 1..25: mol_type = other RNA
acagtttgccgctgcccaatgccat

SEQ ID NO: 187 - RNA, 25 nt, synthetic construct
  source 1..25: mol_type = other RNA
tgacaacagtttgccgctgcccaat

SEQ ID NO: 188 - RNA, 25 nt, synthetic construct
  source 1..25: mol_type = other RNA
tgttctgacaacagtttgccgctgc

SEQ ID NO: 189 - RNA, 25 nt, synthetic construct
  source 1..25: mol_type = other RNA
ttcaatgttctgacaacagtttgcc

SEQ ID NO: 190 - RNA, 25 nt, synthetic construct
  source 1..25: mol_type = other RNA
ttgcattcaatgttctgacaacagt

SEQ ID NO: 191 - RNA, 25 nt, synthetic construct
  source 1..25: mol_type = other RNA
cccagttgcattcaatgttctgaca

SEQ ID NO: 192 - RNA, 26 nt, synthetic construct
  source 1..26: mol_type = other RNA
  modified_base 1..26: mod_base = OTHER; note = Morpholino antisense oligonucleotides
ccaatgccatcctggagttcctgtaa

SEQ ID NO: 193 - DNA, 22 bp, synthetic construct
  source 1..22: mol_type = other DNA
caatgccatcctggagttcctg

SEQ ID NO: 194 - DNA, 21 bp, synthetic construct
  source 1..21: mol_type = other DNA
atgggatccagtatacttaca

SEQ ID NO: 195 - DNA, 20 bp, synthetic construct
  source 1..20: mol_type = other DNA
atgggatccagtatacttac

SEQ ID NO: 196 - DNA, 19 bp, synthetic construct
  source 1..19: mol_type = other DNA
tgggatccagtatacttac

SEQ ID NO: 197 - DNA, 19 bp, synthetic construct
  source 1..19: mol_type = other DNA
atgggatccagtatactta

SEQ ID NO: 198 - DNA, 19 bp, synthetic construct
  source 1..19: mol_type = other DNA
gatccagtatacttacagg

SEQ ID NO: 199 - DNA, 19 bp, synthetic construct
  source 1..19: mol_type = other DNA
aatgggatccagtatactt

SEQ ID NO: 200 - DNA, 30 bp, synthetic construct
  source 1..30: mol_type = other DNA
ctccaacatcaaggaagatggcatttctag

SEQ ID NO: 201 - DNA, 20 bp, synthetic construct
  source 1..20: mol_type = other DNA
tcaaggaagatggcatttct

SEQ ID NO: 202 - DNA, 30 bp, synthetic construct
  source 1..30: mol_type = other DNA
gtgtcaccagagtaacagtctgagtaggag

SEQ ID NO: 203 - DNA, 30 bp, synthetic construct
  source 1..30: mol_type = other DNA
aggttgtgtcaccagagtaacagtctgagt

SEQ ID NO: 204 - DNA, 30 bp, synthetic construct
  source 1..30: mol_type = other DNA
ggcagtttccttagtaaccacaggttgtgt

SEQ ID NO: 205 - DNA, 30 bp, synthetic construct
  source 1..30: mol_type = other DNA
agatggcagtttccttagtaaccacaggtt

SEQ ID NO: 206 - DNA, 30 bp, synthetic construct
  source 1..30: mol_type = other DNA
atggcatttctagtttggagatggcagttt

SEQ ID NO: 207 - RNA, 30 nt, synthetic construct
  source 1..30: mol_type = other RNA
  modified_base 1..30: mod_base = OTHER; note = Morpholino antisense oligonucleotides
ctccaacatcaaggaagatggcatttctag

SEQ ID NO: 208 - DNA, 30 bp, synthetic construct; identical to SEQ ID NO: 200
  source 1..30: mol_type = other DNA
  modified_base 1..30: mod_base = OTHER; note = Morpholino antisense oligonucleotides

SEQ ID NO: 209 - DNA, 21 bp, synthetic construct
  source 1..21: mol_type = other DNA
cctccggttctgaaggtgttc

SEQ ID NO: 210 - RNA, 30 nt, synthetic construct
  source 1..30: mol_type = other RNA
attcaactgttgcctccggttctgaaggtg

SEQ ID NO: 211 - RNA, 25 nt, synthetic construct
  source 1..25: mol_type = other RNA
  modified_base 1..25: mod_base = OTHER; note = Morpholino antisense oligonucleotides
ctgaaggtgttcttgtacttcatcc

SEQ ID NO: 212 - RNA, 31 nt, synthetic construct
  source 1..31: mol_type = other RNA
  modified_base 1..31: mod_base = OTHER; note = Morpholino antisense oligonucleotides
cattcaactgttgcctccggttctgaaggtg

SEQ ID NO: 213 - DNA, 21 bp, synthetic construct
  source 1..21: mol_type = other DNA
  modified_base 1..21: mod_base = OTHER; note = Morpholino antisense oligonucleotides
cctccggttctgaaggtgttc

SEQ ID NO: 214 - DNA, 25 bp, synthetic construct
  source 1..25: mol_type = other DNA
gttgcctccggttctgaaggtgttc

SEQ ID NO: 215 - RNA, 21 nt, synthetic construct
  source 1..21: mol_type = other RNA
gcagcctctcgctcactcacc

SEQ ID NO: 216 - RNA, 21 nt, synthetic construct
  source 1..21: mol_type = other RNA
ccaaagcagcctctcgctcac

SEQ ID NO: 217 - RNA, 21 nt, synthetic construct
  source 1..21: mol_type = other RNA
ttcttccaaagcagcctctcg

SEQ ID NO: 218 - RNA, 21 nt, synthetic construct
  source 1..21: mol_type = other RNA
atctatgagtttcttccaaag

SEQ ID NO: 219 - RNA, 21 nt, synthetic construct
  source 1..21: mol_type = other RNA
tgttgcagtaatctatgagtt

SEQ ID NO: 220 - RNA, 21 nt, synthetic construct
  source 1..21: mol_type = other RNA
cagggggaactgttgcagtaa

SEQ ID NO: 221 - RNA, 21 nt, synthetic construct
  source 1..21: mol_type = other RNA
tttccaggtccagggggaact

SEQ ID NO: 222 - RNA, 21 nt, synthetic construct
  source 1..21: mol_type = other RNA
gcaagaaacttttccaggtcc

SEQ ID NO: 223 - skipped

SEQ ID NO: 224 - RNA, 12 nt, synthetic construct
  source 1..12: mol_type = other RNA
cagcagcagcag

SEQ ID NO: 225 - RNA, 15 nt, synthetic construct
  source 1..15: mol_type = other RNA
cagcagcagcagcag

SEQ ID NO: 226 - RNA, 18 nt, synthetic construct
  source 1..18: mol_type = other RNA
cagcagcagcagcagcag

SEQ ID NO: 227 - skipped

SEQ ID NO: 228 - RNA, 12 nt, synthetic construct
  source 1..12: mol_type = other RNA
caggcaggcagg

SEQ ID NO: 229 - skipped

SEQ ID NO: 230 - RNA, 12 nt, synthetic construct
  source 1..12: mol_type = other RNA
aggcaggcaggc

SEQ ID NO: 231 - skipped

SEQ ID NO: 232 - RNA, 12 nt, synthetic construct
  source 1..12: mol_type = other RNA
ggcaggcaggca

SEQ ID NO: 233 - DNA, 18 bp, synthetic construct
  source 1..18: mol_type = other DNA
tcactttcataatgctgg

SEQ ID NO: 234 - RNA, 25 nt, synthetic construct
  source 1..25: mol_type = other RNA
ctgccaaggagggccctgctcagcc

SEQ ID NO: 235 - RNA, 25 nt, synthetic construct
  source 1..25: mol_type = other RNA
cgcactcttgcccacaccgccggcg

SEQ ID NO: 236 - RNA, 25 nt, synthetic construct
  source 1..25: mol_type = other RNA
ctcacctctatagtggggtcgtatt

SEQ ID NO: 237 - RNA, 21 nt, synthetic construct
  source 1..21: mol_type = other RNA
ttgcccacaccgccggcgccc

SEQ ID NO: 238 - RNA, 21 nt, synthetic construct
  source 1..21: mol_type = other RNA
ttgcccacaccgacggcgccc

SEQ ID NO: 239 - RNA, 25 nt, synthetic construct
  source 1..25: mol_type = other RNA
tatagtggggtcgtattcgtccaca

SEQ ID NO: 240 - RNA, 25 nt, synthetic construct
  source 1..25: mol_type = other RNA
tggttctggatcagctggatggtca

SEQ ID NO: 241 - RNA, 25 nt, synthetic construct
  source 1..25: mol_type = other RNA
cggggtcctcctacagggtctcctg

SEQ ID NO: 242 - RNA, 25 nt, synthetic construct
  source 1..25: mol_type = other RNA
gtcatcgctcctcaggggcctgcgg

SEQ ID NO: 243 - RNA, 25 nt, synthetic construct
  source 1..25: mol_type = other RNA
cccaccaccaccagcttatattccg

SEQ ID NO: 244 - RNA, 25 nt, synthetic construct
  source 1..25: mol_type = other RNA
gggtctcctgccccacctgccaagg

SEQ ID NO: 245 - RNA, 25 nt, synthetic construct
  source 1..25: mol_type = other RNA
ttcagtcattttcagcaggccttat

SEQ ID NO: 246 - RNA, 25 nt, synthetic construct
  source 1..25: mol_type = other RNA
tttacctctattgttggatcatatt

SEQ ID NO: 247 - RNA, 25 nt, synthetic construct
  source 1..25: mol_type = other RNA
atgaaaatggtcagagaaaccttta

SEQ ID NO: 248 - RNA, 25 nt, synthetic construct
  source 1..25: mol_type = other RNA
tctgtatcaaagaatggtcctgcac

SEQ ID NO: 249 - RNA, 25 nt, synthetic construct
  source 1..25: mol_type = other RNA
cagtaatatgcatattaaaacaaga

SEQ ID NO: 250 - RNA, 25 nt, synthetic construct
  source 1..25: mol_type = other RNA
aataaaaataatgaaaatgtgacta

SEQ ID NO: 251 - RNA, 25 nt, synthetic construct
  source 1..25: mol_type = other RNA
tattagaacatgtcacacataaggt

SEQ ID NO: 252 - RNA, 25 nt, synthetic construct
  source 1..25: mol_type = other RNA
agtttatattcagtcattttcagca

SEQ ID NO: 253 - RNA, 25 nt, synthetic construct
  source 1..25: mol_type = other RNA
tacgccaccagctccaactaccaca

SEQ ID NO: 254 - RNA, 25 nt, synthetic construct
  source 1..25: mol_type = other RNA
gctgtatcgtcaaggcactcttgcc

SEQ ID NO: 255 - RNA, 25 nt, synthetic construct
  source 1..25: mol_type = other RNA
tcgtccacaaaatgattctgaatta

SEQ ID NO: 256 - RNA, 25 nt, synthetic construct
  source 1..25: mol_type = other RNA
tttcacaccagcaagaacctgttgg

SEQ ID NO: 257 - RNA, 25 nt, synthetic construct
  source 1..25: mol_type = other RNA
cctcacctctatggtgggatcatat

SEQ ID NO: 258 - RNA, 25 nt, synthetic construct
  source 1..25: mol_type = other RNA
accaccagtttgtactcagtcattt

SEQ ID NO: 259 - RNA, 25 nt, synthetic construct
  source 1..25: mol_type = other RNA
caacaccacctgctccaaccaccac

SEQ ID NO: 260 - RNA, 25 nt, synthetic construct
  source 1..25: mol_type = other RNA
gtgcgcttttcccaacaccacctgc

SEQ ID NO: 261 - RNA, 25 nt, synthetic construct
  source 1..25: mol_type = other RNA
tggttctggattagctggattgtca

SEQ ID NO: 262 - RNA, 25 nt, synthetic construct
  source 1..25: mol_type = other RNA
ggtgggatcatattcatctacaaag

SEQ ID NO: 263 - RNA, 25 nt, synthetic construct
  source 1..25: mol_type = other RNA
aatcagggttaattggcgagccaca

SEQ ID NO: 264 - RNA, 25 nt, synthetic construct
  source 1..25: mol_type = other RNA
caggatcaggtcagcgggctaccac

SEQ ID NO: 265 - RNA, 25 nt, synthetic construct
  source 1..25: mol_type = other RNA
aaaaatgtctcctgcgagagcaaac

SEQ ID NO: 266 - RNA, 25 nt, synthetic construct
  source 1..25: mol_type = other RNA
ccaccatggacaagaaaaatgtctc

SEQ ID NO: 267 - RNA, 22 nt, synthetic construct
  source 1..22: mol_type = other RNA
cagcacgcatcccaccatggac

SEQ ID NO: 268 - RNA, 25 nt, synthetic construct
  source 1..25: mol_type = other RNA
atcactgtgtctggagcaacaatgc

SEQ ID NO: 269 - RNA, 25 nt, synthetic construct
  source 1..25: mol_type = other RNA
tcgtttgataggctccccttcgcag

SEQ ID NO: 270 - RNA, 25 nt, synthetic construct
  source 1..25: mol_type = other RNA
atcagggagaaaactcacctctcgt

SEQ ID NO: 271 - RNA, 22 nt, synthetic construct
  source 1..22: mol_type = other RNA
acctacccagttcggaatgatc

SEQ ID NO: 272 - RNA, 18 nt, synthetic construct
  source 1..18: mol_type = other RNA
ctacccagttcggaatga

SEQ ID NO: 273 - RNA, 15 nt, synthetic construct
  source 1..15: mol_type = other RNA
tacccagttcggaat

SEQ ID NO: 274 - DNA, 22 bp, synthetic construct
  source 1..22: mol_type = other DNA
atgtctcctctgttgtctaatg

SEQ ID NO: 275 - DNA, 22 bp, synthetic construct
  source 1..22: mol_type = other DNA
taacagcaaagacgatcattcc

SEQ ID NO: 276 - DNA, 20 bp, synthetic construct
  source 1..20: mol_type = other DNA
gggctctgggctcctgagcc

SEQ ID NO: 277 - DNA, 20 bp, synthetic construct
  source 1..20: mol_type = other DNA
ccgagctgctgcagtgggag

SEQ ID NO: 278 - DNA, 20 bp, synthetic construct
  source 1..20: mol_type = other DNA
gcaggtcccgcacagcacgg

SEQ ID NO: 279 - DNA, 20 bp, synthetic construct
  source 1..20: mol_type = other DNA
tggcagatgccttgtcggca

SEQ ID NO: 280 - DNA, 20 bp, synthetic construct
  source 1..20: mol_type = other DNA
gagccgctggcagatgcctt

SEQ ID NO: 281 - DNA, 20 bp, synthetic construct
  source 1..20: mol_type = other DNA
ctgagccgctggcagatgcc

SEQ ID NO: 282 - DNA, 20 bp, synthetic construct
  source 1..20: mol_type = other DNA
aggagatgggtccgcccacc

SEQ ID NO: 283 - DNA, 20 bp, synthetic construct
  source 1..20: mol_type = other DNA
cagaggagatgggtccgccc

SEQ ID NO: 284 - DNA, 20 bp, synthetic construct
  source 1..20: mol_type = other DNA
gccagaggagatgggtccgc

SEQ ID NO: 285 - RNA, 18 nt, synthetic construct
  source 1..18: mol_type = other RNA
aggtcccgggagaaggga

SEQ ID NO: 286 - RNA, 18 nt, synthetic construct
  source 1..18: mol_type = other RNA
cagccaggtcccgggaga

SEQ ID NO: 287 - RNA, 18 nt, synthetic construct
  source 1..18: mol_type = other RNA
ggagacagccaggtcccg

SEQ ID NO: 288 - RNA, 18 nt, synthetic construct
  source 1..18: mol_type = other RNA
agtctggagacagccagg

SEQ ID NO: 289 - RNA, 18 nt, synthetic construct
  source 1..18: mol_type = other RNA
gagcaagtctggagacag

SEQ ID NO: 290 - RNA, 18 nt, synthetic construct
  source 1..18: mol_type = other RNA
aaaaggagcaagtctgga

SEQ ID NO: 291 - RNA, 18 nt, synthetic construct
  source 1..18: mol_type = other RNA
gggacaaaaggagcaagt

SEQ ID NO: 292 - RNA, 18 nt, synthetic construct
  source 1..18: mol_type = other RNA
gcagtggtagtaagtggg

SEQ ID NO: 293 - RNA, 18 nt, synthetic construct
  source 1..18: mol_type = other RNA
gggatcagatcacagcca

SEQ ID NO: 294 - RNA, 18 nt, synthetic construct
  source 1..18: mol_type = other RNA
cagatcacagccatgcac

SEQ ID NO: 295 - RNA, 18 nt, synthetic construct
  source 1..18: mol_type = other RNA
cccagcgtcgccctgggc

SEQ ID NO: 296 - RNA, 18 nt, synthetic construct
  source 1..18: mol_type = other RNA
cccatcccagcgtcgccc

SEQ ID NO: 297 - RNA, 18 nt, synthetic construct
  source 1..18: mol_type = other RNA
cgtcacccatcccagcgt

SEQ ID NO: 298 - RNA, 18 nt, synthetic construct
  source 1..18: mol_type = other RNA
cctgacgtcacccatccc

SEQ ID NO: 299 - RNA, 18 nt, synthetic construct
  source 1..18: mol_type = other RNA
ggcagcctgacgtcaccc

SEQ ID NO: 300 - RNA, 18 nt, synthetic construct
  source 1..18: mol_type = other RNA
cagtgggcagcctgacgt

SEQ ID NO: 301 - RNA, 18 nt, synthetic construct
  source 1..18: mol_type = other RNA
acagtcagtgggcagcct

SEQ ID NO: 302 - RNA, 18 nt, synthetic construct
  source 1..18: mol_type = other RNA
acaggacagtcagtgggc

SEQ ID NO: 303 - RNA, 18 nt, synthetic construct
  source 1..18: mol_type = other RNA
acaggatcagcagagcct

SEQ ID NO: 304 - RNA, 18 nt, synthetic construct
  source 1..18: mol_type = other RNA
atcagcagagcctgccag

SEQ ID NO: 305 - RNA, 21 nt, synthetic construct
  source 1..21: mol_type = other RNA
agggcttaaaatttctcctgc

SEQ ID NO: 306 - RNA, 21 nt, synthetic construct
  source 1..21: mol_type = other RNA
ccaagggcttaaaatttctcc

SEQ ID NO: 307 - RNA, 21 nt, synthetic construct
  source 1..21: mol_type = other RNA
tttgaatcagagtccaagggc

SEQ ID NO: 308 - RNA, 21 nt, synthetic construct
  source 1..21: mol_type = other RNA
aagtttctcagtccacttggg

SEQ ID NO: 309 - RNA, 21 nt, synthetic construct
  source 1..21: mol_type = other RNA
ccggccattctctttctgttc

SEQ ID NO: 310 - RNA, 20 nt, synthetic construct
  source 1..20: mol_type = other RNA
atgcccggccattctctttc

SEQ ID NO: 311 - RNA, 20 nt, synthetic construct
  source 1..20: mol_type = other RNA
tgaccactgtagtagcaatg

SEQ ID NO: 312 - RNA, 20 nt, synthetic construct
  source 1..20: mol_type = other RNA
acaccattggttctcatagg

SEQ ID NO: 313 - RNA, 20 nt, synthetic construct
  source 1..20: mol_type = other RNA
atggtttgcagccacaacac

SEQ ID NO: 314 - RNA, 20 nt, synthetic construct
  source 1..20: mol_type = other RNA
ccacaacaccaatgcgatat

SEQ ID NO: 315 - RNA, 20 nt, synthetic construct
  source 1..20: mol_type = other RNA
gcatggtttgcagccacaac

SEQ ID NO: 316 - RNA, 20 nt, synthetic construct
  source 1..20: mol_type = other RNA
cttggggaagattctaaggt

SEQ ID NO: 317 - RNA, 18 nt, synthetic construct
  source 1..18: mol_type = other RNA
gtatccgctgagggaata

SEQ ID NO: 318 - RNA, 18 nt, synthetic construct
  source 1..18: mol_type = other RNA
gtgtagtatccgctgagg

SEQ ID NO: 319 - RNA, 18 nt, synthetic construct
  source 1..18: mol_type = other RNA
gatgggtgtagtatccgc

SEQ ID NO: 320 - RNA, 18 nt, synthetic construct
  source 1..18: mol_type = other RNA
aaatggatgggtgtagta

SEQ ID NO: 321 - RNA, 18 nt, synthetic construct
  source 1..18: mol_type = other RNA
ccttgaaatggatgggtg

SEQ ID NO: 322 - RNA, 18 nt, synthetic construct
  source 1..18: mol_type = other RNA
ggcgcagaaccaccaggt

SEQ ID NO: 323 - RNA, 18 nt, synthetic construct
  source 1..18: mol_type = other RNA
agggagttgcaatatcga

SEQ ID NO: 324 - RNA, 18 nt, synthetic construct
  source 1..18: mol_type = other RNA
gcctccacagattcaggg

SEQ ID NO: 325 - RNA, 18 nt, synthetic construct
  source 1..18: mol_type = other RNA
aaacttcattctggaaca

SEQ ID NO: 326 - RNA, 18 nt, synthetic construct
  source 1..18: mol_type = other RNA
agggctttggccattttc

SEQ ID NO: 327 - RNA, 18 nt, synthetic construct
  source 1..18: mol_type = other RNA
acagtttgcacttccggg

SEQ ID NO: 328 - RNA, 18 nt, synthetic construct
  source 1..18: mol_type = other RNA
cgcctaaggaattcactc

SEQ ID NO: 329 - RNA, 18 nt, synthetic construct
  source 1..18: mol_type = other RNA
cgcctaaggaattcactc

SEQ ID NO: 330 - RNA, 18 nt, synthetic construct
  source 1..18: mol_type = other RNA
ggatacgcctaaggaatt

SEQ ID NO: 331 - RNA, 18 nt, synthetic construct
  source 1..18: mol_type = other RNA
gccagtgcccatagcggg

SEQ ID NO: 332 - RNA, 18 nt, synthetic construct
  source 1..18: mol_type = other RNA
cttatgccagtgcccata

SEQ ID NO: 333 - RNA, 18 nt, synthetic construct
  source 1..18: mol_type = other RNA
cccaagggagccacgcgt

SEQ ID NO: 334 - RNA, 18 nt, synthetic construct
  source 1..18: mol_type = other RNA
aagggagccacgcgttac

SEQ ID NO: 335 - RNA, 18 nt, synthetic construct
  source 1..18: mol_type = other RNA
agcccaagggagccacgc

SEQ ID NO: 336 - RNA, 18 nt, synthetic construct
  source 1..18: mol_type = other RNA
cttccgggacagtgtgga

SEQ ID NO: 337 - RNA, 18 nt, synthetic construct
  source 1..18: mol_type = other RNA
ttcttggcagctggaaca

SEQ ID NO: 338 - RNA, 18 nt, synthetic construct
  source 1..18: mol_type = other RNA
gtagtcctggtggcatgc

SEQ ID NO: 339 - RNA, 18 nt, synthetic construct
  source 1..18: mol_type = other RNA
cccctgtagtcctggtgg

SEQ ID NO: 340 - RNA, 18 nt, synthetic construct
  source 1..18: mol_type = other RNA
ctgtagtcctggtggcat

SEQ ID NO: 341 - RNA, 18 nt, synthetic construct
  source 1..18: mol_type = other RNA
tggcatcacggacaatgc

SEQ ID NO: 342 - RNA, 18 nt, synthetic construct
  source 1..18: mol_type = other RNA
cgacaaagagattcagca

SEQ ID NO: 343 - RNA, 18 nt, synthetic construct
  source 1..18: mol_type = other RNA
gacggcgacaaagagatt

SEQ ID NO: 344 - RNA, 18 nt, synthetic construct
  source 1..18: mol_type = other RNA
atgatgacggcgacaaag

SEQ ID NO: 345 - RNA, 18 nt, synthetic construct
  source 1..18: mol_type = other RNA
tgtccatgatgacggcga

SEQ ID NO: 346 - RNA, 18 nt, synthetic construct
  source 1..18: mol_type = other RNA
aaagttgtccatgatgac

SEQ ID NO: 347 - RNA, 18 nt, synthetic construct
  source 1..18: mol_type = other RNA
gacttgtgggtgttctca

SEQ ID NO: 348 - RNA, 18 nt, synthetic construct
  source 1..18: mol_type = other RNA
gagtggacttgtgggtgt

SEQ ID NO: 349 - RNA, 18 nt, synthetic construct
  source 1..18: mol_type = other RNA
cgctggagtggacttgtg

SEQ ID NO: 350 - RNA, 18 nt, synthetic construct
  source 1..18: mol_type = other RNA
tagctcgctggagtggac

SEQ ID NO: 351 - RNA, 18 nt, synthetic construct
  source 1..18: mol_type = other RNA
gtcttctgccttgggctt

SEQ ID NO: 352 - RNA, 18 nt, synthetic construct
  source 1..18: mol_type = other RNA
gataggtcttctgccttg

SEQ ID NO: 353 - RNA, 18 nt, synthetic construct
  source 1..18: mol_type = other RNA
atctaaaatctgcttctt

SEQ ID NO: 354 - RNA, 18 nt, synthetic construct
  source 1..18: mol_type = other RNA
atgcagataggtcttctg

SEQ ID NO: 355 - RNA, 18 nt, synthetic construct
  source 1..18: mol_type = other RNA
gtcttctgccttgggctt

SEQ ID NO: 356 - RNA, 18 nt, synthetic construct
  source 1..18: mol_type = other RNA
ctcaaatgcagataggtc

SEQ ID NO: 357 - RNA, 18 nt, synthetic construct
  source 1..18: mol_type = other RNA
tcttttccatgtattcca

SEQ ID NO: 358 - RNA, 18 nt, synthetic construct
  source 1..18: mol_type = other RNA
accacgttcaccccgttc

SEQ ID NO: 359 - RNA, 18 nt, synthetic construct
  source 1..18: mol_type = other RNA
ggccggtcaggagggcgg

SEQ ID NO: 360 - RNA, 18 nt, synthetic construct
  source 1..18: mol_type = other RNA
gagccggccggtcaggag

SEQ ID NO: 361 - RNA, 18 nt, synthetic construct
  source 1..18: mol_type = other RNA
accgggagccggccggtc

SEQ ID NO: 362 - RNA, 18 nt, synthetic construct
  source 1..18: mol_type = other RNA
tccgaaccgggagccggc

SEQ ID NO: 363 - RNA, 18 nt, synthetic construct
  source 1..18: mol_type = other RNA
tccgaaccgggagccggc

SEQ ID NO: 364 - RNA, 21 nt, synthetic construct
  source 1..21: mol_type = other RNA
ccagaaaaaataaatactttc

SEQ ID NO: 365 - RNA, 18 nt, synthetic construct
  source 1..18: mol_type = other RNA
aatgttccagaaaaaata

SEQ ID NO: 366 - RNA, 18 nt, synthetic construct
  source 1..18: mol_type = other RNA
tctaaatgttccagaaaa

SEQ ID NO: 367 - RNA, 18 nt, synthetic construct
  source 1..18: mol_type = other RNA
ccactgttcatagggatc

SEQ ID NO: 368 - RNA, 26 nt, synthetic construct
  source 1..26: mol_type = other RNA
ccatacctgtataggagagattcata

SEQ ID NO: 369 - RNA, 26 nt, synthetic construct
  source 1..26: mol_type = other RNA
ccatacctgtataggagagaattaga

SEQ ID NO: 370 - RNA, 32 nt, synthetic construct
  source 1..32: mol_type = other RNA
ccatacctgtataggagagaattagattcata

SEQ ID NO: 371 - DNA, 25 bp, synthetic construct
  source 1..25: mol_type = other DNA
atcctctttggtaacctcacctcac

SEQ ID NO: 372 - RNA, 30 nt, synthetic construct
  source 1..30: mol_type = other RNA
tctccacggtcgcccttcagcccgcgttct

SEQ ID NO: 373 - RNA, 25 nt, synthetic construct
  source 1..25: mol_type = other RNA
tctccacggtcgcccttcagcccgc

SEQ ID NO: 374 - RNA, 25 nt, synthetic construct
  source 1..25: mol_type = other RNA
cctttctctccccgttctccctgaa

SEQ ID NO: 375 - RNA, 25 nt, synthetic construct
  source 1..25: mol_type = other RNA
cctttctctccccattctccctgaa

SEQ ID NO: 376 - RNA, 26 nt, synthetic construct
  source 1..26: mol_type = other RNA
ccacctgttctccacgttctcctttc

SEQ ID NO: 377 - RNA, 24 nt, synthetic construct
  source 1..24: mol_type = other RNA
ggcctcttggaccctgcagaccct

SEQ ID NO: 378 - RNA, 24 nt, synthetic construct
  source 1..24: mol_type = other RNA
ggcctcttggaccctacagaccct

SEQ ID NO: 379 - RNA, 22 nt, synthetic construct
  source 1..22: mol_type = other RNA
acctacccagttcggaatgatc

SEQ ID NO: 380 - RNA, 18 nt, synthetic construct
  source 1..18: mol_type = other RNA
ctacccagttcggaatga

SEQ ID NO: 381 - RNA, 15 nt, synthetic construct
  source 1..15: mol_type = other RNA
tacccagttcggaat

SEQ ID NO: 382 - DNA, 22 bp, synthetic construct
  source 1..22: mol_type = other DNA
atgtctcctctgttgtctaatg

SEQ ID NO: 383 - DNA, 22 bp, synthetic construct
  source 1..22: mol_type = other DNA
taacagcaaagacgatcattcc

SEQ ID NO: 384 - DNA, 22 bp, synthetic construct
  source 1..22: mol_type = other DNA
gaaacaactttttgtttgagaa

SEQ ID NO: 385 - DNA, 22 bp, synthetic construct
  source 1..22: mol_type = other DNA
gtttttccagagtgcacaacta

SEQ ID NO: 386 - RNA, 19 nt, synthetic construct
  source 1..19: mol_type = other RNA
agtccaagtgtggctcaaa

SEQ ID NO: 387 - RNA, 19 nt, synthetic construct
  source 1..19: mol_type = other RNA
ggctcaaaggataatatca

SEQ ID NO: 388 - RNA, 19 nt, synthetic construct
  source 1..19: mol_type = other RNA
gcaacgtccagtccaagtg

SEQ ID NO: 389 - DNA, 25 bp, synthetic construct
  source 1..25: mol_type = other DNA
agacagacctgatcacctacctggt

SEQ ID NO: 390 - DNA, 25 bp, synthetic construct
  source 1..25: mol_type = other DNA
ggtatctgccagaaaatgcacagga

SEQ ID NO: 391 - DNA, 25 bp, synthetic construct
  source 1..25: mol_type = other DNA
ctaccagcctaccatgttaccttga

SEQ ID NO: 392 - protein, 115 aa, synthetic construct; identical to SEQ ID NO: 148

SEQ ID NO: 393 - protein, 132 aa, synthetic construct; identical to SEQ ID NO: 149

SEQ ID NO: 394 - RNA, 720 nt, synthetic construct
  source 1..720: mol_type = other RNA
  mRNA 1..720: note = mRNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 395 - RNA, 22 nt, synthetic construct
  source 1..22: mol_type = other RNA
  modified_base 1..22: mod_base = OTHER; note = Morpholino antisense oligonucleotides
caatgccatcctggagttcctg

SEQ ID NO: 396 - RNA, 34 nt, synthetic construct
  source 1..34: mol_type = other RNA
  modified_base 1..34: mod_base = OTHER; note = Morpholino antisense oligonucleotides
gcccaatgccatcctggagttcctgtaagatacc

SEQ ID NO: 397 - RNA, 31 nt, synthetic construct
  source 1..31: mol_type = other RNA
  modified_base 1..31: mod_base = OTHER; note = Morpholino antisense oligonucleotides
gcccaatgccatcctggagttcctgtaagat

SEQ ID NO: 398 - RNA, 39 nt, synthetic construct
  source 1..39: mol_type = other RNA
  modified_base 1..39: mod_base = OTHER; note = Morpholino antisense oligonucleotides
ttgccgctgcccaatgccatcctggagttcctgtaagat

SEQ ID NO: 399 - RNA, 22 nt, synthetic construct
  source 1..22: mol_type = other RNA
  modified_base order(4,9,12,17,18,21): mod_base = m5u
caatgccatcctggagttcctg

SEQ ID NO: 400 - RNA, 30 nt, synthetic construct; identical to SEQ ID NO: 207
  source 1..30: mol_type = other RNA
  modified_base order(2,9,19,24..26,28): mod_base = m5u

SEQ ID NO: 401 - RNA, 25 nt, synthetic construct
  source 1..25: mol_type = other RNA
gttgcctccggttctgaaggtgttc

SEQ ID NO: 402 - RNA, 21 nt, synthetic construct
  source 1..21: mol_type = other RNA
cctccggttctgaaggtgttc

SEQ ID NO: 403 - RNA, 25 nt, synthetic construct
  source 1..25: mol_type = other RNA
  modified_base order(2,3,7,12,13,15,21,23,24): mod_base = m5u
gttgcctccggttctgaaggtgttc

SEQ ID NO: 404 - RNA, 21 nt, synthetic construct
  source 1..21: mol_type = other RNA
  modified_base order(3,8,9,11,17,19,20): mod_base = m5u
cctccggttctgaaggtgttc

SEQ ID NO: 405 - DNA, 31 bp, synthetic construct
  source 1..31: mol_type = other DNA
  misc_feature order(1..3,29..31): note = Lysine residues
nnnggccaaacctcggcttacctgaaatnnn

SEQ ID NO: 406 - RNA, 19 nt, synthetic construct
  source 1..19: mol_type = other RNA
tttgagccacacttggact

SEQ ID NO: 407 - RNA, 19 nt, synthetic construct
  source 1..19: mol_type = other RNA
tgatattatcctttgagcc

SEQ ID NO: 408 - RNA, 19 nt, synthetic construct
  source 1..19: mol_type = other RNA
cacttggactggacgttgc